SEQUENCE LISTING (485 sequences)

SEQ ID NO: 1 - DNA, 357 bp, synthetic construct
  source 1..357: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcaactcttagtggtagtggtgctagcacatactacgcagactccgtgaagggccggttcaccatctccagagacaattccaagaacgcgctgtatctgcaaatgaacagcctgagagccgaggacacggccgtatattactgtgcgaaagaagggttcggggactggtacttcgatctctggggccgtggcaccctggtcactgtctcctca

SEQ ID NO: 2 - protein, 119 aa, synthetic construct
EVQLVESGGGLVQPGGSLRLSCAASGFTFSSYAMSWVRQAPGKGLEWVSTLSGSGASTYYADSVKGRFTISRDNSKNALYLQMNSLRAEDTAVYYCAKEGFGDWYFDLWGRGTLVTVSS

SEQ ID NO: 3 - protein, 8 aa, synthetic construct
GFTFSSYA

SEQ ID NO: 4 - protein, 8 aa, synthetic construct
LSGSGAST

SEQ ID NO: 5 - protein, 12 aa, synthetic construct
AKEGFGDWYFDL

SEQ ID NO: 6 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagtcgtaagttagcctggttccagcagaaacctggccaggctcccaggctcctcatctatgttgcatccaccagggccactggtatcccagccaggttcagtggcagtgggtctgggacagacttcactctcaccattagtagcctgcagtctgaagattttgcaatttattactgtcagcagtataataactggccgctcactttcggcggagggaccaaggtggagatcaaa

SEQ ID NO: 7 - protein, 107 aa, synthetic construct
EIVMTQSPATLSVSPGERATLSCRASQSVSRKLAWFQQKPGQAPRLLIYVASTRATGIPARFSGSGSGTDFTLTISSLQSEDFAIYYCQQYNNWPLTFGGGTKVEIK

SEQ ID NO: 8 - protein, 6 aa, synthetic construct
QSVSRK

SEQ ID NO: 9 - skipped

SEQ ID NO: 10 - protein, 9 aa, synthetic construct
QQYNNWPLT

SEQ ID NO: 11 - DNA, 357 bp, synthetic construct
  source 1..357: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcaactattagtggtagtggtggtagcacatactacgcagactccgtgaagggccggttctccatctccagagacaattccaagaacacgctgtatctgcaaatgaacagcctgagagccgaggacacggccgtatattactgtgcgaaaggggcctacggaaattggtacttcgatatctggggccgtggcaccctggtcactgtctcctca

SEQ ID NO: 12 - protein, 119 aa, synthetic construct
EVQLVESGGGLVQPGGSLRLSCAASGFTFSSYAMSWVRQAPGKGLEWVSTISGSGGSTYYADSVKGRFSISRDNSKNTLYLQMNSLRAEDTAVYYCAKGAYGNWYFDIWGRGTLVTVSS

SEQ ID NO: 13 - protein, 8 aa, synthetic construct
ISGSGGST

SEQ ID NO: 14 - protein, 12 aa, synthetic construct
AKGAYGNWYFDI

SEQ ID NO: 15 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccatcagggccactggcatcccagccaggttcagtggcagtgggtctgggacagagttcactctcaccatcagcatcctgcagtctgaagattttgcagtttattactgtcaccaatataataactggccgctcactttcggcggggggaccaaggtggagatcaaa

SEQ ID NO: 16 - protein, 107 aa, synthetic construct
EIVMTQSPATLSVSPGERATLSCRASQSVSSNLAWYQQKPGQAPRLLIYGASIRATGIPARFSGSGSGTEFTLTISILQSEDFAVYYCHQYNNWPLTFGGGTKVEIK

SEQ ID NO: 17 - protein, 6 aa, synthetic construct
QSVSSN

SEQ ID NO: 18 - skipped

SEQ ID NO: 19 - protein, 9 aa, synthetic construct
HQYNNWPLT

SEQ ID NO: 20 - DNA, 357 bp, synthetic construct
  source 1..357: mol_type = other DNA
gaagtgcagctggtggagtctgggggaggctttgtacagcctggcaggtccctgagactctcctgtatagcctctggattcacctttgatgattatgccatgcactgggtccggcaaactccagggaagggcctggagtgggtctcagttattagttggaatagtgttagcataggctatgcggactctgtgaagggccgattcaccatctccagagacaacgccaagaactccctgtatctgcaaatgaacagtctgagagctgaggacacggccttgtattactgtgctcggtatagcagtggccccgatgcttttgatatctggggccaagggacaatggtcaccgtctcttca

SEQ ID NO: 21 - protein, 119 aa, synthetic construct
EVQLVESGGGFVQPGRSLRLSCIASGFTFDDYAMHWVRQTPGKGLEWVSVISWNSVSIGYADSVKGRFTISRDNAKNSLYLQMNSLRAEDTALYYCARYSSGPDAFDIWGQGTMVTVSS

SEQ ID NO: 22 - protein, 8 aa, synthetic construct
GFTFDDYA

SEQ ID NO: 23 - protein, 8 aa, synthetic construct
ISWNSVSI

SEQ ID NO: 24 - protein, 12 aa, synthetic construct
ARYSSGPDAFDI

SEQ ID NO: 25 - DNA, 321 bp, synthetic construct
  source 1..321: mol_type = other DNA
gacatccagatgacccagtctccatcctccctgtctgcatctgtaggagacagagtcaccatcacttgccgggcaagtcaggacattagaaatgatttaggctggtatcagcagaaaccagggaaagcccctaagcgcctgatctatgctgcatccagtttgcaaagtggggtcccatcaaggttcagcggcagtggatttgggacagaattcactctcacaatcagcagcctgcagcctgaagattttgcaacttattactgtctacaacattatacttaccctcctactttcggcggagggaccagggtggagatcaaa

SEQ ID NO: 26 - protein, 107 aa, synthetic construct
DIQMTQSPSSLSASVGDRVTITCRASQDIRNDLGWYQQKPGKAPKRLIYAASSLQSGVPSRFSGSGFGTEFTLTISSLQPEDFATYYCLQHYTYPPTFGGGTRVEIK

SEQ ID NO: 27 - protein, 6 aa, synthetic construct
QDIRND

SEQ ID NO: 28 - skipped

SEQ ID NO: 29 - protein, 9 aa, synthetic construct
LQHYTYPPT

SEQ ID NO: 30 - DNA, 372 bp, synthetic construct
  source 1..372: mol_type = other DNA
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattgggatgatgataagcgctacagcccatctctgaagagcaggctcaccatcaccaaggacacctccaaaaaccaggtggtccttacaatgaccaacatggaccctgtggacacagccacatattactgtgcacacagttattactatgatagtagtgattcttactactttgactactggggccagggaaccctggtcaccgtctcctca

SEQ ID NO: 31 - protein, 124 aa, synthetic construct
QITLKESGPTLVKPTQTLTLTCTFSGFSLSTSGVGVGWIRQPPGKALEWLALIYWDDDKRYSPSLKSRLTITKDTSKNQVVLTMTNMDPVDTATYYCAHSYYYDSSDSYYFDYWGQGTLVTVSS

SEQ ID NO: 32 - protein, 10 aa, synthetic construct
GFSLSTSGVG

SEQ ID NO: 33 - protein, 7 aa, synthetic construct
IYWDDDK

SEQ ID NO: 34 - protein, 16 aa, synthetic construct
AHSYYYDSSDSYYFDY

SEQ ID NO: 35 - DNA, 321 bp, synthetic construct
  source 1..321: mol_type = other DNA
gacatccagttgacccagtctccatccttcctgtctgcatctgtaggagacagagtcaccatcacttgctgggccagtcagggcattagcagtcgtttagcctggtatcagcaaaaaccagggaaagccccgaaactcctgatctatgctgcatccactttgcaaagtggggtcccatcaaggttcagcggcagtggatctgggacagaattcactctcacaatcagcagcctgcagcctgaagattttggaaattattactgtcaacagtttaatacttacccattcactttcggccctgggaccaaagtggatatcaaa

SEQ ID NO: 36 - protein, 107 aa, synthetic construct
DIQLTQSPSFLSASVGDRVTITCWASQGISSRLAWYQQKPGKAPKLLIYAASTLQSGVPSRFSGSGSGTEFTLTISSLQPEDFGNYYCQQFNTYPFTFGPGTKVDIK

SEQ ID NO: 37 - protein, 6 aa, synthetic construct
QGISSR

SEQ ID NO: 38 - protein, 9 aa, synthetic construct
QQFNTYPFT

SEQ ID NO: 39 - DNA, 357 bp, synthetic construct
  source 1..357: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacacactacgcagactccgtgaagggccggttcaccatctccagagacaattccaagaacacgctgtatctgcaaatgaacagcctgagagccgaggacacggccgtatattactgtgcgaaagaaggctacagtaactggtacttcgatctctggggccgtggcaccctggtcactgtctcctca

SEQ ID NO: 40 - protein, 119 aa, synthetic construct
EVQLVESGGGLVQPGGSLRLSCAASGFTFSSYAMSWVRQAPGKGLEWVSAISGSGGSTHYADSVKGRFTISRDNSKNTLYLQMNSLRAEDTAVYYCAKEGYSNWYFDLWGRGTLVTVSS

SEQ ID NO: 41 - protein, 12 aa, synthetic construct
AKEGYSNWYFDL

SEQ ID NO: 42 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccatcagggccactggcatcccagccaggttcagtggcagtgggtctgggacagagttcactctcaccatcagcatcctgcagtctgaagattttgcagtttattactgtcaccagtataataactggccgctcactttcggcggagggaccaaggtggagatcaaa

SEQ ID NO: 43 - DNA, 354 bp, synthetic construct
  source 1..354: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcaactattagtggtagtggtggtagcacatactacgcagactccgtgaagggccggttcaccatctccagggacaattccaagaacacgctgtatctgcaaatgaacagcctgagagccgaggacacggccgtatattactgtgcgaaagcgccgtataactggaactttgactactggggccagggaaccctggtcaccgtctcctca

SEQ ID NO: 44 - protein, 118 aa, synthetic construct
EVQLVESGGGLVQPGGSLRLSCAASGFTFSSYAMSWVRQAPGKGLEWVSTISGSGGSTYYADSVKGRFTISRDNSKNTLYLQMNSLRAEDTAVYYCAKAPYNWNFDYWGQGTLVTVSS

SEQ ID NO: 45 - protein, 11 aa, synthetic construct
AKAPYNWNFDY

SEQ ID NO: 46 - DNA, 321 bp, synthetic construct
  source 1..321: mol_type = other DNA
gaagtagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctctcactttcggcggagggaccaaggtggagatcaaa

SEQ ID NO: 47 - protein, 107 aa, synthetic construct
EVVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIK

SEQ ID NO: 48 - DNA, 357 bp, synthetic construct
  source 1..357: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcatctattagtggtagtggtggtagcacatactacgcagactccgtgaagggccggttcaccatctccagagacaattccaagaacacgctgtatctgcaaatgagcagcctgagagccgaggacacggccgtttattactgtgcgaaagatggttacagtaactggtacttcgatctctggggccgtggcaccctggtcactgtctcctca

SEQ ID NO: 49 - protein, 119 aa, synthetic construct
EVQLVESGGGLVQPGGSLRLSCAASGFTFSSYAMSWVRQAPGKGLEWVSSISGSGGSTYYADSVKGRFTISRDNSKNTLYLQMSSLRAEDTAVYYCAKDGYSNWYFDLWGRGTLVTVSS

SEQ ID NO: 50 - protein, 12 aa, synthetic construct
AKDGYSNWYFDL

SEQ ID NO: 51 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggcgcatccaacagggccactggtatcccagccaggttcagtggcagtgggtctgggacagagttcactctcaccatcagcagcctgcagtctgaagatcttgcagtttattactgtcaccagtataataactggccgctcactttcggcggagggaccaaggtggagatcaaa

SEQ ID NO: 52 - protein, 107 aa, synthetic construct
EIVMTQSPATLSVSPGERATLSCRASQSVSSNLAWYQQKPGQAPRLLIYGASNRATGIPARFSGSGSGTEFTLTISSLQSEDLAVYYCHQYNNWPLTFGGGTKVEIK

SEQ ID NO: 53 - DNA, 357 bp, synthetic construct
  source 1..357: mol_type = other DNA
gaggtgcagctggtggagtctgggggaggcttggtacagcctggggggtccctgagactctcctgtgcagcctctggattcacctttagcagctatgccatgaactgggtccgccaggctccagggaaggggctggagtgggtctcaactattagtggtagtggtggtagcacatattccgcagactccgtgaagggccggttcaccatctccagagacaattccaagaatacgctgtatctgcaaatgaacagcctgagagccgaggacacggccgtatattactgtgcgaaagcttatcgcaacaactggtactttgagtgctggggccagggaaccctggtcaccgtctcctca

SEQ ID NO: 54 - protein, 119 aa, synthetic construct
EVQLVESGGGLVQPGGSLRLSCAASGFTFSSYAMNWVRQAPGKGLEWVSTISGSGGSTYSADSVKGRFTISRDNSKNTLYLQMNSLRAEDTAVYYCAKAYRNNWYFECWGQGTLVTVSS

SEQ ID NO: 55 - protein, 12 aa, synthetic construct
AKAYRNNWYFEC

SEQ ID NO: 56 - DNA, 321 bp, synthetic construct
  source 1..321: mol_type = other DNA
gaaatagtgatgacgcagtctccagccaccctgtctgtgtctccaggggaaagagtcaccctctcctgcagggccagtcagagtgttaacagcaacttagcctggtaccagcagaaacctggccaggctcccaggctcctcatctatggtgcatccaccagggccactggtatcccagccaggttcagtggcagtgggtctgggacagagttcactctcaccatcaacagcctgcagtctgaagactttgtagtttattactgtcagcagtataataattggccattcactttcggccctgggaccaaagtggatatcaaa

SEQ ID NO: 57 - protein, 107 aa, synthetic construct
EIVMTQSPATLSVSPGERVTLSCRASQSVNSNLAWYQQKPGQAPRLLIYGASTRATGIPARFSGSGSGTEFTLTINSLQSEDFVVYYCQQYNNWPFTFGPGTKVDIK

SEQ ID NO: 58 - protein, 6 aa, synthetic construct
QSVNSN

SEQ ID NO: 59 - protein, 9 aa, synthetic construct
QQYNNWPFT

SEQ ID NO: 60 - DNA, 357 bp, synthetic construct
  source 1..357: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcaactattagtggtagtggtggtagcacatactacgcagactccgtgaagggccggttcaccatctccagagacaattccaagaacacgctgtatctgcaaatgaacagcctgagagccgaggacacggccgtatattactgtgcgaaaggggcctacggaacttggtacttcggtctctggggccgtggcaccctggtcactgtctcctca

SEQ ID NO: 61 - protein, 119 aa, synthetic construct
EVQLVESGGGLVQPGGSLRLSCAASGFTFSSYAMSWVRQAPGKGLEWVSTISGSGGSTYYADSVKGRFTISRDNSKNTLYLQMNSLRAEDTAVYYCAKGAYGTWYFGLWGRGTLVTVSS

SEQ ID NO: 62 - protein, 12 aa, synthetic construct
AKGAYGTWYFGL

SEQ ID NO: 63 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccatcagggccactggcatcccagccaggttcagtggcagtgggtctgggacagagttcactctcaccatcagcatcctgcagtctgaagattttgcagtttattactgtcagcagtataataactggccgttcactttcggcggagggaccaaggtggagatcaaa

SEQ ID NO: 64 - protein, 107 aa, synthetic construct
EIVMTQSPATLSVSPGERATLSCRASQSVSSNLAWYQQKPGQAPRLLIYGASIRATGIPARFSGSGSGTEFTLTISILQSEDFAVYYCQQYNNWPFTFGGGTKVEIK

SEQ ID NO: 65 - DNA, 354 bp, synthetic construct
  source 1..354: mol_type = other DNA
gaggtgcagctggtggagtctgggggaggcttggtacagccgggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaaatagcagaagctgggtgtttgactactggggccagggaaccctggtcaccgtctcctca

SEQ ID NO: 66 - protein, 118 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKNSRSWVFDYWGQGTLVTVSS

SEQ ID NO: 67 - protein, 11 aa, synthetic construct
AKNSRSWVFDY

SEQ ID NO: 68 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccagactcccaggctcctcatgtatggtgcatccaccagggccactggtatcccagccaggttcagtggcagtgggtctgggacagagttcactctcaccatcagcagcctgcagtctgaagattttgcagtttattactgtcagcagtataataactggccattcactttcggccctgggaccaaagtggatatcaaa

SEQ ID NO: 69 - protein, 107 aa, synthetic construct
EIVMTQSPATLSVSPGERATLSCRASQSVSSNLAWYQQKPGQTPRLLMYGASTRATGIPARFSGSGSGTEFTLTISSLQSEDFAVYYCQQYNNWPFTFGPGTKVDIK

SEQ ID NO: 70 - DNA, 354 bp, synthetic construct
  source 1..354: mol_type = other DNA
gaggtgcagttggtggagtctgggggaggcttggtacagccgggggggtccctgagactctcctgtgcagcctctggattcacctttagcagctatgccatgagctgggtccgccaggctccagggaaggggctggagtgggtctcaactattagtggtagtggtggtagcacacactacgcagactccgtgaagggccggttcaccatctccagagacaattccaagaacacgctgtatctgcaaatgaacagcctgagagccgaggacacggccgtatattactgtgcgaaagatagcagcacctgggtgtttgactactggggccagggaaccctggtcaccgtctcctca

SEQ ID NO: 71 - protein, 118 aa, synthetic construct
EVQLVESGGGLVQPGGSLRLSCAASGFTFSSYAMSWVRQAPGKGLEWVSTISGSGGSTHYADSVKGRFTISRDNSKNTLYLQMNSLRAEDTAVYYCAKDSSTWVFDYWGQGTLVTVSS

SEQ ID NO: 72 - protein, 11 aa, synthetic construct
AKDSSTWVFDY

SEQ ID NO: 73 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtctcccagccaggttcagtggcagtgggtctgggacagagttcactctcaccatcagcagcctgcagtctgaagattttgcagtttattactgtcagcagtataataactggccattcactttcggccctgggaccaaagtggatatcaaa

SEQ ID NO: 74 - protein, 107 aa, synthetic construct
EIVMTQSPATLSVSPGERATLSCRASQSVSSNLAWYQQKPGQAPRLLIYGASTRATGLPARFSGSGSGTEFTLTISSLQSEDFAVYYCQQYNNWPFTFGPGTKVDIK

SEQ ID NO: 75 - DNA, 357 bp, synthetic construct
  source 1..357: mol_type = other DNA
gaggtgcaactggtggagtctgggggaggcttggtacagcctggggggtccctgagactctcctgtgcagcctctggattcacctttagcagctatgccatgaactgggtccgccaggctccagggaaggggctggagtgggtctcagctattagtggtagtggtggtagcacaaactacgcagactccgtgaagggccggttcaccatctccagagacaatgccaagaacacgctgtatctgcaaatgaacagcctgagagccgaggacacggccgtatattactgtgcgaaagatatcttctctaactgggccattgactactggggccagggaaccctggtcaccgtctcctca

SEQ ID NO: 76 - protein, 119 aa, synthetic construct
EVQLVESGGGLVQPGGSLRLSCAASGFTFSSYAMNWVRQAPGKGLEWVSAISGSGGSTNYADSVKGRFTISRDNAKNTLYLQMNSLRAEDTAVYYCAKDIFSNWAIDYWGQGTLVTVSS

SEQ ID NO: 77 - protein, 12 aa, synthetic construct
AKDIFSNWAIDY

SEQ ID NO: 78 - DNA, 321 bp, synthetic construct
  source 1..321: mol_type = other DNA
gaaatcgtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccatcagggccactggcatcccagccaggttcagtggcagtgggtctgggacagagttcactctcaccatcagcatcctgcagtctgaagattttgcagtttattactgtcttcagtatcataactggcctctcactttcggcggagggaccaaggtggagatcaaa

SEQ ID NO: 79 - protein, 107 aa, synthetic construct
EIVMTQSPATLSVSPGERATLSCRASQSVSSNLAWYQQKPGQAPRLLIYGASIRATGIPARFSGSGSGTEFTLTISILQSEDFAVYYCLQYHNWPLTFGGGTKVEIK

SEQ ID NO: 80 - protein, 9 aa, synthetic construct
LQYHNWPLT

SEQ ID NO: 81 - DNA, 357 bp, synthetic construct
  source 1..357: mol_type = other DNA
gaggtgcagctggtggagtctgggggaggcttggtacagcctggggggtccctgagactctcctgtgcagcctctggattcacctttagcagctatgccatgaactgggtccgccaggctccagggaaggggctggaatgggtctcagctattagtggtagtggtggtagcacatactacgcagactccgtgaagggccggttcaccatctccagagacaattccaagaacacgctgtatctgcaaatgaacagcctgagagccgaagacacgaccgtatattactgtgcgaaagggggtagcaggaactggtactttgactactgggcccagggaaccctggtcaccgtctcctca

SEQ ID NO: 82 - protein, 119 aa, synthetic construct
EVQLVESGGGLVQPGGSLRLSCAASGFTFSSYAMNWVRQAPGKGLEWVSAISGSGGSTYYADSVKGRFTISRDNSKNTLYLQMNSLRAEDTTVYYCAKGGSRNWYFDYWAQGTLVTVSS

SEQ ID NO: 83 - protein, 12 aa, synthetic construct
AKGGSRNWYFDY

SEQ ID NO: 84 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtctgcagtataataactggccgctcactttcggcggagggaccaaggtggagatcaaa

SEQ ID NO: 85 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCLQYNNWPLTFGGGTKVEIK

SEQ ID NO: 86 - protein, 9 aa, synthetic construct
LQYNNWPLT

SEQ ID NO: 87 - DNA, 381 bp, synthetic construct
  source 1..381: mol_type = other DNA
gaagtgcagctggtggagtctgggggaggcttggtacagcctggcaggtccctgagactctcctgtgcagcctctggattcacctttgatgattatgccatgcactgggtccggcaagttccagggaagggcctggagtgggtctcaggtattaattggaatagtggtagcttagtctatgcggactctgtgaagggccgattcaccatctccagagacaacgccaagaagttcctgtatctgcaaatgaacagtctgagagctgaggacacggccttgtattactgtgcaaaagatatccgtagagcagtggctggtcgttactactactactacggtatggacgtctggggccaagggaccacggtcaccgtctcctca

SEQ ID NO: 88 - protein, 127 aa, synthetic construct
EVQLVESGGGLVQPGRSLRLSCAASGFTFDDYAMHWVRQVPGKGLEWVSGINWNSGSLVYADSVKGRFTISRDNAKKFLYLQMNSLRAEDTALYYCAKDIRRAVAGRYYYYYGMDVWGQGTTVTVSS

SEQ ID NO: 89 - protein, 8 aa, synthetic construct
INWNSGSL

SEQ ID NO: 90 - protein, 20 aa, synthetic construct
AKDIRRAVAGRYYYYYGMDV

SEQ ID NO: 91 - DNA, 321 bp, synthetic construct
  source 1..321: mol_type = other DNA
gacatccagatgacccagtctccatcctccctgtctgcttctgtaggagacagagtcaccatcacttgccgggcaagtcagagcattagcagctatttaaattggtatcagcagaaatcagggaaagcccctaatctcctgatctatgctgcatccagtttgcaaagtggggtcccatcaaggttcagtggcagtggatctgggacagatttcactctcaccatcagcagtctgcaacctgaagattttgcaacttactactgtcaacagagttacattaccccgctcactttcggcggagggaccaaggtggagatcaaa

SEQ ID NO: 92 - protein, 107 aa, synthetic construct
DIQMTQSPSSLSASVGDRVTITCRASQSISSYLNWYQQKSGKAPNLLIYAASSLQSGVPSRFSGSGSGTDFTLTISSLQPEDFATYYCQQSYITPLTFGGGTKVEIK

SEQ ID NO: 93 - protein, 6 aa, synthetic construct
QSISSY

SEQ ID NO: 94 - protein, 9 aa, synthetic construct
QQSYITPLT

SEQ ID NO: 95 - DNA, 354 bp, synthetic construct
  source 1..354: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtcgtgacacacactacgcagactccgttaagggccggttcaccatctccagagacaattccaagaacacgctgtttctgcaaatgaacagcctgagagccgaggacacggccgtgtattactgtgcgaaagccgagtataactggaacttcaactactggggccagggaaccctggtcaccgtctcctca

SEQ ID NO: 96 - protein, 118 aa, synthetic construct
EVQLVESGGGLVQPGGSLRLSCAASGFTFSSYAMSWVRQAPGKGLEWVSAISGSGRDTHYADSVKGRFTISRDNSKNTLFLQMNSLRAEDTAVYYCAKAEYNWNFNYWGQGTLVTVSS

SEQ ID NO: 97 - protein, 8 aa, synthetic construct
ISGSGRDT

SEQ ID NO: 98 - protein, 11 aa, synthetic construct
AKAEYNWNFNY

SEQ ID NO: 99 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagaaatttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccattcactttcggccctgggaccaaagtggatatcaaa

SEQ ID NO: 100 - protein, 107 aa, synthetic construct
EIVMTQSPATLSVSPGERATLSCRASQSVSRNLAWYQQKPGQAPRLLIYGASTRATGIPARFSGSGSGTEFTLTISSLQSEDFAVYYCQQYNNWPFTFGPGTKVDIK

SEQ ID NO: 101 - protein, 6 aa, synthetic construct
QSVSRN

SEQ ID NO: 102 - DNA, 366 bp, synthetic construct
  source 1..366: mol_type = other DNA
cagatcaccttgaaggagtctggtcctacgctggtgaaacccacacagaccctcacgctgacctgcaccttctctgggttctcactcagtattagtggagtgggtgtgggctggatccgtcagcccccaggaaaggccctggagtggcttgcactcatttattgggatgatgataagcgctacagcccatctctgaagaacagactcaccatcaccaaggacacctccaaaaaccaggtggtccttacaatgaccgacatggaccctgtggacacagccacatattactgtgcacacctacgatattttgacttggcgtactactttgactactggggccagggaaccctggtcaccgtctcctca

SEQ ID NO: 103 - protein, 122 aa, synthetic construct
QITLKESGPTLVKPTQTLTLTCTFSGFSLSISGVGVGWIRQPPGKALEWLALIYWDDDKRYSPSLKNRLTITKDTSKNQVVLTMTDMDPVDTATYYCAHLRYFDLAYYFDYWGQGTLVTVSS

SEQ ID NO: 104 - protein, 10 aa, synthetic construct
GFSLSISGVG

SEQ ID NO: 105 - protein, 14 aa, synthetic construct
AHLRYFDLAYYFDY

SEQ ID NO: 106 - DNA, 321 bp, synthetic construct
  source 1..321: mol_type = other DNA
gacatccagatgacccagtctccatcctccctgtctgcatctataggagacagagtcaccatcacttgccgggcaagtcaggacattagaaatgatttaggctggtatcagcagaaaccagggaaagcccctaagcgcctgatctatgctgcatccagtttgcagagtggggtcccatccaggttcagcgccagtggatctgggacagaattcactctcacaatcagcagcctgcagcctgaagattttgcaacttattactgtctacagtctaatagttacccattcactttcggccctgggaccaaagtggatatcaaa

SEQ ID NO: 107 - protein, 107 aa, synthetic construct
DIQMTQSPSSLSASIGDRVTITCRASQDIRNDLGWYQQKPGKAPKRLIYAASSLQSGVPSRFSASGSGTEFTLTISSLQPEDFATYYCLQSNSYPFTFGPGTKVDIK

SEQ ID NO: 108 - protein, 9 aa, synthetic construct
LQSNSYPFT

SEQ ID NO: 109 - DNA, 366 bp, synthetic construct
  source 1..366: mol_type = other DNA
cagatcaccttgaaggagtctggtcctacgctggtgaaacccacacagaccctcaccctgacctgcaccttctctgggttctcactcagacctagtggagtgggtgtgggctggatccgtcagcccccaggaaaggccctggagtggcttgcaatcatttattgggatgatgataagcgctacagcccatctctgaagagcaggctcaccatcaccaaggacacctccaaaaaccaggtggtccttacaatgaccaacatggaccctgtggacacagccatatattactgtgcacacagttactatgataatagtggttattactttgactactggggccagggaaccctggtcaccgtctcctca

SEQ ID NO: 110 - protein, 122 aa, synthetic construct
QITLKESGPTLVKPTQTLTLTCTFSGFSLRPSGVGVGWIRQPPGKALEWLAIIYWDDDKRYSPSLKSRLTITKDTSKNQVVLTMTNMDPVDTAIYYCAHSYYDNSGYYFDYWGQGTLVTVSS

SEQ ID NO: 111 - protein, 10 aa, synthetic construct
GFSLRPSGVG

SEQ ID NO: 112 - protein, 14 aa, synthetic construct
AHSYYDNSGYYFDY

SEQ ID NO: 113 - DNA, 321 bp, synthetic construct
  source 1..321: mol_type = other DNA
gacatccagatgacccagtctccatcctcactgtctgcatctgtaggagacagagtcaccatcacttgtcgggcgagtcagggcattagcaattttttagcctggtttcagcagaaaccagggaaagccccgaagtccctgatccatgctgcatccagtttgcaaagtggggtcccatcaaagttcagcggcagtggatctgggacagatttcactctcaccatcagcagcctgcagcctgaagattttgcaacttattactgccaacagtattatacttacccattcactttcggccctgggaccatactggatatcaga

SEQ ID NO: 114 - protein, 107 aa, synthetic construct
DIQMTQSPSSLSASVGDRVTITCRASQGISNFLAWFQQKPGKAPKSLIHAASSLQSGVPSKFSGSGSGTDFTLTISSLQPEDFATYYCQQYYTYPFTFGPGTILDIR

SEQ ID NO: 115 - protein, 6 aa, synthetic construct
QGISNF

SEQ ID NO: 116 - protein, 9 aa, synthetic construct
QQYYTYPFT

SEQ ID NO: 117 - DNA, 369 bp, synthetic construct
  source 1..369: mol_type = other DNA
cagatcaccttgaaggagtctggtcctacgctggtgaaacccacacagaccctcacgctgacctgtaccttctctgggttctcactcagtattagtggagtgggtgtgggctggatccgtcagcccccaggaaaggccctggagtggcttgcactcatttattgggatgatgataagcgctacagcccgtctctgaagagcaggctcaccatcaccaaggacacctccaaaaaccaggtggtccttacaatgaccaacatggaccctgtggacacagccacatattactgtgcacacagccattactatgctagtactggttattactttgactactggggccagggaaccctggtcaccgtctcctca

SEQ ID NO: 118 - protein, 123 aa, synthetic construct
QITLKESGPTLVKPTQTLTLTCTFSGFSLSISGVGVGWIRQPPGKALEWLALIYWDDDKRYSPSLKSRLTITKDTSKNQVVLTMTNMDPVDTATYYCAHSHYYASTGYYFDYWGQGTLVTVSS

SEQ ID NO: 119 - protein, 15 aa, synthetic construct
AHSHYYASTGYYFDY

SEQ ID NO: 120 - DNA, 321 bp, synthetic construct
  source 1..321: mol_type = other DNA
gccatccagatgacccagtctccatcctccctgtctgcatctgtaggagacagagtcaccatcacttgccgggcaagtcagggcattagaaataatttaggctggtatcagcagaaaccagggaaagcccctaaactcctgatctatgctgcatccggtttacaaagtggggtcccttcaaggttcagcggcagtggatctggcacagatttcactctcaccatcagcagcctgcagcctgaagattttgcaacttattactgtctacaagattacaattacccattcactttcggccctgggaccaaagtggatatcaaa

SEQ ID NO: 121 - protein, 107 aa, synthetic construct
AIQMTQSPSSLSASVGDRVTITCRASQGIRNNLGWYQQKPGKAPKLLIYAASGLQSGVPSRFSGSGSGTDFTLTISSLQPEDFATYYCLQDYNYPFTFGPGTKVDIK

SEQ ID NO: 122 - protein, 6 aa, synthetic construct
QGIRNN

SEQ ID NO: 123 - protein, 9 aa, synthetic construct
LQDYNYPFT

SEQ ID NO: 124 - DNA, 357 bp, synthetic construct
  source 1..357: mol_type = other DNA
gaggtgcaactggtggagtctgggggaggcttggtacagcctggggggtccctgagactctccggtgcagcctctggattcacctttagcagctatgccatgagctgggtccgccaggttccagggaaggggctggagtgggtctcaactattagtggtagtggtggtagcacatactacgcagactccgtgaagggccggttcaccatctccagagacaattccaagaacacgctgtatctgcaaatgaacagcctgagagccgaggacacggccgtatattactgtgcgaaaggggggatggggacctggtacttcgatgtctggggccgtggcaccctggtcactgtctcctca

SEQ ID NO: 125 - protein, 119 aa, synthetic construct
EVQLVESGGGLVQPGGSLRLSGAASGFTFSSYAMSWVRQVPGKGLEWVSTISGSGGSTYYADSVKGRFTISRDNSKNTLYLQMNSLRAEDTAVYYCAKGGMGTWYFDVWGRGTLVTVSS

SEQ ID NO: 126 - protein, 12 aa, synthetic construct
AKGGMGTWYFDV

SEQ ID NO: 127 - DNA, 321 bp, synthetic construct
  source 1..321: mol_type = other DNA
gaaatagtgatgacgcagtctccagccaccctgtctgtgtctccaggggaaagagccaccctctcctgcacgaccagtcagagtgttagcagcaacttagcctggtgccaacagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtatcataagtggccattcactttcggccctgggaccaaagtggatatcaaa

SEQ ID NO: 128 - protein, 107 aa, synthetic construct
EIVMTQSPATLSVSPGERATLSCTTSQSVSSNLAWCQQKPGQAPRLLIYGASTRATGIPARFSGSGSGTEFTLTISSLQSEDFAVYYCQQYHKWPFTFGPGTKVDIK

SEQ ID NO: 129 - protein, 9 aa, synthetic construct
QQYHKWPFT

SEQ ID NO: 130 - DNA, 357 bp, synthetic construct
  source 1..357: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacattctacgcagactccgtgaagggccggttcaccatctccagagacaattccaagaacacgctttatctgcaaatgaacagcctgagagccgaggacacggccgtatattactgtgcgaaagatccggggaataactggaatatggagtactggggccagggaaccctggtcaccgtctcttca

SEQ ID NO: 131 - protein, 119 aa, synthetic construct
EVQLVESGGGLVQPGGSLRLSCAASGFTFSSYAMSWVRQAPGKGLEWVSAISGSGGSTFYADSVKGRFTISRDNSKNTLYLQMNSLRAEDTAVYYCAKDPGNNWNMEYWGQGTLVTVSS

SEQ ID NO: 132 - protein, 12 aa, synthetic construct
AKDPGNNWNMEY

SEQ ID NO: 133 - DNA, 321 bp, synthetic construct
  source 1..321: mol_type = other DNA
gaaatagtgatgacgcagtctccagccaccctgtctgtgtctccaggagaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctggagattttgcagtttattactgtcaccagtatcataactggccgctcactttcggcggagggaccaaggtggagatcaaa

SEQ ID NO: 134 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSGDFAVYYCHQYHNWPLTFGGGTKVEIK

SEQ ID NO: 135 - protein, 9 aa, synthetic construct
HQYHNWPLT

SEQ ID NO: 136 - DNA, 357 bp, synthetic construct
  source 1..357: mol_type = other DNA
ggggtgcagctggtggactctgggggaggcttggtacagcctggggggtccctgagactctcctgtgcagcctctggattcacctttagcagctatgccatgaactgggtccgccaggctccagggaaggggctggagtgggtctcaactattagtggtagtggtggtaccacatactacgcagactccgtgaagggccggttcaccatctccagagacaattccaagaacacgctgtatctgcaaatgaacagcctgagagccgaggacacggccgtatattactgtgcgaaagcgacctacagtaactggccttttgactactggggccagggagccctggtcaccgtctcctca

SEQ ID NO: 137 - protein, 119 aa, synthetic construct
GVQLVDSGGGLVQPGGSLRLSCAASGFTFSSYAMNWVRQAPGKGLEWVSTISGSGGTTYYADSVKGRFTISRDNSKNTLYLQMNSLRAEDTAVYYCAKATYSNWPFDYWGQGALVTVSS

SEQ ID NO: 138 - protein, 8 aa, synthetic construct
ISGSGGTT

SEQ ID NO: 139 - protein, 12 aa, synthetic construct
AKATYSNWPFDY

SEQ ID NO: 140 - DNA, 321 bp, synthetic construct
  source 1..321: mol_type = other DNA
gaaatagtgatgacgcagtctccagccaccctgtctgtgtctccaggggaaagagccaccctctcctgcagggccagtcagggtgttagcagcaacttagcctggtaccagcagaaacctggccaggctcccaggctcctcatctatggtgcatccatcagggccactggcatcccagccaggttcagtggcagtgggtctgggacagagttcactctcaccatcagcctcctgcagtctgaagattttgcagtttattactgtctgcagtataataactggcctctcactttcggccctgggaccaaagtggatatcaaa

SEQ ID NO: 141 - protein, 107 aa, synthetic construct
EIVMTQSPATLSVSPGERATLSCRASQGVSSNLAWYQQKPGQAPRLLIYGASIRATGIPARFSGSGSGTEFTLTISLLQSEDFAVYYCLQYNNWPLTFGPGTKVDIK

SEQ ID NO: 142 - protein, 6 aa, synthetic construct
QGVSSN

SEQ ID NO: 143 - DNA, 369 bp, synthetic construct
  source 1..369: mol_type = other DNA
cagatcaccttgagggagtctggtcctccgctggtgaagcccacacagaccctcacgctgacctgcaccttctctgggttctcactcaggactaatggagtgggtgtgggctggacccgtcagcccccaggaaaggccctggagtggcttgcactcatttattgggatgatgataagcgctacagcccatctctgaagagcaggctcaccatcaccaagggcacctccaaaaaccaggtggtccttacaatgaccaacatggaccctgtggacacaggcacatatttctgtgcacacgcgtattactataattcggggagttatttctttgactactggggccagggaaccctggtcaccgtctcctca

SEQ ID NO: 144 - protein, 123 aa, synthetic construct
QITLRESGPPLVKPTQTLTLTCTFSGFSLRTNGVGVGWTRQPPGKALEWLALIYWDDDKRYSPSLKSRLTITKGTSKNQVVLTMTNMDPVDTGTYFCAHAYYYNSGSYFFDYWGQGTLVTVSS

SEQ ID NO: 145 - protein, 10 aa, synthetic construct
GFSLRTNGVG

SEQ ID NO: 146 - protein, 15 aa, synthetic construct
AHAYYYNSGSYFFDY

SEQ ID NO: 147 - DNA, 321 bp, synthetic construct
  source 1..321: mol_type = other DNA
gacatccagatgacccagtctccatcctccctgtctgcatctgtaggagacagagtcaccatcatttgccgggcaagtcagggcattagaaatgatttaggctggtatcagcagaaaccagggaaagcccctaagcgcctgatctctgttgtatccagtttgcaaagtggggtcccatcaaggttcagcggcagtggatctgggacagaattcactctcacaatcagcaacctacagcctgaagattttgcaacttattactgtctacagcataatagttacccgtggacgttcggccaagggaccaaggtggaaatcaaa

SEQ ID NO: 148 - protein, 107 aa, synthetic construct
DIQMTQSPSSLSASVGDRVTIICRASQGIRNDLGWYQQKPGKAPKRLISVVSSLQSGVPSRFSGSGSGTEFTLTISNLQPEDFATYYCLQHNSYPWTFGQGTKVEIK

SEQ ID NO: 149 - protein, 6 aa, synthetic construct
QGIRND

SEQ ID NO: 150 - skipped

SEQ ID NO: 151 - protein, 9 aa, synthetic construct
LQHNSYPWT

SEQ ID NO: 152 - DNA, 354 bp, synthetic construct
  source 1..354: mol_type = other DNA
gaggtgcagctggtggagtctgggggaggcttggtacagcctggggggtccctgagactctcctgtgcagcctctggattcacctttagcagctatcccatgagctgggtccgccaggctccagggaaggggctggagtgggtctcaactgttagtggtagtggtggtgacacacactacgcagactccgtgaagggccggttcaccatctccagagacaattccaagaacacgctgtatctgcaaatgaacagcctgagagccgacgacacggccgtatatttctgtgcgaaaggccctcataactggaactttgactactggggccagggaaccctggtcaccgtctcctca

SEQ ID NO: 153 - protein, 118 aa, synthetic construct
EVQLVESGGGLVQPGGSLRLSCAASGFTFSSYPMSWVRQAPGKGLEWVSTVSGSGGDTHYADSVKGRFTISRDNSKNTLYLQMNSLRADDTAVYFCAKGPHNWNFDYWGQGTLVTVSS

SEQ ID NO: 154 - protein, 8 aa, synthetic construct
GFTFSSYP

SEQ ID NO: 155 - protein, 8 aa, synthetic construct
VSGSGGDT

SEQ ID NO: 156 - protein, 11 aa, synthetic construct
AKGPHNWNFDY

SEQ ID NO: 157 - DNA, 321 bp, synthetic construct
  source 1..321: mol_type = other DNA
gaaatagtgatgacgcagtctccagccaccctgtctgtgttttcaggggaaagagccaccctctcctgcagggccagtcagagtgttagtagcaacttagcctggtaccagcagaaacctggccaggctcccaggctcctcatctatggtgcatccaccagggccactggtatcccagtcaggttcagtggcagtgggtctgggacagagttcactctcaccatcagcagcctgctgtctgaagattttgcagtttattactgtcagcagtataataagtggcctctcactttcggcggagggaccatggtggagatcaga

SEQ ID NO: 158 - protein, 107 aa, synthetic construct
EIVMTQSPATLSVFSGERATLSCRASQSVSSNLAWYQQKPGQAPRLLIYGASTRATGIPVRFSGSGSGTEFTLTISSLLSEDFAVYYCQQYNKWPLTFGGGTMVEIR

SEQ ID NO: 159 - protein, 9 aa, synthetic construct
QQYNKWPLT

SEQ ID NO: 160 - DNA, 357 bp, synthetic construct
  source 1..357: mol_type = other DNA
gaggtgcaggtggtggagtctgggggaggcttggtacagcctggggggtccctgagactctcctgtgcagcctctggattcacctttagcagctatgccatgagctgggtccgccaggctccagggaaggggctggagtgggtctcagctcttagtggcagtggtggtcgcacatactacgcagactccgtgaagggccggttcaccatctccagagacaattccaagaacacgctgtatctgcaaatggacagtctgagagtcgaggacacggccgtatattactgtgcgaaagatggcatggggaactggtacttcgatctctggggccgtggcaccctggtcactgtctcctca

SEQ ID NO: 161 - protein, 119 aa, synthetic construct
EVQVVESGGGLVQPGGSLRLSCAASGFTFSSYAMSWVRQAPGKGLEWVSALSGSGGRTYYADSVKGRFTISRDNSKNTLYLQMDSLRVEDTAVYYCAKDGMGNWYFDLWGRGTLVTVSS

SEQ ID NO: 162 - protein, 8 aa, synthetic construct
LSGSGGRT

SEQ ID NO: 163 - protein, 12 aa, synthetic construct
AKDGMGNWYFDL

SEQ ID NO: 164 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggatcagtggcagtgggtctgggacagagttcactctcgccatcagcagcctgcagtctgaagattttgcagtttattactgtcatcagtatcataactggcctctcactttcggcggagggaccaaggtggagatcaaa

SEQ ID NO: 165 - protein, 107 aa, synthetic construct
EIVMTQSPATLSVSPGERATLSCRASQSVSSNLAWYQQKPGQAPRLLIYGASTRATGIPARISGSGSGTEFTLAISSLQSEDFAVYYCHQYHNWPLTFGGGTKVEIK

SEQ ID NO: 166 - DNA, 357 bp, synthetic construct
  source 1..357: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcaactattagtggtagtggtggtagcacacactacgcagactccgtgaagggccggttcaccatctccagagacaattccaagaacacgctgtttctgcaaatgaacagcctgagagccgaggacacggccgtatattactgtgcgaaagatgcctttgggaactggtacatcgatctctggggccgtggcaccctggtcactgtctcctca

SEQ ID NO: 167 - protein, 119 aa, synthetic construct
EVQLVESGGGLVQPGGSLRLSCAASGFTFSSYAMSWVRQAPGKGLEWVSTISGSGGSTHYADSVKGRFTISRDNSKNTLFLQMNSLRAEDTAVYYCAKDAFGNWYIDLWGRGTLVTVSS

SEQ ID NO: 168 - protein, 12 aa, synthetic construct
AKDAFGNWYIDL

SEQ ID NO: 169 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaatattttgcaatttattactgtcagcagtataataactggcctctcactttcggcggaggaaccaaggtggagatcaaa

SEQ ID NO: 170 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEYFAIYYCQQYNNWPLTFGGGTKVEIK

SEQ ID NO: 171 - DNA, 351 bp, synthetic construct
  source 1..351: mol_type = other DNA
caggtgcagctggtggagtctgggggaggcgtggtccagcctgggaggtccctgagactctcctgtgcagcctctggattcaccttcagtagctatgccatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgggctacggtggtaactcgctcgacccctggggccagggaaccctggtcaccgtctcctca

SEQ ID NO: 172 - protein, 117 aa, synthetic construct
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCAGYGGNSLDPWGQGTLVTVSS

SEQ ID NO: 173 - protein, 8 aa, synthetic construct
ISYDGSNK

SEQ ID NO: 174 - protein, 10 aa, synthetic construct
AGYGGNSLDP

SEQ ID NO: 175 - DNA, 321 bp, synthetic construct
  source 1..321: mol_type = other DNA
gccatccagatgacccagtctccatcctccctgtctgcatctgtaggagacagagtcaccatcacttgccgggcaagtcagggcattagaaatgatttagtctggtatcagcagaaaccagggaaagcccctaaggtcctgatctatgatgcgtccagtttacaaagtggggtcccatcaaggttcagcggcagtggatttggcacagatttcactctcaccatcagcagcctgcagcctgaagattttgcaacttattactgtctacaggattacaattacccgtgcagttttggccaggggaccaagctggagatcaaa

SEQ ID NO: 176 - protein, 107 aa, synthetic construct
AIQMTQSPSSLSASVGDRVTITCRASQGIRNDLVWYQQKPGKAPKVLIYDASSLQSGVPSRFSGSGFGTDFTLTISSLQPEDFATYYCLQDYNYPCSFGQGTKLEIK

SEQ ID NO: 177 - skipped

SEQ ID NO: 178 - protein, 9 aa, synthetic construct
LQDYNYPCS

SEQ ID NO: 179 - DNA, 366 bp, synthetic construct
  source 1..366: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagagagccttactatggttcggggagaggggttcgacccctggggccagggaaccctggtcaccgtctcctca

SEQ ID NO: 180 - protein, 122 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKESLTMVRGEGFDPWGQGTLVTVSS

SEQ ID NO: 181 - protein, 8 aa, synthetic construct
GFTFSSYG

SEQ ID NO: 182 - protein, 15 aa, synthetic construct
AKESLTMVRGEGFDP

SEQ ID NO: 183 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccgtgcagttttggccaggggaccaagctggagatcaaa

SEQ ID NO: 184 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFPCSFGQGTKLEIK

SEQ ID NO: 185 - protein, 6 aa, synthetic construct
QGISSW

SEQ ID NO: 186 - protein, 9 aa, synthetic construct
QQANSFPCS

SEQ ID NO: 187 - DNA, 372 bp, synthetic construct
  source 1..372: mol_type = other DNA
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcagtcatttattgggatgatgataagcgctacagcccatctctgaagagcagggtcaccatcaccaaggacacctccaaaaaccaggtggtccttacaatgaccaatatggaccctgtggacacagccacatattactgtgcacacagttattaccatgatagcagtgattcttactactttgactactgggcccagggaaccctggtcaccgtctcctca

SEQ ID NO: 188 - protein, 124 aa, synthetic construct
QITLKESGPTLVKPTQTLTLTCTFSGFSLSTSGVGVGWIRQPPGKALEWLAVIYWDDDKRYSPSLKSRVTITKDTSKNQVVLTMTNMDPVDTATYYCAHSYYHDSSDSYYFDYWAQGTLVTVSS

SEQ ID NO: 189 - protein, 16 aa, synthetic construct
AHSYYHDSSDSYYFDY

SEQ ID NO: 190 - DNA, 321 bp, synthetic construct
  source 1..321: mol_type = other DNA
gacatccagttgacccagtctccatccttcctgtctgcatctgtaggagacagagtcaccatcacttgctgggccagtcagggcattagcagtcgtttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttactagttacccattcactttcggccctgggaccaaagtggatatcaaa

SEQ ID NO: 191 - protein, 107 aa, synthetic construct
DIQLTQSPSFLSASVGDRVTITCWASQGISSRLAWYQQKPGKAPKLLIYAASTLQSGVPSRFSGSGSGTEFTLTISSLQPEDFATYYCQQLTSYPFTFGPGTKVDIK

SEQ ID NO: 192 - protein, 9 aa, synthetic construct
QQLTSYPFT

SEQ ID NO: 193 - DNA, 366 bp, synthetic construct
  source 1..366: mol_type = other DNA
cagatcaccttgaaggagtctggtcctacgctggtgaaacccacacagaccctcacgctgacctgcaccttctctgggttctcactcagcactaatggagtgggtgtgggctggatccgtcagcccccaggaaaggccctggagtggcttgcactcatttattgggatgatgataagcgctacagcccatctctgaagagcaggctcaccatcaccaaggacacctccaaaaaccaggtggtccttacaatgaccaacatggaccctgtggacacagccacatattactgtgcacacctttactatgttattagtggttattactttgactactggggccagggaaccctggtcaccgtctcctca

SEQ ID NO: 194 - protein, 122 aa, synthetic construct
QITLKESGPTLVKPTQTLTLTCTFSGFSLSTNGVGVGWIRQPPGKALEWLALIYWDDDKRYSPSLKSRLTITKDTSKNQVVLTMTNMDPVDTATYYCAHLYYVISGYYFDYWGQGTLVTVSS

SEQ ID NO: 195 - protein, 10 aa, synthetic construct
GFSLSTNGVG

SEQ ID NO: 196 - protein, 14 aa, synthetic construct
AHLYYVISGYYFDY

SEQ ID NO: 197 - DNA, 321 bp, synthetic construct
  source 1..321: mol_type = other DNA
gacatccagatgacccagtctccatcctcactgtctgcatctgtaggagacagagtcaccatcacttgtcgggcgagtcagggtatcagcagcagggtagcctggtatcaggagaaaccagagaaagcccctaagtccctgatctatgttgcatccagattgcaaagaggggtcccatcaaggttcagcggcagtggatctgggacagatttcactctcaccatcagcagcctgcagcctgaagattttgcaacttattactgccaacagtataatagttacccattcactttcggccctgggaccaaagtagatatcaaa

SEQ ID NO: 198 - protein, 107 aa, synthetic construct
DIQMTQSPSSLSASVGDRVTITCRASQGISSRVAWYQEKPEKAPKSLIYVASRLQRGVPSRFSGSGSGTDFTLTISSLQPEDFATYYCQQYNSYPFTFGPGTKVDIK

SEQ ID NO: 199 - protein, 9 aa, synthetic construct
QQYNSYPFT

SEQ ID NO: 200 - protein, 273 aa, Acinetobacter baumannii
MNKYFTCYVVASLFLSGCTVQHNLINETPSQIVQGHNQVIHQYFDEKNTSGVLVIQTDKKINLYGNALSRANTEYVPASTFKMLNALIGLENQKTDINEIFKWKGEKRSFTAWEKDMTLGEAMKLSAVPVYQELARRIGLDLMQKEVKRIGFGNAEIGQQVDNFWLVGPLKVTPIQEVEFVSQLAHTQLPFSEKVQANVKNMLLLEESNGYKIFGKTGWAMDIKPQVGWLTGWVEQPDGKIVAFALNMEMRSEMPASIRNELLMKSLKQLNII

SEQ ID NO: 201-416 - skipped (216 entries)

SEQ ID NO: 417 - DNA, 990 bp, Homo sapiens
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 418 - protein, 330 aa, Homo sapiens [sequence omitted]

SEQ ID NO: 419 - DNA, 990 bp, Homo sapiens
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 420 - protein, 330 aa, Homo sapiens [sequence omitted]

SEQ ID NO: 421 - DNA, 990 bp, Homo sapiens
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 422 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 423 - DNA, 990 bp, Homo sapiens
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacatcttctcatgctccgtgatgcatgaggctctgcacaaccactacacgcagaagagcctctccctgtctccgggtaaa

SEQ ID NO: 424 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 425 - DNA, 990 bp, Homo sapiens
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtggagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 426 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 427 - DNA, 978 bp, Homo sapiens
  source 1..978: mol_type = other DNA
gcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 428 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 429 - DNA, 978 bp, Homo sapiens
  source 1..978: mol_type = other DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgacctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 430 - protein, 326 aa, Homo sapiens
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 431 - DNA, 978 bp, Homo sapiens
  source 1..978: mol_type = other DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcagcttgggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 432 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 433 - DNA, 978 bp, Homo sapiens
  source 1..978: mol_type = other DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 434 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 435 - DNA, 981 bp, Homo sapiens
  source 1..981: mol_type = other DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 436 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 437 - DNA, 981 bp, Homo sapiens
  source 1..981: mol_type = other DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccgtgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcgtgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacgcagaagagcctctccctgtctctgggtaaa

SEQ ID NO: 438 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 439 - DNA, 981 bp, Homo sapiens
  source 1..981: mol_type = other DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctcaccgtggacaagagcaggtggcaggaggggaacgtcttctcatgctccgtgatgcatgaggctctgcacaaccactacacgcagaagagcctctccctgtctctgggtaaa

SEQ ID NO: 440 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 441 - DNA, 981 bp, Homo sapiens
  source 1..981: mol_type = other DNA
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctgaatttgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 442 - DNA, 981 bp, Homo sapiens
  source 1..981: mol_type = other DNA
gcctccaccaagggacctagcgtgttccctctcgccccctgttccaggtccacaagcgagtccaccgctgccctcggctgtctggtgaaagactactttcccgagcccgtgaccgtctcctggaatagcggagccctgacctccggcgtgcacacatttcccgccgtgctgcagagcagcggactgtatagcctgagcagcgtggtgaccgtgcccagctccagcctcggcaccaaaacctacacctgcaacgtggaccacaagccctccaacaccaaggtggacaagcgggtggagagcaagtacggccccccttgccctccttgtcctgcccctgagttcgagggaggaccctccgtgttcctgtttccccccaaacccaaggacaccctgatgatctcccggacacccgaggtgacctgtgtggtcgtggacgtcagccaggaggaccccgaggtgcagttcaactggtatgtggacggcgtggaggtgcacaatgccaaaaccaagcccagggaggagcagttcaattccacctacagggtggtgagcgtgctgaccgtcctgcatcaggattggctgaacggcaaggagtacaagtgcaaggtgtccaacaagggactgcccagctccatcgagaagaccatcagcaaggctaagggccagccgagggagccccaggtgtataccctgcctcctagccaggaagagatgaccaagaaccaagtgtccctgacctgcctggtgaagggattctacccctccgacatcgccgtggagtgggagagcaatggccagcccgagaacaactacaaaacaacccctcccgtgctcgatagcgacggcagcttctttctctacagccggctgacagtggacaagagcaggtggcaggagggcaacgtgttctcctgttccgtgatgcacgaggccctgcacaatcactacacccagaagagcctctccctgtccctgggcaag

SEQ ID NO: 443 - DNA, 981 bp, Homo sapiens
  source 1..981: mol_type = other DNA
gccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 444 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 445 - DNA, 981 bp, Homo sapiens
  source 1..981: mol_type = other DNA
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctccagttgcggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 446 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 447 - DNA, 321 bp, Homo sapiens
  source 1..321: mol_type = other DNA
cgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 448 - protein, 107 aa, Homo sapiens [sequence omitted]

SEQ ID NO: 449 - DNA, 321 bp, Homo sapiens
  source 1..321: mol_type = other DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggagagcaaggacagcacctacagcctcagcagcaccctgacgctgagcaaagcagactacgagaaacacaaagtctacgccggcgaagtcacccatcagggcctgagctcgcccgtcacaaagagcttcaacaggggagagtgt

SEQ ID NO: 450 - protein, 107 aa, Homo sapiens
RTVAAPSVFIFPPSDEQLKSGTASVVCLLNNFYPREAKVQWKVDNALQSGNSQESVTEQESKDSTYSLSSTLTLSKADYEKHKVYAGEVTHQGLSSPVTKSFNRGEC

SEQ ID NO: 451 - DNA, 321 bp, Homo sapiens
  source 1..321: mol_type = other DNA
cgaactgtggctgcaccatctgtcttcatcttcccgccatctgatgagcagttgaaatctggaactgcctctgttgtgtgcctgctgaataacttctatcccagagaggccaaagtacagcggaaggtggataacgccctccaatcgggtaactcccaggagagtgtcacagagcaggagagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 452 - protein, 107 aa, Homo sapiens
RTVAAPSVFIFPPSDEQLKSGTASVVCLLNNFYPREAKVQRKVDNALQSGNSQESVTEQESKDSTYSLSSTLTLSKADYEKHKVYACEVTHQGLSSPVTKSFNRGEC

SEQ ID NO: 453 - DNA, 321 bp, Homo sapiens
  source 1..321: mol_type = other DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 454 - protein, 107 aa, Homo sapiens
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 455 - DNA, 321 bp, Homo sapiens
  source 1..321: mol_type = other DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcaacaccctgacgctgagcaaagcagactacgagaaacacaaagtctacgcctgcgaagtcacccatcagggcctgagctcgcccgtcacaaagagcttcaacaggggagagtgc

SEQ ID NO: 456 - protein, 107 aa, Homo sapiens
RTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 457 - DNA, 312 bp, Homo sapiens
  source 1..312: mol_type = other DNA
cccaaggccaaccccacggtcactctgttcccgccctcctctgaggagctccaagccaacaaggccacactagtgtgtctgatcagtgacttctacccgggagctgtgacagtggcttggaaggcagatggcagccccgtcaaggcgggagtggagacgaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 458 - protein, 104 aa, Homo sapiens
PKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 459 - DNA, 318 bp, Homo sapiens
  source 1..318: mol_type = other DNA
ggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 460 - DNA, 318 bp, Homo sapiens; identical to SEQ ID NO: 459
  source 1..318: mol_type = other DNA

SEQ ID NO: 461 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 462 - DNA, 318 bp, Homo sapiens
  source 1..318: mol_type = other DNA
ggccagcctaaggccgctccttctgtgaccctgttccccccatcctccgaggaactgcaggctaacaaggccaccctcgtgtgcctgatcagcgacttctaccctggcgccgtgaccgtggcctggaaggctgatagctctcctgtgaaggccggcgtggaaaccaccaccccttccaagcagtccaacaacaaatacgccgcctcctcctacctgtccctgacccctgagcagtggaagtcccaccggtcctacagctgccaagtgacccacgagggctccaccgtggaaaagaccgtggctcctaccgagtgctcc

SEQ ID NO: 463 - DNA, 318 bp, Homo sapiens
  source 1..318: mol_type = other DNA
ggccagcctaaagctgcccccagcgtcaccctgtttcctccctccagcgaggagctccaggccaacaaggccaccctcgtgtgcctgatctccgacttctatcccggcgctgtgaccgtggcttggaaagccgactccagccctgtcaaagccggcgtggagaccaccacaccctccaagcagtccaacaacaagtacgccgcctccagctatctctccctgacccctgagcagtggaagtcccaccggtcctactcctgtcaggtgacccacgagggctccaccgtggaaaagaccgtcgcccccaccgagtgctcc

SEQ ID NO: 464 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 465 - DNA, 318 bp, Homo sapiens
  source 1..318: mol_type = other DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 466 - skipped

SEQ ID NO: 467 - protein, 106 aa, Homo sapiens; identical to SEQ ID NO: 464

SEQ ID NO: 468 - DNA, 312 bp, Homo sapiens
  source 1..312: mol_type = other DNA
cccaaggctgccccctcggtcactctgttcccaccctcctctgaggagcttcaagccaacaaggccacactggtgtgtctcataagtgacttctacccgggagccgtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagttgcccctacggaatgttca

SEQ ID NO: 469 - protein, 104 aa, Homo sapiens
PKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 470 - DNA, 318 bp, Homo sapiens
  source 1..318: mol_type = other DNA
ggtcagcccaaggctgccccctcggtcactctgttcccaccctcctctgaggagcttcaagccaacaaggccacactggtgtgtctcataagtgacttctacccggggccagtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacggaatgttca

SEQ ID NO: 471 - protein, 106 aa, Homo sapiens
GQPKAAPSVTLFPPSSEELQANKATLVCLISDFYPGPVTVAWKADSSPVKAGVETTTPSKQSNNKYAASSYLSLTPEQWKSHKSYSCQVTHEGSTVEKTVAPTECS

SEQ ID NO: 472 - DNA, 318 bp, Homo sapiens
  source 1..318: mol_type = other DNA
ggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 473 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 474 - DNA, 318 bp, Homo sapiens
  source 1..318: mol_type = other DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 475 - protein, 106 aa, Homo sapiens; identical to SEQ ID NO: 464

SEQ ID NO: 476 - DNA, 318 bp, Homo sapiens
  source 1..318: mol_type = other DNA
ggtcagcccaaggctgccccatcggtcactctgttcccgccctcctctgaggagcttcaagccaacaaggccacactggtgtgcctgatcagtgacttctacccgggagctgtgaaagtggcctggaaggcagatggcagccccgtcaacacgggagtggagaccaccacaccctccaaacagagcaacaacaagtacgcggccagcagctacctgagcctgacgcctgagcagtggaagtcccacagaagctacagctgccaggtcacgcatgaagggagcaccgtggagaagacagtggcccctgcagaatgttca

SEQ ID NO: 477 - protein, 106 aa, Homo sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 478 - DNA, 318 bp, Homo sapiens
  source 1..318: mol_type = other DNA
ggtcagcccaaggctgccccatcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 479 - protein, 106 aa, Homo sapiens
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 480 - DNA, 318 bp, Homo sapiens
  source 1..318: mol_type = other DNA
ggtcagcccaaggctgccccctcggtcactctgttcccaccctcctctgaggagcttcaagccaacaaggccacactggtgtgtctcgtaagtgacttcaacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 481 - protein, 106 aa, Homo sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 482 - DNA, 978 bp, Homo sapiens
  source 1..978: mol_type = other DNA
gcttctaccaagggacccagcgtgttccctctggctccttgctccagatccacctccgagtctacagctgctctgggctgcctggtcaaggactactttcctgagcctgtgaccgtgtcttggaactctggcgctctgacatctggcgtgcacacattccctgctgtgctgcagtcctccggcctgtactctctgtcctctgtcgtgaccgtgccttcctctagcctgggcaccaagacctacacctgtaatgtggaccacaagccttccaacaccaaggtggacaagcgcgtggaatctaagtacggccctccttgtcctccatgtcctgctccagagtttgaaggcggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgcctagctccatcgaaaagaccatctccaaggccaagggccagcctcgagaaccccaggtttacaccctgcctccaagccaagaggaaatgaccaagaaccaggtgtccctgacctgcctcgtgaagggattctacccctccgatatcgccgtggaatgggagtctaatggccagccagagaacaactacaagacaacccctcctgtgctggactccgacggctccttctttctgtattcccgcctgaccgtggacaagtccagatggcaagagggcaacgtgttctcctgcagcgtgatgcacgaggccctgcacaatcactacacccagaagtccctgtctctgtccctgggc

SEQ ID NO: 483 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 484 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 485 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

(19) United States
(12) Patent Application Publication
(10) Pub. No.: US 2025/0026816 A1
(43) Pub. Date: Jan. 23, 2025

(54) Antibodies for Use as Therapeutics Against Bacterial Infections

(71) Applicant: KYMAB LIMITED, Cambridge (GB)

(72) Inventors: Stephen REECE, Cambridge (GB); Aisha KRISHNA, Cambridge (GB); Siobhan O'LEARY, Cambridge (GB); Josefin BARTHOLDSON SCOTT, Cambridge (GB); Simon WATSON, Cambridge (GB); Stephen BAKER, Cambridge (GB)

(73) Assignee: KYMAB LIMITED, Cambridge (GB)

(21) Appl. No.: 18/713,540

(22) PCT Filed: Nov. 25, 2022
(86) PCT No.: PCT/EP2022/083353

§ 371 Date: May 24, 2024

(30) Foreign Application Priority Data
Nov. 26, 2021 (GB) 2117111.1

(51) Int. Cl. C07K 16/12; A61K 31/407; A61K 38/12; A61K 39/00

(52) U.S. Cl. CPC C07K 16/1218; A61K 31/407; A61K 38/12; A61K 2039/505; C07K 2317/33

(57) ABSTRACT

The present invention relates to antibodies that bind Acinetobacter baumannii, and their use in the diagnosis of, and the prevention and treatment of, bacterial infection caused by Acinetobacter baumannii. In particular, the present invention relates to monoclonal antibodies that specifically bind at the bacterial wall of Acinetobacter baumannii bacteria, such as on, e.g., living bacteria.

### 1. FIELD OF THE INVENTION

The present invention relates to antibodies that bind Acinetobacter baumannii, and their use in the diagnosis of, and the prevention and treatment of, bacterial infection caused by Acinetobacter baumannii.

### 2. BACKGROUND

The Acinetobacter genus consists of 26 named species and nine genomic species. Acinetobacter baumannii is a bacterial pathogen primarily associated with hospital-acquired infections. It is a gram-negative bacillus.

Acinetobacter baumannii specifically targets moist tissues such as mucous membranes or areas of the skin that are exposed. If left untreated, this infection can lead to septicemia and death. Once Acinetobacter baumannii is in a hospital environment, this poses a significant risk, particularly in ICU wards where patients are chronically ill.

Carbapenems are beta-lactam antibiotics that are structurally distinct from other commonly used beta-lactam antibiotics such as penicillin and cephalosporin classes. Carbapenems have proved the drug of choice to treat extended spectrum beta-lactamase (ESBL) producing bacteria, including A. baumannii strains, which are associated with poor clinical outcomes in severe infections in hospital settings such as pneumonia and bacteremia.

The World Health Organisation lists carbapenem-resistant Acinetobacter baumannii (CRAB) as one of the highest priority pathogens for threat to human health. Indeed, CRAB is a significant cause of ventilator associated pneumonia (VAP) in many countries in the developing and developed world and was found to cause greater than 30% of neonatal sepsis cases in some studies in the developing world.

Currently, Acinetobacter baumannii classifications comprise 3 European clones (ECI-III), 8 Worldwide clones (WW1-8-which have effectively become 8 International clones, IC1-8) and 2 Global clones (GC1 and 2). ECI=WW1=IC1, ECII=WW2=IC2 and ECIII=WW3=IC3 and all are defined by Multi-locus sequence typing (MLST), whereas Global clones 1 and 2 which are equal to IC1 and 2 respectively are also supported by full genome phylogenetics (Pathog Dis. 2014 August; 71(3):292-301. doi: 10.1111/2049-632X.12125. Epub 2014 Jan. 27 the contents of which are incorporated herein by reference).

Studies have indicated that GC2 is the predominant global lineage associated with carbapenem resistance.

The present invention aims to provide monoclonal antibodies for diagnosis and treatment of bacterial infection caused by Acinetobacter baumannii.

### 3. SUMMARY OF THE INVENTION

The present invention provides monoclonal antibodies suitable for diagnosis, prevention and treatment of bacterial infection caused by Acinetobacter baumannii. In particular, the present invention provides monoclonal antibodies that specifically bind at the bacterial wall of Acinetobacter baumannii bacteria, such as on, e.g., living bacteria. Advantageously, the present invention provides monoclonal antibodies that specifically bind at the bacterial wall of Acinetobacter baumannii bacteria and induce complement activation (e.g. as measured by flow cytometry-based assay).

The present invention identifies novel antibody targets at the surface of Acinetobacter baumannii bacteria for diagnosis, prevention and treatment of bacterial infection caused by Acinetobacter baumannii. In particular, the present invention demonstrates for the first time that the Oxa-23 enzyme is present at the surface of live bacteria and can be targeted by diagnostic and therapeutic antibodies. Further, the present invention demonstrates for the first time that the lipooligosaccharide (LOS) type OC1 at the surface of live bacteria and can be targeted by diagnostic and therapeutic antibodies. Finally, the present invention demonstrates for the first time that the KL49 carbohydrate capsular antigen at the surface of live bacteria can be targeted by diagnostic and therapeutic antibodies. The validation of these new targets at the surface of Acinetobacter baumannii bacteria provides an important advancement in the diagnosis, prevention and treatment of bacterial infection caused by Acinetobacter baumannii. Whilst not so limited, the present invention provides a particularly important advancement for the diagnosis, prevention and treatment of bacterial infection caused by carbapenem-resistant Acinetobacter baumannii (CRAB), where the existing carbapenem treatment will be ineffective. As set out above, CRAB is particularly associated with the G2 lineage of Acinetobacter baumannii.

The present invention further provides novel monoclonal antibodies that bind Oxa-23 at the surface of Acinetobacter baumannii bacteria. Such antibodies find particular use in the diagnosis of Acinetobacter baumannii. The present invention further provides novel antibodies that bind Oxa-23 at the surface of Acinetobacter baumannii bacteria and induce complement activation, thereby killing the bacteria. Such antibodies find particular use in the prevention and treatment of bacterial infection caused by Acinetobacter baumannii.

The present invention further provides novel monoclonal antibodies that bind OC1 LOS at the surface of Acinetobacter baumannii bacteria. Such antibodies find particular use in the diagnosis of Acinetobacter baumannii. The present invention further provides novel antibodies that bind OC1 LOS at the surface of Acinetobacter baumannii bacteria and induce complement activation, thereby killing the bacteria. Such antibodies find particular use in the prevention and treatment of bacterial infection caused by Acinetobacter baumannii.

The present invention further provides novel monoclonal antibodies that bind KL49 at the surface of Acinetobacter baumannii bacteria. Such antibodies find particular use in the diagnosis of Acinetobacter baumannii. The present invention further provides novel antibodies that bind KL49 at the surface of Acinetobacter baumannii bacteria and induce complement activation, thereby killing the bacteria. Such antibodies find particular use in the prevention and treatment of bacterial infection caused by Acinetobacter baumannii.

Antibody sequences are referred to herein by reference to the internal reference number of the relevant antibody, as well as by SEQ ID NOs. Any disclosure herein reciting antibody reference numbers is intended to include disclosure with the corresponding nucleic acid or amino acid sequence number (SEQ ID NO), as presented in Tables 1 and 2.

### 3.1 Anti-Oxa-23 Antibodies

Antibodies are provided herein which specifically bind Oxa-23. For example, antibodies 1348, 1349, 1540, 1548, and 1550, as exemplified herein have been found to specifically bind to Oxa-23 on outer membrane vesicles (OMVs) of Acinetobacter baumannii bacteria.

The present invention provides an antibody that specifically binds to Oxa-23 at the surface of Acinetobacter baumannii bacteria, such as on, e.g., living bacteria In one embodiment, the antibody specifically binds Oxa-23 at the surface of Acinetobacter baumannii bacteria as measured by an indirect whole bacterial cell ELISA. Due to high correlation between outer membrane vesicles (OMVs) of Acinetobacter baumannii bacteria and live Acinetobacter baumannii bacteria, such antibodies have been found to also specifically bind to Oxa-23 on outer membrane vesicles (OMVs) of Acinetobacter baumannii bacteria.

In one embodiment, the antibody induces complement activation (e.g. as measured by flow cytometry-based assay).

In one embodiment, the antibody specifically binds Oxa-23 at the surface of Acinetobacter baumannii bacteria and induces complement activation (e.g. as measured by flow cytometry-based assay), and HCDR3 is the HCDR3 of antibody 1348, 1349, 1540, 1548, or 1550.

In one embodiment, the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3,
wherein the HCDR3 is the HCDR3 of antibody 1348, 1349, 1540, 1548, or 1550.

In one embodiment, HCDR3 is the HCDR3 of antibody 1348.

In one embodiment, HCDR3 is the HCDR3 of antibody 1349.

In one embodiment, HCDR3 is the HCDR3 of antibody 1540.

In one embodiment, HCDR3 is the HCDR3 of antibody 1548.

In one embodiment, HCDR3 is the HCDR3 of antibody 1550.

In one embodiment, the present invention provides an anti-Oxa-23 antibody,
wherein the antibody specifically binds Oxa-23 at the surface of Acinetobacter baumannii bacteria and induces complement activation (e.g. as measured by flow cytometry-based assay), and
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the 6 CDRs are those of antibody 1348, 1349, 1540, 1548, or 1550.

In one embodiment, the present invention provides an anti-Oxa-23 antibody,
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the 6 CDRs are those of antibody 1348, 1349, 1540, 1548, or 1550.

In one embodiment, the antibody has the 6 CDRs of antibody 1348.

In one embodiment, the antibody has the 6 CDRs of antibody 1349.

In one embodiment, the antibody has the 6 CDRs of antibody 1540.

In one embodiment, the antibody has the 6 CDRs of antibody 1548.

In one embodiment, the antibody has the 6 CDRs of antibody 1550.

In one embodiment, the antibody specifically binds Oxa-23 at the surface of Acinetobacter baumannii bacteria and induces complement activation (e.g. as measured by flow cytometry-based assay), and
the variable heavy (VH) domain and variable light (VL) domain sequences respectively comprise the variable heavy (VH) domain and variable light (VL) domain sequences of antibody 1348, 1349, 1540, 1548, or 1550, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody 1348, 1349, 1540, 1548, or 1550, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence of antibody 1348, optionally with 1, 2, 3, 4 or 5 amino acid alterations outside the complementarity determining regions (CDRs), and a variable light (VL) domain sequence of antibody 1348, optionally with 1, 2, 3, 4 or 5 amino acid alterations outside the complementarity determining regions (CDRs).

In one embodiment, the antibody comprises a variable heavy (VH) domain sequence of antibody 1349, optionally with 1, 2, 3, 4 or 5 amino acid alterations outside the complementarity determining regions (CDRs), and a variable light (VL) domain sequence of antibody 1349, optionally with 1, 2, 3, 4 or 5 amino acid alterations outside the complementarity determining regions (CDRs).

In one embodiment, the antibody comprises a variable heavy (VH) domain sequence of antibody 1540, optionally with 1, 2, 3, 4 or 5 amino acid alterations outside the complementarity determining regions (CDRs), and a variable light (VL) domain sequence of antibody 1540, optionally with 1, 2, 3, 4 or 5 amino acid alterations outside the complementarity determining regions (CDRs).

In one embodiment, the antibody comprises a variable heavy (VH) domain sequence of antibody 1548, optionally with 1, 2, 3, 4 or 5 amino acid alterations outside the complementarity determining regions (CDRs), and a variable light (VL) domain sequence of antibody 1548, optionally with 1, 2, 3, 4 or 5 amino acid alterations outside the complementarity determining regions (CDRs).

In one embodiment, the antibody comprises a variable heavy (VH) domain sequence of antibody 1550, optionally with 1, 2, 3, 4 or 5 amino acid alterations outside the complementarity determining regions (CDRs), and a variable light (VL) domain sequence of antibody 1550, optionally with 1, 2, 3, 4 or 5 amino acid alterations outside the complementarity determining regions (CDRs).

In one embodiment, the antibody specifically binds Oxa-23 at the surface of Acinetobacter baumannii bacteria and induces complement activation (e.g. as measured by flow cytometry-based assay), and
the antibody comprises a variable heavy (VH) domain sequence and a variable light (VL) domain sequence and wherein the variable heavy (VH) domain and variable light (VL) domain sequences respectively comprise a sequence having at least 90%, at least 95%, at least 98%, or at least 99% identity to the variable heavy (VH) and variable light (VL) domain sequences of antibody 1348, 1349, 1540, 1548, or 1550,
provided that the antibody has the CDRs of antibody 1348, 1349, 1540, 1548, or 1550, respectively.

In one embodiment, the antibody comprises a variable heavy (VH) domain sequence and a variable light (VL) domain sequence and wherein the variable heavy (VH) domain and variable light (VL) domain sequences respectively comprise a sequence having at least 90%, at least 95%, at least 98%, or at least 99% identity to the variable heavy (VH) and variable light (VL) domain sequences of antibody 1348, 1349, 1540, 1548, or 1550
provided that the antibody has the CDRs of antibody 1348, 1349, 1540, 1548, or 1550, respectively.

In one embodiment, the variable heavy (VH) domain sequence comprises a sequence having at least 90%, at least 95%, at least 98%, or at least 99% identity to the VH domain sequence of antibody 1348 and the variable light (VL) domain sequence comprises a sequence having at least 90%, at least 95%, at least 98%, or at least 99% identity to the VL domain sequence of antibody 1348, provided that the antibody has the CDRs of antibody 1348.

In one embodiment, the variable heavy (VH) domain sequence comprises a sequence having at least 90%, at least 95%, at least 98%, or at least 99% identity to the VH domain sequence of antibody 1349 and the variable light (VL) domain sequence comprises a sequence having at least 90%, at least 95%, at least 98%, or at least 99% identity to the VL domain sequence of antibody 1349, provided that the antibody has the CDRs of antibody 1349.

In one embodiment, the variable heavy (VH) domain sequence comprises a sequence having at least 90%, at least 95%, at least 98%, or at least 99% identity to the VH domain sequence of antibody 1540 and the variable light (VL) domain sequence comprises a sequence having at least 90%, at least 95%, at least 98%, or at least 99% identity to the VL domain sequence of antibody 1540, provided that the antibody has the CDRs of antibody 1540.

In one embodiment, the variable heavy (VH) domain sequence comprises a sequence having at least 90%, at least 95%, at least 98%, or at least 99% identity to the VH domain sequence of antibody 1548 and the variable light (VL) domain sequence comprises a sequence having at least 90%, at least 95%, at least 98%, or at least 99% identity to the VL domain sequence of antibody 1548, provided that the antibody has the CDRs of antibody 1548.

In one embodiment, the variable heavy (VH) domain sequence comprises a sequence having at least 90%, at least 95%, at least 98%, or at least 99% identity to the VH domain sequence of antibody 1550 and the variable light (VL) domain sequence comprises a sequence having at least 90%, at least 95%, at least 98%, or at least 99% identity to the VL domain sequence of antibody 1550, provided that the antibody has the CDRs of antibody 1550.

In one embodiment, the present invention provides an antibody that specifically binds Oxa-23 at the surface of Acinetobacter baumannii bacteria,
wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody 1348, 1349, 1540, 1548, or 1550.

In one embodiment, the antibody specifically binds Oxa-23 at the surface of Acinetobacter baumannii bacteria, wherein the antibody comprises the VH and VL domain sequences of antibody 1348.

In one embodiment, the antibody specifically binds Oxa-23 at the surface of Acinetobacter baumannii bacteria, wherein the antibody comprises the VH and VL domain sequences of antibody 1349.

In one embodiment, the antibody specifically binds Oxa-23 at the surface of Acinetobacter baumannii bacteria, wherein the antibody comprises the VH and VL domain sequences of antibody 1540.

In one embodiment, the antibody specifically binds Oxa-23 at the surface of Acinetobacter baumannii bacteria, wherein the antibody comprises the VH and VL domain sequences of antibody 1548.

In one embodiment, the antibody specifically binds Oxa-23 at the surface of Acinetobacter baumannii bacteria, wherein the antibody comprises the VH and VL domain sequences of antibody 1550.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody 1348, 1349, 1540, 1548, or 1550.

In one embodiment, the antibody comprises the VH and VL domain sequences of antibody 1348.

In one embodiment, the antibody comprises the VH and VL domain sequences of antibody 1349.

In one embodiment, the antibody comprises the VH and VL domain sequences of antibody 1540.

In one embodiment, the antibody comprises the VH and VL domain sequences of antibody 1548.

In one embodiment, the antibody comprises the VH and VL domain sequences of antibody 1550.

In one example, the antibody is selected from the group consisting of antibodies 1348, 1349, 1540, 1548, or 1550.

In one embodiment, the antibody is antibody 1348.

In one embodiment, the antibody is antibody 1349.

In one embodiment, the antibody is antibody 1540.

In one embodiment, the antibody is antibody 1548.

In one embodiment, the antibody is antibody 1550.

Antibodies are provided which bind to the same epitope on Oxa-23 as an antibody described anywhere herein.

An antibody is provided which binds to the same epitope as antibody 1348, 1349, 1540, 1548, or 1550, e.g. as defined by its VH and VL sequences.

In one embodiment, an antibody is provided which binds to the same epitope as antibody 1348, e.g. as defined by its VH and VL sequences.

In one embodiment, an antibody is provided which binds to the same epitope as antibody 1349, e.g. as defined by its VH and VL sequences.

In one embodiment, an antibody is provided which binds to the same epitope as antibody 1540, e.g. as defined by its VH and VL sequences.

In one embodiment, an antibody is provided which binds to the same epitope as antibody 1548, e.g. as defined by its VH and VL sequences.

In one embodiment, an antibody is provided which binds to the same epitope as antibody 1550, e.g. as defined by its VH and VL sequences.

An antibody may contact Oxa-23 with a footprint that fully or partly overlaps with that of an antibody disclosed anywhere herein. As described elsewhere herein, competition between antibodies may be determined, for example using SPR, and antibodies are provided which compete for binding to Oxa-23 (compete for binding to their epitope) with an IgG antibody as described anywhere herein.

An antibody of the present invention may be one which competes for binding to Oxa-23 with any anti-Oxa-23 antibody described herein, such as antibodies 1348, 1349, 1540, 1548, and 1550, e.g. as defined by their respective VH and VL sequences.

An antibody of the present invention may be one which competes for binding to Oxa-23 with antibody 1348.

An antibody of the present invention may be one which competes for binding to Oxa-23 with antibody 1349.

An antibody of the present invention may be one which competes for binding to Oxa-23 with antibody 1540.

An antibody of the present invention may be one which competes for binding to Oxa-23 with antibody 1548.

An antibody of the present invention may be one which competes for binding to Oxa-23 with antibody 1550.

A nucleic acid sequence provided by the invention may comprise a sequence that encodes a VH domain and/or an VL domain of an anti-Oxa-23 antibody as defined anywhere herein.

The nucleic acid may comprise a sequence that encodes a VH domain and/or a VL domain of antibody 1348.

The nucleic acid may comprise a sequence that encodes a VH domain and/or a VL domain of antibody 1349.

The nucleic acid may comprise a sequence that encodes a VH domain and/or a VL domain of antibody 1540.

The nucleic acid may comprise a sequence that encodes a VH domain and/or a VL domain of antibody 1548.

The nucleic acid may comprise a sequence that encodes a VH domain and/or a VL domain of antibody 1550.

### 3.2 Anti-OC1 LOS Antibodies

Antibodies are provided herein which specifically bind OC1 lipooligosaccharide (LOS). For example, antibodies 1042, 1043, 1403, 1405, 1407, 1408, and 1413 as exemplified herein have been found to specifically bind to OC1 LOS at the surface of Acinetobacter baumannii bacteria.

The present invention provides an antibody that specifically binds to OC1 LOS at the surface of Acinetobacter baumannii bacteria, such as on, e.g., living bacteria In one embodiment, the antibody specifically binds OC1 LOS at the surface of Acinetobacter baumannii bacteria as measured by an indirect whole bacterial cell ELISA. Due to high correlation between outer membrane vesicles (OMVs) of Acinetobacter baumannii bacteria and live Acinetobacter baumannii bacteria, such antibodies have been found to also specifically bind to OC1 LOS on outer membrane vesicles (OMVs) of Acinetobacter baumannii bacteria.

In one embodiment, the antibody induces complement activation (e.g. as measured by flow cytometry-based assay).

In one embodiment, the antibody specifically binds OC1 LOS at the surface of Acinetobacter baumannii bacteria and induces complement activation (e.g. as measured by flow cytometry-based assay), and HCDR3 is the HCDR3 of antibody 1042, 1043, 1403, 1405, 1407, 1408, or 1413.

In one embodiment, the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3,
wherein the HCDR3 is the HCDR3 of antibody 1042, 1043, 1403, 1405, 1407, 1408, or 1413.

In one embodiment, HCDR3 is the HCDR3 of antibody 1042.

In one embodiment, HCDR3 is the HCDR3 of antibody 1043.

In one embodiment, HCDR3 is the HCDR3 of antibody 1403.

In one embodiment, HCDR3 is the HCDR3 of antibody 1405.

In one embodiment, HCDR3 is the HCDR3 of antibody 1407.

In one embodiment, HCDR3 is the HCDR3 of antibody 1408.

In one embodiment, HCDR3 is the HCDR3 of antibody 1413.

In one embodiment, the present invention provides an anti-OC1 LOS antibody,
wherein the antibody specifically binds OC1 LOS at the surface of Acinetobacter baumannii bacteria and induces complement activation (e.g. as measured by flow cytometry-based assay), and
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the 6 CDRs are those of antibody 1042, 1043, 1403, 1405, 1407, 1408, or 1413.

In one embodiment, the present invention provides an anti-OC1 LOS antibody,
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the 6 CDRs are those of antibody 1042, 1043, 1403, 1405, 1407, 1408, or 1413.

In one embodiment, the antibody has the 6 CDRs of antibody 1042.

In one embodiment, the antibody has the 6 CDRs of antibody 1043.

In one embodiment, the antibody has the 6 CDRs of antibody 1403.

In one embodiment, the antibody has the 6 CDRs of antibody 1405.

In one embodiment, the antibody has the 6 CDRs of antibody 1407.

In one embodiment, the antibody has the 6 CDRs of antibody 1408.

In one embodiment, the antibody has the 6 CDRs of antibody 1413.

In one embodiment, the antibody specifically binds OC1 LOS at the surface of Acinetobacter baumannii bacteria and induces complement activation (e.g. as measured by flow cytometry-based assay), and
the variable heavy (VH) domain and variable light (VL) domain sequences respectively comprise the variable heavy (VH) domain and variable light (VL) domain sequences of antibody 1042, 1043, 1403, 1405, 1407, 1408, or 1413, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody 1042, 1043, 1403, 1405, 1407, 1408, or 1413, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence of antibody 1042, optionally with 1, 2, 3, 4 or 5 amino acid alterations outside the complementarity determining regions (CDRs), and a variable light (VL) domain sequence of antibody 1042, optionally with 1, 2, 3, 4 or 5 amino acid alterations outside the complementarity determining regions (CDRs).

In one embodiment, the antibody comprises a variable heavy (VH) domain sequence of antibody 1043, optionally with 1, 2, 3, 4 or 5 amino acid alterations outside the complementarity determining regions (CDRs), and a variable light (VL) domain sequence of antibody 1043, optionally with 1, 2, 3, 4 or 5 amino acid alterations outside the complementarity determining regions (CDRs).

In one embodiment, the antibody comprises a variable heavy (VH) domain sequence of antibody 1403, optionally with 1, 2, 3, 4 or 5 amino acid alterations outside the complementarity determining regions (CDRs), and a variable light (VL) domain sequence of antibody 1403, optionally with 1, 2, 3, 4 or 5 amino acid alterations outside the complementarity determining regions (CDRs).

In one embodiment, the antibody comprises a variable heavy (VH) domain sequence of antibody 1405, optionally with 1, 2, 3, 4 or 5 amino acid alterations outside the complementarity determining regions (CDRs), and a variable light (VL) domain sequence of antibody 1405, optionally with 1, 2, 3, 4 or 5 amino acid alterations outside the complementarity determining regions (CDRs).

In one embodiment, the antibody comprises a variable heavy (VH) domain sequence of antibody 1407, optionally with 1, 2, 3, 4 or 5 amino acid alterations outside the complementarity determining regions (CDRs), and a variable light (VL) domain sequence of antibody 1407, optionally with 1, 2, 3, 4 or 5 amino acid alterations outside the complementarity determining regions (CDRs).

In one embodiment, the antibody comprises a variable heavy (VH) domain sequence of antibody 1408, optionally with 1, 2, 3, 4 or 5 amino acid alterations outside the complementarity determining regions (CDRs), and a variable light (VL) domain sequence of antibody 1408, optionally with 1, 2, 3, 4 or 5 amino acid alterations outside the complementarity determining regions (CDRs).

In one embodiment, the antibody comprises a variable heavy (VH) domain sequence of antibody 1413, optionally with 1, 2, 3, 4 or 5 amino acid alterations outside the complementarity determining regions (CDRs), and a variable light (VL) domain sequence of antibody 1413, optionally with 1, 2, 3, 4 or 5 amino acid alterations outside the complementarity determining regions (CDRs).

In one embodiment, the antibody specifically binds OC1 LOS at the surface of Acinetobacter baumannii and induces complement activation (e.g. as measured by flow cytometry-based assay), and
the antibody comprises a variable heavy (VH) domain sequence and a variable light (VL) domain sequence and wherein the variable heavy (VH) domain and variable light (VL) domain sequences respectively comprise a sequence having at least 90%, at least 95%, at least 98%, or at least 99% identity to the variable heavy (VH) and variable light (VL) domain sequences of antibody 1042, 1043, 1403, 1405, 1407, 1408, or 1413,
provided that the antibody has the 6 CDRs of antibody 1042, 1043, 1403, 1405, 1407, 1408, or 1413, respectively.

In one embodiment, the antibody comprises a variable heavy (VH) domain sequence and a variable light (VL) domain sequence and wherein the variable heavy (VH) domain and variable light (VL) domain sequences respectively comprise a sequence having at least 90%, at least 95%, at least 98%, or at least 99% identity to the variable heavy (VH) and variable light (VL) domain sequences of antibody 1042, 1043, 1403, 1405, 1407, 1408, or 1413
provided that the antibody has the 6 CDRs of antibody 1042, 1043, 1403, 1405, 1407, 1408, or 1413, respectively.

In one embodiment, the variable heavy (VH) domain sequence comprises a sequence having at least 90%, at least 95%, at least 98%, or at least 99% identity to the VH domain sequence of antibody 1042 and the variable light (VL) domain sequence comprises a sequence having at least 90%, at least 95%, at least 98%, or at least 99% identity to the VL domain sequence of antibody 1042, provided that the antibody has the 6 CDRs of antibody 1042.

In one embodiment, the variable heavy (VH) domain sequence comprises a sequence having at least 90%, at least 95%, at least 98%, or at least 99% identity to the VH domain sequence of antibody 1043 and the variable light (VL) domain sequence comprises a sequence having at least 90%, at least 95%, at least 98%, or at least 99% identity to the VL domain sequence of antibody 1043, provided that the antibody has the 6 CDRs of antibody 1043.

In one embodiment, the variable heavy (VH) domain sequence comprises a sequence having at least 90%, at least 95%, at least 98%, or at least 99% identity to the VH domain sequence of antibody 1403 and the variable light (VL) domain sequence comprises a sequence having at least 90%, at least 95%, at least 98%, or at least 99% identity to the VL domain sequence of antibody 1403, provided that the antibody has the 6 CDRs of antibody 1403.

In one embodiment, the variable heavy (VH) domain sequence comprises a sequence having at least 90%, at least 95%, at least 98%, or at least 99% identity to the VH domain sequence of antibody 1405 and the variable light (VL) domain sequence comprises a sequence having at least 90%, at least 95%, at least 98%, or at least 99% identity to the VL domain sequence of antibody 1405, provided that the antibody has the 6 CDRs of antibody 1405.

In one embodiment, the variable heavy (VH) domain sequence comprises a sequence having at least 90%, at least 95%, at least 98%, or at least 99% identity to the VH domain sequence of antibody 1407 and the variable light (VL) domain sequence comprises a sequence having at least 90%, at least 95%, at least 98%, or at least 99% identity to the VL domain sequence of antibody 1407, provided that the antibody has the 6 CDRs of antibody 1407.

In one embodiment, the variable heavy (VH) domain sequence comprises a sequence having at least 90%, at least 95%, at least 98%, or at least 99% identity to the VH domain sequence of antibody 1408 and the variable light (VL) domain sequence comprises a sequence having at least 90%, at least 95%, at least 98%, or at least 99% identity to the VL domain sequence of antibody 1408, provided that the antibody has the 6 CDRs of antibody 1408.

In one embodiment, the variable heavy (VH) domain sequence comprises a sequence having at least 90%, at least 95%, at least 98%, or at least 99% identity to the VH domain sequence of antibody 1413 and the variable light (VL) domain sequence comprises a sequence having at least 90%, at least 95%, at least 98%, or at least 99% identity to the VL domain sequence of antibody 1413, provided that the antibody has the 6 CDRs of antibody 1413.

In one embodiment, the present invention provides an antibody that specifically binds OC1 LOS at the surface of Acinetobacter baumannii bacteria,
wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody 1042, 1043, 1403, 1405, 1407, 1408, or 1413.

In one embodiment, the antibody specifically binds OC1 LOS at the surface of Acinetobacter baumannii bacteria, wherein the antibody comprises the VH and VL domain sequences of antibody 1042.

In one embodiment, the antibody specifically binds OC1 LOS at the surface of Acinetobacter baumannii bacteria, wherein the antibody comprises the VH and VL domain sequences of antibody 1043.

In one embodiment, the antibody specifically binds OC1 LOS at the surface of Acinetobacter baumannii bacteria, wherein the antibody comprises the VH and VL domain sequences of antibody 1403.

In one embodiment, the antibody specifically binds OC1 LOS at the surface of Acinetobacter baumannii bacteria, wherein the antibody comprises the VH and VL domain sequences of antibody 1405.

In one embodiment, the antibody specifically binds OC1 LOS at the surface of Acinetobacter baumannii bacteria, wherein the antibody comprises the VH and VL domain sequences of antibody 1407.

In one embodiment, the antibody specifically binds OC1 LOS at the surface of Acinetobacter baumannii bacteria, wherein the antibody comprises the VH and VL domain sequences of antibody 1408.

In one embodiment, the antibody specifically binds OC1 LOS at the surface of Acinetobacter baumannii bacteria, wherein the antibody comprises the VH and VL domain sequences of antibody 1413.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody 1042, 1043, 1403, 1405, 1407, 1408, or 1413.

In one embodiment, the antibody comprises the VH and VL domain sequences of antibody 1042.

In one embodiment, the antibody comprises the VH and VL domain sequences of antibody 1043.

In one embodiment, the antibody comprises the VH and VL domain sequences of antibody 1403.

In one embodiment, the antibody comprises the VH and VL domain sequences of antibody 1405.

In one embodiment, the antibody comprises the VH and VL domain sequences of antibody 1407.

In one embodiment, the antibody comprises the VH and VL domain sequences of antibody 1408.

In one embodiment, the antibody comprises the VH and VL domain sequences of antibody 1413.

In one example, the antibody is selected from the group consisting of antibodies 1042, 1043, 1403, 1405, 1407, 1408, and 1413.

In one embodiment, the antibody is antibody 1042.

In one embodiment, the antibody is antibody 1043.

In one embodiment, the antibody is antibody 1403.

In one embodiment, the antibody is antibody 1405.

In one embodiment, the antibody is antibody 1407.

In one embodiment, the antibody is antibody 1408.

In one embodiment, the antibody is antibody 1413.

Antibodies are provided which bind to the same epitope on OC1 LOS as an antibody described anywhere herein.

An antibody is provided which bind to the same epitope as antibody 1042, 1043, 1403, 1405, 1407, 1408, or 1413, e.g. as defined by its VH and VL sequences.

In one embodiment, an antibody is provided which binds to the same epitope as antibody 1042.

In one embodiment, an antibody is provided which binds to the same epitope as antibody 1043.

In one embodiment, an antibody is provided which binds to the same epitope as antibody 1403.

In one embodiment, an antibody is provided which binds to the same epitope as antibody 1405.

In one embodiment, an antibody is provided which binds to the same epitope as antibody 1407.

In one embodiment, an antibody is provided which binds to the same epitope as antibody 1408.

In one embodiment, an antibody is provided which binds to the same epitope as antibody 1413.

An antibody may contact OC1 LOS with a footprint that fully or partly overlaps with that of an antibody disclosed anywhere herein. As described elsewhere herein, competition between antibodies may be determined, for example using SPR, and antibodies are provided which compete for binding to OC1 LOS (compete for binding to their epitope) with an IgG antibody as described anywhere herein.

An antibody of the present invention may be one which competes for binding to OC1 LOS at the surface of Acinetobacter baumannii bacteria with any anti-OC1 LOS antibody described herein, such as antibodies 1042, 1043, 1403, 1405, 1407, 1408, and 1413, e.g. as defined by its VH and VL sequences.

An antibody of the present invention may be one which competes for binding to OC1 LOS with antibody 1042.

An antibody of the present invention may be one which competes for binding to OC1 LOS with antibody 1043.

An antibody of the present invention may be one which competes for binding to OC1 LOS with antibody 1403.

An antibody of the present invention may be one which competes for binding to OC1 LOS with antibody 1405.

An antibody of the present invention may be one which competes for binding to OC1 LOS with antibody 1407.

An antibody of the present invention may be one which competes for binding to OC1 LOS with antibody 1408.

An antibody of the present invention may be one which competes for binding to OC1 LOS with antibody 1413.

A nucleic acid sequence provided by the invention may comprise a sequence that encodes a VH domain and/or an VL domain of an anti-OC1 LOS antibody as defined anywhere herein.

The nucleic acid may comprise a sequence that encodes a VH domain and/or a VL domain of antibody 1042.

The nucleic acid may comprise a sequence that encodes a VH domain and/or a VL domain of antibody 1043.

The nucleic acid may comprise a sequence that encodes a VH domain and/or a VL domain of antibody 1403.

The nucleic acid may comprise a sequence that encodes a VH domain and/or a VL domain of antibody 1405.

The nucleic acid may comprise a sequence that encodes a VH domain and/or a VL domain of antibody 1407.

The nucleic acid may comprise a sequence that encodes a VH domain and/or a VL domain of antibody 1408.

The nucleic acid may comprise a sequence that encodes a VH domain and/or a VL domain of antibody 1413.

### 3.3 Anti-KL49 Antibodies

Antibodies are provided herein which specifically bind KL49. For example, antibodies 1345, 1347, 1350, 1351, 1363, 1364, 1397, 1398, 1400, 1401, 1402, 1404, 1409, 1410, 1412, 1414, 1415, and 1416 as exemplified herein have been found to specifically bind KL49 at the surface of Acinetobacter baumannii bacteria.

The present invention provides an antibody that specifically binds to KL49 at the surface of Acinetobacter baumannii bacteria, such as on, e.g., living bacteria In one embodiment, the antibody specifically binds KL49 at the surface of Acinetobacter baumannii bacteria as measured by an indirect whole bacterial cell ELISA. Due to high correlation between outer membrane vesicles (OMVs) of Acinetobacter baumannii bacteria and live Acinetobacter baumannii bacteria, such antibodies have been found to also specifically bind to KL49 on outer membrane vesicles (OMVs) of Acinetobacter baumannii bacteria.

In one embodiment, the antibody induces complement activation (e.g. as measured by flow cytometry-based assay).

In one embodiment, the antibody specifically binds KL49 at the surface of Acinetobacter baumannii and induces complement activation (e.g. as measured by flow cytometry-based assay), and HCDR3 is the HCDR3 of antibody 1345, 1347, 1350, 1351, 1363, 1364, 1397, 1398, 1400, 1401, 1402, 1404, 1409, 1410, 1412, 1414, 1415, or 1416.

In one embodiment, the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3,
wherein the HCDR3 is the HCDR3 of antibody 1345, 1347, 1350, 1351, 1363, 1364, 1397, 1398, 1400, 1401, 1402, 1404, 1409, 1410, 1412, 1414, 1415, or 1416.

In one embodiment, HCDR3 is the HCDR3 of antibody1345.

In one embodiment, HCDR3 is the HCDR3 of antibody1347.

In one embodiment, HCDR3 is the HCDR3 of antibody1350.

In one embodiment, HCDR3 is the HCDR3 of antibody1351.

In one embodiment, HCDR3 is the HCDR3 of antibody1363.

In one embodiment, HCDR3 is the HCDR3 of antibody1364.

In one embodiment, HCDR3 is the HCDR3 of antibody1397.

In one embodiment, HCDR3 is the HCDR3 of antibody1398.

In one embodiment, HCDR3 is the HCDR3 of antibody1400.

In one embodiment, HCDR3 is the HCDR3 of antibody1401.

In one embodiment, HCDR3 is the HCDR3 of antibody1402.

In one embodiment, HCDR3 is the HCDR3 of antibody1404.

In one embodiment, HCDR3 is the HCDR3 of antibody1409.

In one embodiment, HCDR3 is the HCDR3 of antibody1410.

In one embodiment, HCDR3 is the HCDR3 of antibody1412.

In one embodiment, HCDR3 is the HCDR3 of antibody1414.

In one embodiment, HCDR3 is the HCDR3 of antibody1415.

In one embodiment, HCDR3 is the HCDR3 of antibody1416.

In one embodiment, the present invention provides an anti-KL49 antibody,
wherein the antibody specifically binds KL49 at the surface of Acinetobacter baumannii bacteria as and induces complement activation (e.g. as measured by flow cytometry-based assay), and
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the 6 CDRs are those of antibody 1345, 1347, 1350, 1351, 1363, 1364, 1397, 1398, 1400, 1401, 1402, 1404, 1409, 1410, 1412, 1414, 1415, or 1416.

In one embodiment, the present invention provides an anti-KL49 antibody,
wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3,
wherein the 6 CDRs are those of antibody 1345, 1347, 1350, 1351, 1363, 1364, 1397, 1398, 1400, 1401, 1402, 1404, 1409, 1410, 1412, 1414, 1415, or 1416.

In one embodiment, the antibody has the 6 CDRs of antibody1345.

In one embodiment, the antibody has the 6 CDRs of antibody1347.

In one embodiment, the antibody has the 6 CDRs of antibody1350.

In one embodiment, the antibody has the 6 CDRs of antibody1351.

In one embodiment, the antibody has the 6 CDRs of antibody1363.

In one embodiment, the antibody has the 6 CDRs of antibody1364.

In one embodiment, the antibody has the 6 CDRs of antibody1397.

In one embodiment, the antibody has the 6 CDRs of antibody1398.

In one embodiment, the antibody has the 6 CDRs of antibody1400.

In one embodiment, the antibody has the 6 CDRs of antibody1401.

In one embodiment, the antibody has the 6 CDRs of antibody1402.

In one embodiment, the antibody has the 6 CDRs of antibody1404.

In one embodiment, the antibody has the 6 CDRs of antibody1409.

In one embodiment, the antibody has the 6 CDRs of antibody1410.

In one embodiment, the antibody has the 6 CDRs of antibody1412.

In one embodiment, the antibody has the 6 CDRs of antibody1414.

In one embodiment, the antibody has the 6 CDRs of antibody1415.

In one embodiment, the antibody has the 6 CDRs of antibody1416.

In one embodiment, the antibody specifically binds KL49 at the surface of Acinetobacter baumannii bacteria and induces complement activation (e.g. as measured by flow cytometry-based assay), and
the variable heavy (VH) domain and variable light (VL) domain sequences respectively comprise the variable heavy (VH) domain and variable light (VL) domain sequences of antibody 1345, 1347, 1350, 1351, 1363, 1364, 1397, 1398, 1400, 1401, 1402, 1404, 1409, 1410, 1412, 1414, 1415, or 1416, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody 1345, 1347, 1350, 1351, 1363, 1364, 1397, 1398, 1400, 1401, 1402, 1404, 1409, 1410, 1412, 1414, 1415, or 1416, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

In one embodiment, the antibody comprises a variable heavy (VH) domain sequence of antibody 1345, optionally with 1, 2, 3, 4 or 5 amino acid alterations outside the complementarity determining regions (CDRs), and a variable light (VL) domain sequence of antibody 1345, optionally with 1, 2, 3, 4 or 5 amino acid alterations outside the complementarity determining regions (CDRs).

In one embodiment, the antibody comprises a variable heavy (VH) domain sequence of antibody 1347, optionally with 1, 2, 3, 4 or 5 amino acid alterations outside the complementarity determining regions (CDRs), and a variable light (VL) domain sequence of antibody 1347, optionally with 1, 2, 3, 4 or 5 amino acid alterations outside the complementarity determining regions (CDRs).

In one embodiment, the antibody comprises a variable heavy (VH) domain sequence of antibody 1350, optionally with 1, 2, 3, 4 or 5 amino acid alterations outside the complementarity determining regions (CDRs), and a variable light (VL) domain sequence of antibody 1350, optionally with 1, 2, 3, 4 or 5 amino acid alterations outside the complementarity determining regions (CDRs).

In one embodiment, the antibody comprises a variable heavy (VH) domain sequence of antibody 1351, optionally with 1, 2, 3, 4 or 5 amino acid alterations outside the complementarity determining regions (CDRs), and a variable light (VL) domain sequence of antibody 1351, optionally with 1, 2, 3, 4 or 5 amino acid alterations outside the complementarity determining regions (CDRs).

In one embodiment, the antibody comprises a variable heavy (VH) domain sequence of antibody 1363, optionally with 1, 2, 3, 4 or 5 amino acid alterations outside the complementarity determining regions (CDRs), and a variable light (VL) domain sequence of antibody 1363, optionally with 1, 2, 3, 4 or 5 amino acid alterations outside the complementarity determining regions (CDRs).

In one embodiment, the antibody comprises a variable heavy (VH) domain sequence of antibody 1364, optionally with 1, 2, 3, 4 or 5 amino acid alterations outside the complementarity determining regions (CDRs), and a variable light (VL) domain sequence of antibody 1364, optionally with 1, 2, 3, 4 or 5 amino acid alterations outside the complementarity determining regions (CDRs).

In one embodiment, the antibody comprises a variable heavy (VH) domain sequence of antibody 1397, optionally with 1, 2, 3, 4 or 5 amino acid alterations outside the complementarity determining regions (CDRs), and a variable light (VL) domain sequence of antibody 1397, optionally with 1, 2, 3, 4 or 5 amino acid alterations outside the complementarity determining regions (CDRs).

In one embodiment, the antibody comprises a variable heavy (VH) domain sequence of antibody 1398, optionally with 1, 2, 3, 4 or 5 amino acid alterations outside the complementarity determining regions (CDRs), and a variable light (VL) domain sequence of antibody 1398, optionally with 1, 2, 3, 4 or 5 amino acid alterations outside the complementarity determining regions (CDRs).

In one embodiment, the antibody comprises a variable heavy (VH) domain sequence of antibody 1400, optionally with 1, 2, 3, 4 or 5 amino acid alterations outside the complementarity determining regions (CDRs), and a variable light (VL) domain sequence of antibody 1400, optionally with 1, 2, 3, 4 or 5 amino acid alterations outside the complementarity determining regions (CDRs).

In one embodiment, the antibody comprises a variable heavy (VH) domain sequence of antibody 1401, optionally with 1, 2, 3, 4 or 5 amino acid alterations outside the complementarity determining regions (CDRs), and a variable light (VL) domain sequence of antibody 1401, optionally with 1, 2, 3, 4 or 5 amino acid alterations outside the complementarity determining regions (CDRs).

In one embodiment, the antibody comprises a variable heavy (VH) domain sequence of antibody 1402, optionally with 1, 2, 3, 4 or 5 amino acid alterations outside the complementarity determining regions (CDRs), and a variable light (VL) domain sequence of antibody 1402, optionally with 1, 2, 3, 4 or 5 amino acid alterations outside the complementarity determining regions (CDRs).

In one embodiment, the antibody comprises a variable heavy (VH) domain sequence of antibody 1404, optionally with 1, 2, 3, 4 or 5 amino acid alterations outside the complementarity determining regions (CDRs), and a variable light (VL) domain sequence of antibody 1404, optionally with 1, 2, 3, 4 or 5 amino acid alterations outside the complementarity determining regions (CDRs).

In one embodiment, the antibody comprises a variable heavy (VH) domain sequence of antibody 1409, optionally with 1, 2, 3, 4 or 5 amino acid alterations outside the complementarity determining regions (CDRs), and a variable light (VL) domain sequence of antibody 1409, optionally with 1, 2, 3, 4 or 5 amino acid alterations outside the complementarity determining regions (CDRs).

In one embodiment, the antibody comprises a variable heavy (VH) domain sequence of antibody 1410, optionally with 1, 2, 3, 4 or 5 amino acid alterations outside the complementarity determining regions (CDRs), and a variable light (VL) domain sequence of antibody 1410, optionally with 1, 2, 3, 4 or 5 amino acid alterations outside the complementarity determining regions (CDRs).

In one embodiment, the antibody comprises a variable heavy (VH) domain sequence of antibody 1412, optionally with 1, 2, 3, 4 or 5 amino acid alterations outside the complementarity determining regions (CDRs), and a variable light (VL) domain sequence of antibody 1412, optionally with 1, 2, 3, 4 or 5 amino acid alterations outside the complementarity determining regions (CDRs).

In one embodiment, the antibody comprises a variable heavy (VH) domain sequence of antibody 1414, optionally with 1, 2, 3, 4 or 5 amino acid alterations outside the complementarity determining regions (CDRs), and a variable light (VL) domain sequence of antibody 1414, optionally with 1, 2, 3, 4 or 5 amino acid alterations outside the complementarity determining regions (CDRs).

In one embodiment, the antibody comprises a variable heavy (VH) domain sequence of antibody 1415, optionally with 1, 2, 3, 4 or 5 amino acid alterations outside the complementarity determining regions (CDRs), and a variable light (VL) domain sequence of antibody 1415, optionally with 1, 2, 3, 4 or 5 amino acid alterations outside the complementarity determining regions (CDRs).

In one embodiment, the antibody comprises a variable heavy (VH) domain sequence of antibody 1416, optionally with 1, 2, 3, 4 or 5 amino acid alterations outside the complementarity determining regions (CDRs), and a variable light (VL) domain sequence of antibody 1416, optionally with 1, 2, 3, 4 or 5 amino acid alterations outside the complementarity determining regions (CDRs).

In one embodiment, the antibody specifically binds KL49 at the surface of Acinetobacter baumannii bacteria and induces complement activation (e.g. as measured by flow cytometry-based assay), and
the antibody comprises a variable heavy (VH) domain sequence and a variable light (VL) domain sequence and wherein the variable heavy (VH) domain and variable light (VL) domain sequences respectively comprise a sequence having at least 90%, at least 95%, at least 98%, or at least 99% identity to the variable heavy (VH) and variable light (VL) domain sequences of antibody 1345, 1347, 1350, 1351, 1363, 1364, 1397, 1398, 1400, 1401, 1402, 1404, 1409, 1410, 1412, 1414, 1415, or 1416,
provided that the antibody has the 6 CDRs of antibody 1345, 1347, 1350, 1351, 1363, 1364, 1397, 1398, 1400, 1401, 1402, 1404, 1409, 1410, 1412, 1414, 1415, or 1416, respectively.

In one embodiment, the antibody comprises a variable heavy (VH) domain sequence and a variable light (VL) domain sequence and wherein the variable heavy (VH) domain and variable light (VL) domain sequences respectively comprise a sequence having at least 90%, at least 95%, at least 98%, or at least 99% identity to the variable heavy (VH) and variable light (VL) domain sequences of antibody 1345, 1347, 1350, 1351, 1363, 1364, 1397, 1398, 1400, 1401, 1402, 1404, 1409, 1410, 1412, 1414, 1415, or 1416
provided that the antibody has the 6 CDRs of antibody 1345, 1347, 1350, 1351, 1363, 1364, 1397, 1398, 1400, 1401, 1402, 1404, 1409, 1410, 1412, 1414, 1415, or 1416, respectively.

In one embodiment, the variable heavy (VH) domain sequence comprises a sequence having at least 90%, at least 95%, at least 98%, or at least 99% identity to the VH domain sequence of antibody 1345 and the variable light (VL) domain sequence comprises a sequence having at least 90%, at least 95%, at least 98%, or at least 99% identity to the VL domain sequence of antibody 1345, provided that the antibody has the 6 CDRs of antibody 1345.

In one embodiment, the variable heavy (VH) domain sequence comprises a sequence having at least 90%, at least 95%, at least 98%, or at least 99% identity to the VH domain sequence of antibody 1347 and the variable light (VL) domain sequence comprises a sequence having at least 90%, at least 95%, at least 98%, or at least 99% identity to the VL domain sequence of antibody 1347, provided that the antibody has the 6 CDRs of antibody 1347.

In one embodiment, the variable heavy (VH) domain sequence comprises a sequence having at least 90%, at least 95%, at least 98%, or at least 99% identity to the VH domain sequence of antibody 1350 and the variable light (VL) domain sequence comprises a sequence having at least 90%, at least 95%, at least 98%, or at least 99% identity to the VL domain sequence of antibody 1350, provided that the antibody has the 6 CDRs of antibody 1350.

In one embodiment, the variable heavy (VH) domain sequence comprises a sequence having at least 90%, at least 95%, at least 98%, or at least 99% identity to the VH domain sequence of antibody 1351 and the variable light (VL) domain sequence comprises a sequence having at least 90%, at least 95%, at least 98%, or at least 99% identity to the VL domain sequence of antibody 1351, provided that the antibody has the 6 CDRs of antibody 1351.

In one embodiment, the variable heavy (VH) domain sequence comprises a sequence having at least 90%, at least 95%, at least 98%, or at least 99% identity to the VH domain sequence of antibody 1363 and the variable light (VL) domain sequence comprises a sequence having at least 90%, at least 95%, at least 98%, or at least 99% identity to the VL domain sequence of antibody 1363, provided that the antibody has the 6 CDRs of antibody 1363.

In one embodiment, the variable heavy (VH) domain sequence comprises a sequence having at least 90%, at least 95%, at least 98%, or at least 99% identity to the VH domain sequence of antibody 1364 and the variable light (VL) domain sequence comprises a sequence having at least 90%, at least 95%, at least 98%, or at least 99% identity to the VL domain sequence of antibody 1364, provided that the antibody has the 6 CDRs of antibody 1364.

In one embodiment, the variable heavy (VH) domain sequence comprises a sequence having at least 90%, at least 95%, at least 98%, or at least 99% identity to the VH domain sequence of antibody 1397 and the variable light (VL) domain sequence comprises a sequence having at least 90%, at least 95%, at least 98%, or at least 99% identity to the VL domain sequence of antibody 1397, provided that the antibody has the 6 CDRs of antibody 1397.

In one embodiment, the variable heavy (VH) domain sequence comprises a sequence having at least 90%, at least 95%, at least 98%, or at least 99% identity to the VH domain sequence of antibody 1398 and the variable light (VL) domain sequence comprises a sequence having at least 90%, at least 95%, at least 98%, or at least 99% identity to the VL domain sequence of antibody 1398, provided that the antibody has the 6 CDRs of antibody 1398.

In one embodiment, the variable heavy (VH) domain sequence comprises a sequence having at least 90%, at least 95%, at least 98%, or at least 99% identity to the VH domain sequence of antibody 1400 and the variable light (VL) domain sequence comprises a sequence having at least 90%, at least 95%, at least 98%, or at least 99% identity to the VL domain sequence of antibody 1400, provided that the antibody has the 6 CDRs of antibody 1400.

In one embodiment, the variable heavy (VH) domain sequence comprises a sequence having at least 90%, at least 95%, at least 98%, or at least 99% identity to the VH domain sequence of antibody 1401 and the variable light (VL) domain sequence comprises a sequence having at least 90%, at least 95%, at least 98%, or at least 99% identity to the VL domain sequence of antibody 1401, provided that the antibody has the 6 CDRs of antibody 1401.

In one embodiment, the variable heavy (VH) domain sequence comprises a sequence having at least 90%, at least 95%, at least 98%, or at least 99% identity to the VH domain sequence of antibody 1402 and the variable light (VL) domain sequence comprises a sequence having at least 90%, at least 95%, at least 98%, or at least 99% identity to the VL domain sequence of antibody 1402, provided that the antibody has the 6 CDRs of antibody 1402.

In one embodiment, the variable heavy (VH) domain sequence comprises a sequence having at least 90%, at least 95%, at least 98%, or at least 99% identity to the VH domain sequence of antibody 1404 and the variable light (VL) domain sequence comprises a sequence having at least 90%, at least 95%, at least 98%, or at least 99/o identity to the VL domain sequence of antibody 1404, provided that the antibody has the 6 CDRs of antibody 1404.

In one embodiment, the variable heavy (VH) domain sequence comprises a sequence having at least 90%, at least 95%, at least 98%, or at least 99% identity to the VH domain sequence of antibody 1409 and the variable light (VL) domain sequence comprises a sequence having at least 90%, at least 95%, at least 98%, or at least 99% identity to the VL domain sequence of antibody 1409, provided that the antibody has the 6 CDRs of antibody 1409.

In one embodiment, the variable heavy (VH) domain sequence comprises a sequence having at least 90%, at least 95%, at least 98%, or at least 99% identity to the VH domain sequence of antibody 1410 and the variable light (VL) domain sequence comprises a sequence having at least 90%, at least 95%, at least 98%, or at least 99% identity to the VL domain sequence of antibody 1410, provided that the antibody has the 6 CDRs of antibody 1410.

In one embodiment, the variable heavy (VH) domain sequence comprises a sequence having at least 90%, at least 95%, at least 98%, or at least 99% identity to the VH domain sequence of antibody 1412 and the variable light (VL) domain sequence comprises a sequence having at least 90%, at least 95%, at least 98%, or at least 99% identity to the VL domain sequence of antibody 1412, provided that the antibody has the 6 CDRs of antibody 1412.

In one embodiment, the variable heavy (VH) domain sequence comprises a sequence having at least 90%, at least 95%, at least 98%, or at least 99% identity to the VH domain sequence of antibody 1414 and the variable light (VL) domain sequence comprises a sequence having at least 90%, at least 95%, at least 98%, or at least 99% identity to the VL domain sequence of antibody 1414, provided that the antibody has the 6 CDRs of antibody 1414.

In one embodiment, the variable heavy (VH) domain sequence comprises a sequence having at least 90%, at least 95%, at least 98%, or at least 99% identity to the VH domain sequence of antibody 1415 and the variable light (VL) domain sequence comprises a sequence having at least 90%, at least 95%, at least 98%, or at least 99% identity to the VL domain sequence of antibody 1415, provided that the antibody has the 6 CDRs of antibody 1415.

In one embodiment, the variable heavy (VH) domain sequence comprises a sequence having at least 90%, at least 95%, at least 98%, or at least 99% identity to the VH domain sequence of antibody 1416 and the variable light (VL) domain sequence comprises a sequence having at least 90%, at least 95%, at least 98%, or at least 99% identity to the VL domain sequence of antibody 1416, provided that the antibody has the 6 CDRs of antibody 1416.

In one embodiment, the present invention provides an antibody that specifically binds KL49 at the surface of Acinetobacter baumannii bacteria,
wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody 1345, 1347, 1350, 1351, 1363, 1364, 1397, 1398, 1400, 1401, 1402, 1404, 1409, 1410, 1412, 1414, 1415, or 1416.

In one embodiment, the antibody specifically binds KL49 at the surface of Acinetobacter baumannii bacteria, wherein the antibody comprises the VH and VL domain sequences of antibody 1345.

In one embodiment, the antibody specifically binds KL49 at the surface of Acinetobacter baumannii bacteria, wherein the antibody comprises the VH and VL domain sequences of antibody 1347.

In one embodiment, the antibody specifically binds KL49 at the surface of Acinetobacter baumannii bacteria, wherein the antibody comprises the VH and VL domain sequences of antibody 1350.

In one embodiment, the antibody specifically binds KL49 at the surface of Acinetobacter baumannii bacteria, wherein the antibody comprises the VH and VL domain sequences of antibody 1351.

In one embodiment, the antibody specifically binds KL49 at the surface of Acinetobacter baumannii bacteria, wherein the antibody comprises the VH and VL domain sequences of antibody 1363.

In one embodiment, the antibody specifically binds KL49 at the surface of Acinetobacter baumannii bacteria, wherein the antibody comprises the VH and VL domain sequences of antibody 1364.

In one embodiment, the antibody specifically binds KL49 at the surface of Acinetobacter baumannii bacteria, wherein the antibody comprises the VH and VL domain sequences of antibody 1397.

In one embodiment, the antibody specifically binds KL49 at the surface of Acinetobacter baumannii bacteria, wherein the antibody comprises the VH and VL domain sequences of antibody 1398.

In one embodiment, the antibody specifically binds KL49 at the surface of Acinetobacter baumannii bacteria, wherein the antibody comprises the VH and VL domain sequences of antibody 1400.

In one embodiment, the antibody specifically binds KL49 at the surface of Acinetobacter baumannii bacteria, wherein the antibody comprises the VH and VL domain sequences of antibody 1401.

In one embodiment, the antibody specifically binds KL49 at the surface of Acinetobacter baumannii bacteria, wherein the antibody comprises the VH and VL domain sequences of antibody 1402.

In one embodiment, the antibody specifically binds KL49 at the surface of Acinetobacter baumannii bacteria, wherein the antibody comprises the VH and VL domain sequences of antibody 1404.

In one embodiment, the antibody specifically binds KL49 at the surface of Acinetobacter baumannii bacteria, wherein the antibody comprises the VH and VL domain sequences of antibody 1409.

In one embodiment, the antibody specifically binds KL49 at the surface of Acinetobacter baumannii bacteria, wherein the antibody comprises the VH and VL domain sequences of antibody 1410.

In one embodiment, the antibody specifically binds KL49 at the surface of Acinetobacter baumannii bacteria, wherein the antibody comprises the VH and VL domain sequences of antibody 1412.

In one embodiment, the antibody specifically binds KL49 at the surface of Acinetobacter baumannii bacteria, wherein the antibody comprises the VH and VL domain sequences of antibody 1414.

In one embodiment, the antibody specifically binds KL49 at the surface of Acinetobacter baumannii bacteria, wherein the antibody comprises the VH and VL domain sequences of antibody 1415.

In one embodiment, the antibody specifically binds KL49 at the surface of Acinetobacter baumannii bacteria, wherein the antibody comprises the VH and VL domain sequences of antibody 1416.

In one embodiment, the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody 1345, 1347, 1350, 1351, 1363, 1364, 1397, 1398, 1400, 1401, 1402, 1404, 1409, 1410, 1412, 1414, 1415, or 1416.

In one embodiment, the antibody comprises the VH and VL domain sequences of antibody 1345.

In one embodiment, the antibody comprises the VH and VL domain sequences of antibody 1347.

In one embodiment, the antibody comprises the VH and VL domain sequences of antibody 1350.

In one embodiment, the antibody comprises the VH and VL domain sequences of antibody 1351.

In one embodiment, the antibody comprises the VH and VL domain sequences of antibody 1363.

In one embodiment, the antibody comprises the VH and VL domain sequences of antibody 1364.

In one embodiment, the antibody comprises the VH and VL domain sequences of antibody 1397.

In one embodiment, the antibody comprises the VH and VL domain sequences of antibody 1398.

In one embodiment, the antibody comprises the VH and VL domain sequences of antibody 1400.

In one embodiment, the antibody comprises the VH and VL domain sequences of antibody 1401.

In one embodiment, the antibody comprises the VH and VL domain sequences of antibody 1402.

In one embodiment, the antibody comprises the VH and VL domain sequences of antibody 1404.

In one embodiment, the antibody comprises the VH and VL domain sequences of antibody 1409.

In one embodiment, the antibody comprises the VH and VL domain sequences of antibody 1410.

In one embodiment, the antibody comprises the VH and VL domain sequences of antibody 1412.

In one embodiment, the antibody comprises the VH and VL domain sequences of antibody 1414.

In one embodiment, the antibody comprises the VH and VL domain sequences of antibody 1415.

In one embodiment, the antibody comprises the VH and VL domain sequences of antibody 1416.

In one example, the antibody is selected from the group consisting of antibodies 1345, 1347, 1350, 1351, 1363, 1364, 1397, 1398, 1400, 1401, 1402, 1404, 1409, 1410, 1412, 1414, 1415, and 1416.

In one embodiment, the antibody is antibody 1345.

In one embodiment, the antibody is antibody 1347.

In one embodiment, the antibody is antibody 1350.

In one embodiment, the antibody is antibody 1351.

In one embodiment, the antibody is antibody 1363.

In one embodiment, the antibody is antibody 1364.

In one embodiment, the antibody is antibody 1397.

In one embodiment, the antibody is antibody 1398.

In one embodiment, the antibody is antibody 1400.

In one embodiment, the antibody is antibody 1401.

In one embodiment, the antibody is antibody 1402.

In one embodiment, the antibody is antibody 1404.

In one embodiment, the antibody is antibody 1409.

In one embodiment, the antibody is antibody 1410.

In one embodiment, the antibody is antibody 1412.

In one embodiment, the antibody is antibody 1414.

In one embodiment, the antibody is antibody 1415.

In one embodiment, the antibody is antibody 1416.

Antibodies are provided which bind to the same epitope on KL49 as an antibody described anywhere herein.

An antibody is provided which binds to the same epitope as antibody 1345, 1347, 1350, 1351, 1363, 1364, 1397, 1398, 1400, 1401, 1402, 1404, 1409, 1410, 1412, 1414, 1415, or 1416, e.g. as defined by its VH and VL sequences.

In one embodiment, an antibody is provided which binds to the same epitope as antibody 1345, e.g. as defined by its VH and VL sequences.

In one embodiment, an antibody is provided which binds to the same epitope as antibody 1347, e.g. as defined by its VH and VL sequences.

In one embodiment, an antibody is provided which binds to the same epitope as antibody 1350, e.g. as defined by its VH and VL sequences.

In one embodiment, an antibody is provided which binds to the same epitope as antibody 1351, e.g. as defined by its VH and VL sequences.

In one embodiment, an antibody is provided which binds to the same epitope as antibody 1363, e.g. as defined by its VH and VL sequences.

In one embodiment, an antibody is provided which binds to the same epitope as antibody 1364, e.g. as defined by its VH and VL sequences.

In one embodiment, an antibody is provided which binds to the same epitope as antibody 1397, e.g. as defined by its VH and VL sequences.

In one embodiment, an antibody is provided which binds to the same epitope as antibody 1398, e.g. as defined by its VH and VL sequences.

In one embodiment, an antibody is provided which binds to the same epitope as antibody 1400, e.g. as defined by its VH and VL sequences.

In one embodiment, an antibody is provided which binds to the same epitope as antibody 1401, e.g. as defined by its VH and VL sequences.

In one embodiment, an antibody is provided which binds to the same epitope as antibody 1402, e.g. as defined by its VH and VL sequences.

In one embodiment, an antibody is provided which binds to the same epitope as antibody 1404, e.g. as defined by its VH and VL sequences.

In one embodiment, an antibody is provided which binds to the same epitope as antibody 1409, e.g. as defined by its VH and VL sequences.

In one embodiment, an antibody is provided which binds to the same epitope as antibody 1410, e.g. as defined by its VH and VL sequences.

In one embodiment, an antibody is provided which binds to the same epitope as antibody 1412, e.g. as defined by its VH and VL sequences.

In one embodiment, an antibody is provided which binds to the same epitope as antibody 1414, e.g. as defined by its VH and VL sequences.

In one embodiment, an antibody is provided which binds to the same epitope as antibody 1415, e.g. as defined by its VH and VL sequences.

In one embodiment, an antibody is provided which binds to the same epitope as antibody 1416, e.g. as defined by its VH and VL sequences.

An antibody may contact KL49 with a footprint that fully or partly overlaps with that of an antibody disclosed anywhere herein. As described elsewhere herein, competition between antibodies may be determined, for example using SPR, and antibodies are provided which compete for binding to KL49 (compete for binding to their epitope) with an IgG antibody as described anywhere herein.

An antibody of the present invention may be one which competes for binding to KL49 with any anti-KL49 antibody described herein, such as antibodies 1345, 1347, 1350, 1351, 1363, 1364, 1397, 1398, 1400, 1401, 1402, 1404, 1409, 1410, 1412, 1414, 1415, and 1416, e.g. as defined by their respective VH and VL sequences.

An antibody of the present invention may be one which competes for binding to KL49 with antibody 1345.

An antibody of the present invention may be one which competes for binding to KL49 with antibody 1347.

An antibody of the present invention may be one which competes for binding to KL49 with antibody 1350.

An antibody of the present invention may be one which competes for binding to KL49 with antibody 1351.

An antibody of the present invention may be one which competes for binding to KL49 with antibody 1363.

An antibody of the present invention may be one which competes for binding to KL49 with antibody 1364.

An antibody of the present invention may be one which competes for binding to KL49 with antibody 1397.

An antibody of the present invention may be one which competes for binding to KL49 with antibody 1398.

An antibody of the present invention may be one which competes for binding to KL49 with antibody 1400.

An antibody of the present invention may be one which competes for binding to KL49 with antibody 1401.

An antibody of the present invention may be one which competes for binding to KL49 with antibody 1402.

An antibody of the present invention may be one which competes for binding to KL49 with antibody 1404.

An antibody of the present invention may be one which competes for binding to KL49 with antibody 1409.

An antibody of the present invention may be one which competes for binding to KL49 with antibody 1410.

An antibody of the present invention may be one which competes for binding to KL49 with antibody 1412.

An antibody of the present invention may be one which competes for binding to KL49 with antibody 1414.

An antibody of the present invention may be one which competes for binding to KL49 with antibody 1415.

An antibody of the present invention may be one which competes for binding to KL49 with antibody 1416.

A nucleic acid sequence provided by the invention may comprise a sequence that encodes a VH domain and/or an VL domain of an anti-KL49 antibody as defined anywhere herein.

The nucleic acid may comprise a sequence that encodes a VH domain and/or a VL domain of antibody 1345.

The nucleic acid may comprise a sequence that encodes a VH domain and/or a VL domain of antibody 1347.

The nucleic acid may comprise a sequence that encodes a VH domain and/or a VL domain of antibody 1350.

The nucleic acid may comprise a sequence that encodes a VH domain and/or a VL domain of antibody 1351.

The nucleic acid may comprise a sequence that encodes a VH domain and/or a VL domain of antibody 1363.

The nucleic acid may comprise a sequence that encodes a VH domain and/or a VL domain of antibody 1364.

The nucleic acid may comprise a sequence that encodes a VH domain and/or a VL domain of antibody 1397.

The nucleic acid may comprise a sequence that encodes a VH domain and/or a VL domain of antibody 1398.

The nucleic acid may comprise a sequence that encodes a VH domain and/or a VL domain of antibody 1400.

The nucleic acid may comprise a sequence that encodes a VH domain and/or a VL domain of antibody 1401.

The nucleic acid may comprise a sequence that encodes a VH domain and/or a VL domain of antibody 1402.

The nucleic acid may comprise a sequence that encodes a VH domain and/or a VL domain of antibody 1404.

The nucleic acid may comprise a sequence that encodes a VH domain and/or a VL domain of antibody 1409.

The nucleic acid may comprise a sequence that encodes a VH domain and/or a VL domain of antibody 1410.

The nucleic acid may comprise a sequence that encodes a VH domain and/or a VL domain of antibody 1412.

The nucleic acid may comprise a sequence that encodes a VH domain and/or a VL domain of antibody 1414.

The nucleic acid may comprise a sequence that encodes a VH domain and/or a VL domain of antibody 1415.

The nucleic acid may comprise a sequence that encodes a VH domain and/or a VL domain of antibody 1416.

### 3.4 Antibodies—General

In an embodiment, the antibody as defined anywhere herein shows complement-dependent cytotoxic activity (CDC) activity.

An antibody as defined anywhere herein may be a human IgG1 or human IgG4. In one embodiment, the antibody is a human IgG1. In one embodiment, the antibody is a human IgG1 comprising a constant region sequence of SEQ ID NO: 418. In one embodiment, the antibody is a human IgG4. In one embodiment, the antibody is a human IgG4 comprising a constant region sequence of SEQ ID NO: 436.

An antibody as defined anywhere herein may be a human IgA1 (e.g., comprising a constant region sequence SEQ ID NO: 484) or human IgA2 (e.g., comprising a constant region sequence SEQ ID NO: 485).

An antibody as defined anywhere herein may comprise kappa (κ) light chain constant regions, preferably constant domain sequence SEQ ID NO: 448. The present invention provides a vector comprising a nucleic acid as defined anywhere herein; optionally wherein the vector is a CHO vector.

The present invention provides a host cell comprising a nucleic acid as defined anywhere herein or a vector as defined anywhere herein.

The present invention provides a pharmaceutical composition comprising an antibody as defined anywhere herein and a pharmaceutically acceptable excipient.

The present invention provides a pharmaceutical composition comprising an isolated nucleic acid encoding an antibody as defined anywhere herein, or an isolated nucleic acid as defined anywhere herein, and a pharmaceutically acceptable excipient.

In one embodiment, the pharmaceutical composition is formulated for administration via injection.

In one embodiment, the pharmaceutical composition is formulated for intravenous, intramuscular or subcutaneous administration.

In one embodiment, the pharmaceutical composition further comprises at least one further therapeutic agent.

In one embodiment, the further therapeutic agent is at least one, preferably one or two, further antibodies.

In one embodiment, the further therapeutic agent is carbapenem.

In one embodiment, the further therapeutic agent is colistin.

In one embodiment, the pharmaceutical composition comprises a first antibody that specifically binds to Oxa-23 and a second antibody that specifically binds to OC1 LOS.

In one embodiment, the pharmaceutical composition comprises a first antibody that specifically binds Oxa-23 and a second antibody that specifically binds KL49.

In one embodiment, the pharmaceutical composition comprises a first antibody that specifically binds OC1 LOS and a second antibody that specifically binds KL49.

In one embodiment, the pharmaceutical composition comprises a first antibody that specifically binds Oxa-23, a second antibody that specifically binds OC1 LOS, and a third antibody that specifically binds KL49.

The present invention provides a diagnostic kit comprising at least one antibody as defined anywhere herein. In one embodiment, the diagnostic kit comprises a first antibody that specifically binds Oxa-23 and a second antibody that specifically binds OC1 LOS. In one embodiment, the diagnostic kit comprises a first antibody that specifically binds Oxa-23 and a second antibody that specifically binds KL49. In one embodiment, the diagnostic kit comprises a first antibody that specifically binds OC1 LOS and a second antibody that specifically binds KL49. In one embodiment, the diagnostic kit comprises a first antibody that specifically binds Oxa-23, a second antibody that specifically binds OC1 LOS, and a third antibody that specifically binds KL49.

The present invention provides a kit comprising a pharmaceutical composition as defined anywhere herein. In one embodiment, the kit further comprises at least one further therapeutic agent. In one embodiment, the further therapeutic agent is a further pharmaceutical composition comprising at least one, preferably one or two, further antibodies. In one embodiment, the kit further comprises carbapenem. In one embodiment, the kit further comprises colistin.

In one embodiment, the at least one further antibody is selected from: an antibody that specifically binds Oxa-23; an antibody that specifically binds OC1 LOS; and an antibody that specifically binds KL49.

In one embodiment, the kit comprises a first antibody that specifically binds Oxa-23 and a second antibody that specifically binds OC1 LOS.

In one embodiment, the kit comprises a first antibody that specifically binds Oxa-23 and a second antibody that specifically binds KL49.

In one embodiment, the kit comprises a first antibody that specifically binds OC1 LOS and a second antibody that specifically binds KL49.

In one embodiment, the kit comprises a first antibody that specifically binds Oxa-23, a second antibody that specifically binds OC1 LOS, and a third antibody that specifically binds KL49.

In one embodiment, the kit further comprises a label or instructions for use to prevent and/or treat a bacterial infection caused by Acinetobacter baumannii in a human; optionally wherein the label or instructions comprise a marketing authorisation number (e.g., an FDA or EMA authorisation number); optionally wherein the kit comprises an IV or injection device that comprises the antibody. Preferably, the antibody is contained in a sealed container.

Optionally, the kit contains instructions for simultaneous, separate or sequential administration of the therapeutic agents therein.

An antibody as defined anywhere herein or a composition as defined anywhere herein may be provided for use as a medicament.

An antibody as defined anywhere herein or a composition as defined anywhere herein may be provided for use in a method of treating a bacterial infection caused by Acinetobacter baumannii, said method comprising administering the antibody or composition to a patient.

The antibody as defined anywhere herein, or the composition as defined anywhere herein, may be provided for use in a method of preventing a bacterial infection caused by Acinetobacter baumannii, said method comprising administering the antibody or composition to a patient.

Where an antibody as defined anywhere herein that specifically binds to Oxa-23 is administered to a patient, the presence of Oxa-23 may already have been determined in a sample from that patient, although this is not essential.

Where an antibody as defined anywhere herein that specifically binds to OC1 LOS is administered to a patient, the presence of OC1 LOS may already have been determined in a sample from that patient, although this is not essential.

Where an antibody as defined anywhere herein that specifically binds to KL49 is administered to a patient, the presence of KL49 may already have been determined in a sample from that patient, although this is not essential.

Also provided is the use of an antibody as defined anywhere herein, or the use of a composition as defined anywhere herein, in the manufacture of a medicament for use in a method of treating a bacterial infection caused by Acinetobacter baumannii.

Also provided is the use of an antibody as defined anywhere herein, or the use of a composition as defined anywhere herein, in the manufacture of a medicament for use in a method of preventing a bacterial infection caused by Acinetobacter baumannii.

The present invention provides a method of treating a bacterial infection caused by Acinetobacter baumannii in a patient comprising administering to said patient a therapeutically effective amount of an antibody as defined anywhere herein, or a composition as defined anywhere herein.

The present invention provides a method of preventing a bacterial infection caused by Acinetobacter baumannii in a patient comprising administering to said patient a therapeutically effective amount of an antibody as defined anywhere herein, or a composition as defined anywhere herein.

In one embodiment, the bacterial infection caused by Acinetobacter baumannii is a nosocomial bacterial infection caused by Acinetobacter baumannii.

In one embodiment, the patient has a lower respiratory tract infection, for example pneumonia.

In one embodiment, the patient has sepsis.

In one embodiment, the patient has bacteremia.

In one embodiment, the method further comprises administering at least one further therapeutic agent.

In one embodiment, the administration of the further therapeutic agent is simultaneous, separate or sequential.

In one embodiment, the further therapeutic agent is at least one, preferably one or two, further antibodies.

In one embodiment, the method comprises administering a first antibody that specifically binds Oxa-23 and a second antibody that specifically binds OC1 LOS.

In one embodiment, the method comprises administering a first antibody that specifically binds Oxa-23 and a second antibody that specifically binds KL49.

In one embodiment, the method comprises administering a first antibody that specifically binds OC1 LOS and a second antibody that specifically binds KL49.

In one embodiment, the method comprises administering a first antibody that specifically binds Oxa-23, a second antibody that specifically binds OC1 LOS, and a third antibody that specifically binds KL49.

In one embodiment, the method further comprises administering carbapenem.

In one embodiment, the method further comprises administering colistin.

Also provided is the use of an antibody as defined anywhere herein, for determining the presence or absence of Acinetobacter baumannii in a sample. Determining the presence of Acinetobacter baumannii in a sample can be used for diagnosis of an infection caused by Acinetobacter baumannii in a patient.

Also provided is a method of determining the presence or absence of Acinetobacter baumannii in a sample may comprise contacting the sample with an antibody as defined anywhere herein; and testing for binding between the antibody and Acinetobacter baumannii in the sample; wherein detection of binding indicates the presence of Acinetobacter baumannii in the sample and wherein absence of binding indicates the absence of Acinetobacter baumannii in the sample.

Also provided is the use of an antibody as defined anywhere herein that specifically binds to Oxa-23, for determining the presence or absence of Oxa-23 in a sample. Determining the presence of Oxa-23 in a sample can be used for determining a treatment protocol in a patient. For example, a patient may be identified for treatment with an antibody that specifically binds to Oxa-23 if the presence of Oxa-23 has been determined in a sample from that patient. The patient may or may not have already been diagnosed as having been infected with Acinetobacter baumannii.

Also provided is the use of an antibody as defined anywhere herein that specifically binds to OC1 LOS, for determining the presence or absence of OC1 LOS in a sample. Determining the presence of OC1 LOS in a sample can be used for determining a treatment protocol in a patient. For example, a patient may be identified for treatment with an antibody that specifically binds to OC1 LOS if the presence of OC1 LOS has been determined in a sample from that patient. The patient may or may not have already been diagnosed as having been infected with Acinetobacter baumannii.

Also provided is the use of an antibody as defined anywhere herein that specifically binds to KL49, for determining the presence or absence of KL49 in a sample. Determining the presence of KL49 in a sample can be used for determining a treatment protocol in a patient. For example, a patient may be identified for treatment with an antibody that specifically binds to KL49 if the presence of KL49 has been determined in a sample from that patient. The patient may or may not have already been diagnosed as having been infected with Acinetobacter baumannii.

In one embodiment, the antibody is conjugated to a detectable label.

In one embodiment, the sample has been obtained from a human who has been or is suspected of having been infected with Acinetobacter baumannii. In one embodiment, the sample has been obtained from a human who has been or is suspected of having been infected with Acinetobacter baumannii who exhibits one or more symptoms of a bacterial infection. In one embodiment, the sample is a serum, plasma, or whole blood sample, an oral or nasal swab, urine, faeces, or cerebrospinal fluid (CFS), or wherein the sample is from any suspected Acinetobacter baumannii infected organ or tissue.

A diagnostic kit is provided for the use as defined anywhere herein, or the method as defined anywhere herein. The diagnostic kit may comprise an antibody as defined anywhere herein, and optionally one or more buffering solutions. In one embodiment, the diagnostic kit comprises a first reagent comprising the antibody as defined anywhere herein, and a second reagent comprising a detector molecule that binds to the first reagent. In one embodiment, the detector molecule is an antibody that comprises or is conjugated to a detectable label.

### 4. FIGURES

FIG. 1a. Time resolved core genome phylogeny for GC2 A. baumannii isolates in a Ho Chi Minh ICU. Shading indicates the period during which the carbapenem antibiotic imipenem was used for the empirical treatment of VAP in the ICU. Labelled A-E are the five carbapenem resistant genomic subclades of GC2. The capsule K-locus subtype for the 10 strains tested for OMV production are also listed on the right. Marked with asterisks (*) are 4 strains from which OMVs have been successfully generated and used for immunisation of Kymab Intellisellect Transgenic mice.

FIG. 1b. Scanning electron microscope images of un-fixed OMV preparations generated from a A. baumannii clinical isolates used for immunisation. OMVs prepared from A. baumannii BAL_191, BAL_276, BAL_215 and BAL_084. Approximately 95% of OMVs in this preparation are between 25-70 nm in diameter, while larger OMVs with a diameter>80 nm comprise 2% of the preparation. Additional, amorphic structures can also be observed and constitute 3% of the preparation.

FIG. 2a. Regime used for OMV immunisations. 10 mice received a prime dose of ˜1 μg of OMVs on day 0 of the study. Following this, the mice were split into 2 study groups, with 5 mice in group 1 sacrificed on day 7, with their spleens and lymph nodes harvested. The 5 mice in group 2 received an additional boost on day 36 before being sacrificed on day 43. Bleeds were taken prior to prime and boost doses, and on day 7. These sera were assessed for reactivity against the immunising OMVs via ELISA. Organs harvested on day 7 can be analysed using IntelliSelect Screening Technologies to B cell responses using organs harvested on day 43. PB=pre-bleed; SB=serial bleed; TB=terminal bleed.

FIG. 2b. Polyclonal serum responses of Kymab Intellisellect Transgenic mice immunised with a mixed pool of 4 GC2 A. baumannii OMVs to the immunising OMVs as determined by ELISA. (a) Kinetics of the antigen specific IgG1 response to the 4 GC2 A. baumannii OMVs used for immunisation. The total antigen specific IgG1 response to the 4 individual GC2 A. baumannii OMVs used for immunisation (BAL_084, BAL_191, BAL_215 and BAL_276 in bold) is comparable to the response to 2 additional GC2 A. baumannii OMVs that were absent from the immunising pool (BAL_339 and BAL_377) on day 43 of the study. Specific IgG1 responses were comparable across all mice immunised and all human Ig Isotypes were represented by specific antibodies present in the polyclonal serum. Antigen specific relative endpoint titres have been calculated relative to the pre-bleed taken prior to prime immunisation.

FIG. 3. Gating strategy for sorting antigen-specific IgG+ B cells from the spleens of GC2 CRAB OMV-immunised mice.

FIG. 4a. Sunspot plot of the 951 paired VH and VL chain sequences recovered from the spleens of 4 Kymab Intellisellect Transgenic mice immunised with GC2 CRAB OMVs. Each VH and VL paired sequence is represented by a circle, coloured by the individual mouse from which it was derived. Sequences within a lineage, inferred to have derived from a common B cell progenitor are connected by black lines.

FIG. 4b. Table indicating number of IgH&L paired sequences, proportion mapping to high-quality sequences and apportioned to clusters of related sequences indicative of an actively evolving B cell response to an immunogen.

FIG. 5a. Comparison of mAb binding to GC2 A. baumannii OMVs and whole, unfixed bacterial cells as determined by ELISA. The mAbs identified as binders in both ELISA binding assays: mAbs that bound to both whole, unfixed GC2 A. baumannii bacterial cells and OMVs at levels above the assay thresholds indicated on the plot, are indicated in green. In orange are 2 mAbs that only bound whole, unfixed bacterial cells. The mAbs unable to bind to intact bacterial cell wall are indicated in white.

FIG. 5b. Selection of mAbs for CHO expression and purification based on binding of both intact bacterial cell wall and ability to trigger deposition of C3b as described above. Examples of selections for subsequent CHO expression are depicted in bold.

FIG. 7a. Table indicating identified targets for each mAb. Methods used include phylogenetic information gleaned from HCl (see above), serological expression cloning using a genomic phage library (SEC) and western blot with specific mAb (WB). The mAbs in bold (*) were tested in an in vivo challenge model of A. baumannii to demonstrate therapeutic efficacy.

FIG. 7b. Example western blot analysis reveals mAb binding three distinct targets; a high molecular weight target (KL49 capsule-upper panel), 10kD target (OC1 LOS-middle panel), and a 30kD protein (Oxa-23-lower panel).

### 5. DETAILED DESCRIPTION OF THE INVENTION

The present invention provides monoclonal antibodies for diagnosis and treatment in patients of bacterial infection caused by Acinetobacter baumannii.

The antibodies described herein are described with respect to the following concepts, aspects, sentences, arrangements and embodiments. Unless otherwise stated, all concepts, embodiments, sentences, arrangements and aspects are to be read as being able to be combined with any other concept, aspect, sentence, arrangement or embodiment, unless such combination would not make technical sense or is explicitly stated otherwise.

The CDR, VH, and VL sequences of antibodies referred to herein are provided in Table 1.

### 5.1 Definitions

Unless otherwise defined herein, scientific and technical terms shall have the meanings that are commonly understood by those of ordinary skill in the art. Further, unless otherwise required by context, singular terms shall include pluralities and plural terms shall include the singular.

The singular terms “a,” “an,” and “the” include plural referents unless context clearly indicates otherwise.

Similarly, the word “or” is intended to include “and” unless the context clearly indicates otherwise.

Although methods and materials similar or equivalent to those described herein can be used in the practice or testing of this disclosure, suitable methods and materials are described below. The abbreviation, “e.g.” is derived from the Latin exempli gratia and is used herein to indicate a non-limiting example. Thus, the abbreviation “e.g.” is synonymous with the term “for example.”

In the specification and claims, the term “about” is used to modify, for example, the quantity of an ingredient in a composition, concentration, volume, process temperature, process time, yield, flow rate, pressure, and like values, and ranges thereof, employed in describing the examples of the disclosure. The term “about” refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture and amounts that differ due to mixing or processing a formulation with a particular initial concentration or mixture. Where modified by the term “about” the claims appended hereto include equivalents to these quantities.

As used herein, “administer” or “administration” refers to the act of injecting or otherwise physically delivering a substance as it exists outside the body (e.g., an antibody provided herein, or its encoding nucleic acid e.g. in an expression vector) into a patient, such as by mucosal, intradermal, intravenous, intramuscular delivery, inhalation e.g. nebulisation and/or any other method of physical delivery described herein or known in the art. When an infection, or a symptom thereof, is being treated, administration of the substance typically occurs after the onset of the infection or symptoms thereof. When an infection, or symptoms thereof, are being prevented, administration of the substance typically occurs before the onset of the infection or symptoms thereof.

The term “antibody”, “immunoglobulin” or “Ig” may be used interchangeably herein and means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies (including dual binding antibodies), chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. The term “antibody” can also refer to a Y-shaped glycoprotein with a molecular weight of approximately 150 kDa that is made up of four polypeptide chains: two light (L) chains and two heavy (H) chains. There are five types of mammalian Ig heavy chain isotypes denoted by the Greek letters alpha (α), delta (δ), epsilon (ε), gamma (γ), and mu (μ). The type of heavy chain defines the class of antibody, i.e., IgA, IgD, IgE, IgG, and IgM, respectively. The γ and α classes are further divided into subclasses on the basis of differences in the constant domain sequence and function, e.g., IgG1, hIgG2, mIgG2A, mIgG2B, IgG3, IgG4, IgA1 and IgA2. In mammals, there are two types of immunoglobulin light chains, λ and κ. The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites. An example of antibodies are heavy chain-only (i.e., H2) antibodies that comprise a dimer of a heavy chain (5′-VH-(optional Hinge)-CH2-CH3-3′) and are devoid of a light chain.

The antibodies described herein may be oligoclonal, polyclonal, monoclonal (including full-length monoclonal antibodies), camelised, chimeric, CDR-grafted, multi-specific, bi-specific (including dual-binding antibodies), catalytic, chimeric, humanized, fully human, anti-idiotypic, including antibodies that can be labelled in soluble or bound form as well as fragments, variants or derivatives thereof, either alone or in combination with other amino acid sequences provided by known techniques. An antibody may be from any species. Antibodies described herein can be naked or conjugated to other molecules such as toxins, radioisotopes, etc.

The term “antigen binding domain,” “antigen binding region,” “antigen binding fragment,” and similar terms refer to that portion of an antibody which comprises the amino acid residues that interact with an antigen and confer on the binding agent its specificity and affinity for the antigen (e.g. the complementarity determining regions (CDRs)). The antigen binding region can be derived from any animal species, such as rodents (e.g. rabbit, rat or hamster) and humans. Preferably, the antigen binding region will be of human origin.

Antigen binding fragments described herein can include single-chain Fvs (scFv), single-chain antibodies, single domain antibodies, domain antibodies, Fv fragments, Fab fragments, F(ab′) fragments, F(ab′)2 fragments, antibody fragments that exhibit the desired biological activity, disulfide-stabilised variable region (dsFv), dimeric variable region (diabody), anti-idiotypic (anti-Id) antibodies (including, e.g. anti-Id antibodies to antibodies), intrabodies, linear antibodies, single-chain antibody molecules and multispecific antibodies formed from antibody fragments and epitope-binding fragments of any of the above. In particular, antibodies and antibody fragments described herein can include immunoglobulin molecules and immunologically active fragments of immunoglobulin molecules, i.e., molecules that contain an antigen-binding site. Digestion of antibodies with the enzyme, papain, results in two identical antigen-binding fragments, known also as “Fab” fragments, and a “Fc” fragment, having no antigen-binding activity but having the ability to crystallize. “Fab” when used herein refers to a fragment of an antibody that includes one constant and one variable domain of each of the heavy and light chains. The term “Fc region” herein is used to define a C-terminal region of an immunoglobulin heavy chain, including native-sequence Fc regions and variant Fc regions. The “Fc fragment” refers to the carboxy-terminal portions of both H chains held together by disulfides. The effector functions of antibodies are determined by sequences in the Fc region, the region which is also recognized by Fc receptors (FcR) found on certain types of cells. Digestion of antibodies with the enzyme, pepsin, results in a F(ab′)2 fragment in which the two arms of the antibody molecule remain linked and comprise two-antigen binding sites. The F(ab′)2 fragment has the ability to crosslink antigen.

“Fv” when used herein refers to the minimum fragment of an antibody that retains both antigen-recognition and antigen-binding sites. This region consists of a dimer of one heavy and one light chain variable domain in tight, non-covalent or covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The term “monoclonal antibody” as used herein refers to an antibody obtained from a population of substantially homogeneous antibodies, i.e. the individual antibodies comprising the population are identical except for possible naturally occurring mutations and/or post-translation modifications (e.g. isomerizations, amidations) that may be present in minor amounts. Monoclonal antibodies are highly specific and are directed against a single antigenic determinant or epitope. In contrast, polyclonal antibody preparations typically include different antibodies directed against different antigenic determinants (or epitopes). The term “monoclonal antibody” as used herein encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, hybridoma, phage selection, recombinant expression, and transgenic animals.

The monoclonal antibodies herein can include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies that exhibit the desired biological activity.

The term “humanized antibody” refers to a subset of chimeric antibodies in which a “hypervariable region” from a non-human immunoglobulin (the donor antibody) replaces residues from a hypervariable region in a human immunoglobulin (recipient antibody). In general, a humanized antibody will include substantially all of at least one, and typically two, variable domains, in which all or substantially all of the hypervariable loops correspond to those of a non-human immunoglobulin sequence, and all or substantially all of the framework regions are those of a human immunoglobulin sequence, although the framework regions may include one or more substitutions that improve antibody performance, such as binding affinity, isomerization, immunogenicity, etc.

The term “hypervariable region”, “CDR region” or “CDR” refers to the regions of an antibody variable domain which are hypervariable in sequence and/or form structurally defined loops. Generally, antigen binding sites of an antibody include six hypervariable regions: three in the VH (CDRH1, CDRH2, CDRH3), and three in the VL (CDRL 1, CDRL2, CDRL3). These regions of the heavy and light chains of an antibody confer antigen-binding specificity to the antibody. CDRs may be defined according to the Kabat system (see Kabat, E. A.et al., 1991, “Sequences of Proteins of Immunological Interest”, 5th edit., NIH Publication no. 91-3242, U.S. Department of Health and Human Services). Other systems may be used to define CDRs, which as the system devised by Chothia et al (see Chothia, C. & Lesk, A. M., 1987, “Canonical structures for the hypervariable regions of immunoglobulins”, J. Mol. Biol., 196, 901-917) and the IMGT system (see Lefranc, M. P., 1997, “Unique database numbering system for immunogenetic analysis”, Immunol. Today, 18, 50). An antibody typically contains 3 heavy chain CDRs and 3 light chain CDRs. The term CDR or CDRs is used here to indicate one or several of these regions. A person skilled in the art is able to readily compare the different systems of nomenclature and determine whether a particular sequence may be defimed as a CDR.

A “human antibody” is an antibody that possesses an amino-acid sequence corresponding to that of an antibody produced by a human and/or has been made using any of the techniques for making human antibodies and specifically excludes a humanized antibody comprising non-human antigen-binding residues. The term “specifically binds to” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one example, the extent of binding of an antibody to an unrelated target is less than about 10/6 of the binding of the antibody to the target as measured, e.g. by a radioimmunoassay (RIA).

As used herein, “authorization number” or “marketing authorization number” refers to a number issued by a regulatory agency upon that agency determining that a particular medical product and/or composition may be marketed and/or offered for sale in the area under the agency's jurisdiction. As used herein “regulatory agency” refers to one of the agencies responsible for evaluating, e.g. the safety and efficacy of a medical product and/or composition and controlling the sales/marketing of such products and/or compositions in a given area. The Food and Drug Administration (FDA) in the US and the European Medicines Agency (EPA) in Europe are but two examples of such regulatory agencies. Other non-limiting examples can include SDA, MPA, MHPRA, IMA, ANMAT, Hong Kong Department of Health-Drug Office, CDSCO, Medsafe, and KFDA.

As used herein, the term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

As used herein, the term “composition” is intended to encompass a product containing the specified ingredients (e.g. an antibody) in, optionally, the specified amounts, as well as any product which results, directly or indirectly, from combination of the specified ingredients in, optionally, the specified amounts.

As used herein the term “comprising” or “comprises” is used with reference to antibodies, uses, compositions, methods, and respective component(s) thereof, that are essential to the method or composition, yet open to the inclusion of unspecified elements, whether essential or not.

The term “consisting of” refers to antibodies, uses, compositions, methods, and respective components thereof as described herein, which are exclusive of any element not recited in that description of the example.

As used herein the term “consisting essentially of” refers to those elements required for a given example. The term permits the presence of elements that do not materially affect the basic and novel or functional characteristic(s) of that example.

An “effective amount” refers to an amount effective, at dosages and for periods of time necessary, to achieve the desired effect, including a therapeutic or prophylactic result. A “therapeutically effective amount” refers to the minimum concentration required to effect a measurable improvement or prevention of a particular disorder. A therapeutically effective amount herein may vary according to factors such as the infection state, age, sex, and weight of the patient, and the ability of the antibody to elicit a desired response in the individual. A therapeutically effective amount is also one in which toxic or detrimental effects of the antibody are outweighed by the therapeutically beneficial effects. A “prophylactically effective amount” refers to an amount effective, at the dosages and for periods of time necessary, to achieve the desired prophylactic result. In some examples, the effective amount of an antibody is from about 0.1 mg/kg (mg of antibody per kg weight of the subject) to about 100 mg/kg. In certain examples, an effective amount of an antibody provided therein is about 0.1 mg/kg, about 0.5 mg/kg, about 1 mg/kg, 3 mg/kg, 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg about 90 mg/kg or about 100 mg/kg (or a range therein). In some examples, “effective amount” as used herein also refers to the amount of an antibody to achieve a specified result (e.g. inducing complement activation).

The term “epitope” as used herein refers to a localized region on the surface of an antigen that is capable of being bound to one or more antigen binding regions of an antibody, and that has antigenic or immunogenic activity in an animal, preferably a mammal, and most preferably in a human, that is capable of eliciting an immune response. An epitope having immunogenic activity is a portion of an antigen (e.g., a polypeptide, carbohydrate or lipid molecule) that elicits an antibody response in an animal. An epitope having antigenic activity is a portion of an antigen to which an antibody specifically binds as determined by any method well known in the art, for example, by the immunoassays described herein. Antigenic epitopes need not necessarily be immunogenic. Epitopes usually consist of chemically active surface groupings of molecules such as amino acids or sugar side chains and have specific three-dimensional structural characteristics as well as specific charge characteristics. A region of a polypeptide contributing to an epitope may be contiguous amino acids of the polypeptide or the epitope may come together from two or more non-contiguous regions of the polypeptide. The epitope may or may not be a three-dimensional surface feature of the antigen. As described elsewhere herein, competition between antibodies may also be determined, for example using SPR.

The term “excipients” as used herein refers to inert substances which are commonly used as a diluent, vehicle, preservatives, binders, or stabilizing agent for drugs and includes, but not limited to, proteins (e.g. serum albumin, etc.), amino acids (e.g. aspartic acid, glutamic acid, lysine, arginine, glycine, histidine, etc.), fatty acids and phospholipids (e.g. alkyl sulfonates, caprylate, etc.), surfactants (e.g. SDS, polysorbate, non-ionic surfactant, etc.), saccharides (e.g. sucrose, maltose, trehalose, etc.) and polyols (e.g. mannitol, sorbitol, etc.). See, also, Remington's Pharmaceutical Sciences (1990) Mack Publishing Co., Easton, Pa., which is hereby incorporated by reference in its entirety.

The term “heavy chain” when used with reference to an antibody refers to five distinct types, called alpha (α), delta (δ), epsilon (ε), gamma (γ) and mu (μ), based on the amino acid sequence of the heavy chain constant domain. These distinct types of heavy chains are well known and give rise to five classes of antibodies, IgA, IgD, IgE, IgG and IgM, respectively, including two subclasses of IgA, namely IgA1 and IgA2 and four subclasses of IgG, namely IgG1, IgG2, IgG3 and IgG4. Preferably the heavy chain is a human heavy chain. In the human population, multiple heavy chain constant region alleles, of each immunoglobulin or immunoglobulin subclass, exist. The nucleotide and amino acid sequences of these allelic variants are accessible on publicly available databases such as IMGT, ENSEMBL Swiss-Prot and Uniprot. Allelic variants may also be identified in various genome sequencing projects. In one example, the antibodies disclosed herein comprise a heavy chain encoded by a IgG1 constant region allele, which includes, but is not limited to, human IGHG1*01, IGHG1*02, IGHG1*03, IGHG1*04 and IGHG1*05 (the nucleotide and corresponding amino acid sequences of which are set out in Table 2). In one example, the antibodies disclosed herein comprise a protein encoded by a IgG4 constant region allele, which includes but is not limited to human IGHG4*01, IGHG4*02, IGHG4*03 and IGHG4*04 (the nucleotide and corresponding amino acid sequences of which are set out in Table 2). In another example, the heavy chain is an IgA isotype, human IgA1 or human IgA2, example amino acid sequences for which are shown in Table 2. In another example, the heavy chain is a disabled IgG isotype, e.g. a disabled IgG4. In certain examples, the antibodies comprise a human gamma 4 constant region. In another example, the heavy chain constant region does not bind Fc-γ receptors, and e.g. comprises a Leu235Glu mutation. In another example, the heavy chain constant region comprises a Ser228Pro mutation to increase stability. In another example, the heavy chain constant region is IgG4-PE. In another example, the antibodies disclosed herein comprise a heavy chain constant region encoded by a murine IgG1 constant region allele, which includes but is not limited to mouse IGHG1*01 or IGHG1*02.

The term “host” as used herein refers to an animal, preferably a mammal, and most preferably a human.

The term “host cell” as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “in combination” in the context of the administration of other therapies refers to the use of more than one therapy. The use of the term “in combination” does not restrict the order in which therapies are administered to a subject with an infection. A first therapy can be administered before (e.g. 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g. 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject. Any additional therapy can be administered in any order with the other additional therapies. In certain examples, the antibodies can be administered in combination with one or more therapies.

As used herein, “injection device” refers to a device that is designed for carrying out injections, an injection including the steps of temporarily fluidically coupling the injection device to a person's tissue, typically the subcutaneous tissue. An injection further includes administering an amount of liquid drug into the tissue and decoupling or removing the injection device from the tissue. In some examples, an injection device can be an intravenous device or IV device, which is a type of injection device used when the target tissue is the blood within the circulatory system, e.g. the blood in a vein. A common, but non-limiting example of an injection device is a needle and syringe.

As used herein, “instructions” refers to a display of written, printed or graphic matter on the immediate container of an article, for example the written material displayed on a vial containing a pharmaceutically active agent, or details on the composition and use of a product of interest included in a kit containing a composition of interest. Instructions set forth the method of the treatment as contemplated to be administered or performed.

An “isolated” or “purified” antibody or protein is one that has been identified, separated and/or recovered from a component of its production environment (e.g. natural or recombinant). For example, the antibody or protein is substantially free of cellular material or other contaminating proteins from the cell or tissue source from which the antibody is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, or 5% (by dry weight) of heterologous protein (also referred to herein as a “contaminating protein”). When the antibody is recombinantly produced, it is also preferably substantially free of culture medium, i.e. culture medium represents less than about 20%, 10%, or 5% of the volume of the protein preparation. When the antibody is produced by chemical synthesis, it is preferably substantially free of chemical precursors or other chemicals, i.e., it is separated from chemical precursors or other chemicals which are involved in the synthesis of the protein. Accordingly, such preparations of the antibody have less than about 30%, 20%, 10%, 5% (by dry weight) of chemical precursors or compounds other than the antibody of interest. In a preferred example, antibodies are isolated or purified.

The terms “Kabat numbering,” and like terms are recognized in the art and refer to a system of numbering amino acid residues which are more variable (i.e. hypervariable) than other amino acid residues in the heavy chain variable regions of an antibody, or an antigen binding portion thereof (Kabat et al., (1971) Ann. NY Acad. Sci., 190:382-391 and, Kabat et al. (1991) Sequences of Proteins of Immunological Interest, Fifth Edition, U.S. Department of Health and Human Services, NIH Publication No. 91-3242). For the heavy chain variable region, the hypervariable region typically ranges from amino acid positions 31 to 35 for CDR1, amino acid positions 50 to 65 for CDR2, and amino acid positions 95 to 102 for CDR3.

“Label” or “labelled” as used herein refers to the addition of a detectable moiety to a polypeptide, for example, a radiolabel, fluorescent label, enzymatic label, chemiluminescent label or a biotinyl group or gold. Radioisotopes or radionuclides may include 3H, 14C, 15N, 35S, 90Y, 99Tc, 115In, 125I, 131I, fluorescent labels may include rhodamine, lanthanide phosphors or FITC and enzymatic labels may include horseradish peroxidase, β-galactosidase, luciferase, alkaline phosphatase. Additional labels include, by way of illustration and not limitation: enzymes, such as glucose-6-phosphate dehydrogenase (“G6PDH”), alpha-D-galactosidase, glucose oxydase, glucose amylase, carbonic anhydrase, acetylcholinesterase, lysozyme, malate dehydrogenase and peroxidase; dyes (e.g. cyanine dyes, e.g. Cy5TM, Cy5.5TM. or Cy7TM); additional fluorescent labels or fluorescers include, such as fluorescein and its derivatives, fluorochrome, GFP (GFP for “Green Fluorescent Protein”), other fluorescent proteins (e.g. mCherry, mTomato), dansyl, umbelliferone, phycoerythrin, phycocyanin, allophycocyanin, o-phthaldehyde, and fiuorescamine; fluorophores such as lanthanide cryptates and chelates e.g. Europium etc (Perkin Elmer and Cisbio Assays); chemoluminescent labels or chemiluminescers, such as isoluminol, luminol and the dioxetanes; sensitisers; coenzymes; enzyme substrates; particles, such as latex or carbon particles; metal sol; crystallite; liposomes; cells, etc., which may be further labelled with a dye, catalyst or other detectable group; molecules such as biotin, digoxygenin or 5-bromodeoxyuridine; toxin moieties, such as for example a toxin moiety selected from a group of Pseudomonas exotoxin (PE or a cytotoxic fragment or mutant thereof), Diptheria toxin or a cytotoxic fragment or mutant thereof, a botulinum toxin A, B, C, D, E or F, ricin or a cytotoxic fragment thereof e.g. ricin A, abrin or a cytotoxic fragment thereof, saporin or a cytotoxic fragment thereof, pokeweed antiviral toxin or a cytotoxic fragment thereof and bryodin 1 or a cytotoxic fragment thereof.

The term “light chain” when used in reference to an antibody refers to the immunoglobulin light chains, of which there are two types in mammals, lambda (λ) and kappa (κ). Preferably, the light chain is a human light chain. Preferably the light chain constant region is a human constant region. In the human population, multiple light chain constant region alleles exist. The nucleotide and amino acid sequences of these allelic variants are accessible on publicly available databases such as IMGT, ENSEMBL, Swiss-Prot and Uniprot. In one example, the antibodies disclosed herein comprise a protein encoded by a human κ constant region allele, which includes, but is not limited to, IGKC*01, IGKC*02, IGKC*03, IGKC*04 and IGKC*05 (the nucleotide and corresponding amino acid sequences of which are set out in Table 2). In one example, the antibodies disclosed herein comprise a protein encoded by a human κ constant region allele, which includes but is not limited to IGLC1*01, IGLC1*02, IGLC2*01, IGLC2*02, IGLC2*03, IGLC3*01, IGLC3*02, IGLC3*03, IGLC3*04, IGLC6*01, IGLC7*01, IGLC7*02, and IGLC7*03 (the nucleotide and corresponding amino acid sequences of which are set out in Table 2). In another example, the antibodies disclosed herein comprise a light chain constant region encoded by a mouse κ constant region allele, which includes, but is not limited to, IGKC*01, IGKC*03 or IGKC*03 (the nucleotide and corresponding amino acid sequences of which are set out in Table 2). In another example, the antibodies disclosed herein comprise a light chain constant region encoded by a mouse λ constant region allele, which includes, but is not limited to, IGLC1*01, IGLC2*01 or IGLC3*01 (the nucleotide and corresponding amino acid sequences of which are set out in Table 2).

“Percent (%) amino acid sequence identity” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEG ALIGN™ (DNASTAR) software. In one example, the % identity is about 70%. In one example, the % identity is about 75%. In one example, the % identity is about 80%. In one example, the % identity is about 85%. In one example, the % identity is about 90%. In one example, the % identity is about 92%. In one example, the % identity is about 95%. In one example, the % identity is about 97%. In one example, the % identity is about 98%. In one example, the % identity is about 99%. In one example, the % identity is 100%.

The term “naturally occurring” or “native” when used in connection with biological materials such as nucleic acid molecules, polypeptides, host cells, and the like, refers to those which are found in nature and not manipulated by a human being.

As used herein, “packaging” refers to how the components are organized and/or restrained into a unit fit for distribution and/or use. Packaging can include, e.g. boxes, bags, syringes, ampoules, vials, tubes, clamshell packaging, barriers and/or containers to maintain sterility, labelling, etc.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

As used herein, the term “polynucleotide,” “nucleotide,” nucleic acid” “nucleic acid molecule” and other similar terms are used interchangeable and include DNA, RNA, mRNA and the like.

As used herein, the terms “prevent”, “preventing”, and “prevention” refer to the total or partial inhibition of the development, recurrence, onset or spread of infection, resulting from the administration of a therapy or combination of therapies provided herein (e.g. a combination of prophylactic or therapeutic agents, such as an antibody).

The term “soluble” refers to a polypeptide that is lacking one or more transmembrane or cytoplasmic domains found in the native or membrane-associated form. In one example, the “soluble” form of a polypeptide lacks both the transmembrane domain and the cytoplasmic domain.

The term “subject” or “patient” refers to any animal, including, but not limited to, mammals. As used herein, the term “mammal” refers to any vertebrate animal that suckle their young and either give birth to living young (eutharian or placental mammals) or are egg-laying (metatharian or nonplacental mammals). Examples of mammalian species include, but are not limited to, humans and other primates, including non-human primates such as chimpanzees and other apes and monkey species; farm animals such as cattle, sheep, pigs, goats and horses; domestic mammals such as dogs and cats; laboratory animals including rodents such as mice, rats (including cotton rats) and guinea pigs; birds, including domestic, wild and game birds such as chickens, turkeys and other gallinaceous birds, ducks, geese, and the like.

As used herein “substantially all” refers to refers to at least about 60%, at least about 70%, at least about 75%, at least about 80%, at least about 85%, at least about 90%, at least about 95%, at least about 98%, at least about 99%, or about 100%.

The term “surfactant” as used herein refers to organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and non-ionic surfactants. Surfactants are often used as wetting, emulsifying, solubilizing, and dispersing agents for various pharmaceutical compositions and preparations of biological materials.

As used herein, the term “tag” refers to any type of moiety that is attached to, e.g. a polypeptide and/or a polynucleotide that encodes an antibody as described herein. For example, a polynucleotide that encodes a an antibody as described herein can contain one or more additional tag-encoding nucleotide sequences that encode e.g. a detectable moiety or a moiety that aids in affinity purification. When translated, the tag and the antibody can be in the form of a fusion protein. The term “detectable” or “detection” with reference to a tag refers to any tag that is capable of being visualized or wherein the presence of the tag is otherwise able to be determined and/or measured (e.g. by quantitation). A non-limiting example of a detectable tag is a fluorescent tag.

As used herein, the term “therapeutic agent” refers to any agent that can be used in the treatment, management or amelioration of a bacterial infection caused by Acinetobacter baumannii and/or a symptom related thereto. In certain examples, the term “therapeutic agent” refers to an antibody. In certain other examples, the term “therapeutic agent” refers to an agent other than an antibody. Preferably, a therapeutic agent is an agent which is known to be useful for, or has been or is currently being used for the treatment, management or amelioration of a bacterial infection caused by Acinetobacter baumannii and/or one or more symptoms related thereto.

As used herein, the term “therapy” refers to any protocol, method and/or agent that can be used in the prevention, management, treatment and/or amelioration of a bacterial infection caused by Acinetobacter baumannii. In certain examples, the terms “therapies” and “therapy” refer to a biological therapy, supportive therapy, and/or other therapies useful in the prevention, management, treatment and/or amelioration of a bacterial infection caused by Acinetobacter baumannii known to one of skill in the art such as medical personnel.

The terms “treat”, “treatment” and “treating” refer to the reduction or amelioration of the progression, severity, and/or duration of a bacterial infection caused by Acinetobacter baumannii resulting from the administration of one or more therapies (including, but not limited to, the administration of one or more prophylactic or therapeutic agents, such as an antibody). In specific examples, such terms refer to the reduction of the bacterial load, and/or the inhibition or reduction of one or more symptoms associated with a bacterial infection caused by Acinetobacter baumannii. Reduction of bacterial load may be measured via enumeration of cultured bacterial colonies on nutrient agar plates after plating of organ homogenates. IL-6 level is measured by ELISA.

The term “variable region” or “variable domain” refers to a portion of the light and heavy chains, typically about the amino-terminal 120 to 130 amino acids in the heavy chain and about 100 to 110 amino acids in the light chain, which differ extensively in sequence among antibodies and are used in the binding and specificity of each particular antibody for its particular antigen. The variability in sequence is concentrated in those regions called complimentarily determining regions (CDRs) while the more highly conserved regions in the variable domain are called framework regions (FR). The CDRs are primarily responsible for the interaction of the antibody with antigen. Numbering of amino acid positions used herein is according to IMGT (Lefranc MP “IMGT unique numbering for immunoglobulin and T cell receptor variable domains and Ig superfamily V-like domains”, Dev. Comp. Immunol. 27(1):55-77 (2003)). In preferred examples, the variable region is a human variable region.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (Eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present disclosure was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et al., ed., John Wiley and Sons, Inc.), Current Protocols in Cell Biology (CPCB) (Juan S. Bonifacino et al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various examples of the disclosure.

In a disclosure herein relating to an antibody which is defined by antibody sequence, the antibody sequence may be defined by reference to an antibody internal reference number or a SEQ ID NO. Any disclosure herein reciting an antibody internal reference number should also be taken as disclosure of the same subject-matter where the sequence defined by the antibody internal reference number is replaced by the corresponding SEQ ID NO.

### 5.2 Anti-Oxa-23 Antibodies

The present antibodies bind to Oxa-23 at the surface of intact bacteria (i.e., with non-ruptured membrane/cell wall), such as on, e.g., living bacteria.

The Oxa-23 carbapenemase enzyme is a 30kD protein expressed by the oxa-23 gene. Oxa-23 is a class D β-lactamase; a major group of enzymes involved in resistance to carbapenem antibiotics (Docquier J D, Mangani S. Structure-Function Relationships of Class D Carbapenemases. Curr Drug Targets. 2016; 17(9):1061-71. doi: 10.2174/1389450116666150825115824. PMID: 26302798., the contents of which are incorporated herein by reference).

The Oxa-23 gene encoding the Oxa-23 carbapenemase enzyme is widespread in Acinetobacter baumannii clinical isolates and compromises treatment with carbapenem antibiotics.

The sequence of the wild-type Oxa-23 enzyme is provided under GenBank accession no. AJ132105.1 and shown below:

(SEQ ID NO: 200)
MNKYFTCYVVASLFLSGCTVQHNLINETPSQIVQGHNQVIHQYFDEKNTS
GVLVIQTDKKINLYGNALSRANTEYVPASTFKMLNALIGLENQKTDINEI
FKWKGEKRSFTAWEKDMTLGEAMKLSAVPVYQELARRIGLDLMQKEVKRI
GFGNAEIGQQVDNFWLVGPLKVTPIQEVEFVSQLAHTQLPFSEKVQANVK
NMLLLEESNGYKIFGKTGWAMDIKPQVGWLTGWVEQPDGKIVAFALNMEM
RSEMPASIRNELLMKSLKQLNII.

The identification of mAbs binding to Oxa-23 is highly significant. The present inventors have unexpectedly found Oxa-23 is targetable by antibody in or closely associated with the Acinetobacter baumannii cell membrane which was previously unknown. In other words, the present inventors have unexpectedly found that the Oxa-23 enzyme is a membrane protein, present at the surface of live bacteria. Further, the present inventors have unexpectedly found that it is possible to use antibodies to block enzymatic function of Oxa-23, to render Acinetobacter baumannii susceptible to carbapenems. Still further, the present inventors have unexpectedly found that Oxa-23 binding epitopes are highly conserved across strains of the G2 clone of Acinetobacter baumannii. In particular, it is thought that its binding epitopes are conserved across at least 60% of strains of the G2 clone of Acinetobacter baumannii. As such, the present invention has demonstrated that targeting the Oxa-23 enzyme is highly attractive as a target for antibody-based diagnosis and treatment of Acinetobacter baumannii. In other words, an antibody targeting Oxa-23 at the surface of live bacteria can be considered as a pan-Acinetobacter baumannii antibody.

Accordingly, the antibodies of the invention that specifically bind to Oxa-23 are of great importance for diagnosis of an infection caused by Acinetobacter baumannii and for treatment of an infection caused by Acinetobacter baumannii in patients, particularly where Oxa-23 has been determined to be present in the patient.

Until the present invention, Oxa-23 had not been considered as a potential antibody target at the surface of bacteria since it was widely considered to be associated with the periplasm of the bacteria (Chiu C H, Liu Y H, Wang Y C, Lee Y T, Kuo S C, Chen T L, Lin J C, Wang F D. In vitro activity of SecA inhibitors in combination with carbapenems against carbapenem-hydrolysing class D s-lactamase-producing Acinetobacter baumannii. J Antimicrob Chemother. 2016 December; 71(12):3441-3448. doi: 10.1093/jac/dkw331. Epub 2016 Aug. 19. PMID: 27543656 the contents of which are incorporated herein by reference). This target has only been unexpectedly identified and validated by the methodology used by the present inventors for antibody discovery.

In specific examples, the antibody is a fully human antibody, such as a fully human monoclonal antibody.

Antibody 1348 has a heavy chain variable region (VH) amino acid sequence of SEQ ID No: 21, comprising the CDRH1 amino acid sequence of SEQ ID No: 22 (IMGT), the CDRH2 amino acid sequence of SEQ ID No: 23 (IMGT), and the CDRH3 amino acid sequence of SEQ ID No: 24 (IMGT). The heavy chain nucleic acid sequence of the VH domain is SEQ ID No: 20. Antibody 1348 has a light chain variable region (VL) amino acid sequence of SEQ ID No: 26, comprising the CDRL1 amino acid sequence of SEQ ID No: 27 (IMGT), the CDRL2 amino acid sequence of SEQ ID No: 28 (IMGT), and the CDRL3 amino acid sequence of SEQ ID No: 29 (IMGT). The light chain nucleic acid sequence of the VL domain is SEQ ID No: 25. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1349 has a heavy chain variable region (VH) amino acid sequence of SEQ ID No: 31, comprising the CDRH1 amino acid sequence of SEQ ID No: 32 (IMGT), the CDRH2 amino acid sequence of SEQ ID No: 33 (IMGT), and the CDRH3 amino acid sequence of SEQ ID No: 34 (IMGT). The heavy chain nucleic acid sequence of the VH domain is SEQ ID No: 30. Antibody 1349 has a light chain variable region (VL) amino acid sequence of SEQ ID No: 36, comprising the CDRL1 amino acid sequence of SEQ ID No: 37 (IMGT), the CDRL2 amino acid sequence of SEQ ID No: 28 (IMGT), and the CDRL3 amino acid sequence of SEQ ID No: 38 (IMGT). The light chain nucleic acid sequence of the VL domain is SEQ ID No: 35. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). In one embodiment, the heavy chain constant region sequence is SEQ ID NO: 418. In one embodiment, the light chain constant region sequence is SEQ ID NO: 448. In one embodiment, the antibody comprises a heavy chain comprising heavy chain variable region (VH) amino acid sequence of SEQ ID NO: 31 and heavy chain constant region amino acid sequence of SEQ ID NO: 418. In one embodiment, the antibody comprises a light chain comprising light chain variable region (VL) amino acid sequence of SEQ ID NO: 36 and light chain constant region amino acid sequence of SEQ ID NO: 448. In one embodiment, the antibody is antibody 1349. Antibody 1349 has heavy chain variable region (VH) amino acid sequence SEQ ID NO: 31, heavy chain constant region amino acid sequence SEQ ID NO: 418, light chain variable region (VL) amino acid sequence SEQ ID NO: 36, and light chain constant region amino acid sequence SEQ ID NO: 448).Antibody 1540 has a heavy chain variable region (VH) amino acid sequence of SEQ ID No: 21, comprising the CDRH1 amino acid sequence of SEQ ID No: 22 (IMGT), the CDRH2 amino acid sequence of SEQ ID No: 23 (IMGT), and the CDRH3 amino acid sequence of SEQ ID No: 24 (IMGT). The heavy chain nucleic acid sequence of the VH domain is SEQ ID No: 20. Antibody 1540 has a light chain variable region (VL) amino acid sequence of SEQ ID No: 26, comprising the CDRL1 amino acid sequence of SEQ ID No: 27 (IMGT), the CDRL2 amino acid sequence of SEQ ID No: 28 (IMGT), and the CDRL3 amino acid sequence of SEQ ID No: 29 (IMGT). The light chain nucleic acid sequence of the VL domain is SEQ ID No: 25. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1548 has a heavy chain variable region (VH) amino acid sequence of SEQ ID No: 172, comprising the CDRH1 amino acid sequence of SEQ ID No: 3 (IMGT), the CDRH2 amino acid sequence of SEQ ID No: 173 (IMGT), and the CDRH3 amino acid sequence of SEQ ID No: 174 (IMGT). The heavy chain nucleic acid sequence of the VH domain is SEQ ID No: 171. Antibody 1548 has a light chain variable region (VL) amino acid sequence of SEQ ID No: 176, comprising the CDRL1 amino acid sequence of SEQ ID No: 149 (IMGT), the CDRL2 amino acid sequence of SEQ ID No: 177 (IMGT), and the CDRL3 amino acid sequence of SEQ ID No: 178 (IMGT). The light chain nucleic acid sequence of the VL domain is SEQ ID No: 175. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1550 has a heavy chain variable region (VH) amino acid sequence of SEQ ID No: 180, comprising the CDRH1 amino acid sequence of SEQ ID No: 181 (IMGT), the CDRH2 amino acid sequence of SEQ ID No: 173 (IMGT), and the CDRH3 amino acid sequence of SEQ ID No: 182 (IMGT). The heavy chain nucleic acid sequence of the VH domain is SEQ ID No: 179. Antibody 1550 has a light chain variable region (VL) amino acid sequence of SEQ ID No: 184, comprising the CDRL1 amino acid sequence of SEQ ID No: 185 (IMGT), the CDRL2 amino acid sequence of SEQ ID No: 28 (IMGT), and the CDRL3 amino acid sequence of SEQ ID No: 186 (IMGT). The light chain nucleic acid sequence of the VL domain is SEQ ID No: 183. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

### 5.3 Anti-OC1 LOS Antibodies

The present antibodies bind to OC1 LOS at the surface of intact bacteria (i.e., with non-ruptured membrane/cell wall), such as on, e.g., living bacteria.

Bacterial lipopolysaccharides (LPS) and lipooligosaccharides (LOS) are unique and complex glycolipids that provide characteristic components of the outer membranes of Gram-negative bacteria. LPS and LOS include a core oligosaccharide combined with repeated saccharide units that extend out from the cell surface. LPS and LOS constitute a protective barrier known to cover the wall of bacteria.

Until the present invention, such LPS and LOS have not been considered as potential antibody targets at the surface of bacteria due to their structural and antigenic diversity. In part, this is due to work carried out relating to the KDO sugar molecule. This target has only been unexpectedly identified and validated by the methodology used by the present inventors for antibody discovery.

OC1 LOS is a 10kD lipooligosaccharide expressed by the OC1 gene. Detail of its structure can be found in Kenyon J J, Nigro S J, Hall R M. Variation in the OC locus of Acinetobacter baumannii genomes predicts extensive structural diversity in the lipooligosaccharide. PLoS One. 2014 Sep. 23; 9(9):e107833. doi: 10.1371/journal.pone.0107833. PMID: 25247305; PMCID: PMC4172580, the contents of which are incorporated herein by reference.

The present inventors have unexpectedly found not only that OC1 LOS can be targeted by diagnostic and therapeutic antibodies but also that its binding epitopes are highly conserved across strains of the G2 clone of Acinetobacter baumannii. In particular, it is thought that its binding epitopes are conserved across at least 70% of strains of the G2 clone of Acinetobacter baumannii. As such, targeting the OC1 LOS is highly attractive as a target for antibody-based diagnosis and treatment of Acinetobacter baumannii. In other words, an antibody targeting OC1 LOS at the surface of live bacteria can be considered as a pan-Acinetobacter baumannii antibody.

Without wishing to be bound by theory, it is thought that the antibodies of the invention bind to the core oligosaccharide of OC1 LOS.

The antibodies of the invention that specifically bind to OC1 LOS are of great importance for diagnosis of an infection caused by Acinetobacter baumannii and for treatment of an infection caused by Acinetobacter baumannii in patients, particularly where OC1 LOS has been determined to be present in the patient.

In specific examples, the antibody is a fully human antibody, such as a fully human monoclonal antibody.

Antibody 1042 has a heavy chain variable region (VH) amino acid sequence of SEQ ID No: 188, comprising the CDRH1 amino acid sequence of SEQ ID No: 32 (IMGT), the CDRH2 amino acid sequence of SEQ ID No: 33 (IMGT), and the CDRH3 amino acid sequence of SEQ ID No: 189 (IMGT). The heavy chain nucleic acid sequence of the VH domain is SEQ ID No: 187. Antibody 1042 has a light chain variable region (VL) amino acid sequence of SEQ ID No: 191, comprising the CDRL1 amino acid sequence of SEQ ID No: 37 (IMGT), the CDRL2 amino acid sequence of SEQ ID No: 28 (IMGT), and the CDRL3 amino acid sequence of SEQ ID No: 192 (IMGT). The light chain nucleic acid sequence of the VL domain is SEQ ID No: 190. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1043 has a heavy chain variable region (VH) amino acid sequence of SEQ ID No: 194, comprising the CDRH1 amino acid sequence of SEQ ID No: 195 (IMGT), the CDRH2 amino acid sequence of SEQ ID No: 33 (IMGT), and the CDRH3 amino acid sequence of SEQ ID No: 196 (IMGT). The heavy chain nucleic acid sequence of the VH domain is SEQ ID No: 193. Antibody 1043 has a light chain variable region (VL) amino acid sequence of SEQ ID No: 198, comprising the CDRL1 amino acid sequence of SEQ ID No: 37 (IMGT), the CDRL2 amino acid sequence of SEQ ID No: 9 (IMGT), and the CDRL3 amino acid sequence of SEQ ID No: 199 (IMGT). The light chain nucleic acid sequence of the VL domain is SEQ ID No: 197. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1403 has a heavy chain variable region (VH) amino acid sequence of SEQ ID No: 88, comprising the CDRH1 amino acid sequence of SEQ ID No: 22 (IMGT), the CDRH2 amino acid sequence of SEQ ID No: 89 (IMGT), and the CDRH3 amino acid sequence of SEQ ID No: 90 (IMGT). The heavy chain nucleic acid sequence of the VH domain is SEQ ID No: 87. Antibody 1403 has a light chain variable region (VL) amino acid sequence of SEQ ID No: 92, comprising the CDRL1 amino acid sequence of SEQ ID No: 93 (IMGT), the CDRL2 amino acid sequence of SEQ ID No: 28 (IMGT), and the CDRL3 amino acid sequence of SEQ ID No: 94 (IMGT). The light chain nucleic acid sequence of the VL domain is SEQ ID No: 91. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1405 has a heavy chain variable region (VH) amino acid sequence of SEQ ID No: 103, comprising the CDRH1 amino acid sequence of SEQ ID No: 104 (IMGT), the CDRH2 amino acid sequence of SEQ ID No: 33 (IMGT), and the CDRH3 amino acid sequence of SEQ ID No: 105 (IMGT). The heavy chain nucleic acid sequence of the VH domain is SEQ ID No: 102. Antibody 1405 has a light chain variable region (VL) amino acid sequence of SEQ ID No: 107, comprising the CDRL1 amino acid sequence of SEQ ID No: 27 (IMGT), the CDRL2 amino acid sequence of SEQ ID No: 28 (IMGT), and the CDRL3 amino acid sequence of SEQ ID No: 108 (IMGT). The light chain nucleic acid sequence of the VL domain is SEQ ID No: 106. The VH domain may be combined with any of the heavy chain constant region sequences described herein (the nucleotide and corresponding amino acid sequences of which are set out in Table 2).

The VL domain may be combined with any of the light chain constant region sequences described herein (the nucleotide and corresponding amino acid sequences of which are set out in Table 2).

Antibody 1407 has a heavy chain variable region (VH) amino acid sequence of SEQ ID No: 110, comprising the CDRH1 amino acid sequence of SEQ ID No: 111 (IMGT), the CDRH2 amino acid sequence of SEQ ID No: 33 (IMGT), and the CDRH3 amino acid sequence of SEQ ID No: 112 (IMGT). The heavy chain nucleic acid sequence of the VH domain is SEQ ID No: 109. Antibody 1407 has a light chain variable region (VL) amino acid sequence of SEQ ID No: 114, comprising the CDRL1 amino acid sequence of SEQ ID No: 115 (IMGT), the CDRL2 amino acid sequence of SEQ ID No: 28 (IMGT), and the CDRL3 amino acid sequence of SEQ ID No: 116 (IMGT). The light chain nucleic acid sequence of the VL domain is SEQ ID No: 113. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1408 has a heavy chain variable region (VH) amino acid sequence of SEQ ID No: 118, comprising the CDRH1 amino acid sequence of SEQ ID No: 104 (IMGT), the CDRH2 amino acid sequence of SEQ ID No: 33 (IMGT), and the CDRH3 amino acid sequence of SEQ ID No: 119 (IMGT). The heavy chain nucleic acid sequence of the VH domain is SEQ ID No: 117. Antibody 1408 has a light chain variable region (VL) amino acid sequence of SEQ ID No: 121, comprising the CDRL1 amino acid sequence of SEQ ID No: 122 (IMGT), the CDRL2 amino acid sequence of SEQ ID No: 28 (IMGT), and the CDRL3 amino acid sequence of SEQ ID No: 123 (IMGT). The light chain nucleic acid sequence of the VL domain is SEQ ID No: 120. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1413 has a heavy chain variable region (VH) amino acid sequence of SEQ ID No: 144, comprising the CDRH1 amino acid sequence of SEQ ID No: 145 (IMGT), the CDRH2 amino acid sequence of SEQ ID No: 33 (IMGT), and the CDRH3 amino acid sequence of SEQ ID No: 146 (IMGT). The heavy chain nucleic acid sequence of the VH domain is SEQ ID No: 143. Antibody 1413 has a light chain variable region (VL) amino acid sequence of SEQ ID No: 148, comprising the CDRL1 amino acid sequence of SEQ ID No: 149 (IMGT), the CDRL2 amino acid sequence of SEQ ID No: 150 (IMGT), and the CDRL3 amino acid sequence of SEQ ID No: 151 (IMGT). The light chain nucleic acid sequence of the VL domain is SEQ ID No: 147. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

### 5.4 Anti-KL49 Antibodies

The present antibodies bind to KL49 at the surface of intact bacteria (i.e., with non-ruptured membrane/cell wall), such as on, e.g., living bacteria.

KL49 is a form of capsular polysaccharide (CPS) produced by strains of A. baumannii and surrounding the outer cell membrane. CPS is a densely packed high-molecular weight carbohydrate composed of repeating oligosaccharide units, providing a barrier against environmental stressors and confers resistance against antimicrobial compounds and immunological protective mechanisms.

The structure of the KL49 capsule antigen is described in Singh J K, Adams F G, Brown M H. Diversity and Function of Capsular Polysaccharide in <i>Acinetobacter baumannii</i>. Front Microbiol. 2019 Jan. 9; 9:3301. doi: 10.3389/fmicb.2018.03301. PMID: 30687280; PMCID: PMC6333632, the contents of which are incorporated herein by reference.

The presence of the KL49 capsule is associated with hyper virulence and increased mortality, and Acinetobacter baumannii KL49 is currently emerging as the major clone in South East Asia (Zhou K, Tang X, Wang L, Guo Z, Xiao S, Wang Q, Zhuo C. An Emerging Clone (ST457) of Acinetobacter baumannii Clonal Complex 92 With Enhanced Virulence and Increasing Endemicity in South China. Clin Infect Dis. 2018 Nov. 13; 67(suppl_2):S179-S188. doi: 10.1093/cid/ciy691. PMID: 30423046, the contents of which are incorporated herein by reference). Thus, the discovery by the present inventors that the KL49 protein is present at the surface of live bacteria and can be targeted by diagnostic and therapeutic antibodies is of significant value for enabling early diagnosis and treatment of these patients.

Accordingly, the antibodies of the invention that specifically bind to KL49 are of great importance for diagnosis of an infection caused by Acinetobacter baumannii and for treatment of an infection caused by Acinetobacter baumannii in patients, particularly where KL49 has been determined to be present in the patient.

In specific examples, the antibody is a fully human antibody, such as a fully human monoclonal antibody.

Antibody 1345 has a heavy chain variable region (VH) amino acid sequence of SEQ ID No: 2, comprising the CDRH1 amino acid sequence of SEQ ID No: 3 (IMGT), the CDRH2 amino acid sequence of SEQ ID No: 4 (IMGT), and the CDRH3 amino acid sequence of SEQ ID No: 5 (IMGT). The heavy chain nucleic acid sequence of the VH domain is SEQ ID No: 1. Antibody 1345 has a light chain variable region (VL) amino acid sequence of SEQ ID No: 7, comprising the CDRL1 amino acid sequence of SEQ ID No: 8 (IMGT), the CDRL2 amino acid sequence of SEQ ID No: 9 (IMGT), and the CDRL3 amino acid sequence of SEQ ID No: 10 (IMGT). The light chain nucleic acid sequence of the VL domain is SEQ ID No: 6. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1347 has a heavy chain variable region (VH) amino acid sequence of SEQ ID No: 12, comprising the CDRH1 amino acid sequence of SEQ ID No: 3 (IMGT), the CDRH2 amino acid sequence of SEQ ID No: 13 (IMGT), and the CDRH3 amino acid sequence of SEQ ID No: 14 (IMGT). The heavy chain nucleic acid sequence of the VH domain is SEQ ID No: 11. Antibody 1347 has a light chain variable region (VL) amino acid sequence of SEQ ID No: 16, comprising the CDRL1 amino acid sequence of SEQ ID No: 17 (IMGT), the CDRL2 amino acid sequence of SEQ ID No: 18 (IMGT), and the CDRL3 amino acid sequence of SEQ ID No: 19 (IMGT). The light chain nucleic acid sequence of the VL domain is SEQ ID No: 15. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1350 has a heavy chain variable region (VH) amino acid sequence of SEQ ID No: 40, comprising the CDRH1 amino acid sequence of SEQ ID No: 3 (IMGT), the CDRH2 amino acid sequence of SEQ ID No: 13 (IMGT), and the CDRH3 amino acid sequence of SEQ ID No: 41 (IMGT). The heavy chain nucleic acid sequence of the VH domain is SEQ ID No: 39. Antibody 1350 has a light chain variable region (VL) amino acid sequence of SEQ ID No: 16, comprising the CDRL1 amino acid sequence of SEQ ID No: 17 (IMGT), the CDRL2 amino acid sequence of SEQ ID No: 18 (IMGT), and the CDRL3 amino acid sequence of SEQ ID No: 19 (IMGT). The light chain nucleic acid sequence of the VL domain is SEQ ID No: 42. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1351 has a heavy chain variable region (VH) amino acid sequence of SEQ ID No: 44, comprising the CDRH1 amino acid sequence of SEQ ID No: 3 (IMGT), the CDRH2 amino acid sequence of SEQ ID No: 13 (IMGT), and the CDRH3 amino acid sequence of SEQ ID No: 45 (IMGT). The heavy chain nucleic acid sequence of the VH domain is SEQ ID No: 43. Antibody 1351 has a light chain variable region (VL) amino acid sequence of SEQ ID No: 47, comprising the CDRL1 amino acid sequence of SEQ ID No: 17 (IMGT), the CDRL2 amino acid sequence of SEQ ID No: 18 (IMGT), and the CDRL3 amino acid sequence of SEQ ID No: 10 (IMGT). The light chain nucleic acid sequence of the VL domain is SEQ ID No: 46. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1363 has a heavy chain variable region (VH) amino acid sequence of SEQ ID No: 49, comprising the CDRH1 amino acid sequence of SEQ ID No: 3 (IMGT), the CDRH2 amino acid sequence of SEQ ID No: 13 (IMGT), and the CDRH3 amino acid sequence of SEQ ID No: 50 (IMGT). The heavy chain nucleic acid sequence of the VH domain is SEQ ID No: 48. Antibody 1363 has a light chain variable region (VL) amino acid sequence of SEQ ID No: 52, comprising the CDRL1 amino acid sequence of SEQ ID No: 17 (IMGT), the CDRL2 amino acid sequence of SEQ ID No: 18 (IMGT), and the CDRL3 amino acid sequence of SEQ ID No: 19 (IMGT). The light chain nucleic acid sequence of the VL domain is SEQ ID No: 51. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1364 has a heavy chain variable region (VH) amino acid sequence of SEQ ID No: 54, comprising the CDRH1 amino acid sequence of SEQ ID No: 3 (IMGT), the CDRH2 amino acid sequence of SEQ ID No: 13 (IMGT), and the CDRH3 amino acid sequence of SEQ ID No: 55 (IMGT). The heavy chain nucleic acid sequence of the VH domain is SEQ ID No: 53. Antibody 1364 has a light chain variable region (VL) amino acid sequence of SEQ ID No: 57, comprising the CDRL1 amino acid sequence of SEQ ID No: 58 (IMGT), the CDRL2 amino acid sequence of SEQ ID No: 18 (IMGT), and the CDRL3 amino acid sequence of SEQ ID No: 59 (IMGT). The light chain nucleic acid sequence of the VL domain is SEQ ID No: 56. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1397 has a heavy chain variable region (VH) amino acid sequence of SEQ ID No: 61, comprising the CDRH1 amino acid sequence of SEQ ID No: 3 (IMGT), the CDRH2 amino acid sequence of SEQ ID No: 13 (IMGT), and the CDRH3 amino acid sequence of SEQ ID No: 62 (IMGT). The heavy chain nucleic acid sequence of the VH domain is SEQ ID No: 60. Antibody 1397 has a light chain variable region (VL) amino acid sequence of SEQ ID No: 64, comprising the CDRL1 amino acid sequence of SEQ ID No: 17 (IMGT), the CDRL2 amino acid sequence of SEQ ID No: 18 (IMGT), and the CDRL3 amino acid sequence of SEQ ID No: 59 (IMGT). The light chain nucleic acid sequence of the VL domain is SEQ ID No: 63. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1398 has a heavy chain variable region (VH) amino acid sequence of SEQ ID No: 66, comprising the CDRH1 amino acid sequence of SEQ ID No: 3 (IMGT), the CDRH2 amino acid sequence of SEQ ID No: 13 (IMGT), and the CDRH3 amino acid sequence of SEQ ID No: 67 (IMGT). The heavy chain nucleic acid sequence of the VH domain is SEQ ID No: 65. Antibody 1398 has a light chain variable region (VL) amino acid sequence of SEQ ID No: 69, comprising the CDRL1 amino acid sequence of SEQ ID No: 17 (IMGT), the CDRL2 amino acid sequence of SEQ ID No: 18 (IMGT), and the CDRL3 amino acid sequence of SEQ ID No: 59 (IMGT). The light chain nucleic acid sequence of the VL domain is SEQ ID No: 68. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1400 has a heavy chain variable region (VH) amino acid sequence of SEQ ID No: 71, comprising the CDRH1 amino acid sequence of SEQ ID No: 3 (IMGT), the CDRH2 amino acid sequence of SEQ ID No: 13 (IMGT), and the CDRH3 amino acid sequence of SEQ ID No: 72 (IMGT). The heavy chain nucleic acid sequence of the VH domain is SEQ ID No: 70. Antibody 1400 has a light chain variable region (VL) amino acid sequence of SEQ ID No: 74, comprising the CDRL1 amino acid sequence of SEQ ID No: 17 (IMGT), the CDRL2 amino acid sequence of SEQ ID No: 18 (IMGT), and the CDRL3 amino acid sequence of SEQ ID No: 59 (IMGT). The light chain nucleic acid sequence of the VL domain is SEQ ID No: 73. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1401 has a heavy chain variable region (VH) amino acid sequence of SEQ ID No: 76, comprising the CDRH1 amino acid sequence of SEQ ID No: 3 (IMGT), the CDRH2 amino acid sequence of SEQ ID No: 13 (IMGT), and the CDRH3 amino acid sequence of SEQ ID No: 77 (IMGT). The heavy chain nucleic acid sequence of the VH domain is SEQ ID No: 75. Antibody 1401 has a light chain variable region (VL) amino acid sequence of SEQ ID No: 79, comprising the CDRL1 amino acid sequence of SEQ ID No: 17 (IMGT), the CDRL2 amino acid sequence of SEQ ID No: 18 (IMGT), and the CDRL3 amino acid sequence of SEQ ID No: 80 (IMGT). The light chain nucleic acid sequence of the VL domain is SEQ ID No: 78. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1402 has a heavy chain variable region (VH) amino acid sequence of SEQ ID No: 82, comprising the CDRH1 amino acid sequence of SEQ ID No: 3(IMGT), the CDRH2 amino acid sequence of SEQ ID No: 13 (IMGT), and the CDRH3 amino acid sequence of SEQ ID No: 83 (IMGT). The heavy chain nucleic acid sequence of the VH domain is SEQ ID No: 81. Antibody 1402 has a light chain variable region (VL) amino acid sequence of SEQ ID No: 85, comprising the CDRL1 amino acid sequence of SEQ ID No: 17 (IMGT), the CDRL2 amino acid sequence of SEQ ID No: 18 (IMGT), and the CDRL3 amino acid sequence of SEQ ID No: 86 (IMGT). The light chain nucleic acid sequence of the VL domain is SEQ ID No: 84. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1404 has a heavy chain variable region (VH) amino acid sequence of SEQ ID No: 96, comprising the CDRH1 amino acid sequence of SEQ ID No: 3 (IMGT), the CDRH2 amino acid sequence of SEQ ID No: 97 (IMGT), and the CDRH3 amino acid sequence of SEQ ID No: 98 (IMGT). The heavy chain nucleic acid sequence of the VH domain is SEQ ID No: 95. Antibody 1404 has a light chain variable region (VL) amino acid sequence of SEQ ID No: 100, comprising the CDRL1 amino acid sequence of SEQ ID No: 101 (IMGT), the CDRL2 amino acid sequence of SEQ ID No: 18 (IMGT), and the CDRL3 amino acid sequence of SEQ ID No: 59 (IMGT). The light chain nucleic acid sequence of the VL domain is SEQ ID No: 99. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1409 has a heavy chain variable region (VH) amino acid sequence of SEQ ID No: 125, comprising the CDRH1 amino acid sequence of SEQ ID No: 3 (IMGT), the CDRH2 amino acid sequence of SEQ ID No: 13 (IMGT), and the CDRH3 amino acid sequence of SEQ ID No: 126 (IMGT). The heavy chain nucleic acid sequence of the VH domain is SEQ ID No: 124. Antibody 1409 has a light chain variable region (VL) amino acid sequence of SEQ ID No: 128, comprising the CDRL1 amino acid sequence of SEQ ID No: 17 (IMGT), the CDRL2 amino acid sequence of SEQ ID No: 18 (IMGT), and the CDRL3 amino acid sequence of SEQ ID No: 129 (IMGT). The light chain nucleic acid sequence of the VL domain is SEQ ID No: 127. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1410 has a heavy chain variable region (VH) amino acid sequence of SEQ ID No: 131, comprising the CDRH1 amino acid sequence of SEQ ID No: 3 (IMGT), the CDRH2 amino acid sequence of SEQ ID No: 13 (IMGT), and the CDRH3 amino acid sequence of SEQ ID No: 132 (IMGT). The heavy chain nucleic acid sequence of the VH domain is SEQ ID No: 130. Antibody 1410 has a light chain variable region (VL) amino acid sequence of SEQ ID No: 134, comprising the CDRL1 amino acid sequence of SEQ ID No: 17 (IMGT), the CDRL2 amino acid sequence of SEQ ID No: 18 (IMGT), and the CDRL3 amino acid sequence of SEQ ID No: 135 (IMGT). The light chain nucleic acid sequence of the VL domain is SEQ ID No: 133. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1412 has a heavy chain variable region (VH) amino acid sequence of SEQ ID No: 137, comprising the CDRH1 amino acid sequence of SEQ ID No: 3 (IMGT), the CDRH2 amino acid sequence of SEQ ID No: 138 (IMGT), and the CDRH3 amino acid sequence of SEQ ID No: 139 (IMGT). The heavy chain nucleic acid sequence of the VH domain is SEQ ID No: 136. Antibody 1412 has a light chain variable region (VL) amino acid sequence of SEQ ID No: 141, comprising the CDRL1 amino acid sequence of SEQ ID No: 142 (IMGT), the CDRL2 amino acid sequence of SEQ ID No: 18 (IMGT), and the CDRL3 amino acid sequence of SEQ ID No: 86 (IMGT). The light chain nucleic acid sequence of the VL domain is SEQ ID No: 140. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1414 has a heavy chain variable region (VH) amino acid sequence of SEQ ID No: 153, comprising the CDRH1 amino acid sequence of SEQ ID No: 154 (IMGT), the CDRH2 amino acid sequence of SEQ ID No: 155 (IMGT), and the CDRH3 amino acid sequence of SEQ ID No: 156 (IMGT). The heavy chain nucleic acid sequence of the VH domain is SEQ ID No: 152. Antibody 1414 has a light chain variable region (VL) amino acid sequence of SEQ ID No: 158, comprising the CDRL1 amino acid sequence of SEQ ID No: 17 (IMGT), the CDRL2 amino acid sequence of SEQ ID No: 18 (IMGT), and the CDRL3 amino acid sequence of SEQ ID No: 159 (IMGT). The light chain nucleic acid sequence of the VL domain is SEQ ID No: 157. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1415 has a heavy chain variable region (VH) amino acid sequence of SEQ ID No: 161, comprising the CDRH1 amino acid sequence of SEQ ID No: 3 (IMGT), the CDRH2 amino acid sequence of SEQ ID No: 162(IMGT), and the CDRH3 amino acid sequence of SEQ ID No: 163 (IMGT). The heavy chain nucleic acid sequence of the VH domain is SEQ ID No: 160. Antibody 1415 has a light chain variable region (VL) amino acid sequence of SEQ ID No: 165, comprising the CDRL1 amino acid sequence of SEQ ID No: 17 (IMGT), the CDRL2 amino acid sequence of SEQ ID No: 18 (IMGT), and the CDRL3 amino acid sequence of SEQ ID No: 135 (IMGT). The light chain nucleic acid sequence of the VL domain is SEQ ID No: 164. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2).

Antibody 1416 has a heavy chain variable region (VH) amino acid sequence of SEQ ID No: 167, comprising the CDRH1 amino acid sequence of SEQ ID No: 3 (IMGT), the CDRH2 amino acid sequence of SEQ ID No: 13 (IMGT), and the CDRH3 amino acid sequence of SEQ ID No: 168 (IMGT). The heavy chain nucleic acid sequence of the VH domain is SEQ ID No: 166. Antibody 1416 has a light chain variable region (VL) amino acid sequence of SEQ ID No: 170, comprising the CDRL1 amino acid sequence of SEQ ID No: 17 (IMGT), the CDRL2 amino acid sequence of SEQ ID No: 18 (IMGT), and the CDRL3 amino acid sequence of SEQ ID No: 10 (IMGT). The light chain nucleic acid sequence of the VL domain is SEQ ID No: 169. The VH domain may be combined with any of the heavy chain constant region sequences described herein (the nucleotide and corresponding amino acid sequences of which are set out in Table 2). The VL domain may be combined with any of the light chain constant region sequences described herein (the nucleotide and corresponding amino acid sequences of which are set out in Table 2). In one embodiment, the heavy chain constant region sequence is SEQ ID NO: 418. In one embodiment, the light chain constant region sequence is SEQ ID NO: 448. In one embodiment, the antibody comprises a heavy chain comprising heavy chain variable region (VH) amino acid sequence of SEQ ID NO: 167 and heavy chain constant region amino acid sequence of SEQ ID NO: 418. In one embodiment, the antibody comprises a light chain comprising light chain variable region (VL) amino acid sequence of SEQ ID NO: 170 and light chain constant region amino acid sequence of SEQ ID NO: 448. In one embodiment, the antibody is antibody 1416. Antibody 1416 has heavy chain variable region (VH) amino acid sequence SEQ ID NO: 167, heavy chain constant region amino acid sequence SEQ ID NO: 418, light chain variable region (VL) amino acid sequence SEQ ID NO: 170, and light chain constant region amino acid sequence SEQ ID NO: 448).

### 5.5 Antibody Function—Binding Measurement

An antibody that specifically binds at the surface of Acinetobacter baumannii bacteria can be identified, for example, by immunoassays (e.g. ELISA), BIAcore™, or other techniques known to those of skill in the art.

An antibody binds specifically to a target when it binds with higher affinity than to any cross-reactive antigen as determined using experimental techniques, such as radioimmunoassays (RIA) and enzyme-linked immunosorbent assays (ELISAs). Typically, a specific or selective reaction will be at least twice background signal or noise and more typically more than 10 times (such as more than 15 times, more than 20 times, more than 50 times or more than 100 times) background. See, e.g. Paul, ed., 1989, Fundamental Immunology Second Edition, Raven Press, New York at pages 332-336 for a discussion regarding antibody specificity.

Any suitable method may be used to determine whether an antibody binds at the surface of Acinetobacter baumannii bacteria. Such a method may comprise an ELISA to determine specificity of antibodies. An antibody may be said to bind its antigen if the level of binding to antigen is at least 2.5-fold greater, e.g., at least 10 fold greater, than binding to a control antigen. Binding between an antibody and its cognate antigen is often referred to as specific binding. Precise identification of the residues bound by an antibody can usually be obtained using x-ray crystallography. This technique may be used to determine that an antibody described herein binds one or more residues of Oxa-23 enzyme, OC1 LOS, or KL49 at the surface of Acinetobacter baumannii bacteria.

Measurements for binding assays can conveniently use an indirect whole bacterial cell ELISA. Alternatively, measurements for binding assays can conveniently use OMVs rather than bacterial cells. Thus, when investigating binding to live bacteria e.g. using an ELISA assay, binding to OMVs suitably may be used as the plate-coating antigen.

If the antibody epitope is a linear continuous epitope, then binding and binding affinity can be determined using synthetic purified peptide sequences.

The particular binding antigen (e.g. Oxa-23, OC1 LOS, or KL49) of an antibody may be determined using a phage expression library e.g. as described in Example 5. For example, shotgun A. Baumannii genomic DNA is incorporated into a phage expression library. The antibody is then screened for reactivity with phage plaques from the expression library and the individual phage binding to the antibody is identified. The identified phage is sequenced, and the sequencing information used to elucidate the binding antigen of the antibody. A suitable phage method for antigen identification will be known to a person skilled in the art. For example, a suitable phage method is described in Lodes M J, Dillon D C, Houghton R L, Skeiky Y A. Expression cloning. Methods Mol Med. 2004; 94:91-106. doi: 10.1385/1-59259-679-7:91. PMID: 14959824, the contents of which are incorporated by reference.

Identification of Oxa-23, OC1 LOS, or KL49 as the binding antigen may also be carried out using western blotting on specific GC2 strains genetically identified as producing the respective molecule.

A high-content imaging (HCl) assay and α system for image-based morphological profiling can be used to screen binding of mAbs to panels of clinically relevant CRAB isolates to assess binding affinity and cross-reactivity. (Maes M, Dyson Z A, Smith S E, Goulding D A, Ludden C, Baker S, Kellam P, Reece S T, Dougan G, Bartholdson Scott J. A novel therapeutic antibody screening method using bacterial high-content imaging reveals functional antibody binding phenotypes of Escherichia coli ST131. Sci Rep. 2020 Jul. 24; 10(1):12414. doi: 10.1038/s41598-020-69300-8, the contents of which are incorporated herein by reference).

SPR might be used to determine binding of an antibody to Oxa-23, OC1 LOS, or KL49 out of the cell wall context. A suitable SPR protocol is set out below:
1. Coupling mouse anti-human (or other relevant human, rat or non-human vertebrate antibody constant region species-matched) IgG to a biosensor chip (e.g. dextran-coated gold chip) such as by primary amine coupling. Thus, an anti-Fc antibody may be covalently immobilised on the chip surface using amine coupling.
2. Exposing the mouse anti-human IgG (or other matched species antibody) to the test antibody (e.g., in human IgG format) to capture the test antibody on the chip;
3. Passing the test antigen over the chip's capture surface at a series of concentrations up to a maximum of 100 nM, e.g., at 0.39, 1.56, 6.25, 25 and 100 nM, and a 0 nM (i.e. buffer alone) control run. The buffer may optionally be 0.01 M HEPES (4-(2-hydroxyethyl)-1-piperazineethanesulfonic acid), 0.15 M NaCl and 0.05% v/v surfactant P20 in aqueous solution, buffered to pH 7.4; and
4. Determining the affinity of binding of test antibody to test antigen using surface plasmon resonance. KD, Ka and Kd may then be calculated.

SPR can be carried out using any standard SPR apparatus, such as by Biacore™ or using the ProteOn XPR36™ (Bio-Rad@).

Regeneration of the capture surface can be carried out with 3 M magnesium chloride solution. This removes the captured test antibody and allows the surface to be used for another interaction. The binding data can be fitted to 1:1 model inherent using standard techniques, e.g. using analysis software such as Biacore Insight Evaluation Software.

Methods of determining binding (e.g., ELISA) can also be used to determine competition between molecules such as between a test antibody and α known antibody and may be performed with the test antibody and known antibody in IgG format, or optionally in scFv format.

Methods of determining binding (e.g., ELISA) can also be used to determine cross-reactivity of an antibody with different clones or strains of Acinetobacter baumannii.

### 5.6 Antibody Function—Inducing Complement Activation

The complement cascade is a powerful serum-based mechanism leading to insertion of protein pores into the membrane of gram-negative bacteria resulting in bacterial killing. This protein pathway is triggered by antibody and as such, the ability for an antibody to trigger the complement cascade is a key functional outcome for certain antibodies of the invention.

C3b is cleaved from the serum C3 protein to drive the complement cascade and mAb binding to the bacterial cell membrane initiates formation of an enzymatic complex involving the complement proteins Clq, C2 and C4 that cleave C3. Therefore, detection of C3b on the bacterial membrane is a correlate for successful initiation of the complement cascade.

The ability of an antibody to induce complement activation may be determined by in vitro assays, such as by flow cytometry-based assay to detect mAb-induced deposition of the complement component C3b on the surface of OMVs. Such an assay is described in Fishinger et al., Journal of Immunological Methods, https://doi.oruI0.1016/i.iim.2019.07.002, the contents of which are incorporated by reference. Such an assay is used herein in the Examples herein and involves 5 major steps:
1. FM4-64 stained GC2 A. baumannii OMVs are incubated for 10 mins with compensation beads to which the OMVs non-specifically associate following which the bead-OMV complexes are washed and blocked with PBS 0.1% (w/v) BSA.
2. The bead-OMV complexes are added to the small-scale expressed mAbs in HEK 293 supernatant, normalised to 100 μg/ml and incubated together for 3 hr at 37° C.
3. Complement proteins are prepared by reconstituting lyophilised guinea pig complement in gelatin veronal buffer and added to the bead-OMV-antibody complexes before incubation at 37° C. for 15 mins.
4. A FITC-conjugated anti-C3 detection antibody is added to the bead-based complexes after washing and incubated for a further 15 mins.
5. Following a further wash, fluorescence is assessed on an Attune NxT flow cytometer equipped with a high throughput autosampler to identify and quantify the FM4-64+FITC+bead-based immune complexes..

C3b complement deposition is reported as a fold change/HuIgG1 isotype control of the geomean fluorescence intensity on the FITC channel.

### 5.7 Gene Segments

In some aspects, the antibody comprises VH and/or VL domain and framework regions of human germline gene segment sequences. Gene segment sequences from which the exemplary antibodies described herein are derived are set out in Table 3.

In one example, the antibody comprises an antibody VH domain which is derived from recombination of a human heavy chain V gene segment, a human heavy chain D gene segment and a human heavy chain J gene segment.

In one example, the antibody comprises an antibody VL domain which is derived from recombination of a human light chain V gene segment and a human light chain J gene segment.

In one example, the antibody comprises an antibody VH domain which is derived from recombination of a human heavy chain V gene segment, a human heavy chain D gene segment and a human heavy chain J gene segment, wherein the V gene segment is IGHV3-23*04, IGHV3-9*01, IGHV2-5*10, IGHV3-9*01, or IGHV3-30*18; and/or the J gene segment is IGHJ2*01, IGHJ3*02, IGHJ4*02, IGHJ6*02, or IGHJ5*02.

In one example, the antibody comprises an antibody VH domain which is derived from recombination of a human heavy chain V gene segment, a human heavy chain D gene segment and a human heavy chain J gene segment, wherein the V gene segment is IGHV3-23*04.

In one example, the antibody comprises an antibody VH domain which is derived from recombination of a human heavy chain V gene segment, a human heavy chain D gene segment and a human heavy chain J gene segment, wherein the V gene segment is IGHV3-9*01.

In one example, the antibody comprises an antibody VH domain which is derived from recombination of a human heavy chain V gene segment, a human heavy chain D gene segment and a human heavy chain J gene segment, wherein the V gene segment is IGHV2-5*10.

In one example, the antibody comprises an antibody VH domain which is derived from recombination of a human heavy chain V gene segment, a human heavy chain D gene segment and a human heavy chain J gene segment, wherein the V gene segment is IGHV3-9*01.

In one example, the antibody comprises an antibody VH domain which is derived from recombination of a human heavy chain V gene segment, a human heavy chain D gene segment and a human heavy chain J gene segment, wherein the V gene segment is IGHV3-30*18.

In one example, the antibody comprises an antibody VH domain which is derived from recombination of a human heavy chain V gene segment, a human heavy chain D gene segment and a human heavy chain J gene segment, wherein the J gene segment is IGHJ2*01.

In one example, the antibody comprises an antibody VH domain which is derived from recombination of a human heavy chain V gene segment, a human heavy chain D gene segment and a human heavy chain J gene segment, wherein the J gene segment is IGHJ3*02.

In one example, the antibody comprises an antibody VH domain which is derived from recombination of a human heavy chain V gene segment, a human heavy chain D gene segment and a human heavy chain J gene segment, wherein the J gene segment is IGHJ4*02.

In one example, the antibody comprises an antibody VH domain which is derived from recombination of a human heavy chain V gene segment, a human heavy chain D gene segment and a human heavy chain J gene segment, wherein the J gene segment is IGHJ6*02.

In one example, the antibody comprises an antibody VH domain which is derived from recombination of a human heavy chain V gene segment, a human heavy chain D gene segment and a human heavy chain J gene segment, wherein the J gene segment is IGHJ5*02.

In one example, the antibody comprises an antibody VH domain which is derived from recombination of a human heavy chain V gene segment, a human heavy chain D gene segment and a human heavy chain J gene segment, wherein the V gene segment and the J gene segment for the heavy chain is the combination shown in Table 3 for any one of the antibodies.

In one example, the antibody comprises an antibody VL domain which is derived from recombination of a human light chain V gene segment and a human light chain J gene segment, wherein the V gene segment is IGKV3-15*01, IGKV3D-15*d01, IGKV1-17*01, IGKV1-9*d01, IGKVID-39*01, IGKV1-16*02, IGKV1-6*01, IGKV3D-15*d01, IGKV1-12*01, or IGKVID-16*01; and/or the J gene segment is IGKJ4*01, IGKJ3*01, IGKJ1*01, or IGKJ2*04.

In one example, the antibody comprises an antibody VL domain which is derived from recombination of a human light chain V gene segment and a human light chain J gene segment, wherein the V gene segment is IGKV3-15*01.

In one example, the antibody comprises an antibody VL domain which is derived from recombination of a human light chain V gene segment and a human light chain J gene segment, wherein the V gene segment isIGKV3D-15*d01.

In one example, the antibody comprises an antibody VL domain which is derived from recombination of a human light chain V gene segment and a human light chain J gene segment, wherein the V gene segment is IGKV1-17*01.

In one example, the antibody comprises an antibody VL domain which is derived from recombination of a human light chain V gene segment and a human light chain J gene segment, wherein the V gene segment is IGKV1-9*d01.

In one example, the antibody comprises an antibody VL domain which is derived from recombination of a human light chain V gene segment and a human light chain J gene segment, wherein the V gene segment is IGKV1D-39*01.

In one example, the antibody comprises an antibody VL domain which is derived from recombination of a human light chain V gene segment and a human light chain J gene segment, wherein the V gene segment is IGKV1-16*02.

In one example, the antibody comprises an antibody VL domain which is derived from recombination of a human light chain V gene segment and a human light chain J gene segment, wherein the V gene segment is IGKV1-6*01.

In one example, the antibody comprises an antibody VL domain which is derived from recombination of a human light chain V gene segment and a human light chain J gene segment, wherein the V gene segment is IGKV3D-15*d01.

In one example, the antibody comprises an antibody VL domain which is derived from recombination of a human light chain V gene segment and a human light chain J gene segment, wherein the V gene segment is IGKV1-12*01.

In one example, the antibody comprises an antibody VL domain which is derived from recombination of a human light chain V gene segment and a human light chain J gene segment, wherein the J gene segment is IGKJ4*01.

In one example, the antibody comprises an antibody VL domain which is derived from recombination of a human light chain V gene segment and a human light chain J gene segment, wherein the J gene segment is IGKJ3*01.

In one example, the antibody comprises an antibody VL domain which is derived from recombination of a human light chain V gene segment and a human light chain J gene segment, wherein the J gene segment is IGKJ1*01.

In one example, the antibody comprises an antibody VL domain which is derived from recombination of a human light chain V gene segment and a human light chain J gene segment, wherein the J gene segment is IGKJ2*04.

In one example, the antibody comprises an antibody VL domain which is derived from recombination of a human light chain V gene segment and a human light chain J gene segment, wherein the V gene segment and the J gene segment for the light chain is the combination shown in Table 3 for any one of the antibodies.

### 5.8 Sequence Identity

In some examples, the antibody comprises an amino acid sequence which has a high level of sequence identity to the amino acid sequence of one of the exemplary antibodies described herein and set out in Table 1.

In one example, the amino acid sequence is at least 70% identical to the specified SEQ ID No. In one example, the amino acid sequence is at least 75% identical to the specified SEQ ID No. In one example, the amino acid sequence is at least 95% identical to the specified SEQ ID No. In one example, the amino acid sequence is at least 96% identical to the specified SEQ ID No. In one example, the amino acid sequence is at least 97% identical to the specified SEQ ID No. In one example, the amino acid sequence is at least 98% identical to the specified SEQ ID No. In one example, the amino acid sequence is at least 99% identical to the specified SEQ ID No. In one example, the amino acid sequence is at least 99.5% identical to the specified SEQ ID No.

In one example, an antibody is provided having a full heavy chain sequence that is at least 70% identical to the amino acid sequence of the full heavy chain sequence of any antibody disclosed herein e.g., the combination of the SEQ ID Nos for the heavy chain variable domain and heavy chain constant domain sequences disclosed herein. In one example, an antibody is provided having a full heavy chain sequence that is at least 75% identical to the amino acid sequence of the full heavy chain sequence of any antibody disclosed herein e.g., the combination of the SEQ ID Nos for the heavy chain variable domain and heavy chain constant domain sequences disclosed herein. In one example, an antibody is provided having a full heavy chain sequence that is at least 80% identical to the amino acid sequence of the full heavy chain sequence of any antibody disclosed herein e.g., the combination of the SEQ ID Nos for the heavy chain variable domain and heavy chain constant domain sequences disclosed herein. In one example, an antibody is provided having a full heavy chain sequence that is at least 85% identical to the amino acid sequence of the full heavy chain sequence of any antibody disclosed herein e.g., the combination of the SEQ ID Nos for the heavy chain variable domain and heavy chain constant domain sequences disclosed herein. In one example, an antibody is provided having a full heavy chain sequence that is at least 90% identical to the amino acid sequence of the full heavy chain sequence of any antibody disclosed herein e.g., the combination of the SEQ ID Nos for the heavy chain variable domain and heavy chain constant domain sequences disclosed herein. In one example, an antibody is provided having a full heavy chain sequence that is at least 95% identical to the amino acid sequence of the full heavy chain sequence of any antibody disclosed herein e.g., the combination of the SEQ ID Nos for the heavy chain variable domain and heavy chain constant domain sequences disclosed herein. In one example, an antibody is provided having a full heavy chain sequence that is at least 96% identical to the amino acid sequence of the full heavy chain sequence of any antibody disclosed herein e.g., the combination of the SEQ ID Nos for the heavy chain variable domain and heavy chain constant domain sequences disclosed herein. In one example, an antibody is provided having a full heavy chain sequence that is at least 97% identical to the amino acid sequence of the full heavy chain sequence of any antibody disclosed herein e.g., the combination of the SEQ ID Nos for the heavy chain variable domain and heavy chain constant domain sequences disclosed herein. In one example, an antibody is provided having a full heavy chain sequence that is at least 98% identical to the amino acid sequence of the full heavy chain sequence of any antibody disclosed herein e.g., the combination of the SEQ ID Nos for the heavy chain variable domain and heavy chain constant domain sequences disclosed herein. In one example, an antibody is provided having a full heavy chain sequence that is at least 99% identical to the amino acid sequence of the full heavy chain sequence of any antibody disclosed herein e.g., the combination of the SEQ ID Nos for the heavy chain variable domain and heavy chain constant domain sequences disclosed herein. In one example, an antibody is provided having a full heavy chain sequence that is at least 99.5% identical to the amino acid sequence of the full heavy chain sequence of any antibody disclosed herein e.g., the combination of the SEQ ID Nos for the heavy chain variable domain and heavy chain constant domain sequences disclosed herein.

In one example, an antibody is provided having a full light chain sequence that is at least 70% identical to the amino acid sequence of the full light chain sequence of any antibody disclosed herein e.g., the combination of the SEQ ID Nos for the light chain variable domain and light chain constant domain sequences disclosed herein. In one example, an antibody is provided having a full light chain sequence that is at least 75% identical to the amino acid sequence of the full light chain sequence of any antibody disclosed herein e.g., the combination of the SEQ ID Nos for the light chain variable domain and light chain constant domain sequences disclosed herein. In one example, an antibody is provided having a full light chain sequence that is at least 80% identical to the amino acid sequence of the full light chain sequence of any antibody disclosed herein e.g., the combination of the SEQ ID Nos for the light chain variable domain and light chain constant domain sequences disclosed herein. In one example, an antibody is provided having a full light chain sequence that is at least 85% identical to the amino acid sequence of the full light chain sequence of any antibody disclosed herein e.g., the combination of the SEQ ID Nos for the light chain variable domain and light chain constant domain sequences disclosed herein. In one example, an antibody is provided having a full light chain sequence that is at least 90% identical to the amino acid sequence of the full light chain sequence of any antibody disclosed herein e.g., the combination of the SEQ ID Nos for the light chain variable domain and light chain constant domain sequences disclosed herein. In one example, an antibody is provided having a full light chain sequence that is at least 95% identical to the amino acid sequence of the full light chain sequence of any antibody disclosed herein e.g., the combination of the SEQ ID Nos for the light chain variable domain and light chain constant domain sequences disclosed herein. In one example, an antibody is provided having a full light chain sequence that is at least 96% identical to the amino acid sequence of the full light chain sequence of any antibody disclosed herein e.g., the combination of the SEQ ID Nos for the light chain variable domain and light chain constant domain sequences disclosed herein. In one example, an antibody is provided having a full light chain sequence that is at least 97% identical to the amino acid sequence of the full light chain sequence of any antibody disclosed herein e.g., the combination of the SEQ ID Nos for the light chain variable domain and light chain constant domain sequences disclosed herein. In one example, an antibody is provided having a full light chain sequence that is at least 98% identical to the amino acid sequence of the full light chain sequence of any antibody disclosed herein e.g., the combination of the SEQ ID Nos for the light chain variable domain and light chain constant domain sequences disclosed herein. In one example, an antibody is provided having a full light chain sequence that is at least 99% identical to the amino acid sequence of the full light chain sequence of any antibody disclosed herein e.g., the combination of the SEQ ID Nos for the light chain variable domain and light chain constant domain sequences disclosed herein. In one example, an antibody is provided having a full light chain sequence that is at least 99.5% identical to the amino acid sequence of the full light chain sequence of any antibody disclosed herein e.g., the combination of the SEQ ID Nos for the light chain variable domain and light chain constant domain sequences disclosed herein.

In one example, an antibody is provided having at least 70% sequence identity to the sequence of any antibody disclosed herein across both the full heavy and light chain sequences. In one example, an antibody is provided having at least 75% sequence identity to the sequence of any antibody disclosed herein across both the full heavy and light chain sequences. In one example, an antibody is provided having at least 80% sequence identity to the sequence of any antibody disclosed herein across both the full heavy and light chain sequences. In one example, an antibody is provided having at least 85% sequence identity to the sequence of any antibody disclosed herein across both the full heavy and light chain sequences. In one example, an antibody is provided having at least 90% sequence identity to the sequence of any antibody disclosed herein across both the full heavy and light chain sequences. In one example, an antibody is provided having at least 95% sequence identity to the sequence of any antibody disclosed herein across both the full heavy and light chain sequences. In one example, an antibody is provided having at least 96% sequence identity to the sequence of any antibody disclosed herein across both the full heavy and light chain sequences. In one example, an antibody is provided having at least 97% sequence identity to the sequence of any antibody disclosed herein across both the full heavy and light chain sequences. In one example, an antibody is provided having at least 98% sequence identity to the sequence of any antibody disclosed herein across both the full heavy and light chain sequences. In one example, an antibody is provided having at least 99% sequence identity to the sequence of any antibody disclosed herein across both the full heavy and light chain sequences. In one example, an antibody is provided having at least 99.5% sequence identity to the sequence of any antibody disclosed herein across both the full heavy and light chain sequences.

### 5.9 Substitutions

In some examples, the antibody comprises amino acid substitutions as compared to the sequences defined herein.

In some examples, an antibody is provided that is defined by the same sequence(s) as one of the exemplary antibodies described herein and set out in Table 1, but where the sequence(s) comprises amino acid substitutions.

Amino acid substitutions include alterations in which an amino acid is replaced with a different naturally-occurring amino acid residue. Such substitutions may be classified as “conservative”, in which case an amino acid residue contained in a polypeptide is replaced with another naturally occurring amino acid of similar character either in relation to polarity, side chain functionality or size. Such conservative substitutions are well known in the art. Substitutions encompassed by the present invention may also be “non-conservative”, in which an amino acid residue which is present in a peptide is substituted with an amino acid having different properties, such as naturally-occurring amino acid from a different group (e.g. substituting a charged or hydrophobic amino; acid with alanine), or alternatively, in which a naturally-occurring amino acid is substituted with a non-conventional amino acid.

In one embodiment, the conservative amino acid substitutions are as described herein. For example, the substitution may be of Y with F, T with S or K, P with A, E with D or Q, N with D or G, R with K, G with N or A, T with S or K, D with N or E, I with L or V, F with Y, S with T or A, R with K, G with N or A, K with R, A with S, K or P. In another embodiment, the conservative amino acid substitutions may be wherein Y is substituted with F, T with A or S, I with L or V, W with Y, M with L, N with D, G with A, T with A or S, D with N, I with L or V, F with Y or L, S with A or T and A with S, G, T or V.

In one example, the amino acid substitutions are located outside the CDR sequences.

### 5.10 Light Chains

In some examples, the antibody comprises a kappa light chain. Kappa light chain constant region amino acid and nucleotide sequences are set out in SEQ ID Nos: 447-456.

In one example, the light chain may be a lambda light chain. Lambda light chain constant region amino acid and nucleotide sequences can be found in SEQ ID Nos: 457-481.

### 5.11 Isotypes, Constant Regions and Modifications

Selection of an appropriate format for the antibody (e.g., IgG4 or IgG1), can be used to achieve desirable outcomes.

The format for that antibody could be an antibody with limited Fc effector functions, e.g., an IgG4, e.g., a stabilised IgG4 isotype.

The antibodies described herein may comprise a constant region, such as a human constant region, for example an effector-null human constant region, e.g. an IgG4 constant region or an IgG1 constant region, optionally wherein the constant region is IgG4-PE (SEQ ID Nos: 441-446 and 482-483), or a disabled IgG1 as defined in SEQ ID Nos: 425-426.

In a preferred embodiment, the antibody's constant region is SEQ ID NO: 418.

In other embodiments, the antibody is any of the isotypes or constant regions as defined hereinabove. In one embodiment, the constant region is wild-type human IgG1 (SEQ ID Nos: 417-424). For example, the constant region is an effector-enabled IgG1 constant region, optionally having ADCC and/or CDC activity. In one embodiment, the constant region is engineered for enhanced ADCC and/or CDC and/or ADCP. In another embodiment, the constant region is engineered for enhanced effector function.

In some embodiments, the antibody may comprise modifications that enhance the ability of the antibody to cluster and therefore be a better substrate for complement fixation. The Fc domain of IgG1 may be mutated for example at E345 or E430 to reinforce inter-antibody Fc:Fc interactions, stimulating formation of hexamers, which enhances the induction of CDC and ADCC of target cells (de Jong et al., PloS Biol 14(1) e1002344 2016). Hexamer formation is optionally combined with a bispecific antibody format.

The IgG4 constant region may be any of the IgG4 constant region amino acid sequences, or encoded by any of the nucleic acid sequences (SEQ ID Nos: 435-440). A heavy chain constant region may be an IgG4 comprising both the Leu235Glu mutation and the Ser228Pro mutation. This “IgG4-PE” heavy chain constant region (SEQ ID Nos: 441-446 and 482-483) is effector null.

An alternative effector null human constant region is a disabled IgG1 being an IgG1*01 allele comprising the L235A and/or G237A mutations (e.g. LAGA, SEQ ID Nos: 425-426). In one example, the antibodies or antibody fragments disclosed herein comprise an IgG1 heavy chain constant region, wherein the sequence contains alanine at position 235 and/or 237 (EU index numbering). The antibody-dependent cell phagocytosis (ADCP) mechanism is discussed in Gill et al., “Antibody-Dependent Phagocytosis of Tumor Cells by Macrophages: A Potent Effector Mechanism of Monoclonal Antibody Therapy of Cancer”, Cancer Res., 75(23), Dec. 1, 2015.

The potency of Fc-mediated effects may be enhanced by engineering the Fc domain by various established techniques. Such methods increase the affinity for certain Fc-receptors or decrease the affinity for inhibitory Fc-receptors, thus creating potential diverse profiles of activation enhancement. This can be achieved by modification of one or several amino acid residues (e.g. as described in Lazar et al., 2006, Proc. Natl. Acad. Sci. U.S.A., Mar 14; 103(11):4005-10; the modifications disclosed therein are incorporated herein by reference). Human IgG1 constant regions containing specific mutations or altered glycosylation on residue Asn297 (e.g. N297Q, EU index numbering) have been shown to enhance binding to certain Fc receptors.

In one example, such mutations are one or more of the residues selected from 239, 332 and 330 for human IgG1 constant regions (or the equivalent positions in other IgG isotypes). In one example, the antibody or fragment comprises a human IgG1 constant region having one or more mutations independently selected from N297Q, S239D, I332E and A330L (EU index numbering).

In another example, the increase in affinity for Fc-receptors is achieved by altering the natural glycosylation profile of the Fc domain by, for example, generating under fucosylated or de-fucosylated variants (as described in Natsume et al., 2009, Drug Des. Devel. Ther., 3:7-16 or by Zhou Q., Biotechnol. Bioeng., 2008, Feb. 15, 99(3):652-65, the modifications described therein are incorporated herein by reference). Non-fucosylated antibodies harbour a tri-mannosyl core structure of complex-type N-glycans of Fc without fucose residue. These glycoengineered antibodies that lack core fucose residue from the Fc N-glycans may exhibit stronger ADCC than fucosylated equivalents due to enhancement of FcγRIIIa binding capacity. For example, to increase ADCC, residues in the hinge region can be altered to increase binding to Fc-γRIII (see, for example, Shields et al., 2001, J. Biol. Chem., Mar. 2; 276(9):6591-604; the modifications described therein are incorporated herein by reference). Thus, in one example, the antibody or fragment comprises a human IgG heavy chain constant region that is a variant of a wild-type human IgG heavy chain constant region, wherein the variant human IgG heavy chain constant region binds to human Fcγ receptors selected from the group consisting of FcγRIIB and FcγRIIA with higher affinity than the wild type human IgG heavy chain constant region binds to the human Fcγ receptors. In one example, the antibody or fragment comprises a human IgG heavy chain constant region that is a variant of a wild type human IgG heavy chain constant region, wherein the variant human IgG heavy chain constant region binds to human FcγRIIB with higher affinity than the wild type human IgG heavy chain constant region binds to human FcγRIIB. In one example, the variant human IgG heavy chain constant region is a variant human IgG1, a variant human IgG2, or a variant human IgG4 heavy chain constant region. In one example, the variant human IgG heavy chain constant region comprises one or more amino acid mutations selected from G236D, P238D, S239D, S267E, L328F, and L328E (EU index numbering system). In another example the variant human IgG heavy chain constant region comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F (EU index numbering system). In another example, the variant human IgG heavy chain constant region further comprises one or more amino acid mutations that reduce the affinity of the IgG for human FcγRIIIA, human FcγRIIA, or human FcγRI. In one example, the FcγRIIB is expressed on a cell selected from the group consisting of macrophages, monocytes, B-cells, dendritic cells, endothelial cells, and activated T-cells. In one embodiment, the variant human IgG heavy chain constant region comprises one or more of the following amino acid mutations G236A, S239D, F243L, T256A, K290A, R292P, S298A, Y300L, V305I, A330L, 1332E, E333A, K334A, A339T, and P396L (EU index numbering system). In one example, the variant human IgG heavy chain constant region comprises a set of amino acid mutations selected from the group consisting of: S239D; T256A; K290A; S298A; 1332E; E333A; K334A; A339T; S239D and 1332E; S239D, A330L, and 1332E; S298A, E333A, and K334A; G236A, S239D, and 1332E; and F243L, R292P, Y300L, V305I, and P396L (EU index numbering system). In one example, the variant human IgG heavy chain constant region comprises a S239D, A330L, or 1332E amino acid mutations (EU index numbering system). In one example, the variant human IgG heavy chain constant region comprises an S239D and 1332E amino acid mutations (EU index numbering system). In one example, the variant human IgG heavy chain constant region is a variant human IgG1 heavy chain constant region comprising the S239D and 1332E amino acid mutations (EU index numbering system). In one example, the antibody or fragment comprises an afucosylated Fc region. In another example, the antibody or fragment thereof is defucosylated. In another example, the antibody or fragment is under fucosylated.

In another example, the antibodies and fragments disclosed herein may comprise a triple mutation (M252Y/S254T/T256E) which enhances binding to FcRn. See Dall'Aqua et al., Immunol 2002; 169:5171-5180 for a discussion of mutations affection FcRn binding in table 2, the mutations described therein are incorporated herein by reference.

Equally, the enhancement of CDC may be achieved by amino acid changes that increase affinity for C1q, the first component of the classic complement activation cascade (see Idusogie et al., J. Immunol., 2001, 166:2571-2575; the modifications described are incorporated herein by reference). Another approach is to create a chimeric Fc domain created from human IgG1 and human IgG3 segments that exploit the higher affinity if IgG3 for C1q (Natsume et al., 2008, Cancer Res., 68: 3863-3872; the modifications are incorporated herein by reference). In another example, the antibody or antibody fragments disclosed herein may comprise mutated amino acids at residues 329, 331 and/or 322 to alter the C1q binding and/or reduced or abolished CDC activity. In another example, the antibodies or antibody fragments disclosed herein may contain Fc regions with modifications at residues 231 and 239, whereby the amino acids are replaced to alter the ability of the antibody to fix complement. In one example, the antibody or fragment has a constant region comprising one or more mutations selected from E345K, E430G, R344D and D356R, in particular a double mutation comprising R344D and D356R (EU index numbering system).

An antibody may have a heavy chain constant region that binds one or more types of Fc receptor but does not induce cellular effector functions, i.e. which does not mediate ADCC, CDC or ADCP activity. Such a constant region may be unable to bind the particular Fc receptor(s) responsible for triggering ADCC, CDC or ADCP activity. An antibody may have a heavy chain constant region that does not bind Fcγ receptors. Thus, in one example, the constant region may comprise a Leu235Glu mutation (EU index numbering system).

In another example, the antibodies disclosed herein are modified to increase or decrease serum half-life. In one embodiment, one or more of the following mutations: T252L, T254S or T256F are introduced to increase biological half-life of the antibody. Biological half-life can also be increased by altering the heavy chain constant region CH1 domain or CL region to contain a salvage receptor binding epitope taken from two loops of a CH2 domain of an Fc region of an IgG, as described in U.S. Pat. Nos. 5,869,046 and 6,121,022, the modifications described therein are incorporated herein by reference. In another example, the Fc hinge region of an antibody or antigen-binding fragment of the invention is mutated to decrease the biological half-life of the antibody or fragment. One or more amino acid mutations are introduced into the CH2-CH3 domain interface region of the Fc-hinge fragment such that the antibody or fragment has impaired Staphylococcyl protein A (SpA) binding relative to native Fc-hinge domain SpA binding. Other methods of increasing serum half-life are known to those skilled in the art. Thus, in one example, the antibody or fragment is PEGylated. In another example, the antibody or fragment is fused to an albumin-binding domain, e.g. an albumin binding single domain antibody (dAb). In another example, the antibody or fragment is PASylated (i.e. genetic fusion of polypeptide sequences composed of PAS (XL-Protein GmbH) which forms uncharged random coil structures with large hydrodynamic volume). In another example, the antibody or fragment is XTENylated®/rPEGylated (i.e. genetic fusion of non-exact repeat peptide sequence (Amunix, Versartis) to the therapeutic peptide). In another example, the antibody or fragment is ELPylated (i.e. genetic fusion to ELP repeat sequence (PhaseBio)). These various half-life extending fusions are described in more detail in Strohl, BioDrugs (2015) 29:215-239, which fusions are incorporated herein by reference.

The antibody may have a modified constant region which increases stability. Thus, in one example, the heavy chain constant region comprises a Ser228Pro mutation. In another example, the antibodies and fragments disclosed herein comprise a heavy chain hinge region that has been modified to alter the number of cysteine residues. This modification can be used to facilitate assembly of the light and heavy chains or to increase or decrease the stability of the antibody.

### 5.12 Acinetobacter baumannii Classification Clone G2 and G2 Strains

GC2 strains are defined based on whole genome sequencing data and phylogenetic analysis. GC2 representative strains are available from ATCC and NCTC.

Publicly available strain NCTC 13424 may be used to reproduce all assays for OC1 LOS and Oxa-23 described herein.

(http://www.pheculturecollections.org.uk/products/bacteria/detail.jsp?refld=NCTC+13424&collection=nc tc, the contents of which are incorporated herein by reference)

Prototype strain LAC-4 (GenBank reference: GCA_000786735.1) also is highly cited in the literature and may be used to reproduce all assays for KL49 described herein (https://www.genome.ip/kegg-bin/show organism?org=abal. PMID: 25728466. Sci Rep 5:8643 (2015) DOI: 10.1038/srep08643, the contents of which are incorporated herein by reference).

An antibody that specifically binds to the G2 clone of Acinetobacter baumannii may be cross-reactive with related antigens such as those of other clones of Acinetobacter baumannii.

Methods of determining binding as described herein can also be used to determine cross-reactivity of an antibody with different clones or strains of Acinetobacter baumannii.

### 5.13 Antibody Generation

In some examples, the antibody is a monoclonal antibody. Methods of making monoclonal antibodies are known and include, for example, fusing myeloma cells with the cells from an animal that was immunized with the desired antigen. In other examples, the monoclonal antibodies may be generated using recombinant DNA technology.

In some examples, the antibody is a human antibody. In one example, the antibody is a fully human antibody. In one example, the antibody is a fully human monoclonal antibody.

### 5.14 Nucleic Acids, Vectors, Host Cells

Nucleic acids that encode a VH domain and/or a VL domain of any one of the antibodies described herein are also provided. A nucleic acid that encodes the VH domain of any one of the antibodies described herein is provided. A nucleic acid that encodes the VL domain of any one of the antibodies described herein is provided.

The SEQ ID Nos and the nucleic acid sequences encoding the VH and VL domains of each the exemplary antibodies described herein are set out in Table 1.

In one example, the nucleic acid sequence is at least 70% identical to the specified SEQ ID No. In one example, the nucleic acid sequence is at least 80% identical to the specified SEQ ID No. In one example, the nucleic acid sequence is at least 90% identical to the specified SEQ ID No. In one example, the nucleic acid sequence is at least 95% identical to the specified SEQ ID No. In one example, the nucleic acid sequence is at least 96% identical to the specified SEQ ID No. In one example, the nucleic acid sequence is at least 97% identical to the specified SEQ ID No. In one example, the nucleic acid sequence is at least 98% identical to the specified SEQ ID No. In one example, the nucleic acid sequence is at least 99% identical to the specified SEQ ID No. In one example, the nucleic acid sequence is at least 99.5% identical to the specified SEQ ID No.

In one example, the nucleic acid encodes a heavy chain of any one of the antibodies described herein. In another example, the nucleic acid encodes a light chain of any one of the antibodies described herein.

In one example, the nucleic acid is an isolated and purified nucleic acid.

Vectors comprising the nucleic acids described above are also provided. In one example, the vector may be a CHO cell expression vector. In one example, the vector may be a HEK293 cell expression vector.

Host cells comprising the nucleic acids described above are also provided. In some examples, the host cells are eukaryotic cells, e.g., mammalian cells, preferably CHO cells (e.g., CHO cells grown in suspension culture). HEK293 cells are an alternative cell line for manufacture.

### 5.15 Pharmaceutical Composition

In one example, there is provided a pharmaceutical composition comprising an effective amount of an antibody as described herein and a pharmaceutically acceptable excipient. An effective amount of antibody to be employed therapeutically will depend, for example, upon the therapeutic objectives, the route of administration, and the condition of the patient. In one example, the composition includes other excipients or stabilizers.

Pharmaceutically acceptable excipients are known and include carriers, excipients, or stabilizers that are nontoxic to the cell or mammal being exposed thereto at the dosages and concentrations employed. Often the physiologically acceptable excipient is an aqueous pH buffered solution. Examples of physiologically acceptable excipient include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as Ethylenediaminetetraacetic acid (EDTA); sugar alcohols such as mannitol or sorbitol; salt-forming counterions such as sodium; and/or nonionic surfactants such as TWEEN™, polyethylene glycol (PEG), and PLURONICS™.

The antibodies can be administered intravenously or through the nose, lung, for example, as a liquid or powder aerosol (lyophilized) or by nebulisation of a liquid. The composition can also be administered parenterally or subcutaneously. When administered systemically, the composition should be sterile, pyrogen-free and in a physiologically acceptable solution having due regard for pH, isotonicity and stability. These conditions are known to those skilled in the art.

Methods of administering a prophylactic or therapeutic agent (e.g., an antibody as disclosed herein), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific example, a prophylactic or therapeutic agent (e.g., an antibody as disclosed herein), or a pharmaceutical composition is administered intranasally, intramuscularly, intravenously, or subcutaneously. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. Each dose may or may not be administered by an identical route of administration.

Various delivery systems are known and can be used to administer a prophylactic or therapeutic agent (e.g., an antibody as disclosed herein), including, but not limited to, encapsulation in liposomes, microparticles, microcapsules, recombinant cells capable of expressing the antibody, receptor-mediated endocytosis (see, e.g., Wu and Wu, J. Biol. Chem. 262:4429-4432 (1987)), construction of a nucleic acid as part of a retroviral or other vector, etc. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO92/19244, WO97/32572, WO97/44013, WO98/31346, and WO99/66903, each of which is incorporated herein by reference their entirety.

In a specific example, it may be desirable to administer a prophylactic or therapeutic agent, or a pharmaceutical composition as described herein locally to the area in need of treatment. This may be achieved by, for example, local infusion, by topical administration (e.g., by intranasal spray), by injection, or by means of an implant, said implant being of a porous, non-porous, or gelatinous material, including membranes, such as sialastic membranes, or fibres. When administering an antibody, care must be taken to use materials to which the antibody does not absorb.

In the case of medicaments that are intended for local and/or topical administration, such as by absorption to epithelial or mucocutaneous linings, an antibody may be provided as an IgA isotype antibody. For human patients, human IgA1 or human IgA2 antibodies are preferred. Medicaments formulated for inhalation and/or for delivery of antibody (or its encoding nucleic acid, e.g., in a DNA vector) to the upper and/or lower respiratory tract, including formulations for delivery of a nebulised medicament, may comprise an IgA (e.g., human IgA1 or human IgA2) antibody. Inhalers, nebulisers and similar devices may thus be provided containing a medicament comprising an IgA antibody or its encoding nucleic acid, together with any buffers or other excipients suitable for stabilisation of the medicament and/or for promoting its delivery to the target tissue.

### 5.16 Therapeutic Use

Antibodies described herein may be used to treat or prevent a bacterial infection caused by Acinetobacter baumannii. One aspect includes use of an antibody or composition described herein as a medicament.

Thus, in one example, antibodies described herein or compositions described herein for use in a method of treating a bacterial infection caused by Acinetobacter baumannii are provided, said method comprising administering the antibody or composition to a patient. In another example, antibodies described herein or compositions described herein for use in a method of preventing a bacterial infection caused by Acinetobacter baumannii are provided, said method comprising administering the antibody or composition to a patient.

The patient may be any animal, including, but not limited to, mammals. In one embodiment, the patient is a human. In one embodiment, the patient is an adult human. In one embodiment, the patient is a human over 12 years old.

In one embodiment, the bacterial infection caused by Acinetobacter baumannii is a nosocomial bacterial infection caused by Acinetobacter baumannii.

In one embodiment, the patient has a lower respiratory tract infection, for example pneumonia, e.g. ventilator associated pneumonia.

In one embodiment, the patient has sepsis.

In one embodiment, the patient has bacteremia.

In one example, the antibody for use or the composition for use described above, one or more symptoms of a bacterial infection caused by Acinetobacter baumannii are reduced.

In one example, the progression of a bacterial infection caused by Acinetobacter baumannii is reduced.

In one example, the risk of developing a bacterial infection caused by Acinetobacter baumannii is reduced.

In one example, the risk of transmission of a bacterial infection caused by Acinetobacter baumannii to and/or from a patient is reduced.

Provided herein is the use of an antibody described herein or a composition described herein in the manufacture of a medicament for treating a bacterial infection caused by Acinetobacter baumannii. Use of an antibody described herein or a composition described herein in the manufacture of a medicament for preventing a bacterial infection caused by Acinetobacter baumanniiis also provided. Thus, in one example, one or more symptoms of bacterial infection caused by Acinetobacter baumannii are reduced. In another example, the progression of bacterial infection caused by Acinetobacter baumannii is reduced. In another example, the risk of developing a bacterial infection caused by Acinetobacter baumannii is reduced. In another example, the risk of transmission of a bacterial infection caused by Acinetobacter baumannii to and/or from a human is reduced.

A method of treating a bacterial infection caused by Acinetobacter baumannii in a human, comprising administering to said human κ therapeutically effective amount of an antibody described herein or a composition described herein is provided. A method of preventing a bacterial infection caused by Acinetobacter baumannii in a human, comprising administering to said human κ therapeutically effective amount of an antibody described herein or a composition described herein is also provided. In one example, one or more symptoms of bacterial infection caused by Acinetobacter baumannii are reduced. In one example, the progression of bacterial infection caused by Acinetobacter baumannii is reduced. In one example, the risk of developing a bacterial infection caused by Acinetobacter baumannii is reduced. In one example, the risk of transmission of a bacterial infection caused by Acinetobacter baumannii to and/or from a human is reduced.

In one example, the use of an antibody or composition described herein or the method further comprises administering at least one further therapeutic agent. In one example, the first antibody and further therapeutic agent are administered simultaneously, separately or sequentially. In one example, the further therapeutic agent is a further antibody.

In one example, the further therapeutic agent is carbapenem. In this regard, the present inventors have identified a synergy between administering carbapenem and administering an anti-Oxa-23 mAb as described herein due to the mode of action of the anti-Oxa-23 antibody, namely the antibody inhibiting the activity of the Oxa-23 carbapenemase enzyme, thereby inhibiting the associated resistance to carbapenem.

In one embodiment, the further therapeutic agent is colistin.

In one example, the antibody is administered as an antibody-drug conjugate in which the antibody is linked to a drug moiety. For example, the antibody may be linked to a drug moiety which may be a cytokine, chemokine, or small molecule antiviral.

Antibodies described herein may be used to prevent death and shorten the time to recovery and discharge for patients having a bacterial infection caused by Acinetobacter baumannii. Patients will generally be human patients and may be patients diagnosed as having a bacterial infection caused by Acinetobacter baumannii. Further, patients may be patients that have already been admitted to hospital for another reason.

In another example, a kit for treating a bacterial infection caused by Acinetobacter baumannii is provided, wherein the kit includes an antibody as described herein and instructions to administer the antibody to a subject in need of treatment. There is also provided a pharmaceutical or diagnostic pack or kit comprising one or more containers filled with one or more of the ingredients of the pharmaceutical compositions as disclosed herein, such as one or more anti-Oxa-23 antibodies provided herein. There is also provided a pharmaceutical or diagnostic pack or kit comprising one or more containers filled with one or more of the ingredients of the pharmaceutical compositions as disclosed herein, such as one or more anti-OC1 LOS antibodies provided herein. There is also provided a pharmaceutical or diagnostic pack or kit comprising one or more containers filled with one or more of the ingredients of the pharmaceutical compositions as disclosed herein, such as one or more anti-KL49 antibodies provided herein. Optionally associated with such container(s) can be a notice in the form prescribed by a governmental agency regulating the manufacture, use or sale of pharmaceuticals or biological products, which notice reflects approval by the agency of manufacture, use or sale for human administration, e.g., an authorisation number.

In another example, an article of manufacture that includes a container in which a composition containing an antibody as described herein and a packaging insert or label indicating that the composition can be used to treat a bacterial infection caused by Acinetobacter baumannii is provided. In one example, there is provided a kit for treating and/or preventing a bacterial infection caused by Acinetobacter baumannii, the kit comprising an antibody as disclosed herein in any example or combination of examples (and optionally a further therapeutic agent as described elsewhere herein) optionally in combination with a label or instructions for use to treat and/or prevent said infection in a human; optionally wherein the label or instructions comprise a marketing authorisation number (e.g., an FDA or EMA authorisation number); optionally wherein the kit comprises an IV or injection device that comprises the antibody. In another example, the kit comprises an antibody contained within a container or an IV bag. In another example, the container or IV bag is a sterile container or a sterile IV bag. In another example, the antibody is formulated into a pharmaceutical composition contained within a (sterile) container or contained within a (sterile) IV bag. In a further example, the kit further comprises instructions for use.

In another example, a kit for treating bacterial infection caused by Acinetobacter baumannii is provided, wherein the kit includes an antibody as described herein and instructions to administer the antibody to a subject in need of treatment. The subject in need may be specifically defined in the kit as someone of a specific higher risk group defined by epidemiological data, risk stratification data from the person's health records, risk stratification by the genotype of certain genes of the individual or the presence of certain biomarkers in the person's blood or other tissue sample. Where the risk stratification involves another pharmaceutical or diagnostic pack or kit, the combined product will act as a linked diagnostic/prognostic and treatment kit.

### 5.17 Prevention of Infection—Prophylactic Use

Antibodies as described herein may be used prophylactically. Administration of antibodies may prevent infection or reduce the risk of infection by Acinetobacter baumannii. Antibodies may for example be used to prevent infection in those at risk in high transmission environments, such as for example in hospitals. Antibodies may be administered to a patient connected to a ventilator, to reduce the patient's risk of contracting ventilator associated pneumonia.

### 5.18 Diagnostics

Antibodies as described herein can be used to detect the presence, absence and/or level of Acinetobacter baumannii in a biological sample from a patient. In one example, the biological sample is a tissue sample (e.g., in pathology studies or biopsy samples of tissue used for diagnostics and prognostics). In other examples, the biological sample is blood, plasma, serum, urine, faeces, cerebrospinal fluid (CFS). In other examples, the biological sample is from a nasal or throat swab. Liquid samples are convenient for use in many types of diagnostic assays.

The antibodies described herein can be used to identify the presence, absence and/or level of Acinetobacter baumannii at baseline, i.e., before treatment.

The antibodies described herein can be used to guide therapy, particularly to identify the presence, absence and/or level of Acinetobacter baumannii during or after treatment.

The antibodies described herein can be used for patient monitoring, to help evaluate whether a course of treatment is effective and whether or not treatment should be continued.

In one example, the antibody described herein is labelled with a detectable moiety, for example, a radiolabel, fluorescent label, enzymatic label, chemiluminescent labelled or a biotinyl group. Radioisotopes or radionuclides may include 3H, 14C, 15N, 35S, 90Y, 99Tc, 115In, 125I, 131I, fluorescent labels may include rhodamine, lanthanide phosphors or FITC and enzymatic labels may include horseradish peroxidase, β-galactosidase, luciferase, alkaline phosphatase. Additional labels include, by way of illustration and not limitation: enzymes, such as glucose-6-phosphate dehydrogenase (“G6PDH”), alpha-D-galactosidase, glucose oxydase, glucose amylase, carbonic anhydrase, acetylcholinesterase, lysozyme, malate dehydrogenase and peroxidase; dyes; additional fluorescent labels or fluorescers include, such as fluorescein and its derivatives, fluorochrome, GFP (GFP for “Green Fluorescent Protein”), dansyl, umbelliferone, phycoerythrin, phycocyanin, allophycocyanin, o-phthaldehyde, and fiuorescamine; fluorophores such as lanthanide cryptates and chelates e.g. Europium etc (Perkin Elmer and Cisbio Assays); chemoluminescent labels or chemiluminescers, such as isoluminol, luminol and the dioxetanes; sensitisers; coenzymes; enzyme substrates; particles, such as latex or carbon particles; metal sol; crystallite; liposomes; cells, etc., which may be further labelled with a dye, catalyst or other detectable group; molecules such as biotin, digoxygenin or 5-bromodeoxyuridine; toxin moieties, such as for example a toxin moiety selected from a group of Pseudomonas exotoxin (PE or a cytotoxic fragment or mutant thereof), Diptheria toxin or a cytotoxic fragment or mutant thereof, a botulinum toxin A, B, C, D, E or F, ricin or a cytotoxic fragment thereof e.g. ricin A, abrin or a cytotoxic fragment thereof, saporin or a cytotoxic fragment thereof, pokeweed antiviral toxin or a cytotoxic fragment thereof and bryodin 1 or a cytotoxic fragment thereof.

In one example, the antibody can be administered to a patient, wherein the antibody is conjugated to a label. The presence of the label in the patient can be measured or observed, wherein a relatively high amount of the label may indicate a high risk of infection and a relatively low amount of the label may indicate a relatively low risk of the infection. In one example, the label is a contrast agent, isotopic tag, or fluorescent marker, such as green fluorescent protein.

For use in diagnostics, antibodies with high affinity, especially with fast on-rate and slow off-rate (e.g., as measured by SPR) are particularly valuable.

In some embodiments, it is desirable to include 2 antibodies in a diagnostic assay and these should preferably be directed to different targets on the Acinetobacter baumannii. The diagnostic assay could be a double antigen binding assay (DABA). In a DABA, a first antibody is used as a capture antibody to bind the Acinetobacter baumannii in a sample (for this purpose, a high affinity antibody with fast on-rate and slow off-rate is desirable, as noted above), and a second antibody, specific for an epitope that is different from the capture antibody's epitope, is used for detection. The second antibody may thus be detectably labelled, by direct or indirect labelling. A DABA may comprise providing the first antibody (optionally immobilised on a surface), contacting the surface with a sample to allow capture of antigen, if present, followed by washing to remove unbound antigen and sample, and then exposing the surface to the detection antibody to allow binding to the antigen, if present, washing to remove unbound detection antibody, and detecting the presence of the detection antibody. The presence of the detection antibody indicates that the sample is positive for Acinetobacter baumannii. This type of assay may be used to determine whether a patient is infected with Acinetobacter baumannii.

In one example, a kit for detecting Acinetobacter baumannii in a biological sample is provided. The kit can be used to screen for Acinetobacter baumannii infection. In one example, the kit includes an antibody according to the invention as described anywhere herein and a means for determining whether the antibody is bound to Acinetobacter baumannii in a sample. In one example, the antibody is specific for Acinetobacter baumannii. In one example, the antibody is labelled. In another example, the antibody is an unlabelled primary antibody and the kit includes means for detecting the primary antibody. In one example, the means for detecting includes a labelled secondary antibody that is an anti-immunoglobulin antibody. The antibody may be labelled with any suitable marker, including, for example, a fluorochrome, an enzyme, a radionuclide and a radiopaque material.

In one example, a kit for detecting Acinetobacter baumannii is provided, wherein the kit includes an antibody as described herein. In one example, the kit may also include instructions and one or more reagents for detecting Acinetobacter baumannii.

### 5.19 Antibody Discovery

The present invention represents a completely new approach to antibody generation and discovery for antibodies to be used in the diagnosis and treatment of pathogen, particularly bacterial, infections. The new approach involves immunising a transgenic mouse, such as a humanised mouse, with a bacteria or other pathogen to generate antibodies in a manner that is antigen agnostic in that it has no inherent bias towards any potential target antigen on the pathogen. Furthermore, the new approach provides the opportunity to combine multiple strains of a pathogen in the immunisation step, whereby the present inventors have discovered that it is possible to discover monoclonal antibodies with cross-reactivity across multiple strains of the pathogen. The present inventors have further discovered that the approach is particularly suitable for bacteria producing outer-membrane vesicles (OMVs), where pooled, purified vesicles from multiple bacterial strains can be used to immunise humanised mice. In this regard, the present inventors have discovered excellent correlation between antibodies capable of binding to purified vesicles produced by bacteria and antibodies capable of binding to live bacteria. Furthermore, the present inventors have discovered that pooled OMVs have the potential to induce cross-reactive serum responses to additional bacterial strains, thereby producing cross reactive mAbs within the mouse polyclonal response.

Accordingly, the present invention provides a sample comprising a number of different strains of a pathogen, e.g., a bacteria, where the sample is suitable for use in immunising a transgenic mouse, such as a humanised mouse. In one embodiment, the sample comprises pooled membrane vesicles isolated from a number of strains of the same bacteria.

Further, the present invention provides a method of making a polyclonal human antibodies comprising: a) exposing a transgenic mouse, such as a humanised mouse, to antigenic stimulation, such that the mouse produces polyclonal antibodies against the antigen; b) isolating the polyclonal antibodies from the mouse; wherein the antigen comprises a number of different strains of a pathogen, e.g., a bacteria.

Further, the present invention provides a method comprising a step of preparing a sample comprising a number of different strains of a pathogen, e.g., a bacteria, suitable for use in immunising a transgenic mouse, such as a humanised mouse. In one embodiment, the method further comprises immunising the mouse with the prepared sample and collecting the polyclonal antibodies generated by the mouse.

In one embodiment, the method further comprises characterising the binding of the antibodies generated by the mouse.

In one embodiment, the method further comprises isolating a monoclonal antibody, such as a fully human monoclonal antibody, from the polyclonal antibodies generated by the mouse.

The present invention further provides a transgenic mouse, such as a humanised mouse, that produces antibodies, wherein said mouse has been immunised with a sample comprising a number of different strains of a pathogen, e.g., a bacteria.

In one embodiment, the transgenic mouse is a transgenic mouse that produces hybrid antibodies containing human variable regions and mouse constant regions. In one embodiment, the transgenic mouse is a humanised mouse that produces antibodies with human variable regions e.g., fully human antibodies, such as the Kymab proprietary IntelliSelect Transgenic mouse platform (e.g., “Darwin”).

As an alternative to mice, rats or other non-human animals such as chickens or llamas may be used for antibody discovery by immunisation, as is well known in the art.

### 6. EXAMPLES

### 6.1 Example 1: Antibody Generation

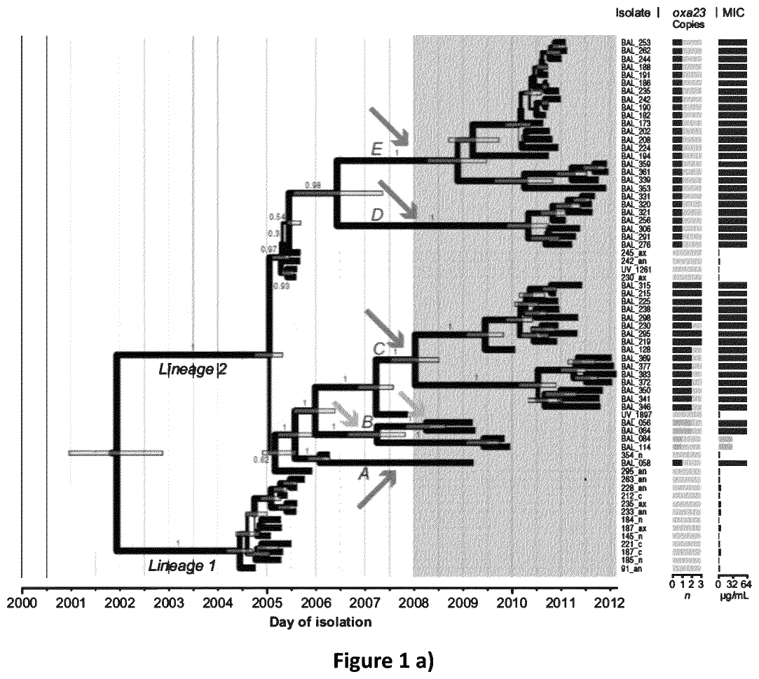

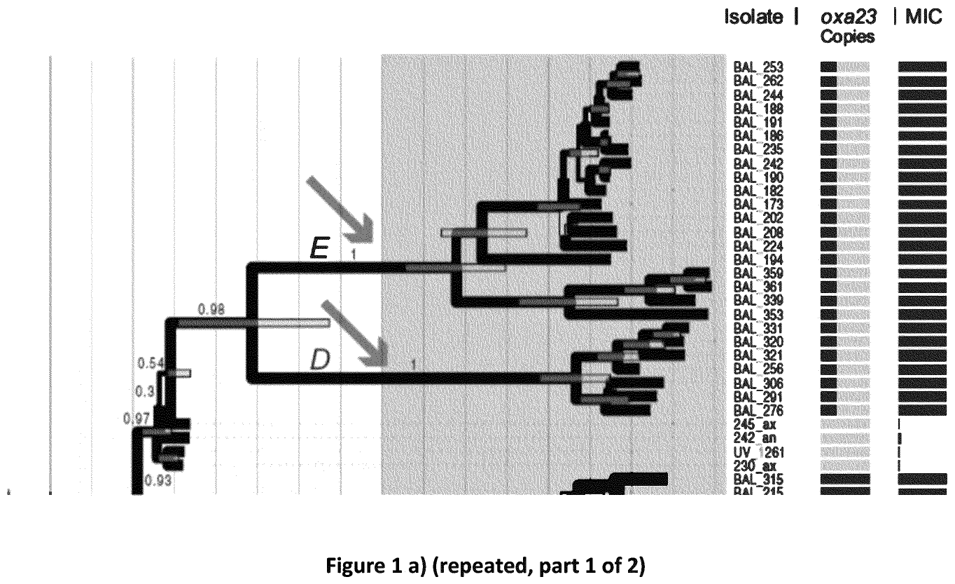

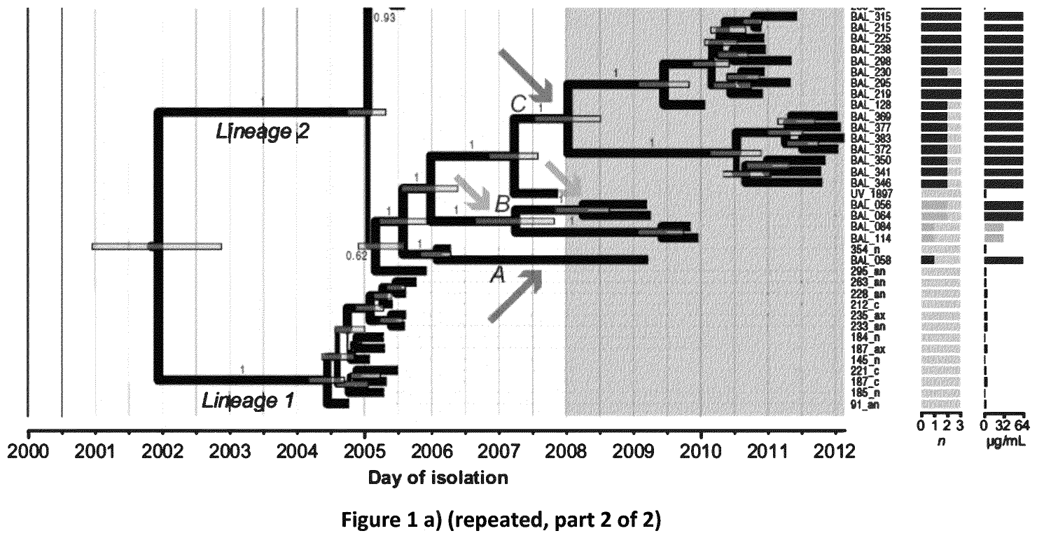

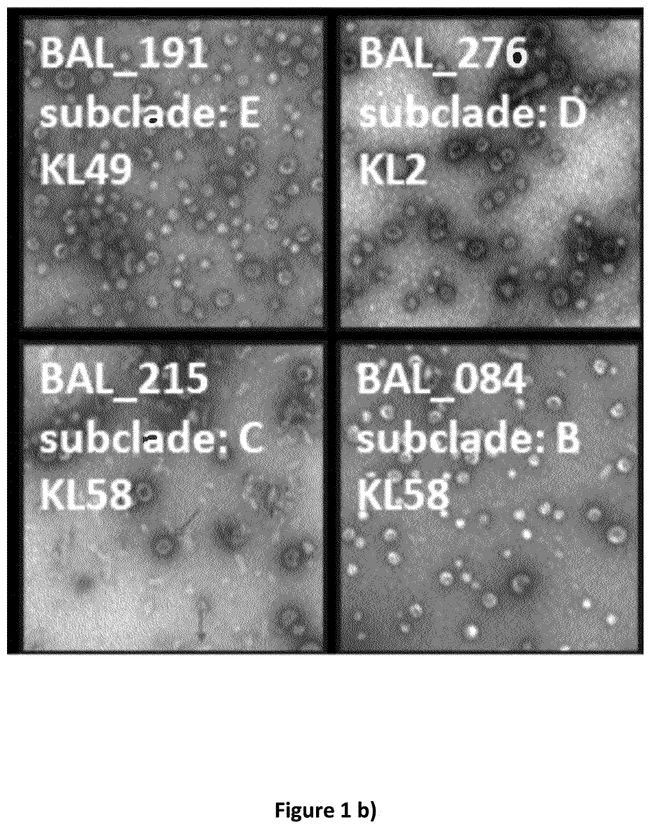

An end-to-end process to enable generation of mAbs to contemporary clinical strains of Acinetobacter baumannii. This process is outlined in FIG. 1. Broadly, our strategy targets the most relevant strains against which mAb-based therapies would be most desirable due to multi-drug resistance (MDR).

Transgenic mice with humanised immunoglobulin loci, producing antibodies with human heavy and light chain variable regions, were immunised with OMV preparations.

FIG. 1a. Time resolved core genome phylogeny for GC2 A. baumannii isolates in a Ho Chi Minh ICU. Shading indicates the period during which the carbapenem antibiotic imipenem was used for the empirical treatment of VAP in the ICU. Labelled A-E are the five carbapenem resistant genomic subclades of GC2. The capsule K-locus subtype for the 10 strains tested for OMV production are also listed on the right. Marked with asterisks (*) are 4 strains from which OMVs have been successfully generated and used for immunisation of Kymab Intellisellect Transgenic mice.

FIG. 1b. Scanning electron microscope images of un-fixed OMV preparations generated from a A. baumannii clinical isolates used for immunisation. OMVs prepared from A. baumannii BAL_191, BAL_276, BAL_215 and BAL_084. Approximately 95% of OMVs in this preparation are between 25-70 nm in diameter, while larger OMVs with a diameter>80 nm comprise 2% of the preparation. Additional, amorphic structures can also be observed and constitute 3% of the preparation.

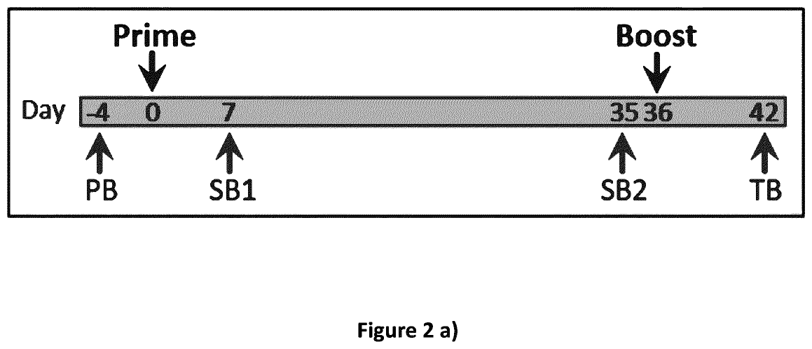

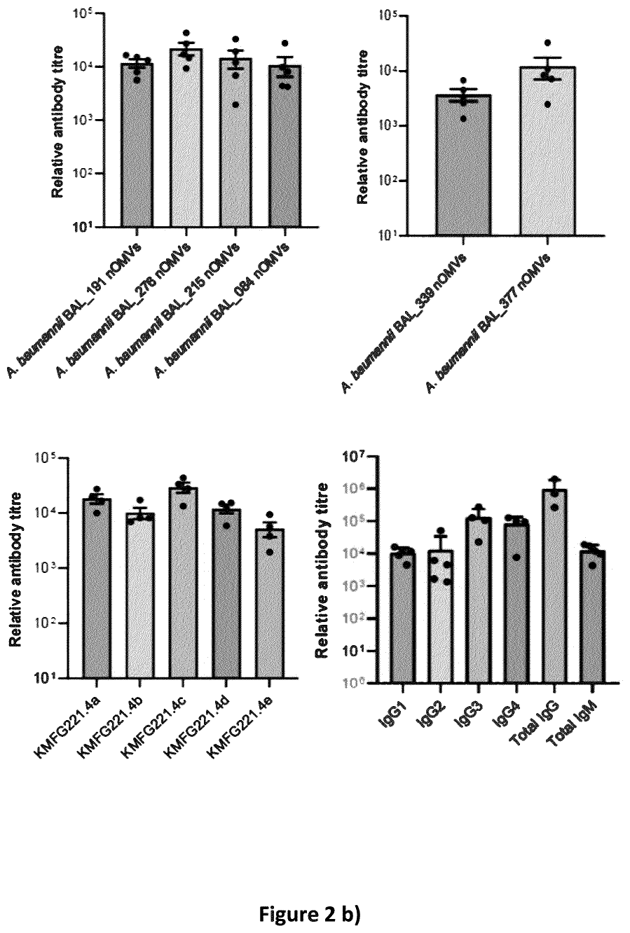

FIG. 2a. Regime used for OMV immunisations. 10 mice received a prime dose of ˜1 μg of OMVs on day 0 of the study. Following this, the mice were split into 2 study groups, with 5 mice in group 1 sacrificed on day 7, with their spleens and lymph nodes harvested. The 5 mice in group 2 received an additional boost on day 36 before being sacrificed on day 43. Bleeds were taken prior to prime and boost doses, and on day 7. These sera were assessed for reactivity against the immunising OMVs via ELISA. Organs harvested on day 7 can be analysed using IntelliSelect Screening Technologies to B cell responses using organs harvested on day 43. PB=pre-bleed; SB=serial bleed; TB=terminal bleed.

FIG. 2b. Polyclonal serum responses of Kymab Intellisellect Transgenic mice immunised with a mixed pool of 4 GC2 A. baumannii OMVs to the immunising OMVs as determined by ELISA. (a) Kinetics of the antigen specific IgG1 response to the 4 GC2 A. baumannii OMVs used for immunisation. The total antigen specific IgG1 response to the 4 individual GC2 A. baumannii OMVs used for immunisation (BAL_084, BAL_191, BAL_215 and BAL_276 in bold) is comparable to the response to 2 additional GC2 A. baumannii OMVs that were absent from the immunising pool (BAL_339 and BAL_377) on day 43 of the study. Specific IgG1 responses were comparable across all mice immunised and all human Ig Isotypes were represented by specific antibodies present in the polyclonal serum. Antigen specific relative endpoint titres have been calculated relative to the pre-bleed taken prior to prime immunisation.

### 6.2 Example 2: Antibody Screening

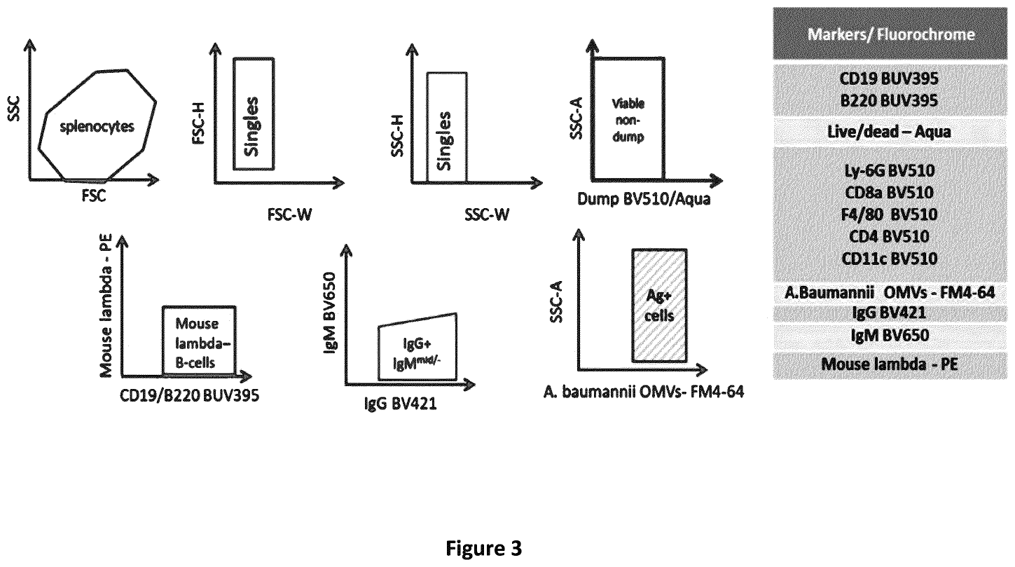

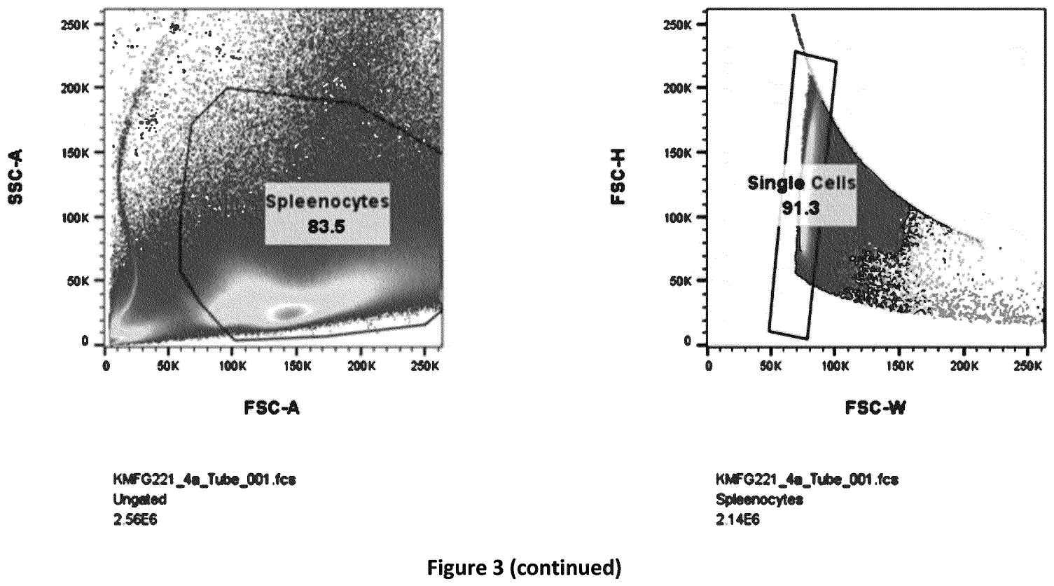

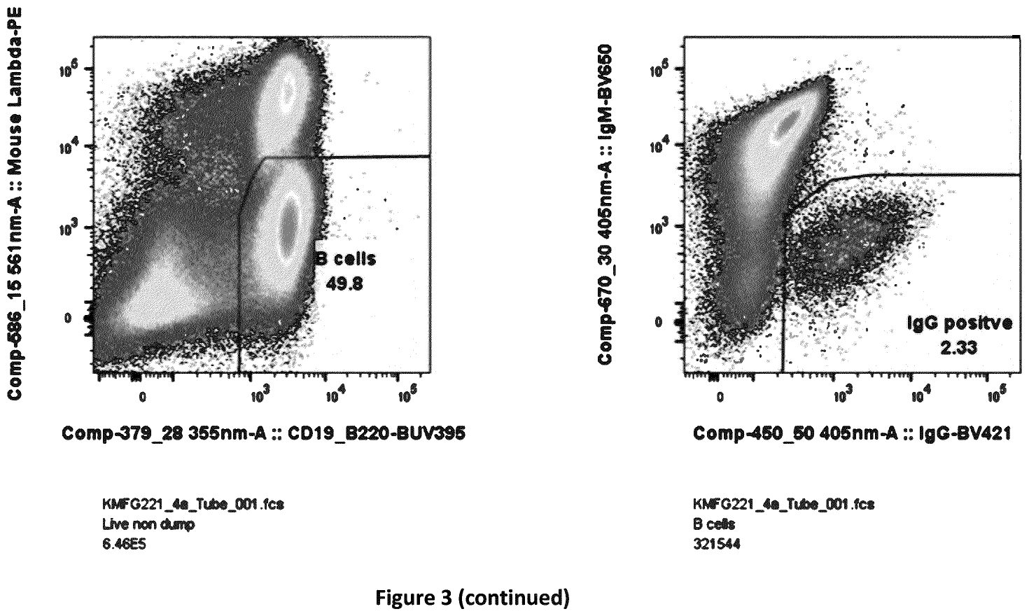

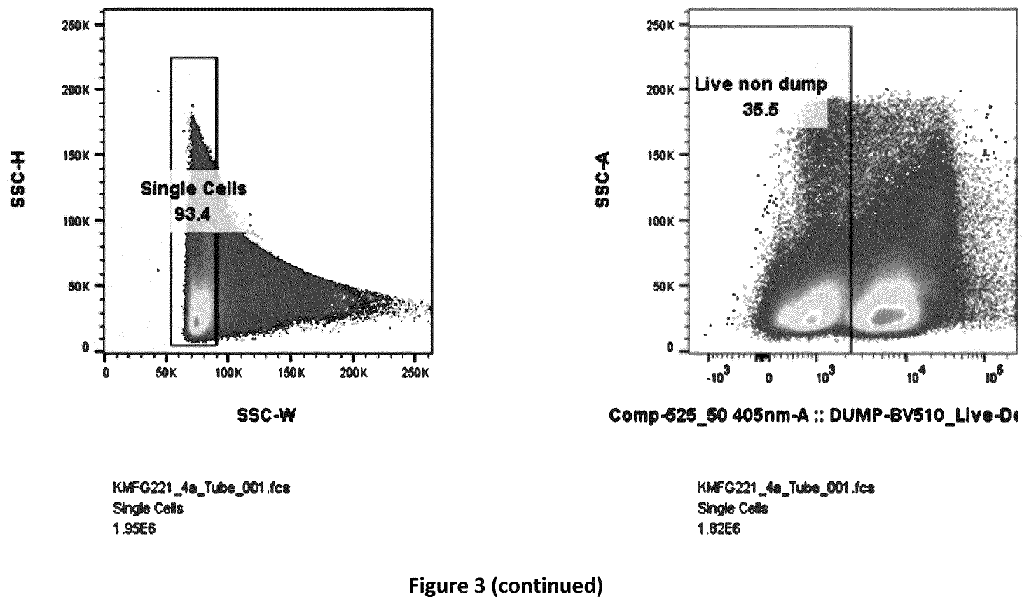

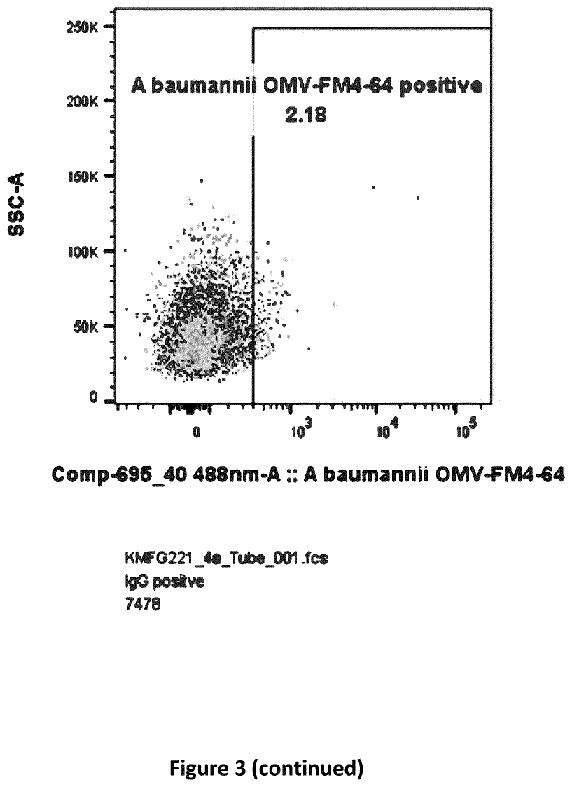

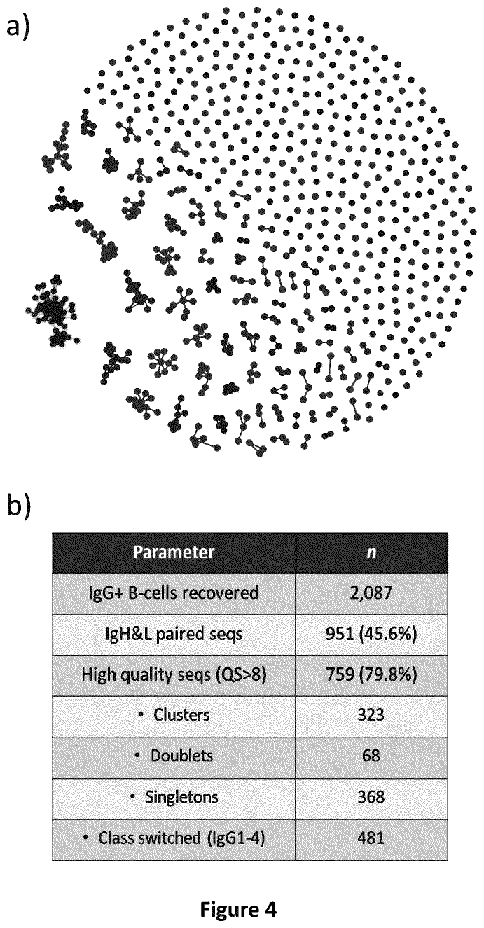

We developed a method for sorting antigen-specific IgG+ B cells from the spleens of GC2 CRAB OMV-immunised mice. Employing the gating strategy illustrated in FIG. 3, we successfully isolated 2,087 OMV-binding IgG+ B cells from the spleens of GC2 CRAB OMV-immunised mice using FM4-64 stained OMVs as a sort bait (FIG. 3). Following successful antigen-specific sorting, sorted single cells were subject to IgH/L sequencing and bioinformatic analysis. This analysis revealed 951 paired heavy and light chain reads, of which 530 were found to be of the IgG subclass (FIG. 4b). In addition, 372 paired sequences were shown to exist within clusters containing>2 sequences arising from a common B cell progenitor. These clusters indicate B cell IgH/L affinity maturation occurring within germinal centres formed in the spleens of immunised mice, and more strikingly, suggest convergent antibody evolution between immunised mice, as evinced by clusters containing related sequences from more than one mouse. Based on these findings, we selected heavy and light chain antibody pairs that met the following criteria: high quality next generation sequencing (NGS) data, 2 or more amino acid mutations across heavy and light chains indicating affinity maturation, FM4-64 GC2 A. baumannii OMV mean fluorescence intensity>7.2 after arcsinh (inverse of hyperbolic sine function) transformation and no duplication at the amino acid level. The 4 most highly mutated sequences were selected from within clusters containing large numbers of related sequences, while the more highly mutated sequence from doublets (containing 2 related sequences) were selected. In addition, we also selected paired sequences that were found to be related to clones that we had previously isolated from bone marrow plasma cells from the same immunised mice and shown to bind to GC2 A. baumannii OMVs via ELISA. Using these criteria, 284 paired sequences were selected for DNA synthesis, allowing subsequent vector-borne expression as human IgG1. An additional 9 sequences were selected from the pool of sorted plasma cells isolated from the bone marrow of the same GC2 A. baumannii OMV immunised mice that clustered with sequences that we had previously found to bind to the immunising OMVs via ELISA. Of the selected clones 252 were IgG, 28 were IgM, 2 were IgE and 11 clones were unassigned a subclass in the original sort. DNA constructs expressing individual IgH and IgL pairs as human IgG1 were transfected into human embryonic Kidney 293 (HEK 293) cells for small-scale antibody expression.

Antigen-binding, IgG+ B cells were sorted from the spleens of GC2 CRAB OMV-immunised mice. FIG. 4. Selection to include only those Antigen binding-enriched B cells that bound to the FM4-64 fluorescently labelled GC2 CRAB OMVs was performed.

FIG. 4a. Sunspot plot of the 951 paired VH and VL chain sequences recovered from the spleens of 4 Kymab Intellisellect Transgenic mice immunised with GC2 CRAB OMVs. Each VH and VL paired sequence is represented by a circle, coloured by the individual mouse from which it was derived. Sequences within a lineage, inferred to have derived from a common B cell progenitor are connected by black lines.

FIG. 4b. Table indicating number of IgH&L paired sequences, proportion mapping to high-quality sequences and apportioned to clusters of related sequences indicative of an actively evolving B cell response to an immunogen.

### 6.3 Example 3: Antibody Binding

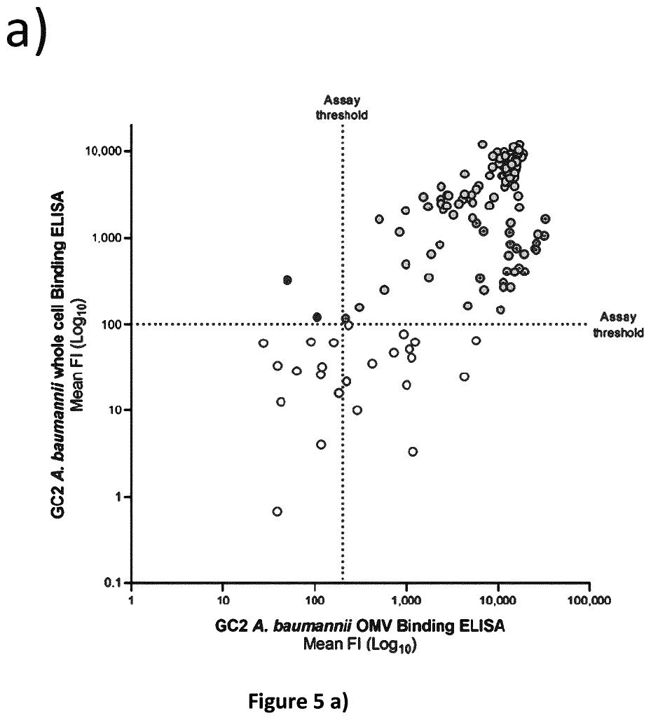

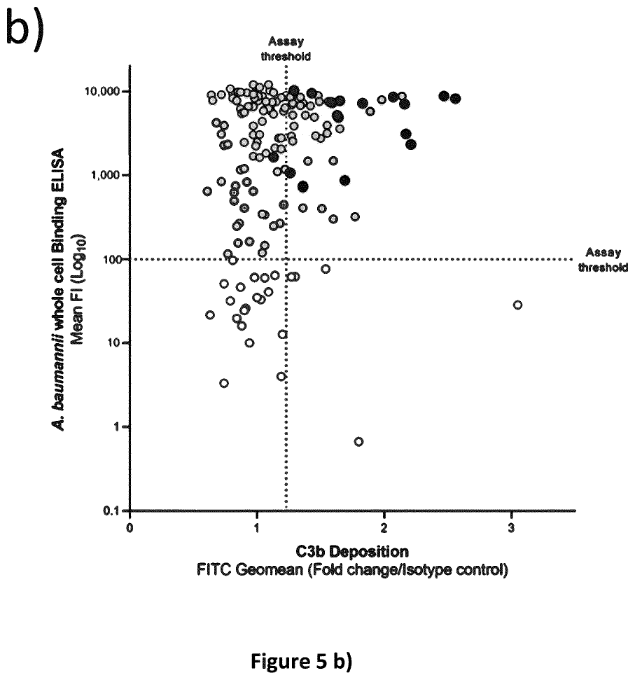

We employed ELISAs to detect binding of mAbs expressed at small-scale (100-500 μg/ml) in human HEK 293 cells to the GC2 A. baumannii OMVs used for immunisations. In this ELISA the OMVs are used as plate coating antigen. Using this ELISA approach, we identified 179 mAbs that bound to the immunising OMVs at a level above that of the threshold, determined by the isotype control: human IgG1+3 standard deviations. It is important to note that mAb binding to immunising GC2 A. baumannii OMVs only represents a correlate for binding to the intact bacterial cell wall and that target proteins may be more abundant in OMVs than in the context of the native bacterial membrane. To address this, we assessed the ability of the mAbs to bind to whole, unfixed GC2 A. baumannii bacterial strains from which the immunising OMVs were derived. The ELISA protocol was adapted to utilise whole, unfixed bacterial cells as the plate coating antigen. Using this assay, the number of mAbs that bound to the bacterial cells at a level above that of the threshold, determined by the isotype control: human IgG1+3 standard deviations was 136, compared to 179 identified as binding to OMVs. To further elucidate how mAb binding to OMVs correlated with mAb binding to the intact native bacterial wall of the GC2 clinical strains from which the OMVs are derived, we compared results of both binding assays, showing a close correlation between binding of mAbs to the target in both contexts. 75% of GC2 A. baumannii OMV binding mAbs also bound whole, unfixed cells of the GC2 A. baumannii strains from which the OMVs were derived (FIG. 5a). Although 25% of OMV-binding mAbs did not bind to the native bacterial membrane, only 2 clones were identified to bind to whole, unfixed bacterial cells and not OMVs. Our data indicate that use of OMVs as a sorting reagent for B cell plasma blasts is highly effective in identifying IgH/L pairs that can also bind the intact, unfixed bacterial cell wall of the clinical strains used to generate the OMVs. Further, these data validate consistency of bacterial target configuration between OMV and native cell wall contexts. Although we are confident that this should also apply to other gram-negative pathogens as well as Acinetobacter spp., the level of this consistency should be determined for each bacterial species used in our process.

The complement cascade is a powerful serum-based mechanism leading to insertion of protein pores into the membrane of gram-negative bacteria resulting in bacterial killing. This protein pathway is triggered by antibody and as such, the ability for an antibody to trigger the complement cascade is a key functional outcome of target engagement in our mAb discovery approach. C3b is cleaved from the serum C3 protein to drive the complement cascade and mAb binding to the bacterial cell membrane initiates formation of an enzymatic complex involving the complement proteins C1q, C2 and C4 that cleave C3. Therefore, detection of C3b on the bacterial membrane is a correlate for successful initiation of the complement cascade. To assess the ability of the HEK 293-expressed mAbs to induce complement activation, we established an in-house high-throughput, flow cytometry-based assay to detect deposition of the complement component C3b on the surface of OMVs. We adapted a published assay to accommodate FM4-64 stained GC2 A. baumannii OMVs, replacing antigen-conjugated beads, as they contain the target antigens in the bacterial membrane context. We had previously observed and assessed the non-specific association of FM4-64 stained OMVs to specific flow cytometry compensation beads and were able to exploit this association for the assay. Using this assay, we identified 76 mAbs that enhanced complement activation above assay threshold determined by the isotype control: human IgG1+3 standard deviations (FIG. 5b). We therefore describe assays we have prioritised for preliminary screening of mAbs as small scale HEK 293 supernatants. We selected assays based on being able to differentiate a binding signal between selected IgH/L chain pairs as an IgG1 antibody and IgG1 control. These assays have enabled us to identify a large panel of mAbs for further functional characterisation. This triage has generated a total of 136 mAbs able to bind to the native bacterial surface of at least one of the four clinical strains used to generate OMVs that were combined in the immunising panel. The next level of analysis involves expression in Chinese Hamster Ovary (CHO) cells and purification of selected mAbs enabling enough pure material to carry out multiple in vitro and in vivo assays. To select clones for downstream analysis and further characterisation, we assessed data from primary assays in conjunction with sequence data to ensure that the selected IgH/L pairs covered a wide spectrum of diversity at a sequence level. By selecting the most diverse sequences at this juncture, we maximise the probability of selecting mAbs with distinct functional properties and the potential for targeting multiple epitopes on the bacterial surface.

Based on this clustering data, we selected clones with small-scale HEK 293 expression level>100 μg/ml, for CHO cell expression, purification at higher quantity (5-10 mg) and subsequent further analysis in in vitro and in vivo assays. FIG. 5 illustrates the screening and selection process.

FIG. 5a. Comparison of mAb binding to GC2 A. baumannii OMVs and whole, unfixed bacterial cells as determined by ELISA. The mAbs identified as binders in both ELISA binding assays: mAbs that bound to both whole, unfixed GC2 A. baumannii bacterial cells and OMVs at levels above the assay thresholds indicated on the plot, are indicated in green. In orange are 2 mAbs that only bound whole, unfixed bacterial cells. The mAbs unable to bind to intact bacterial cell wall are indicated in white.

FIG. 5b. Selection of mAbs for CHO expression and purification based on binding of both intact bacterial cell wall and ability to trigger deposition of C3b as described above. Examples of selections for subsequent CHO expression are depicted in bold.

### 6.4 Example 5: Target Identification

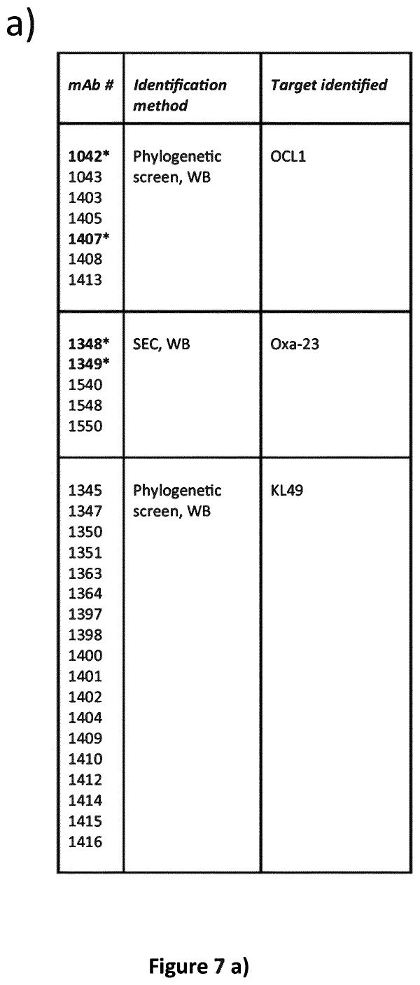

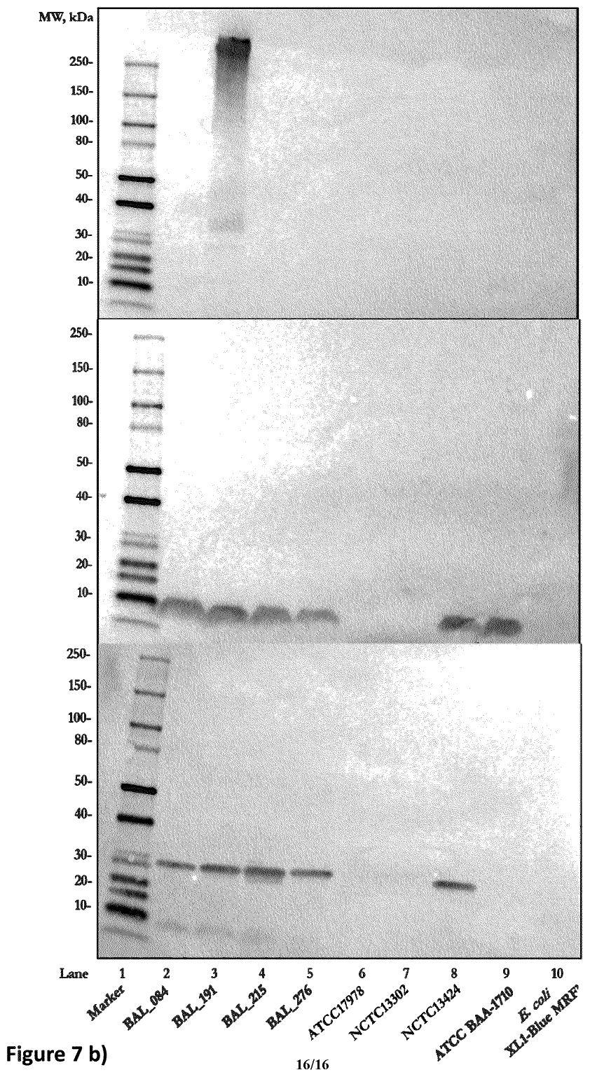

Focusing on the cross-reactive mAbs identified, we used a phage expression library incorporating shotgun A. baumannii genomic DNA from strains BAL 084 and BAL 276. Because the mAbs could potentially bind a polypeptide epitope, we screened mAbs for reactivity with phage plaques from the phage expression library. Using this screening mechanism, we were able to identify individual phage binding to Abs 1348, 1349. By sequencing the phage, we were able to elucidate 1348 and 1349 bound Oxa-23, a class D s-lactamase that is a major group of enzymes involved in resistance to carbapenem antibiotics. That we have identified mAbs binding to Oxa-23 in a target agnostic screen is highly significant. Firstly, this indicates that Oxa-23 is targetable by antibody in or closely associated with the A. baumannii cell membrane which was previously unknown. Secondly, since Oxa-23 is a functional enzyme that inactivates carbapenem and carbapenem resistance is a major clinical issue for the treatment of nosocomial A. baumannii infection, it may be possible to block enzymatic function of Oxa-23 by mAbs and render A. baumannii susceptible to carbapenems. We also interrogated bacterial lysates separated by SDS-PAGE and subjected to Western blot for reactivity to individual mAbs. Blots illustrating 6 major linear targets observed are shown in FIG. 7b. From the cross-reactive binding pattern observed for the mAbs 1042, 1043, 1403, 1405, 1407, 1408 and 1413 and binding pattern to a 10 kDa target in FIG. 7b we speculated this target might be lipooligosaccharide (LOS) type OC1. The LOS types were determined for a selection of isolates by OCL specific PCR. The PCR confirmed that the clinical A. baumannii isolates tested were all OC1 and only ATCC 17978 was OCL2. Next, LOS was extracted (with extensive proteinase K treatment to remove any proteins) and analyzed by Western blot using mAb 1042). E. coli NCTC 13441 LPS was extracted using the same method as a control and probed with a mAb specific for O25b O-antigen (ASN-4). Specific binding was observed with 1-42 to a 10kD target for OC1 isolates A. baumannii BAL_191, BAL_339, BAL_372 and no binding was observed to ATCC 17978 (OCL2) or E. coli NCTC13441 (025b) as expected. This data indicates that the 10kD target is OC1 LOS. We hypothesised that mAbs exclusively binding strains bearing the K49 capsule subtype were likely to be binding directly to capsule antigen. Capsule antigens were extracted from selected strains bearing KL49 and analyzed by subsequent SDS-PAGE and Western blot analysis using the mAb 1416. These results show that mAb 1416 was able to bind KL49 extracted from strains BAL_186 (KL49), BAL_242 (KL49), BAL_191 (KL49), BAL_316 (KL49), CM5132 (KL49), CM4980 (KL49), CM15768 (KL49) but not capsule antigen extracted from 91an (KL2) or BAL_339 (KL58). The presence of the KL49 capsule is associated with hyper virulence and increased mortality, and A. baumannii KL49 is currently emerging as the major clone in South East Asia.We were able to confirm that all mAbs binding to A. baumannii bearing the KL49 capsule subtype 1345, 1347, 1350, 1351, 1363, 1364, 1397, 1398, 1400, 1401, 1402, 1404, 1409, 1410, 1412, 1414, 1415 and 1416 bound to KL49 by western blot and correlated with the intense binding pattern to KL49 isolates observed in the HCl screen.

FIG. 7a. Table indicating identified targets for each mAb. Methods used include phylogenetic information gleaned from HCl (see above), serological expression cloning using a genomic phage library (SEC) and western blot with specific mAb (WB). The mAbs in bold (*) were tested in an in vivo challenge model of A. baumannii to demonstrate therapeutic efficacy.

FIG. 7b. Example western blot analysis reveals mAb binding three distinct targets; a high molecular weight target (KL49 capsule-upper panel), 10kD target (OC1 LOS-middle panel), and a 30kD protein (Oxa-23-lower panel).

### 7. SEQUENCES

TABLE 1
CDR, VH, and VL sequences:
CDRs in the table below are determined according to IMGT.

| SEQ ID NO | Antibody | Description | Sequence |
|---|---|---|---|
| 1 | PPB-1345 | VH domain | GAGGTGCAGCTGGTGGAGTCTGGGGGAGGCTTGGTACA |
| | | nucleic | GCCTGGGGGGTCCCTGAGACTCTCCTGTGCAGCCTCTG |
| | | acid | GATTCACCTTTAGCAGCTATGCCATGAGCTGGGTCCGC |
| | | sequence | CAGGCTCCAGGGAAGGGGCTGGAGTGGGTCTCAACTCT |
| | | | TAGTGGTAGTGGTGCTAGCACATACTACGCAGACTCCG |
| | | | TGAAGGGCCGGTTCACCATCTCCAGAGACAATTCCAAG |
| | | | AACGCGCTGTATCTGCAAATGAACAGCCTGAGAGCCGA |
| | | | GGACACGGCCGTATATTACTGTGCGAAAGAAGGGTTCG |
| | | | GGGACTGGTACTTCGATCTCTGGGGCCGTGGCACCCTG |
| | | | GTCACTGTCTCCTCA |
| 2 | PPB-1345 | VH domain | EVQLVESGGGLVQPGGSLRLSCAASGFTFSSYAMSWVR |
| | | amino acid | QAPGKGLEWVSTLSGSGASTYYADSVKGRFTISRDNSK |
| | | sequence | NALYLQMNSLRAEDTAVYYCAKEGFGDWYFDLWGRGTL |
| | | | VTVSS |
| 3 | PPB-1345 | HCDR1 | GFTFSSYA |
| 4 | PPB-1345 | HCDR2 | LSGSGAST |
| 5 | PPB-1345 | HCDR3 | AKEGFGDWYFDL |
| 6 | PPB-1345 | VL domain | GAAATAGTGATGACGCAGTCTCCAGCCACCCTGTCTGT |
| | | nucleic | GTCTCCAGGGGAAAGAGCCACCCTCTCCTGCAGGGCCA |
| | | acid | GTCAGAGTGTTAGTCGTAAGTTAGCCTGGTTCCAGCAG |
| | | sequence | AAACCTGGCCAGGCTCCCAGGCTCCTCATCTATGTTGC |
| | | | ATCCACCAGGGCCACTGGTATCCCAGCCAGGTTCAGTG |
| | | | GCAGTGGGTCTGGGACAGACTTCACTCTCACCATTAGT |
| | | | AGCCTGCAGTCTGAAGATTTTGCAATTTATTACTGTCA |
| | | | GCAGTATAATAACTGGCCGCTCACTTTCGGCGGAGGGA |
| | | | CCAAGGTGGAGATCAAA |
| 7 | PPB-1345 | VL domain | EIVMTQSPATLSVSPGERATLSCRASQSVSRKLAWFQQ |
| | | amino acid | KPGQAPRLLIYVASTRATGIPARFSGSGSGTDFTLTIS |
| | | sequence | SLQSEDFAIYYCQQYNNWPLTFGGGTKVEIK |
| 8 | PPB-1345 | LCDR1 | QSVSRK |
| 9 | PPB-1345 | LCDR2 | VAS |
| 10 | PPB-1345 | LCDR3 | QQYNNWPLT |
| 11 | PPB-1347 | VH domain | GAGGTGCAGCTGGTGGAGTCTGGGGGAGGCTTGGTACA |
| | | nucleic | GCCTGGGGGGTCCCTGAGACTCTCCTGTGCAGCCTCTG |
| | | acid | GATTCACCTTTAGCAGCTATGCCATGAGCTGGGTCCGC |
| | | sequence | CAGGCTCCAGGGAAGGGGCTGGAGTGGGTCTCAACTAT |
| | | | TAGTGGTAGTGGTGGTAGCACATACTACGCAGACTCCG |
| | | | TGAAGGGCCGGTTCTCCATCTCCAGAGACAATTCCAAG |
| | | | AACACGCTGTATCTGCAAATGAACAGCCTGAGAGCCGA |
| | | | GGACACGGCCGTATATTACTGTGCGAAAGGGGCCTACG |
| | | | GAAATTGGTACTTCGATATCTGGGGCCGTGGCACCCTG |
| | | | GTCACTGTCTCCTCA |
| 12 | PPB-1347 | VH domain | EVQLVESGGGLVQPGGSLRLSCAASGFTFSSYAMSWVR |
| | | amino acid | QAPGKGLEWVSTISGSGGSTYYADSVKGRFSISRDNSK |
| | | sequence | NTLYLQMNSLRAEDTAVYYCAKGAYGNWYFDIWGRGTL |
| | | | VTVSS |
| 3 | PPB-1347 | HCDR1 | GFTFSSYA |
| 13 | PPB-1347 | HCDR2 | ISGSGGST |
| 14 | PPB-1347 | HCDR3 | AKGAYGNWYFDI |
| 15 | PPB-1347 | VL domain | GAAATAGTGATGACGCAGTCTCCAGCCACCCTGTCTGT |
| | | nucleic | GTCTCCAGGGGAAAGAGCCACCCTCTCCTGCAGGGCCA |
| | | acid | GTCAGAGTGTTAGCAGCAACTTAGCCTGGTACCAGCAG |
| | | sequence | AAACCTGGCCAGGCTCCCAGGCTCCTCATCTATGGTGC |
| | | | ATCCATCAGGGCCACTGGCATCCCAGCCAGGTTCAGTG |
| | | | GCAGTGGGTCTGGGACAGAGTTCACTCTCACCATCAGC |
| | | | ATCCTGCAGTCTGAAGATTTTGCAGTTTATTACTGTCA |
| | | | CCAATATAATAACTGGCCGCTCACTTTCGGCGGGGGGA |
| | | | CCAAGGTGGAGATCAAA |
| 16 | PPB-1347 | VL domain | EIVMTQSPATLSVSPGERATLSCRASQSVSSNLAWYQQ |
| | | amino acid | KPGQAPRLLIYGASIRATGIPARFSGSGSGTEFTLTIS |
| | | sequence | ILQSEDFAVYYCHQYNNWPLTFGGGTKVEIK |
| 17 | PPB-1347 | LCDR1 | QSVSSN |
| 18 | PPB-1347 | LCDR2 | GAS |
| 19 | PPB-1347 | LCDR3 | HQYNNWPLT |
| 20 | PPB-1348 | VH domain | GAAGTGCAGCTGGTGGAGTCTGGGGGAGGCTTTGTACA |
| | | nucleic | GCCTGGCAGGTCCCTGAGACTCTCCTGTATAGCCTCTG |
| | | acid | GATTCACCTTTGATGATTATGCCATGCACTGGGTCCGG |
| | | sequence | CAAACTCCAGGGAAGGGCCTGGAGTGGGTCTCAGTTAT |
| | | | TAGTTGGAATAGTGTTAGCATAGGCTATGCGGACTCTG |
| | | | TGAAGGGCCGATTCACCATCTCCAGAGACAACGCCAAG |
| | | | AACTCCCTGTATCTGCAAATGAACAGTCTGAGAGCTGA |
| | | | GGACACGGCCTTGTATTACTGTGCTCGGTATAGCAGTG |
| | | | GCCCCGATGCTTTTGATATCTGGGGCCAAGGGACAATG |
| | | | GTCACCGTCTCTTCA |
| 21 | PPB-1348 | VH domain | EVQLVESGGGFVQPGRSLRLSCIASGFTFDDYAMHWVR |
| | | amino acid | QTPGKGLEWVSVISWNSVSIGYADSVKGRFTISRDNAK |
| | | sequence | NSLYLQMNSLRAEDTALYYCARYSSGPDAFDIWGQGTM |
| | | | VTVSS |
| 22 | PPB-1348 | HCDR1 | GFTFDDYA |
| 23 | PPB-1348 | HCDR2 | ISWNSVSI |
| 24 | PPB-1348 | HCDR3 | ARYSSGPDAFDI |
| 25 | PPB-1348 | VL domain | GACATCCAGATGACCCAGTCTCCATCCTCCCTGTCTGC |
| | | nucleic | ATCTGTAGGAGACAGAGTCACCATCACTTGCCGGGCAA |
| | | acid | GTCAGGACATTAGAAATGATTTAGGCTGGTATCAGCAG |
| | | sequence | AAACCAGGGAAAGCCCCTAAGCGCCTGATCTATGCTGC |
| | | | ATCCAGTTTGCAAAGTGGGGTCCCATCAAGGTTCAGCG |
| | | | GCAGTGGATTTGGGACAGAATTCACTCTCACAATCAGC |
| | | | AGCCTGCAGCCTGAAGATTTTGCAACTTATTACTGTCT |
| | | | ACAACATTATACTTACCCTCCTACTTTCGGCGGAGGGA |
| | | | CCAGGGTGGAGATCAAA |
| 26 | PPB-1348 | VL domain | DIQMTQSPSSLSASVGDRVTITCRASQDIRNDLGWYQQ |
| | | amino acid | KPGKAPKRLIYAASSLQSGVPSRFSGSGFGTEFTLTIS |
| | | sequence | SLQPEDFATYYCLQHYTYPPTFGGGTRVEIK |
| 27 | PPB-1348 | LCDR1 | QDIRND |
| 28 | PPB-1348 | LCDR2 | AAS |
| 29 | PPB-1348 | LCDR3 | LQHYTYPPT |
| 30 | PPB-1349 | VH domain | CAGATCACCTTGAAGGAGTCTGGTCCTACGCTGGTGAA |
| | | nucleic | ACCCACACAGACCCTCACGCTGACCTGCACCTTCTCTG |
| | | acid | GGTTCTCACTCAGCACTAGTGGAGTGGGTGTGGGCTGG |
| | | sequence | ATCCGTCAGCCCCCAGGAAAGGCCCTGGAGTGGCTTGC |
| | | | ACTCATTTATTGGGATGATGATAAGCGCTACAGCCCAT |
| | | | CTCTGAAGAGCAGGCTCACCATCACCAAGGACACCTCC |
| | | | AAAAACCAGGTGGTCCTTACAATGACCAACATGGACCC |
| | | | TGTGGACACAGCCACATATTACTGTGCACACAGTTATT |
| | | | ACTATGATAGTAGTGATTCTTACTACTTTGACTACTGG |
| | | | GGCCAGGGAACCCTGGTCACCGTCTCCTCA |
| 31 | PPB-1349 | VH domain | QITLKESGPTLVKPTQTLTLTCTFSGFSLSTSGVGVGW |
| | | amino acid | IRQPPGKALEWLALIYWDDDKRYSPSLKSRLTITKDTS |
| | | sequence | KNQVVLTMTNMDPVDTATYYCAHSYYYDSSDSYYFDYW |
| | | | GQGTLVTVSS |
| 32 | PPB-1349 | HCDR1 | GFSLSTSGVG |
| 33 | PPB-1349 | HCDR2 | IYWDDDK |
| 34 | PPB-1349 | HCDR3 | AHSYYYDSSDSYYFDY |
| 35 | PPB-1349 | VL domain | GACATCCAGTTGACCCAGTCTCCATCCTTCCTGTCTGC |
| | | nucleic | ATCTGTAGGAGACAGAGTCACCATCACTTGCTGGGCCA |
| | | acid | GTCAGGGCATTAGCAGTCGTTTAGCCTGGTATCAGCAA |
| | | sequence | AAACCAGGGAAAGCCCCGAAACTCCTGATCTATGCTGC |
| | | | ATCCACTTTGCAAAGTGGGGTCCCATCAAGGTTCAGCG |
| | | | GCAGTGGATCTGGGACAGAATTCACTCTCACAATCAGC |
| | | | AGCCTGCAGCCTGAAGATTTTGGAAATTATTACTGTCA |
| | | | ACAGTTTAATACTTACCCATTCACTTTCGGCCCTGGGA |
| | | | CCAAAGTGGATATCAAA |
| 36 | PPB-1349 | VL domain | DIQLTQSPSFLSASVGDRVTITCWASQGISSRLAWYQQ |
| | | amino acid | KPGKAPKLLIYAASTLQSGVPSRFSGSGSGTEFTLTIS |
| | | sequence | SLQPEDFGNYYCQQFNTYPFTFGPGTKVDIK |
| 37 | PPB-1349 | LCDR1 | QGISSR |
| 28 | PPB-1349 | LCDR2 | AAS |
| 38 | PPB-1349 | LCDR3 | QQFNTYPFT |
| 39 | PPB-1350 | VH domain | GAGGTGCAGCTGGTGGAGTCTGGGGGAGGCTTGGTACA |
| | | nucleic | GCCTGGGGGGTCCCTGAGACTCTCCTGTGCAGCCTCTG |
| | | acid | GATTCACCTTTAGCAGCTATGCCATGAGCTGGGTCCGC |
| | | sequence | CAGGCTCCAGGGAAGGGGCTGGAGTGGGTCTCAGCTAT |
| | | | TAGTGGTAGTGGTGGTAGCACACACTACGCAGACTCCG |
| | | | TGAAGGGCCGGTTCACCATCTCCAGAGACAATTCCAAG |
| | | | AACACGCTGTATCTGCAAATGAACAGCCTGAGAGCCGA |
| | | | GGACACGGCCGTATATTACTGTGCGAAAGAAGGCTACA |
| | | | GTAACTGGTACTTCGATCTCTGGGGCCGTGGCACCCTG |
| | | | GTCACTGTCTCCTCA |
| 40 | PPB-1350 | VH domain | EVQLVESGGGLVQPGGSLRLSCAASGFTFSSYAMSWVR |
| | | amino acid | QAPGKGLEWVSAISGSGGSTHYADSVKGRFTISRDNSK |
| | | sequence | NTLYLQMNSLRAEDTAVYYCAKEGYSNWYFDLWGRGTL |
| | | | VTVSS |
| 3 | PPB-1350 | HCDR1 | GFTFSSYA |
| 13 | PPB-1350 | HCDR2 | ISGSGGST |
| 41 | PPB-1350 | HCDR3 | AKEGYSNWYFDL |
| 42 | PPB-1350 | VL domain | GAAATAGTGATGACGCAGTCTCCAGCCACCCTGTCTGT |
| | | nucleic | GTCTCCAGGGGAAAGAGCCACCCTCTCCTGCAGGGCCA |
| | | acid | GTCAGAGTGTTAGCAGCAACTTAGCCTGGTACCAGCAG |
| | | sequence | AAACCTGGCCAGGCTCCCAGGCTCCTCATCTATGGTGC |
| | | | ATCCATCAGGGCCACTGGCATCCCAGCCAGGTTCAGTG |
| | | | GCAGTGGGTCTGGGACAGAGTTCACTCTCACCATCAGC |
| | | | ATCCTGCAGTCTGAAGATTTTGCAGTTTATTACTGTCA |
| | | | CCAGTATAATAACTGGCCGCTCACTTTCGGCGGAGGGA |
| | | | CCAAGGTGGAGATCAAA |
| 16 | PPB-1350 | VL domain | EIVMTQSPATLSVSPGERATLSCRASQSVSSNLAWYQQ |
| | | amino acid | KPGQAPRLLIYGASIRATGIPARFSGSGSGTEFTLTIS |
| | | sequence | ILQSEDFAVYYCHQYNNWPLTFGGGTKVEIK |
| 17 | PPB-1350 | LCDR1 | QSVSSN |
| 18 | PPB-1350 | LCDR2 | GAS |
| 19 | PPB-1350 | LCDR3 | HQYNNWPLT |
| 43 | PPB-1351 | VH domain | GAGGTGCAGCTGGTGGAGTCTGGGGGAGGCTTGGTACA |
| | | nucleic | GCCTGGGGGGTCCCTGAGACTCTCCTGTGCAGCCTCTG |
| | | acid | GATTCACCTTTAGCAGCTATGCCATGAGCTGGGTCCGC |
| | | sequence | CAGGCTCCAGGGAAGGGGCTGGAGTGGGTCTCAACTAT |
| | | | TAGTGGTAGTGGTGGTAGCACATACTACGCAGACTCCG |
| | | | TGAAGGGCCGGTTCACCATCTCCAGGGACAATTCCAAG |
| | | | AACACGCTGTATCTGCAAATGAACAGCCTGAGAGCCGA |
| | | | GGACACGGCCGTATATTACTGTGCGAAAGCGCCGTATA |
| | | | ACTGGAACTTTGACTACTGGGGCCAGGGAACCCTGGTC |
| | | | ACCGTCTCCTCA |
| 44 | PPB-1351 | VH domain | EVQLVESGGGLVQPGGSLRLSCAASGFTFSSYAMSWVR |
| | | amino acid | QAPGKGLEWVSTISGSGGSTYYADSVKGRFTISRDNSK |
| | | sequence | NTLYLQMNSLRAEDTAVYYCAKAPYNWNFDYWGQGTLV |
| | | | TVSS |
| 3 | PPB-1351 | HCDR1 | GFTFSSYA |
| 13 | PPB-1351 | HCDR2 | ISGSGGST |
| 45 | PPB-1351 | HCDR3 | AKAPYNWNFDY |
| 46 | PPB-1351 | VL domain | GAAGTAGTGATGACGCAGTCTCCAGCCACCCTGTCTGT |
| | | nucleic | GTCTCCAGGGGAAAGAGCCACCCTCTCCTGCAGGGCCA |
| | | acid | GTCAGAGTGTTAGCAGCAACTTAGCCTGGTACCAGCAG |
| | | sequence | AAACCTGGCCAGGCTCCCAGGCTCCTCATCTATGGTGC |
| | | | ATCCACCAGGGCCACTGGTATCCCAGCCAGGTTCAGTG |
| | | | GCAGTGGGTCTGGGACAGAGTTCACTCTCACCATCAGC |
| | | | AGCCTGCAGTCTGAAGATTTTGCAGTTTATTACTGTCA |
| | | | GCAGTATAATAACTGGCCTCTCACTTTCGGCGGAGGGA |
| | | | CCAAGGTGGAGATCAAA |
| 47 | PPB-1351 | VL domain | EVVMTQSPATLSVSPGERATLSCRASQSVSSNLAWYQQ |
| | | amino acid | KPGQAPRLLIYGASTRATGIPARFSGSGSGTEFTLTIS |
| | | sequence | SLQSEDFAVYYCQQYNNWPLTFGGGTKVEIK |
| 17 | PPB-1351 | LCDR1 | QSVSSN |
| 18 | PPB-1351 | LCDR2 | GAS |
| 10 | PPB-1351 | LCDR3 | QQYNNWPLT |
| 48 | PPB-1363 | VH domain | GAGGTGCAGCTGGTGGAGTCTGGGGGAGGCTTGGTACA |
| | | nucleic | GCCTGGGGGGTCCCTGAGACTCTCCTGTGCAGCCTCTG |
| | | acid | GATTCACCTTTAGCAGCTATGCCATGAGCTGGGTCCGC |
| | | sequence | CAGGCTCCAGGGAAGGGGCTGGAGTGGGTCTCATCTAT |
| | | | TAGTGGTAGTGGTGGTAGCACATACTACGCAGACTCCG |
| | | | TGAAGGGCCGGTTCACCATCTCCAGAGACAATTCCAAG |
| | | | AACACGCTGTATCTGCAAATGAGCAGCCTGAGAGCCGA |
| | | | GGACACGGCCGTTTATTACTGTGCGAAAGATGGTTACA |
| | | | GTAACTGGTACTTCGATCTCTGGGGCCGTGGCACCCTG |
| | | | GTCACTGTCTCCTCA |
| 49 | PPB-1363 | VH domain | EVQLVESGGGLVQPGGSLRLSCAASGFTFSSYAMSWVR |
| | | amino acid | QAPGKGLEWVSSISGSGGSTYYADSVKGRFTISRDNSK |
| | | sequence | NTLYLQMSSLRAEDTAVYYCAKDGYSNWYFDLWGRGTL |
| | | | VTVSS |
| 3 | PPB-1363 | HCDR1 | GFTFSSYA |
| 13 | PPB-1363 | HCDR2 | ISGSGGST |
| 50 | PPB-1363 | HCDR3 | AKDGYSNWYFDL |
| 51 | PPB-1363 | VL domain | GAAATAGTGATGACGCAGTCTCCAGCCACCCTGTCTGT |
| | | nucleic | GTCTCCAGGGGAAAGAGCCACCCTCTCCTGCAGGGCCA |
| | | acid | GTCAGAGTGTTAGCAGCAACTTAGCCTGGTACCAGCAG |
| | | sequence | AAACCTGGCCAGGCTCCCAGGCTCCTCATCTATGGCGC |
| | | | ATCCAACAGGGCCACTGGTATCCCAGCCAGGTTCAGTG |
| | | | GCAGTGGGTCTGGGACAGAGTTCACTCTCACCATCAGC |
| | | | AGCCTGCAGTCTGAAGATCTTGCAGTTTATTACTGTCA |
| | | | CCAGTATAATAACTGGCCGCTCACTTTCGGCGGAGGGA |
| | | | CCAAGGTGGAGATCAAA |
| 52 | PPB-1363 | VL domain | EIVMTQSPATLSVSPGERATLSCRASQSVSSNLAWYQQ |
| | | amino acid | KPGQAPRLLIYGASNRATGIPARFSGSGSGTEFTLTIS |
| | | sequence | SLQSEDLAVYYCHQYNNWPLTFGGGTKVEIK |
| 17 | PPB-1363 | LCDR1 | QSVSSN |
| 18 | PPB-1363 | LCDR2 | GAS |
| 19 | PPB-1363 | LCDR3 | HQYNNWPLT |
| 53 | PPB-1364 | VH domain | GAGGTGCAGCTGGTGGAGTCTGGGGGAGGCTTGGTACA |
| | | nucleic | GCCTGGGGGGTCCCTGAGACTCTCCTGTGCAGCCTCTG |
| | | acid | GATTCACCTTTAGCAGCTATGCCATGAACTGGGTCCGC |
| | | sequence | CAGGCTCCAGGGAAGGGGCTGGAGTGGGTCTCAACTAT |
| | | | TAGTGGTAGTGGTGGTAGCACATATTCCGCAGACTCCG |
| | | | TGAAGGGCCGGTTCACCATCTCCAGAGACAATTCCAAG |
| | | | AATACGCTGTATCTGCAAATGAACAGCCTGAGAGCCGA |
| | | | GGACACGGCCGTATATTACTGTGCGAAAGCTTATCGCA |
| | | | ACAACTGGTACTTTGAGTGCTGGGGCCAGGGAACCCTG |
| | | | GTCACCGTCTCCTCA |
| 54 | PPB-1364 | VH domain | EVQLVESGGGLVQPGGSLRLSCAASGFTFSSYAMNWVR |
| | | amino acid | QAPGKGLEWVSTISGSGGSTYSADSVKGRFTISRDNSK |
| | | sequence | NTLYLQMNSLRAEDTAVYYCAKAYRNNWYFECWGQGTL |
| | | | VTVSS |
| 3 | PPB-1364 | HCDR1 | GFTFSSYA |
| 13 | PPB-1364 | HCDR2 | ISGSGGST |
| 55 | PPB-1364 | HCDR3 | AKAYRNNWYFEC |
| 56 | PPB-1364 | VL domain | GAAATAGTGATGACGCAGTCTCCAGCCACCCTGTCTGT |
| | | nucleic | GTCTCCAGGGGAAAGAGTCACCCTCTCCTGCAGGGCCA |
| | | acid | GTCAGAGTGTTAACAGCAACTTAGCCTGGTACCAGCAG |
| | | sequence | AAACCTGGCCAGGCTCCCAGGCTCCTCATCTATGGTGC |
| | | | ATCCACCAGGGCCACTGGTATCCCAGCCAGGTTCAGTG |
| | | | GCAGTGGGTCTGGGACAGAGTTCACTCTCACCATCAAC |
| | | | AGCCTGCAGTCTGAAGACTTTGTAGTTTATTACTGTCA |
| | | | GCAGTATAATAATTGGCCATTCACTTTCGGCCCTGGGA |
| | | | CCAAAGTGGATATCAAA |
| 57 | PPB-1364 | VL domain | EIVMTQSPATLSVSPGERVTLSCRASQSVNSNLAWYQQ |
| | | amino acid | KPGQAPRLLIYGASTRATGIPARFSGSGSGTEFTLTIN |
| | | sequence | SLQSEDFVVYYCQQYNNWPFTFGPGTKVDIK |
| 58 | PPB-1364 | LCDR1 | QSVNSN |
| 18 | PPB-1364 | LCDR2 | GAS |
| 59 | PPB-1364 | LCDR3 | QQYNNWPFT |
| 60 | PPB-1397 | VH domain | GAGGTGCAGCTGGTGGAGTCTGGGGGAGGCTTGGTACA |
| | | nucleic | GCCTGGGGGGTCCCTGAGACTCTCCTGTGCAGCCTCTG |
| | | acid | GATTCACCTTTAGCAGCTATGCCATGAGCTGGGTCCGC |
| | | sequence | CAGGCTCCAGGGAAGGGGCTGGAGTGGGTCTCAACTAT |
| | | | TAGTGGTAGTGGTGGTAGCACATACTACGCAGACTCCG |
| | | | TGAAGGGCCGGTTCACCATCTCCAGAGACAATTCCAAG |
| | | | AACACGCTGTATCTGCAAATGAACAGCCTGAGAGCCGA |
| | | | GGACACGGCCGTATATTACTGTGCGAAAGGGGCCTACG |
| | | | GAACTTGGTACTTCGGTCTCTGGGGCCGTGGCACCCTG |
| | | | GTCACTGTCTCCTCA |
| 61 | PPB-1397 | VH domain | EVQLVESGGGLVQPGGSLRLSCAASGFTFSSYAMSWVR |
| | | amino acid | QAPGKGLEWVSTISGSGGSTYYADSVKGRFTISRDNSK |
| | | sequence | NTLYLQMNSLRAEDTAVYYCAKGAYGTWYFGLWGRGTL |
| | | | VTVSS |
| 3 | PPB-1397 | HCDR1 | GFTFSSYA |
| 13 | PPB-1397 | HCDR2 | ISGSGGST |
| 62 | PPB-1397 | HCDR3 | AKGAYGTWYFGL |
| 63 | PPB-1397 | VL domain | GAAATAGTGATGACGCAGTCTCCAGCCACCCTGTCTGT |
| | | nucleic | GTCTCCAGGGGAAAGAGCCACCCTCTCCTGCAGGGCCA |
| | | acid | GTCAGAGTGTTAGCAGCAACTTAGCCTGGTACCAGCAG |
| | | sequence | AAACCTGGCCAGGCTCCCAGGCTCCTCATCTATGGTGC |
| | | | ATCCATCAGGGCCACTGGCATCCCAGCCAGGTTCAGTG |
| | | | GCAGTGGGTCTGGGACAGAGTTCACTCTCACCATCAGC |
| | | | ATCCTGCAGTCTGAAGATTTTGCAGTTTATTACTGTCA |
| | | | GCAGTATAATAACTGGCCGTTCACTTTCGGCGGAGGGA |
| | | | CCAAGGTGGAGATCAAA |
| 64 | PPB-1397 | VL domain | EIVMTQSPATLSVSPGERATLSCRASQSVSSNLAWYQQ |
| | | amino acid | KPGQAPRLLIYGASIRATGIPARFSGSGSGTEFTLTIS |
| | | sequence | ILQSEDFAVYYCQQYNNWPFTFGGGTKVEIK |
| 17 | PPB-1397 | LCDR1 | QSVSSN |
| 18 | PPB-1397 | LCDR2 | GAS |
| 59 | PPB-1397 | LCDR3 | QQYNNWPFT |
| 65 | PPB-1398 | VH domain | GAGGTGCAGCTGGTGGAGTCTGGGGGAGGCTTGGTACA |
| | | nucleic | GCCGGGGGGGTCCCTGAGACTCTCCTGTGCAGCCTCTG |
| | | acid | GATTCACCTTTAGCAGCTATGCCATGAGCTGGGTCCGC |
| | | sequence | CAGGCTCCAGGGAAGGGGCTGGAGTGGGTCTCAGCTAT |
| | | | TAGTGGTAGTGGTGGTAGCACATACTACGCAGACTCCG |
| | | | TGAAGGGCCGGTTCACCATCTCCAGAGACAATTCCAAG |
| | | | AACACGCTGTATCTGCAAATGAACAGCCTGAGAGCCGA |
| | | | GGACACGGCCGTATATTACTGTGCGAAAAATAGCAGAA |
| | | | GCTGGGTGTTTGACTACTGGGGCCAGGGAACCCTGGTC |
| | | | ACCGTCTCCTCA |
| 66 | PPB-1398 | VH domain | EVQLVESGGGLVQPGGSLRLSCAASGFTFSSYAMSWVR |
| | | amino acid | QAPGKGLEWVSAISGSGGSTYYADSVKGRFTISRDNSK |
| | | sequence | NTLYLQMNSLRAEDTAVYYCAKNSRSWVFDYWGQGTLV |
| | | | TVSS |
| 3 | PPB-1398 | HCDR1 | GFTFSSYA |
| 13 | PPB-1398 | HCDR2 | ISGSGGST |
| 67 | PPB-1398 | HCDR3 | AKNSRSWVFDY |
| 68 | PPB-1398 | VL domain | GAAATAGTGATGACGCAGTCTCCAGCCACCCTGTCTGT |
| | | nucleic | GTCTCCAGGGGAAAGAGCCACCCTCTCCTGCAGGGCCA |
| | | acid | GTCAGAGTGTTAGCAGCAACTTAGCCTGGTACCAGCAG |
| | | sequence | AAACCTGGCCAGACTCCCAGGCTCCTCATGTATGGTGC |
| | | | ATCCACCAGGGCCACTGGTATCCCAGCCAGGTTCAGTG |
| | | | GCAGTGGGTCTGGGACAGAGTTCACTCTCACCATCAGC |
| | | | AGCCTGCAGTCTGAAGATTTTGCAGTTTATTACTGTCA |
| | | | GCAGTATAATAACTGGCCATTCACTTTCGGCCCTGGGA |
| | | | CCAAAGTGGATATCAAA |
| 69 | PPB-1398 | VL domain | EIVMTQSPATLSVSPGERATLSCRASQSVSSNLAWYQQ |
| | | amino acid | KPGQTPRLLMYGASTRATGIPARFSGSGSGTEFTLTIS |
| | | sequence | SLQSEDFAVYYCQQYNNWPFTFGPGTKVDIK |
| 17 | PPB-1398 | LCDR1 | QSVSSN |
| 18 | PPB-1398 | LCDR2 | GAS |
| 59 | PPB-1398 | LCDR3 | QQYNNWPFT |
| 70 | PPB-1400 | VH domain | GAGGTGCAGTTGGTGGAGTCTGGGGGAGGCTTGGTACA |
| | | nucleic | GCCGGGGGGGTCCCTGAGACTCTCCTGTGCAGCCTCTG |
| | | acid | GATTCACCTTTAGCAGCTATGCCATGAGCTGGGTCCGC |
| | | sequence | CAGGCTCCAGGGAAGGGGCTGGAGTGGGTCTCAACTAT |
| | | | TAGTGGTAGTGGTGGTAGCACACACTACGCAGACTCCG |
| | | | TGAAGGGCCGGTTCACCATCTCCAGAGACAATTCCAAG |
| | | | AACACGCTGTATCTGCAAATGAACAGCCTGAGAGCCGA |
| | | | GGACACGGCCGTATATTACTGTGCGAAAGATAGCAGCA |
| | | | CCTGGGTGTTTGACTACTGGGGCCAGGGAACCCTGGTC |
| | | | ACCGTCTCCTCA |
| 71 | PPB-1400 | VH domain | EVQLVESGGGLVQPGGSLRLSCAASGFTFSSYAMSWVR |
| | | amino acid | QAPGKGLEWVSTISGSGGSTHYADSVKGRFTISRDNSK |
| | | sequence | NTLYLQMNSLRAEDTAVYYCAKDSSTWVFDYWGQGTLV |
| | | | TVSS |
| 3 | PPB-1400 | HCDR1 | GFTFSSYA |
| 13 | PPB-1400 | HCDR2 | ISGSGGST |
| 72 | PPB-1400 | HCDR3 | AKDSSTWVEDY |
| 73 | PPB-1400 | VL domain | GAAATAGTGATGACGCAGTCTCCAGCCACCCTGTCTGT |
| | | nucleic | GTCTCCAGGGGAAAGAGCCACCCTCTCCTGCAGGGCCA |
| | | acid | GTCAGAGTGTTAGCAGCAACTTAGCCTGGTACCAGCAG |
| | | sequence | AAACCTGGCCAGGCTCCCAGGCTCCTCATCTATGGTGC |
| | | | ATCCACCAGGGCCACTGGTCTCCCAGCCAGGTTCAGTG |
| | | | GCAGTGGGTCTGGGACAGAGTTCACTCTCACCATCAGC |
| | | | AGCCTGCAGTCTGAAGATTTTGCAGTTTATTACTGTCA |
| | | | GCAGTATAATAACTGGCCATTCACTTTCGGCCCTGGGA |
| | | | CCAAAGTGGATATCAAA |
| 74 | PPB-1400 | VL domain | EIVMTQSPATLSVSPGERATLSCRASQSVSSNLAWYQQ |
| | | amino acid | KPGQAPRLLIYGASTRATGLPARFSGSGSGTEFTLTIS |
| | | sequence | SLQSEDFAVYYCQQYNNWPFTFGPGTKVDIK |
| 17 | PPB-1400 | LCDR1 | QSVSSN |
| 18 | PPB-1400 | LCDR2 | GAS |
| 59 | PPB-1400 | LCDR3 | QQYNNWPFT |
| 75 | PPB-1401 | VH domain | GAGGTGCAACTGGTGGAGTCTGGGGGAGGCTTGGTACA |
| | | nucleic | GCCTGGGGGGTCCCTGAGACTCTCCTGTGCAGCCTCTG |
| | | acid | GATTCACCTTTAGCAGCTATGCCATGAACTGGGTCCGC |
| | | sequence | CAGGCTCCAGGGAAGGGGCTGGAGTGGGTCTCAGCTAT |
| | | | TAGTGGTAGTGGTGGTAGCACAAACTACGCAGACTCCG |
| | | | TGAAGGGCCGGTTCACCATCTCCAGAGACAATGCCAAG |
| | | | AACACGCTGTATCTGCAAATGAACAGCCTGAGAGCCGA |
| | | | GGACACGGCCGTATATTACTGTGCGAAAGATATCTTCT |
| | | | CTAACTGGGCCATTGACTACTGGGGCCAGGGAACCCTG |
| | | | GTCACCGTCTCCTCA |
| 76 | PPB-1401 | VH domain | EVQLVESGGGLVQPGGSLRLSCAASGFTFSSYAMNWVR |
| | | amino acid | QAPGKGLEWVSAISGSGGSTNYADSVKGRFTISRDNAK |
| | | sequence | NTLYLQMNSLRAEDTAVYYCAKDIFSNWAIDYWGQGTL |
| | | | VTVSS |
| 3 | PPB-1401 | HCDR1 | GFTFSSYA |
| 13 | PPB-1401 | HCDR2 | ISGSGGST |
| 77 | PPB-1401 | HCDR3 | AKDIFSNWAIDY |
| 78 | PPB-1401 | VL domain | GAAATCGTGATGACGCAGTCTCCAGCCACCCTGTCTGT |
| | | nucleic | GTCTCCAGGGGAAAGAGCCACCCTCTCCTGCAGGGCCA |
| | | acid | GTCAGAGTGTTAGCAGCAACTTAGCCTGGTACCAGCAG |
| | | sequence | AAACCTGGCCAGGCTCCCAGGCTCCTCATCTATGGTGC |
| | | | ATCCATCAGGGCCACTGGCATCCCAGCCAGGTTCAGTG |
| | | | GCAGTGGGTCTGGGACAGAGTTCACTCTCACCATCAGC |
| | | | ATCCTGCAGTCTGAAGATTTTGCAGTTTATTACTGTCT |
| | | | TCAGTATCATAACTGGCCTCTCACTTTCGGCGGAGGGA |
| | | | CCAAGGTGGAGATCAAA |
| 79 | PPB-1401 | VL domain | EIVMTQSPATLSVSPGERATLSCRASQSVSSNLAWYQQ |
| | | amino acid | KPGQAPRLLIYGASIRATGIPARFSGSGSGTEFTLTIS |
| | | sequence | ILQSEDFAVYYCLQYHNWPLTFGGGTKVEIK |
| 17 | PPB-1401 | LCDR1 | QSVSSN |
| 18 | PPB-1401 | LCDR2 | GAS |
| 80 | PPB-1401 | LCDR3 | LQYHNWPLT |
| 81 | PPB-1402 | VH domain | GAGGTGCAGCTGGTGGAGTCTGGGGGAGGCTTGGTACA |
| | | nucleic | GCCTGGGGGGTCCCTGAGACTCTCCTGTGCAGCCTCTG |
| | | acid | GATTCACCTTTAGCAGCTATGCCATGAACTGGGTCCGC |
| | | sequence | CAGGCTCCAGGGAAGGGGCTGGAATGGGTCTCAGCTAT |
| | | | TAGTGGTAGTGGTGGTAGCACATACTACGCAGACTCCG |
| | | | TGAAGGGCCGGTTCACCATCTCCAGAGACAATTCCAAG |
| | | | AACACGCTGTATCTGCAAATGAACAGCCTGAGAGCCGA |
| | | | AGACACGACCGTATATTACTGTGCGAAAGGGGGTAGCA |
| | | | GGAACTGGTACTTTGACTACTGGGCCCAGGGAACCCTG |
| | | | GTCACCGTCTCCTCA |
| 82 | PPB-1402 | VH domain | EVQLVESGGGLVQPGGSLRLSCAASGFTFSSYAMNWVR |
| | | amino acid | QAPGKGLEWVSAISGSGGSTYYADSVKGRFTISRDNSK |
| | | sequence | NTLYLQMNSLRAEDTTVYYCAKGGSRNWYFDYWAQGTL |
| | | | VTVSS |
| 3 | PPB-1402 | HCDR1 | GFTFSSYA |
| 13 | PPB-1402 | HCDR2 | ISGSGGST |
| 83 | PPB-1402 | HCDR3 | AKGGSRNWYFDY |
| 84 | PPB-1402 | VL domain | GAAATAGTGATGACGCAGTCTCCAGCCACCCTGTCTGT |
| | | nucleic | GTCTCCAGGGGAAAGAGCCACCCTCTCCTGCAGGGCCA |
| | | acid | GTCAGAGTGTTAGCAGCAACTTAGCCTGGTACCAGCAG |
| | | sequence | AAACCTGGCCAGGCTCCCAGGCTCCTCATCTATGGTGC |
| | | | ATCCACCAGGGCCACTGGTATCCCAGCCAGGTTCAGTG |
| | | | GCAGTGGGTCTGGGACAGAGTTCACTCTCACCATCAGC |
| | | | AGCCTGCAGTCTGAAGATTTTGCAGTTTATTACTGTCT |
| | | | GCAGTATAATAACTGGCCGCTCACTTTCGGCGGAGGGA |
| | | | CCAAGGTGGAGATCAAA |
| 85 | PPB-1402 | VL domain | EIVMTQSPATLSVSPGERATLSCRASQSVSSNLAWYQQ |
| | | amino acid | KPGQAPRLLIYGASTRATGIPARFSGSGSGTEFTLTIS |
| | | sequence | SLQSEDFAVYYCLQYNNWPLTFGGGTKVEIK |
| 17 | PPB-1402 | LCDR1 | QSVSSN |
| 18 | PPB-1402 | LCDR2 | GAS |
| 86 | PPB-1402 | LCDR3 | LQYNNWPLT |
| 87 | PPB-1403 | VH domain | GAAGTGCAGCTGGTGGAGTCTGGGGGAGGCTTGGTACA |
| | | nucleic | GCCTGGCAGGTCCCTGAGACTCTCCTGTGCAGCCTCTG |
| | | acid | GATTCACCTTTGATGATTATGCCATGCACTGGGTCCGG |
| | | sequence | CAAGTTCCAGGGAAGGGCCTGGAGTGGGTCTCAGGTAT |
| | | | TAATTGGAATAGTGGTAGCTTAGTCTATGCGGACTCTG |
| | | | TGAAGGGCCGATTCACCATCTCCAGAGACAACGCCAAG |
| | | | AAGTTCCTGTATCTGCAAATGAACAGTCTGAGAGCTGA |
| | | | GGACACGGCCTTGTATTACTGTGCAAAAGATATCCGTA |
| | | | GAGCAGTGGCTGGTCGTTACTACTACTACTACGGTATG |
| | | | GACGTCTGGGGCCAAGGGACCACGGTCACCGTCTCCTC |
| | | | A |
| 88 | PPB-1403 | VH domain | EVQLVESGGGLVQPGRSLRLSCAASGFTFDDYAMHWVR |
| | | amino acid | QVPGKGLEWVSGINWNSGSLVYADSVKGRFTISRDNAK |
| | | sequence | KFLYLQMNSLRAEDTALYYCAKDIRRAVAGRYYYYYGM |
| | | | DVWGQGTTVTVSS |
| 22 | PPB-1403 | HCDR1 | GFTFDDYA |
| 89 | PPB-1403 | HCDR2 | INWNSGSL |
| 90 | PPB-1403 | HCDR3 | AKDIRRAVAGRYYYYYGMDV |
| 91 | PPB-1403 | VL domain | GACATCCAGATGACCCAGTCTCCATCCTCCCTGTCTGC |
| | | nucleic | TTCTGTAGGAGACAGAGTCACCATCACTTGCCGGGCAA |
| | | acid | GTCAGAGCATTAGCAGCTATTTAAATTGGTATCAGCAG |
| | | sequence | AAATCAGGGAAAGCCCCTAATCTCCTGATCTATGCTGC |
| | | | ATCCAGTTTGCAAAGTGGGGTCCCATCAAGGTTCAGTG |
| | | | GCAGTGGATCTGGGACAGATTTCACTCTCACCATCAGC |
| | | | AGTCTGCAACCTGAAGATTTTGCAACTTACTACTGTCA |
| | | | ACAGAGTTACATTACCCCGCTCACTTTCGGCGGAGGGA |
| | | | CCAAGGTGGAGATCAAA |
| 92 | PPB-1403 | VL domain | DIQMTQSPSSLSASVGDRVTITCRASQSISSYLNWYQQ |
| | | amino acid | KSGKAPNLLIYAASSLQSGVPSRFSGSGSGTDFTLTIS |
| | | sequence | SLQPEDFATYYCQQSYITPLTFGGGTKVEIK |
| 93 | PPB-1403 | LCDR1 | QSISSY |
| 28 | PPB-1403 | LCDR2 | AAS |
| 94 | PPB-1403 | LCDR3 | QQSYITPLT |
| 95 | PPB-1404 | VH domain | GAGGTGCAGCTGGTGGAGTCTGGGGGAGGCTTGGTACA |
| | | nucleic | GCCTGGGGGGTCCCTGAGACTCTCCTGTGCAGCCTCTG |
| | | acid | GATTCACCTTTAGCAGCTATGCCATGAGCTGGGTCCGC |
| | | sequence | CAGGCTCCAGGGAAGGGGCTGGAGTGGGTCTCAGCTAT |
| | | | TAGTGGTAGTGGTCGTGACACACACTACGCAGACTCCG |
| | | | TTAAGGGCCGGTTCACCATCTCCAGAGACAATTCCAAG |
| | | | AACACGCTGTTTCTGCAAATGAACAGCCTGAGAGCCGA |
| | | | GGACACGGCCGTGTATTACTGTGCGAAAGCCGAGTATA |
| | | | ACTGGAACTTCAACTACTGGGGCCAGGGAACCCTGGTC |
| | | | ACCGTCTCCTCA |
| 96 | PPB-1404 | VH domain | EVQLVESGGGLVQPGGSLRLSCAASGFTFSSYAMSWVR |
| | | amino acid | QAPGKGLEWVSAISGSGRDTHYADSVKGRFTISRDNSK |
| | | sequence | NTLFLQMNSLRAEDTAVYYCAKAEYNWNFNYWGQGTLV |
| | | | TVSS |
| 3 | PPB-1404 | HCDR1 | GFTFSSYA |
| 97 | PPB-1404 | HCDR2 | ISGSGRDT |
| 98 | PPB-1404 | HCDR3 | AKAEYNWNFNY |
| 99 | PPB-1404 | VL domain | GAAATAGTGATGACGCAGTCTCCAGCCACCCTGTCTGT |
| | | nucleic | GTCTCCAGGGGAAAGAGCCACCCTCTCCTGCAGGGCCA |
| | | acid | GTCAGAGTGTTAGCAGAAATTTAGCCTGGTACCAGCAG |
| | | sequence | AAACCTGGCCAGGCTCCCAGGCTCCTCATCTATGGTGC |
| | | | ATCCACCAGGGCCACTGGTATCCCAGCCAGGTTCAGTG |
| | | | GCAGTGGGTCTGGGACAGAGTTCACTCTCACCATCAGC |
| | | | AGCCTGCAGTCTGAAGATTTTGCAGTTTATTACTGTCA |
| | | | GCAGTATAATAACTGGCCATTCACTTTCGGCCCTGGGA |
| | | | CCAAAGTGGATATCAAA |
| 100 | PPB-1404 | VL domain | EIVMTQSPATLSVSPGERATLSCRASQSVSRNLAWYQQ |
| | | amino acid | KPGQAPRLLIYGASTRATGIPARFSGSGSGTEFTLTIS |
| | | sequence | SLQSEDFAVYYCQQYNNWPFTFGPGTKVDIK |
| 101 | PPB-1404 | LCDR1 | QSVSRN |
| 18 | PPB-1404 | LCDR2 | GAS |
| 59 | PPB-1404 | LCDR3 | QQYNNWPFT |
| 102 | PPB-1405 | VH domain | CAGATCACCTTGAAGGAGTCTGGTCCTACGCTGGTGAA |
| | | nucleic | ACCCACACAGACCCTCACGCTGACCTGCACCTTCTCTG |
| | | acid | GGTTCTCACTCAGTATTAGTGGAGTGGGTGTGGGCTGG |
| | | sequence | ATCCGTCAGCCCCCAGGAAAGGCCCTGGAGTGGCTTGC |
| | | | ACTCATTTATTGGGATGATGATAAGCGCTACAGCCCAT |
| | | | CTCTGAAGAACAGACTCACCATCACCAAGGACACCTCC |
| | | | AAAAACCAGGTGGTCCTTACAATGACCGACATGGACCC |
| | | | TGTGGACACAGCCACATATTACTGTGCACACCTACGAT |
| | | | ATTTTGACTTGGCGTACTACTTTGACTACTGGGGCCAG |
| | | | GGAACCCTGGTCACCGTCTCCTCA |
| 103 | PPB-1405 | VH domain | QITLKESGPTLVKPTQTLTLTCTFSGFSLSISGVGVGW |
| | | amino acid | IRQPPGKALEWLALIYWDDDKRYSPSLKNRLTITKDTS |
| | | sequence | KNQVVLTMTDMDPVDTATYYCAHLRYFDLAYYFDYWGQ |
| | | | GTLVTVSS |
| 104 | PPB-1405 | HCDR1 | GFSLSISGVG |
| 33 | PPB-1405 | HCDR2 | IYWDDDK |
| 105 | PPB-1405 | HCDR3 | AHLRYFDLAYYFDY |
| 106 | PPB-1405 | VL domain | GACATCCAGATGACCCAGTCTCCATCCTCCCTGTCTGC |
| | | nucleic | ATCTATAGGAGACAGAGTCACCATCACTTGCCGGGCAA |
| | | acid | GTCAGGACATTAGAAATGATTTAGGCTGGTATCAGCAG |
| | | sequence | AAACCAGGGAAAGCCCCTAAGCGCCTGATCTATGCTGC |
| | | | ATCCAGTTTGCAGAGTGGGGTCCCATCCAGGTTCAGCG |
| | | | CCAGTGGATCTGGGACAGAATTCACTCTCACAATCAGC |
| | | | AGCCTGCAGCCTGAAGATTTTGCAACTTATTACTGTCT |
| | | | ACAGTCTAATAGTTACCCATTCACTTTCGGCCCTGGGA |
| | | | CCAAAGTGGATATCAAA |
| 107 | PPB-1405 | VL domain | DIQMTQSPSSLSASIGDRVTITCRASQDIRNDLGWYQQ |
| | | amino acid | KPGKAPKRLIYAASSLQSGVPSRFSASGSGTEFTLTIS |
| | | sequence | SLQPEDFATYYCLQSNSYPFTFGPGTKVDIK |
| 27 | PPB-1405 | LCDR1 | QDIRND |
| 28 | PPB-1405 | LCDR2 | AAS |
| 108 | PPB-1405 | LCDR3 | LQSNSYPFT |
| 109 | PPB-1407 | VH domain | CAGATCACCTTGAAGGAGTCTGGTCCTACGCTGGTGAA |
| | | nucleic | ACCCACACAGACCCTCACCCTGACCTGCACCTTCTCTG |
| | | acid | GGTTCTCACTCAGACCTAGTGGAGTGGGTGTGGGCTGG |
| | | sequence | ATCCGTCAGCCCCCAGGAAAGGCCCTGGAGTGGCTTGC |
| | | | AATCATTTATTGGGATGATGATAAGCGCTACAGCCCAT |
| | | | CTCTGAAGAGCAGGCTCACCATCACCAAGGACACCTCC |
| | | | AAAAACCAGGTGGTCCTTACAATGACCAACATGGACCC |
| | | | TGTGGACACAGCCATATATTACTGTGCACACAGTTACT |
| | | | ATGATAATAGTGGTTATTACTTTGACTACTGGGGCCAG |
| | | | GGAACCCTGGTCACCGTCTCCTCA |
| 110 | PPB-1407 | VH domain | QITLKESGPTLVKPTQTLTLTCTFSGFSLRPSGVGVGW |
| | | amino acid | IRQPPGKALEWLAIIYWDDDKRYSPSLKSRLTITKDTS |
| | | sequence | KNQVVLTMTNMDPVDTAIYYCAHSYYDNSGYYFDYWGQ |
| | | | GTLVTVSS |
| 111 | PPB-1407 | HCDR1 | GFSLRPSGVG |
| 33 | PPB-1407 | HCDR2 | IYWDDDK |
| 112 | PPB-1407 | HCDR3 | AHSYYDNSGYYFDY |
| 113 | PPB-1407 | VL domain | GACATCCAGATGACCCAGTCTCCATCCTCACTGTCTGC |
| | | nucleic | ATCTGTAGGAGACAGAGTCACCATCACTTGTCGGGCGA |
| | | acid | GTCAGGGCATTAGCAATTTTTTAGCCTGGTTTCAGCAG |
| | | sequence | AAACCAGGGAAAGCCCCGAAGTCCCTGATCCATGCTGC |
| | | | ATCCAGTTTGCAAAGTGGGGTCCCATCAAAGTTCAGCG |
| | | | GCAGTGGATCTGGGACAGATTTCACTCTCACCATCAGC |
| | | | AGCCTGCAGCCTGAAGATTTTGCAACTTATTACTGCCA |
| | | | ACAGTATTATACTTACCCATTCACTTTCGGCCCTGGGA |
| | | | CCATACTGGATATCAGA |
| 114 | PPB-1407 | VL domain | DIQMTQSPSSLSASVGDRVTITCRASQGISNFLAWFQQ |
| | | amino acid | KPGKAPKSLIHAASSLQSGVPSKFSGSGSGTDFTLTIS |
| | | sequence | SLQPEDFATYYCQQYYTYPFTFGPGTILDIR |
| 115 | PPB-1407 | LCDR1 | QGISNF |
| 28 | PPB-1407 | LCDR2 | AAS |
| 116 | PPB-1407 | LCDR3 | QQYYTYPFT |
| 117 | PPB-1408 | VH domain | CAGATCACCTTGAAGGAGTCTGGTCCTACGCTGGTGAA |
| | | nucleic | ACCCACACAGACCCTCACGCTGACCTGTACCTTCTCTG |
| | | acid | GGTTCTCACTCAGTATTAGTGGAGTGGGTGTGGGCTGG |
| | | sequence | ATCCGTCAGCCCCCAGGAAAGGCCCTGGAGTGGCTTGC |
| | | | ACTCATTTATTGGGATGATGATAAGCGCTACAGCCCGT |
| | | | CTCTGAAGAGCAGGCTCACCATCACCAAGGACACCTCC |
| | | | AAAAACCAGGTGGTCCTTACAATGACCAACATGGACCC |
| | | | TGTGGACACAGCCACATATTACTGTGCACACAGCCATT |
| | | | ACTATGCTAGTACTGGTTATTACTTTGACTACTGGGGC |
| | | | CAGGGAACCCTGGTCACCGTCTCCTCA |
| 118 | PPB-1408 | VH domain | QITLKESGPTLVKPTQTLTLTCTFSGFSLSISGVGVGW |
| | | amino acid | IRQPPGKALEWLALIYWDDDKRYSPSLKSRLTITKDTS |
| | | sequence | KNQVVLTMTNMDPVDTATYYCAHSHYYASTGYYFDYWG |
| | | | QGTLVTVSS |
| 104 | PPB-1408 | HCDR1 | GFSLSISGVG |
| 33 | PPB-1408 | HCDR2 | IYWDDDK |
| 119 | PPB-1408 | HCDR3 | AHSHYYASTGYYFDY |
| 120 | PPB-1408 | VL domain | GCCATCCAGATGACCCAGTCTCCATCCTCCCTGTCTGC |
| | | nucleic | ATCTGTAGGAGACAGAGTCACCATCACTTGCCGGGCAA |
| | | acid | GTCAGGGCATTAGAAATAATTTAGGCTGGTATCAGCAG |
| | | sequence | AAACCAGGGAAAGCCCCTAAACTCCTGATCTATGCTGC |
| | | | ATCCGGTTTACAAAGTGGGGTCCCTTCAAGGTTCAGCG |
| | | | GCAGTGGATCTGGCACAGATTTCACTCTCACCATCAGC |
| | | | AGCCTGCAGCCTGAAGATTTTGCAACTTATTACTGTCT |
| | | | ACAAGATTACAATTACCCATTCACTTTCGGCCCTGGGA |
| | | | CCAAAGTGGATATCAAA |
| 121 | PPB-1408 | VL domain | AIQMTQSPSSLSASVGDRVTITCRASQGIRNNLGWYQQ |
| | | amino acid | KPGKAPKLLIYAASGLQSGVPSRFSGSGSGTDFTLTIS |
| | | sequence | SLQPEDFATYYCLQDYNYPFTFGPGTKVDIK |
| 122 | PPB-1408 | LCDR1 | QGIRNN |
| 28 | PPB-1408 | LCDR2 | AAS |
| 123 | PPB-1408 | LCDR3 | LQDYNYPFT |
| 124 | PPB-1409 | VH domain | GAGGTGCAACTGGTGGAGTCTGGGGGAGGCTTGGTACA |
| | | nucleic | GCCTGGGGGGTCCCTGAGACTCTCCGGTGCAGCCTCTG |
| | | acid | GATTCACCTTTAGCAGCTATGCCATGAGCTGGGTCCGC |
| | | sequence | CAGGTTCCAGGGAAGGGGCTGGAGTGGGTCTCAACTAT |
| | | | TAGTGGTAGTGGTGGTAGCACATACTACGCAGACTCCG |
| | | | TGAAGGGCCGGTTCACCATCTCCAGAGACAATTCCAAG |
| | | | AACACGCTGTATCTGCAAATGAACAGCCTGAGAGCCGA |
| | | | GGACACGGCCGTATATTACTGTGCGAAAGGGGGGATGG |
| | | | GGACCTGGTACTTCGATGTCTGGGGCCGTGGCACCCTG |
| | | | GTCACTGTCTCCTCA |
| 125 | PPB-1409 | VH domain | EVQLVESGGGLVQPGGSLRLSGAASGFTFSSYAMSWVR |
| | | amino acid | QVPGKGLEWVSTISGSGGSTYYADSVKGRFTISRDNSK |
| | | sequence | NTLYLQMNSLRAEDTAVYYCAKGGMGTWYFDVWGRGTL |
| | | | VTVSS |
| 3 | PPB-1409 | HCDR1 | GFTFSSYA |
| 13 | PPB-1409 | HCDR2 | ISGSGGST |
| 126 | PPB-1409 | HCDR3 | AKGGMGTWYFDV |
| 127 | PPB-1409 | VL domain | GAAATAGTGATGACGCAGTCTCCAGCCACCCTGTCTGT |
| | | nucleic | GTCTCCAGGGGAAAGAGCCACCCTCTCCTGCACGACCA |
| | | acid | GTCAGAGTGTTAGCAGCAACTTAGCCTGGTGCCAACAG |
| | | sequence | AAACCTGGCCAGGCTCCCAGGCTCCTCATCTATGGTGC |
| | | | ATCCACCAGGGCCACTGGTATCCCAGCCAGGTTCAGTG |
| | | | GCAGTGGGTCTGGGACAGAGTTCACTCTCACCATCAGC |
| | | | AGCCTGCAGTCTGAAGATTTTGCAGTTTATTACTGTCA |
| | | | GCAGTATCATAAGTGGCCATTCACTTTCGGCCCTGGGA |
| | | | CCAAAGTGGATATCAAA |
| 128 | PPB-1409 | VL domain | EIVMTQSPATLSVSPGERATLSCTTSQSVSSNLAWCQQ |
| | | amino acid | KPGQAPRLLIYGASTRATGIPARFSGSGSGTEFTLTIS |
| | | sequence | SLQSEDFAVYYCQQYHKWPFTFGPGTKVDIK |
| 17 | PPB-1409 | LCDR1 | QSVSSN |
| 18 | PPB-1409 | LCDR2 | GAS |
| 129 | PPB-1409 | LCDR3 | QQYHKWPFT |
| 130 | PPB-1410 | VH domain | GAGGTGCAGCTGGTGGAGTCTGGGGGAGGCTTGGTACA |
| | | nucleic | GCCTGGGGGGTCCCTGAGACTCTCCTGTGCAGCCTCTG |
| | | acid | GATTCACCTTTAGCAGCTATGCCATGAGCTGGGTCCGC |
| | | sequence | CAGGCTCCAGGGAAGGGGCTGGAGTGGGTCTCAGCTAT |
| | | | TAGTGGTAGTGGTGGTAGCACATTCTACGCAGACTCCG |
| | | | TGAAGGGCCGGTTCACCATCTCCAGAGACAATTCCAAG |
| | | | AACACGCTTTATCTGCAAATGAACAGCCTGAGAGCCGA |
| | | | GGACACGGCCGTATATTACTGTGCGAAAGATCCGGGGA |
| | | | ATAACTGGAATATGGAGTACTGGGGCCAGGGAACCCTG |
| | | | GTCACCGTCTCTTCA |
| 131 | PPB-1410 | VH domain | EVQLVESGGGLVQPGGSLRLSCAASGFTFSSYAMSWVR |
| | | amino acid | QAPGKGLEWVSAISGSGGSTFYADSVKGRFTISRDNSK |
| | | sequence | NTLYLQMNSLRAEDTAVYYCAKDPGNNWNMEYWGQGTL |
| | | | VTVSS |
| 3 | PPB-1410 | HCDR1 | GFTFSSYA |
| 13 | PPB-1410 | HCDR2 | ISGSGGST |
| 132 | PPB-1410 | HCDR3 | AKDPGNNWNMEY |
| 133 | PPB-1410 | VL domain | GAAATAGTGATGACGCAGTCTCCAGCCACCCTGTCTGT |
| | | nucleic | GTCTCCAGGAGAAAGAGCCACCCTCTCCTGCAGGGCCA |
| | | acid | GTCAGAGTGTTAGCAGCAACTTAGCCTGGTACCAGCAG |
| | | sequence | AAACCTGGCCAGGCTCCCAGGCTCCTCATCTATGGTGC |
| | | | ATCCACCAGGGCCACTGGTATCCCAGCCAGGTTCAGTG |
| | | | GCAGTGGGTCTGGGACAGAGTTCACTCTCACCATCAGC |
| | | | AGCCTGCAGTCTGGAGATTTTGCAGTTTATTACTGTCA |
| | | | CCAGTATCATAACTGGCCGCTCACTTTCGGCGGAGGGA |
| | | | CCAAGGTGGAGATCAAA |
| 134 | PPB-1410 | VL domain | EIVMTQSPATLSVSPGERATLSCRASQSVSSNLAWYQQ |
| | | amino acid | KPGQAPRLLIYGASTRATGIPARFSGSGSGTEFTLTIS |
| | | sequence | SLQSGDFAVYYCHQYHNWPLTFGGGTKVEIK |
| 17 | PPB-1410 | LCDR1 | QSVSSN |
| 18 | PPB-1410 | LCDR2 | GAS |
| 135 | PPB-1410 | LCDR3 | HQYHNWPLT |
| 136 | PPB-1412 | VH domain | GGGGTGCAGCTGGTGGACTCTGGGGGAGGCTTGGTACA |
| | | nucleic | GCCTGGGGGGTCCCTGAGACTCTCCTGTGCAGCCTCTG |
| | | acid | GATTCACCTTTAGCAGCTATGCCATGAACTGGGTCCGC |
| | | sequence | CAGGCTCCAGGGAAGGGGCTGGAGTGGGTCTCAACTAT |
| | | | TAGTGGTAGTGGTGGTACCACATACTACGCAGACTCCG |
| | | | TGAAGGGCCGGTTCACCATCTCCAGAGACAATTCCAAG |
| | | | AACACGCTGTATCTGCAAATGAACAGCCTGAGAGCCGA |
| | | | GGACACGGCCGTATATTACTGTGCGAAAGCGACCTACA |
| | | | GTAACTGGCCTTTTGACTACTGGGGCCAGGGAGCCCTG |
| | | | GTCACCGTCTCCTCA |
| 137 | PPB-1412 | VH domain | GVQLVDSGGGLVQPGGSLRLSCAASGFTFSSYAMNWVR |
| | | amino acid | QAPGKGLEWVSTISGSGGTTYYADSVKGRFTISRDNSK |
| | | sequence | NTLYLQMNSLRAEDTAVYYCAKATYSNWPFDYWGQGAL |
| | | | VTVSS |
| 3 | PPB-1412 | HCDR1 | GFTFSSYA |
| 138 | PPB-1412 | HCDR2 | ISGSGGTT |
| 139 | PPB-1412 | HCDR3 | AKATYSNWPFDY |
| 140 | PPB-1412 | VL domain | GAAATAGTGATGACGCAGTCTCCAGCCACCCTGTCTGT |
| | | nucleic | GTCTCCAGGGGAAAGAGCCACCCTCTCCTGCAGGGCCA |
| | | acid | GTCAGGGTGTTAGCAGCAACTTAGCCTGGTACCAGCAG |
| | | sequence | AAACCTGGCCAGGCTCCCAGGCTCCTCATCTATGGTGC |
| | | | ATCCATCAGGGCCACTGGCATCCCAGCCAGGTTCAGTG |
| | | | GCAGTGGGTCTGGGACAGAGTTCACTCTCACCATCAGC |
| | | | CTCCTGCAGTCTGAAGATTTTGCAGTTTATTACTGTCT |
| | | | GCAGTATAATAACTGGCCTCTCACTTTCGGCCCTGGGA |
| | | | CCAAAGTGGATATCAAA |
| 141 | PPB-1412 | VL domain | EIVMTQSPATLSVSPGERATLSCRASQGVSSNLAWYQQ |
| | | amino acid | KPGQAPRLLIYGASIRATGIPARFSGSGSGTEFTLTIS |
| | | sequence | LLQSEDFAVYYCLQYNNWPLTFGPGTKVDIK |
| 142 | PPB-1412 | LCDR1 | QGVSSN |
| 18 | PPB-1412 | LCDR2 | GAS |
| 86 | PPB-1412 | LCDR3 | LQYNNWPLT |
| 143 | PPB-1413 | VH domain | CAGATCACCTTGAGGGAGTCTGGTCCTCCGCTGGTGAA |
| | | nucleic | GCCCACACAGACCCTCACGCTGACCTGCACCTTCTCTG |
| | | acid | GGTTCTCACTCAGGACTAATGGAGTGGGTGTGGGCTGG |
| | | sequence | ACCCGTCAGCCCCCAGGAAAGGCCCTGGAGTGGCTTGC |
| | | | ACTCATTTATTGGGATGATGATAAGCGCTACAGCCCAT |
| | | | CTCTGAAGAGCAGGCTCACCATCACCAAGGGCACCTCC |
| | | | AAAAACCAGGTGGTCCTTACAATGACCAACATGGACCC |
| | | | TGTGGACACAGGCACATATTTCTGTGCACACGCGTATT |
| | | | ACTATAATTCGGGGAGTTATTTCTTTGACTACTGGGGC |
| | | | CAGGGAACCCTGGTCACCGTCTCCTCA |
| 144 | PPB-1413 | VH domain | QITLRESGPPLVKPTQTLTLTCTFSGFSLRTNGVGVGW |
| | | amino acid | TRQPPGKALEWLALIYWDDDKRYSPSLKSRLTITKGTS |
| | | sequence | KNQVVLTMTNMDPVDTGTYFCAHAYYYNSGSYFFDYWG |
| | | | QGTLVTVSS |
| 145 | PPB-1413 | HCDR1 | GFSLRTNGVG |
| 33 | PPB-1413 | HCDR2 | IYWDDDK |
| 146 | PPB-1413 | HCDR3 | AHAYYYNSGSYFFDY |
| 147 | PPB-1413 | VL domain | GACATCCAGATGACCCAGTCTCCATCCTCCCTGTCTGC |
| | | nucleic | ATCTGTAGGAGACAGAGTCACCATCATTTGCCGGGCAA |
| | | acid | GTCAGGGCATTAGAAATGATTTAGGCTGGTATCAGCAG |
| | | sequence | AAACCAGGGAAAGCCCCTAAGCGCCTGATCTCTGTTGT |
| | | | ATCCAGTTTGCAAAGTGGGGTCCCATCAAGGTTCAGCG |
| | | | GCAGTGGATCTGGGACAGAATTCACTCTCACAATCAGC |
| | | | AACCTACAGCCTGAAGATTTTGCAACTTATTACTGTCT |
| | | | ACAGCATAATAGTTACCCGTGGACGTTCGGCCAAGGGA |
| | | | CCAAGGTGGAAATCAAA |
| 148 | PPB-1413 | VL domain | DIQMTQSPSSLSASVGDRVTIICRASQGIRNDLGWYQQ |
| | | amino acid | KPGKAPKRLISVVSSLQSGVPSRFSGSGSGTEFTLTIS |
| | | sequence | NLQPEDFATYYCLQHNSYPWTFGQGTKVEIK |
| 149 | PPB-1413 | LCDR1 | QGIRND |
| 150 | PPB-1413 | LCDR2 | VVS |
| 151 | PPB-1413 | LCDR3 | LQHNSYPWT |
| 152 | PPB-1414 | VH domain | GAGGTGCAGCTGGTGGAGTCTGGGGGAGGCTTGGTACA |
| | | nucleic | GCCTGGGGGGTCCCTGAGACTCTCCTGTGCAGCCTCTG |
| | | acid | GATTCACCTTTAGCAGCTATCCCATGAGCTGGGTCCGC |
| | | sequence | CAGGCTCCAGGGAAGGGGCTGGAGTGGGTCTCAACTGT |
| | | | TAGTGGTAGTGGTGGTGACACACACTACGCAGACTCCG |
| | | | TGAAGGGCCGGTTCACCATCTCCAGAGACAATTCCAAG |
| | | | AACACGCTGTATCTGCAAATGAACAGCCTGAGAGCCGA |
| | | | CGACACGGCCGTATATTTCTGTGCGAAAGGCCCTCATA |
| | | | ACTGGAACTTTGACTACTGGGGCCAGGGAACCCTGGTC |
| | | | ACCGTCTCCTCA |
| 153 | PPB-1414 | VH domain | EVQLVESGGGLVQPGGSLRLSCAASGFTFSSYPMSWVR |
| | | amino acid | QAPGKGLEWVSTVSGSGGDTHYADSVKGRFTISRDNSK |
| | | sequence | NTLYLQMNSLRADDTAVYFCAKGPHNWNFDYWGQGTLV |
| | | | TVSS |
| 154 | PPB-1414 | HCDR1 | GFTFSSYP |
| 155 | PPB-1414 | HCDR2 | VSGSGGDT |
| 156 | PPB-1414 | HCDR3 | AKGPHNWNFDY |
| 157 | PPB-1414 | VL domain | GAAATAGTGATGACGCAGTCTCCAGCCACCCTGTCTGT |
| | | nucleic | GTTTTCAGGGGAAAGAGCCACCCTCTCCTGCAGGGCCA |
| | | acid | GTCAGAGTGTTAGTAGCAACTTAGCCTGGTACCAGCAG |
| | | sequence | AAACCTGGCCAGGCTCCCAGGCTCCTCATCTATGGTGC |
| | | | ATCCACCAGGGCCACTGGTATCCCAGTCAGGTTCAGTG |
| | | | GCAGTGGGTCTGGGACAGAGTTCACTCTCACCATCAGC |
| | | | AGCCTGCTGTCTGAAGATTTTGCAGTTTATTACTGTCA |
| | | | GCAGTATAATAAGTGGCCTCTCACTTTCGGCGGAGGGA |
| | | | CCATGGTGGAGATCAGA |
| 158 | PPB-1414 | VL domain | EIVMTQSPATLSVFSGERATLSCRASQSVSSNLAWYQQ |
| | | amino acid | KPGQAPRLLIYGASTRATGIPVRFSGSGSGTEFTLTIS |
| | | sequence | SLLSEDFAVYYCQQYNKWPLTFGGGTMVEIR |
| 17 | PPB-1414 | LCDR1 | QSVSSN |
| 18 | PPB-1414 | LCDR2 | GAS |
| 159 | PPB-1414 | LCDR3 | QQYNKWPLT |
| 160 | PPB-1415 | VH domain | GAGGTGCAGGTGGTGGAGTCTGGGGGAGGCTTGGTACA |
| | | nucleic | GCCTGGGGGGTCCCTGAGACTCTCCTGTGCAGCCTCTG |
| | | acid | GATTCACCTTTAGCAGCTATGCCATGAGCTGGGTCCGC |
| | | sequence | CAGGCTCCAGGGAAGGGGCTGGAGTGGGTCTCAGCTCT |
| | | | TAGTGGCAGTGGTGGTCGCACATACTACGCAGACTCCG |
| | | | TGAAGGGCCGGTTCACCATCTCCAGAGACAATTCCAAG |
| | | | AACACGCTGTATCTGCAAATGGACAGTCTGAGAGTCGA |
| | | | GGACACGGCCGTATATTACTGTGCGAAAGATGGCATGG |
| | | | GGAACTGGTACTTCGATCTCTGGGGCCGTGGCACCCTG |
| | | | GTCACTGTCTCCTCA |
| 161 | PPB-1415 | VH domain | EVQVVESGGGLVQPGGSLRLSCAASGFTFSSYAMSWVR |
| | | amino acid | QAPGKGLEWVSALSGSGGRTYYADSVKGRFTISRDNSK |
| | | sequence | NTLYLQMDSLRVEDTAVYYCAKDGMGNWYFDLWGRGTL |
| | | | VTVSS |
| 3 | PPB-1415 | HCDR1 | GFTFSSYA |
| 162 | PPB-1415 | HCDR2 | LSGSGGRT |
| 163 | PPB-1415 | HCDR3 | AKDGMGNWYFDL |
| 164 | PPB-1415 | VL domain | GAAATAGTGATGACGCAGTCTCCAGCCACCCTGTCTGT |
| | | nucleic | GTCTCCAGGGGAAAGAGCCACCCTCTCCTGCAGGGCCA |
| | | acid | GTCAGAGTGTTAGCAGCAACTTAGCCTGGTACCAGCAG |
| | | sequence | AAACCTGGCCAGGCTCCCAGGCTCCTCATCTATGGTGC |
| | | | ATCCACCAGGGCCACTGGTATCCCAGCCAGGATCAGTG |
| | | | GCAGTGGGTCTGGGACAGAGTTCACTCTCGCCATCAGC |
| | | | AGCCTGCAGTCTGAAGATTTTGCAGTTTATTACTGTCA |
| | | | TCAGTATCATAACTGGCCTCTCACTTTCGGCGGAGGGA |
| | | | CCAAGGTGGAGATCAAA |
| 165 | PPB-1415 | VL domain | EIVMTQSPATLSVSPGERATLSCRASQSVSSNLAWYQQ |
| | | amino acid | KPGQAPRLLIYGASTRATGIPARISGSGSGTEFTLAIS |
| | | sequence | SLQSEDFAVYYCHQYHNWPLTFGGGTKVEIK |
| 17 | PPB-1415 | LCDR1 | QSVSSN |
| 18 | PPB-1415 | LCDR2 | GAS |
| 135 | PPB-1415 | LCDR3 | HQYHNWPLT |
| 166 | PPB-1416 | VH domain | GAGGTGCAGCTGGTGGAGTCTGGGGGAGGCTTGGTACA |
| | | nucleic | GCCTGGGGGGTCCCTGAGACTCTCCTGTGCAGCCTCTG |
| | | acid | GATTCACCTTTAGCAGCTATGCCATGAGCTGGGTCCGC |
| | | sequence | CAGGCTCCAGGGAAGGGGCTGGAGTGGGTCTCAACTAT |
| | | | TAGTGGTAGTGGTGGTAGCACACACTACGCAGACTCCG |
| | | | TGAAGGGCCGGTTCACCATCTCCAGAGACAATTCCAAG |
| | | | AACACGCTGTTTCTGCAAATGAACAGCCTGAGAGCCGA |
| | | | GGACACGGCCGTATATTACTGTGCGAAAGATGCCTTTG |
| | | | GGAACTGGTACATCGATCTCTGGGGCCGTGGCACCCTG |
| | | | GTCACTGTCTCCTCA |
| 167 | PPB-1416 | VH domain | EVQLVESGGGLVQPGGSLRLSCAASGFTFSSYAMSWVR |
| | | amino acid | QAPGKGLEWVSTISGSGGSTHYADSVKGRFTISRDNSK |
| | | sequence | NTLFLQMNSLRAEDTAVYYCAKDAFGNWYIDLWGRGTL |
| | | | VTVSS |
| 3 | PPB-1416 | HCDR1 | GFTFSSYA |
| 13 | PPB-1416 | HCDR2 | ISGSGGST |
| 168 | PPB-1416 | HCDR3 | AKDAFGNWYIDL |
| 169 | PPB-1416 | VL domain | GAAATAGTGATGACGCAGTCTCCAGCCACCCTGTCTGT |
| | | nucleic | GTCTCCAGGGGAAAGAGCCACCCTCTCCTGCAGGGCCA |
| | | acid | GTCAGAGTGTTAGCAGCAACTTAGCCTGGTACCAGCAG |
| | | sequence | AAACCTGGCCAGGCTCCCAGGCTCCTCATCTATGGTGC |
| | | | ATCCACCAGGGCCACTGGTATCCCAGCCAGGTTCAGTG |
| | | | GCAGTGGGTCTGGGACAGAGTTCACTCTCACCATCAGC |
| | | | AGCCTGCAGTCTGAATATTTTGCAATTTATTACTGTCA |
| | | | GCAGTATAATAACTGGCCTCTCACTTTCGGCGGAGGAA |
| | | | CCAAGGTGGAGATCAAA |
| 170 | PPB-1416 | VL domain | EIVMTQSPATLSVSPGERATLSCRASQSVSSNLAWYQQ |
| | | amino acid | KPGQAPRLLIYGASTRATGIPARFSGSGSGTEFTLTIS |
| | | sequence | SLQSEYFAIYYCQQYNNWPLTFGGGTKVEIK |
| 17 | PPB-1416 | LCDR1 | QSVSSN |
| 18 | PPB-1416 | LCDR2 | GAS |
| 10 | PPB-1416 | LCDR3 | QQYNNWPLT |
| 20 | PPB-1540 | VH domain | GAAGTGCAGCTGGTGGAGTCTGGGGGAGGCTTTGTACA |
| | | nucleic | GCCTGGCAGGTCCCTGAGACTCTCCTGTATAGCCTCTG |
| | | acid | GATTCACCTTTGATGATTATGCCATGCACTGGGTCCGG |
| | | sequence | CAAACTCCAGGGAAGGGCCTGGAGTGGGTCTCAGTTAT |
| | | | TAGTTGGAATAGTGTTAGCATAGGCTATGCGGACTCTG |
| | | | TGAAGGGCCGATTCACCATCTCCAGAGACAACGCCAAG |
| | | | AACTCCCTGTATCTGCAAATGAACAGTCTGAGAGCTGA |
| | | | GGACACGGCCTTGTATTACTGTGCTCGGTATAGCAGTG |
| | | | GCCCCGATGCTTTTGATATCTGGGGCCAAGGGACAATG |
| | | | GTCACCGTCTCTTCA |
| 21 | PPB-1540 | VH domain | EVQLVESGGGFVQPGRSLRLSCIASGFTFDDYAMHWVR |
| | | amino acid | QTPGKGLEWVSVISWNSVSIGYADSVKGRFTISRDNAK |
| | | sequence | NSLYLQMNSLRAEDTALYYCARYSSGPDAFDIWGQGTM |
| | | | VTVSS |
| 22 | PPB-1540 | HCDR1 | GFTFDDYA |
| 23 | PPB-1540 | HCDR2 | ISWNSVSI |
| 24 | PPB-1540 | HCDR3 | ARYSSGPDAFDI |
| 25 | PPB-1540 | VL domain | GACATCCAGATGACCCAGTCTCCATCCTCCCTGTCTGC |
| | | nucleic | ATCTGTAGGAGACAGAGTCACCATCACTTGCCGGGCAA |
| | | acid | GTCAGGACATTAGAAATGATTTAGGCTGGTATCAGCAG |
| | | sequence | AAACCAGGGAAAGCCCCTAAGCGCCTGATCTATGCTGC |
| | | | ATCCAGTTTGCAAAGTGGGGTCCCATCAAGGTTCAGCG |
| | | | GCAGTGGATTTGGGACAGAATTCACTCTCACAATCAGC |
| | | | AGCCTGCAGCCTGAAGATTTTGCAACTTATTACTGTCT |
| | | | ACAACATTATACTTACCCTCCTACTTTCGGCGGAGGGA |
| | | | CCAGGGTGGAGATCAAA |
| 26 | PPB-1540 | VL domain | DIQMTQSPSSLSASVGDRVTITCRASQDIRNDLGWYQQ |
| | | amino acid | KPGKAPKRLIYAASSLQSGVPSRFSGSGFGTEFTLTIS |
| | | sequence | SLQPEDFATYYCLQHYTYPPTFGGGTRVEIK |
| 27 | PPB-1540 | LCDR1 | QDIRND |
| 28 | PPB-1540 | LCDR2 | AAS |
| 29 | PPB-1540 | LCDR3 | LQHYTYPPT |
| 171 | PPB-1548 | VH domain | CAGGTGCAGCTGGTGGAGTCTGGGGGAGGCGTGGTCCA |
| | | nucleic | GCCTGGGAGGTCCCTGAGACTCTCCTGTGCAGCCTCTG |
| | | acid | GATTCACCTTCAGTAGCTATGCCATGCACTGGGTCCGC |
| | | sequence | CAGGCTCCAGGCAAGGGGCTGGAGTGGGTGGCAGTTAT |
| | | | ATCATATGATGGAAGTAATAAATACTATGCAGACTCCG |
| | | | TGAAGGGCCGATTCACCATCTCCAGAGACAATTCCAAG |
| | | | AACACGCTGTATCTGCAAATGAACAGCCTGAGAGCTGA |
| | | | GGACACGGCTGTGTATTACTGTGCGGGCTACGGTGGTA |
| | | | ACTCGCTCGACCCCTGGGGCCAGGGAACCCTGGTCACC |
| | | | GTCTCCTCA |
| 172 | PPB-1548 | VH domain | QVQLVESGGGVVQPGRSLRLSCAASGFTFSSYAMHWVR |
| | | amino acid | QAPGKGLEWVAVISYDGSNKYYADSVKGRFTISRDNSK |
| | | sequence | NTLYLQMNSLRAEDTAVYYCAGYGGNSLDPWGQGTLVT |
| | | | VSS |
| 3 | PPB-1548 | HCDR1 | GFTFSSYA |
| 173 | PPB-1548 | HCDR2 | ISYDGSNK |
| 174 | PPB-1548 | HCDR3 | AGYGGNSLDP |
| 175 | PPB-1548 | VL domain | GCCATCCAGATGACCCAGTCTCCATCCTCCCTGTCTGC |
| | | nucleic | ATCTGTAGGAGACAGAGTCACCATCACTTGCCGGGCAA |
| | | acid | GTCAGGGCATTAGAAATGATTTAGTCTGGTATCAGCAG |
| | | sequence | AAACCAGGGAAAGCCCCTAAGGTCCTGATCTATGATGC |
| | | | GTCCAGTTTACAAAGTGGGGTCCCATCAAGGTTCAGCG |
| | | | GCAGTGGATTTGGCACAGATTTCACTCTCACCATCAGC |
| | | | AGCCTGCAGCCTGAAGATTTTGCAACTTATTACTGTCT |
| | | | ACAGGATTACAATTACCCGTGCAGTTTTGGCCAGGGGA |
| | | | CCAAGCTGGAGATCAAA |
| 176 | PPB-1548 | VL domain | AIQMTQSPSSLSASVGDRVTITCRASQGIRNDLVWYQQ |
| | | amino acid | KPGKAPKVLIYDASSLQSGVPSRFSGSGFGTDFTLTIS |
| | | sequence | SLQPEDFATYYCLQDYNYPCSFGQGTKLEIK |
| 149 | PPB-1548 | LCDR1 | QGIRND |
| 177 | PPB-1548 | LCDR2 | DAS |
| 178 | PPB-1548 | LCDR3 | LQDYNYPCS |
| 179 | PPB-1550 | VH domain | CAGGTGCAGCTGGTGGAGTCTGGGGGAGGCGTGGTCCA |
| | | nucleic | GCCTGGGAGGTCCCTGAGACTCTCCTGTGCAGCCTCTG |
| | | acid | GATTCACCTTCAGTAGCTATGGCATGCACTGGGTCCGC |
| | | sequence | CAGGCTCCAGGCAAGGGGCTGGAGTGGGTGGCAGTTAT |
| | | | ATCATATGATGGAAGTAATAAATACTATGCAGACTCCG |
| | | | TGAAGGGCCGATTCACCATCTCCAGAGACAATTCCAAG |
| | | | AACACGCTGTATCTGCAAATGAACAGCCTGAGAGCTGA |
| | | | GGACACGGCTGTGTATTACTGTGCGAAAGAGAGCCTTA |
| | | | CTATGGTTCGGGGAGAGGGGTTCGACCCCTGGGGCCAG |
| | | | GGAACCCTGGTCACCGTCTCCTCA |
| 180 | PPB-1550 | VH domain | QVQLVESGGGVVQPGRSLRLSCAASGFTFSSYGMHWVR |
| | | amino acid | QAPGKGLEWVAVISYDGSNKYYADSVKGRFTISRDNSK |
| | | sequence | NTLYLQMNSLRAEDTAVYYCAKESLTMVRGEGFDPWGQ |
| | | | GTLVTVSS |
| 181 | PPB-1550 | HCDR1 | GFTFSSYG |
| 173 | PPB-1550 | HCDR2 | ISYDGSNK |
| 182 | PPB-1550 | HCDR3 | AKESLTMVRGEGFDP |
| 183 | PPB-1550 | VL domain | GACATCCAGATGACCCAGTCTCCATCTTCCGTGTCTGC |
| | | nucleic | ATCTGTAGGAGACAGAGTCACCATCACTTGTCGGGCGA |
| | | acid | GTCAGGGTATTAGCAGCTGGTTAGCCTGGTATCAGCAG |
| | | sequence | AAACCAGGGAAAGCCCCTAAGCTCCTGATCTATGCTGC |
| | | | ATCCAGTTTGCAAAGTGGGGTCCCATCAAGGTTCAGCG |
| | | | GCAGTGGATCTGGGACAGATTTCACTCTCACCATCAGC |
| | | | AGCCTGCAGCCTGAAGATTTTGCAACTTACTATTGTCA |
| | | | ACAGGCTAACAGTTTCCCGTGCAGTTTTGGCCAGGGGA |
| | | | CCAAGCTGGAGATCAAA |
| 184 | PPB-1550 | VL domain | DIQMTQSPSSVSASVGDRVTITCRASQGISSWLAWYQQ |
| | | amino acid | KPGKAPKLLIYAASSLQSGVPSRFSGSGSGTDFTLTIS |
| | | sequence | SLQPEDFATYYCQQANSFPCSFGQGTKLEIK |
| 185 | PPB-1550 | LCDR1 | QGISSW |
| 28 | PPB-1550 | LCDR2 | AAS |
| 186 | PPB-1550 | LCDR3 | QQANSFPCS |
| 187 | PPB-1042 | VH domain | CAGATCACCTTGAAGGAGTCTGGTCCTACGCTGGTGAA |
| | | nucleic | ACCCACACAGACCCTCACGCTGACCTGCACCTTCTCTG |
| | | acid | GGTTCTCACTCAGCACTAGTGGAGTGGGTGTGGGCTGG |
| | | sequence | ATCCGTCAGCCCCCAGGAAAGGCCCTGGAGTGGCTTGC |
| | | | AGTCATTTATTGGGATGATGATAAGCGCTACAGCCCAT |
| | | | CTCTGAAGAGCAGGGTCACCATCACCAAGGACACCTCC |
| | | | AAAAACCAGGTGGTCCTTACAATGACCAATATGGACCC |
| | | | TGTGGACACAGCCACATATTACTGTGCACACAGTTATT |
| | | | ACCATGATAGCAGTGATTCTTACTACTTTGACTACTGG |
| | | | GCCCAGGGAACCCTGGTCACCGTCTCCTCA |
| 188 | PPB-1042 | VH domain | QITLKESGPTLVKPTQTLTLTCTFSGFSLSTSGVGVGW |
| | | amino acid | IRQPPGKALEWLAVIYWDDDKRYSPSLKSRVTITKDTS |
| | | sequence | KNQVVLTMTNMDPVDTATYYCAHSYYHDSSDSYYFDYW |
| | | | AQGTLVTVSS |
| 32 | PPB-1042 | HCDR1 | GFSLSTSGVG |
| 33 | PPB-1042 | HCDR2 | IYWDDDK |
| 189 | PPB-1042 | HCDR3 | AHSYYHDSSDSYYFDY |
| 190 | PPB-1042 | VL domain | GACATCCAGTTGACCCAGTCTCCATCCTTCCTGTCTGC |
| | | nucleic | ATCTGTAGGAGACAGAGTCACCATCACTTGCTGGGCCA |
| | | acid | GTCAGGGCATTAGCAGTCGTTTAGCCTGGTATCAGCAA |
| | | sequence | AAACCAGGGAAAGCCCCTAAGCTCCTGATCTATGCTGC |
| | | | ATCCACTTTGCAAAGTGGGGTCCCATCAAGGTTCAGCG |
| | | | GCAGTGGATCTGGGACAGAATTCACTCTCACAATCAGC |
| | | | AGCCTGCAGCCTGAAGATTTTGCAACTTATTACTGTCA |
| | | | ACAGCTTACTAGTTACCCATTCACTTTCGGCCCTGGGA |
| | | | CCAAAGTGGATATCAAA |
| 191 | PPB-1042 | VL domain | DIQLTQSPSFLSASVGDRVTITCWASQGISSRLAWYQQ |
| | | amino acid | KPGKAPKLLIYAASTLQSGVPSRFSGSGSGTEFTLTIS |
| | | sequence | SLQPEDFATYYCQQLTSYPFTFGPGTKVDIK |
| 37 | PPB-1042 | LCDR1 | QGISSR |
| 28 | PPB-1042 | LCDR2 | AAS |
| 192 | PPB-1042 | LCDR3 | QQLTSYPFT |
| 193 | PPB-1043 | VH domain | CAGATCACCTTGAAGGAGTCTGGTCCTACGCTGGTGAA |
| | | nucleic | ACCCACACAGACCCTCACGCTGACCTGCACCTTCTCTG |
| | | acid | GGTTCTCACTCAGCACTAATGGAGTGGGTGTGGGCTGG |
| | | sequence | ATCCGTCAGCCCCCAGGAAAGGCCCTGGAGTGGCTTGC |
| | | | ACTCATTTATTGGGATGATGATAAGCGCTACAGCCCAT |
| | | | CTCTGAAGAGCAGGCTCACCATCACCAAGGACACCTCC |
| | | | AAAAACCAGGTGGTCCTTACAATGACCAACATGGACCC |
| | | | TGTGGACACAGCCACATATTACTGTGCACACCTTTACT |
| | | | ATGTTATTAGTGGTTATTACTTTGACTACTGGGGCCAG |
| | | | GGAACCCTGGTCACCGTCTCCTCA |
| 194 | PPB-1043 | VH domain | QITLKESGPTLVKPTQTLTLTCTFSGFSLSTNGVGVGW |
| | | amino acid | IRQPPGKALEWLALIYWDDDKRYSPSLKSRLTITKDTS |
| | | sequence | KNQVVLTMTNMDPVDTATYYCAHLYYVISGYYFDYWGQ |
| | | | GTLVTVSS |
| 195 | PPB-1043 | HCDR1 | GFSLSTNGVG |
| 33 | PPB-1043 | HCDR2 | IYWDDDK |
| 196 | PPB-1043 | HCDR3 | AHLYYVISGYYFDY |
| 197 | PPB-1043 | VL domain | GACATCCAGATGACCCAGTCTCCATCCTCACTGTCTGC |
| | | nucleic | ATCTGTAGGAGACAGAGTCACCATCACTTGTCGGGCGA |
| | | acid | GTCAGGGTATCAGCAGCAGGGTAGCCTGGTATCAGGAG |
| | | sequence | AAACCAGAGAAAGCCCCTAAGTCCCTGATCTATGTTGC |
| | | | ATCCAGATTGCAAAGAGGGGTCCCATCAAGGTTCAGCG |
| | | | GCAGTGGATCTGGGACAGATTTCACTCTCACCATCAGC |
| | | | AGCCTGCAGCCTGAAGATTTTGCAACTTATTACTGCCA |
| | | | ACAGTATAATAGTTACCCATTCACTTTCGGCCCTGGGA |
| | | | CCAAAGTAGATATCAAA |
| 198 | PPB-1043 | VL domain | DIQMTQSPSSLSASVGDRVTITCRASQGISSRVAWYQE |
| | | amino acid | KPEKAPKSLIYVASRLQRGVPSRFSGSGSGTDFTLTIS |
| | | sequence | SLQPEDFATYYCQQYNSYPFTFGPGTKVDIK |
| 37 | PPB-1043 | LCDR1 | QGISSR |
| 9 | PPB-1043 | LCDR2 | VAS |
| 199 | PPB-1043 | LCDR3 | QQYNSYPFT |

TABLE 2
Constant region sequences:

| | | | | |
|---|---|---|---|---|
| 417 | Human IgG1 | IGHG1*01 | Human Heavy | gcctccaccaagggcccatcggtcttcc |
| | constant | | Chain Constant | ccctggcaccctcctccaagagcacctc |
| | region | | Region | tgggggcacagcggccctgggctgcctg |
| | | | (IGHG1*01) | gtcaaggactacttccccgaaccggtga |
| | | | Nucleotide | cggtgtcgtggaactcaggcgccctgac |
| | | | Sequence | cagcggcgtgcacaccttcccggctgtc |
| | | | | ctacagtcctcaggactctactccctca |
| | | | | gcagcgtggtgaccgtgccctccagcag |
| | | | | cttgggcacccagacctacatctgcaac |
| | | | | gtgaatcacaagcccagcaacaccaagg |
| | | | | tggacaagaaagttgagcccaaatcttg |
| | | | | tgacaaaactcacacatgcccaccgtgc |
| | | | | ccagcacctgaactcctggggggaccgt |
| | | | | cagtcttcctcttccccccaaaacccaa |
| | | | | ggacaccctcatgatctcccggacccct |
| | | | | gaggtcacatgcgtggtggtggacgtga |
| | | | | gccacgaagaccctgaggtcaagttcaa |
| | | | | ctggtacgtggacggcgtggaggtgcat |
| | | | | aatgccaagacaaagccgcgggaggagc |
| | | | | agtacaacagcacgtaccgggtggtcag |
| | | | | cgtcctcaccgtcctgcaccaggactgg |
| | | | | ctgaatggcaaggagtacaagtgcaagg |
| | | | | tctccaacaaagccctcccagcccccat |
| | | | | cgagaaaaccatctccaaagccaaaggg |
| | | | | cagccccgagaaccacaggtgtacaccc |
| | | | | tgcccccatcccgggatgagctgaccaa |
| | | | | gaaccaggtcagcctgacctgcctggtc |
| | | | | aaaggcttctatcccagcgacatcgccg |
| | | | | tggagtgggagagcaatgggcagccgga |
| | | | | gaacaactacaagaccacgcctcccgtg |
| | | | | ctggactccgacggctccttcttcctct |
| | | | | acagcaagctcaccgtggacaagagcag |
| | | | | gtggcagcaggggaacgtcttctcatgc |
| | | | | tccgtgatgcatgaggctctgcacaacc |
| | | | | actacacgcagaagagcctctccctgtc |
| | | | | tccgggtaaa |
| 418 | | | Human Heavy | ASTKGPSVFPLAPSSKSTSGGTAALGCL |
| | | | Chain Constant | VKDYFPEPVTVSWNSGALTSGVHTFPAV |
| | | | Region | PAPELLGGPSVFLFPPKPKDTLMISRTP |
| | | | (IGHG1*01) | LQSSGLYSLSSVVTVPSSSLGTQTYICN |
| | | | Protein Sequence | VNHKPSNTKVDKKVEPKSCDKTHTCPPC |
| | | | (P01857) | EVTCVVVDVSHEDPEVKFNWYVDGVEVH |
| | | | | NAKTKPREEQYNSTYRVVSVLTVLHQDW |
| | | | | LNGKEYKCKVSNKALPAPIEKTISKAKG |
| | | | | QPREPQVYTLPPSRDELTKNQVSLTCLV |
| | | | | KGFYPSDIAVEWESNGQPENNYKTTPPV |
| | | | | LDSDGSFFLYSKLTVDKSRWQQGNVFSC |
| | | | | SVMHEALHNHYTQKSLSLSPGK |
| 419 | Human IgG1 | IGHG1*02 or | Human Heavy | gcctccaccaagggcccatcggtcttcc |
| | constant | IGHG1*05 | Chain Constant | ccctggcaccctcctccaagagcacctc |
| | region | | Region (IGHG1*02 | tgggggcacagcggccctgggctgcctg |
| | | | or IGHG1*05) | gtcaaggactacttccccgaaccggtga |
| | | | Nucleotide | cggtgtcgtggaactcaggcgccctgac |
| | | | Sequence | cagcggcgtgcacaccttcccggctgtc |
| | | | | ctacagtcctcaggactctactccctca |
| | | | | gcagcgtggtgaccgtgccctccagcag |
| | | | | cttgggcacccagacctacatctgcaac |
| | | | | gtgaatcacaagcccagcaacaccaagg |
| | | | | tggacaagaaagttgagcccaaatcttg |
| | | | | tgacaaaactcacacatgcccaccgtgc |
| | | | | ccagcacctgaactcctggggggaccgt |
| | | | | cagtcttcctcttccccccaaaacccaa |
| | | | | ggacaccctcatgatctcccggacccct |
| | | | | gaggtcacatgcgtggtggtggacgtga |
| | | | | gccacgaagaccctgaggtcaagttcaa |
| | | | | ctggtacgtggacggcgtggaggtgcat |
| | | | | aatgccaagacaaagccgcgggaggagc |
| | | | | agtacaacagcacgtaccgtgtggtcag |
| | | | | cgtcctcaccgtcctgcaccaggactgg |
| | | | | ctgaatggcaaggagtacaagtgcaagg |
| | | | | tctccaacaaagccctcccagcccccat |
| | | | | cgagaaaaccatctccaaagccaaaggg |
| | | | | cagccccgagaaccacaggtgtacaccc |
| | | | | tgcccccatcccgggatgagctgaccaa |
| | | | | gaaccaggtcagcctgacctgcctggtc |
| | | | | aaaggcttctatcccagcgacatcgccg |
| | | | | tggagtgggagagcaatgggcagccgga |
| | | | | gaacaactacaagaccacgcctcccgtg |
| | | | | ctggactccgacggctccttcttcctct |
| | | | | acagcaagctcaccgtggacaagagcag |
| | | | | gtggcagcaggggaacgtcttctcatgc |
| | | | | tccgtgatgcatgaggctctgcacaacc |
| | | | | actacacgcagaagagcctctccctgtc |
| | | | | tccgggtaaa |
| 420 | | | Human Heavy | A S T K G P S V F P L A P S |
| | | | Chain Constant | S K S T S G G T A A L G C L |
| | | | Region | V K D Y F P E P V T V S W N |
| | | | (IGHG1*02) | S G A L T S G V H T F P A V |
| | | | Protein Sequence | L Q S S G L Y S L S S V V T |
| | | | | V P S S S L G T Q T Y I C N |
| | | | | V N H K P S N T K V D K K V |
| | | | | E P K S C D K T H T C P P C |
| | | | | P A P E L L G G P S V F L F |
| | | | | P P K P K D T L M I S R T P |
| | | | | E V T C V V V D V S H E D P |
| | | | | E V K F N W Y V D G V E V H |
| | | | | N A K T K P R E E Q Y N S T |
| | | | | Y R V V S V L T V L H Q D W |
| | | | | L N G K E Y K C K V S N K A |
| | | | | L P A P I E K T I S K A K G |
| | | | | Q P R E P Q V Y T L P P S R |
| | | | | D E L T K N Q V S L T C L V |
| | | | | K G F Y P S D I A V E W E S |
| | | | | N G Q P E N N Y K T T P P V |
| | | | | L D S D G S F F L Y S K L T |
| | | | | V D K S R W Q Q G N V F S C |
| | | | | S V M H E A L H N H Y T Q K |
| | | | | S L S L S P G K |
| 421 | Human IgG1 | IGHG1*03 | Human Heavy | gcctccaccaagggcccatcggtcttcc |
| | constant | | Chain Constant | ccctggcaccctcctccaagagcacctc |
| | region | | Region | tgggggcacagcggccctgggctgcctg |
| | | | (IGHG1*03) | gtcaaggactacttccccgaaccggtga |
| | | | Nucleotide | cggtgtcgtggaactcaggcgccctgac |
| | | | Sequence | cagcggcgtgcacaccttcccggctgtc |
| | | | (Y14737) | ctacagtcctcaggactctactccctca |
| | | | | gcagcgtggtgaccgtgccctccagcag |
| | | | | cttgggcacccagacctacatctgcaac |
| | | | | gtgaatcacaagcccagcaacaccaagg |
| | | | | tggacaagagagttgagcccaaatcttg |
| | | | | tgacaaaactcacacatgcccaccgtgc |
| | | | | ccagcacctgaactcctggggggaccgt |
| | | | | cagtcttcctcttccccccaaaacccaa |
| | | | | ggacaccctcatgatctcccggacccct |
| | | | | gaggtcacatgcgtggtggtggacgtga |
| | | | | gccacgaagaccctgaggtcaagttcaa |
| | | | | ctggtacgtggacggcgtggaggtgcat |
| | | | | aatgccaagacaaagccgcgggaggagc |
| | | | | agtacaacagcacgtaccgtgtggtcag |
| | | | | cgtcctcaccgtcctgcaccaggactgg |
| | | | | ctgaatggcaaggagtacaagtgcaagg |
| | | | | tctccaacaaagccctcccagcccccat |
| | | | | cgagaaaaccatctccaaagccaaaggg |
| | | | | cagccccgagaaccacaggtgtacaccc |
| | | | | tgcccccatcccgggaggagatgaccaa |
| | | | | gaaccaggtcagcctgacctgcctggtc |
| | | | | aaaggcttctatcccagcgacatcgccg |
| | | | | tggagtgggagagcaatgggcagccgga |
| | | | | gaacaactacaagaccacgcctcccgtg |
| | | | | ctggactccgacggctccttcttcctct |
| | | | | atagcaagctcaccgtggacaagagcag |
| | | | | gtggcagcaggggaacgtcttctcatgc |
| | | | | tccgtgatgcatgaggctctgcacaacc |
| | | | | actacacgcagaagagcctctccctgtc |
| | | | | cccgggtaaa |
| 422 | | | Human Heavy | A S T K G P S V F P L A P S |
| | | | Chain Constant | S K S T S G G T A A L G C L |
| | | | Region | V K D Y F P E P V T V S W N |
| | | | (IGHG1*03) | S G A L T S G V H T F P A V |
| | | | Protein Sequence | L Q S S G L Y S L S S V V T |
| | | | | V P S S S L G T Q T Y I C N |
| | | | | V N H K P S N T K V D K R V |
| | | | | E P K S C D K T H T C P P C |
| | | | | P A P E L L G G P S V F L F |
| | | | | P P K P K D T L M I S R T P |
| | | | | E V T C V V V D V S H E D P |
| | | | | E V K F N W Y V D G V E V H |
| | | | | N A K T K P R E E Q Y N S T |
| | | | | Y R V V S V L T V L H Q D W |
| | | | | L N G K E Y K C K V S N K A |
| | | | | L P A P I E K T I S K A K G |
| | | | | Q P R E P Q V Y T L P P S R |
| | | | | E E M T K N Q V S L T C L V |
| | | | | K G F Y P S D I A V E W E S |
| | | | | N G Q P E N N Y K T T P P V |
| | | | | L D S D G S F F L Y S K L T |
| | | | | V D K S R W Q Q G N V F S C |
| | | | | S V M H E A L H N H Y T Q K |
| | | | | S L S L S P G K |
| 423 | Human IgG1 | IGHG1*04 | Human Heavy | gcctccaccaagggcccatcggtcttcc |
| | constant | | Chain Constant | ccctggcaccctcctccaagagcacctc |
| | region | | Region | tgggggcacagcggccctgggctgcctg |
| | | | (IGHG1*04) | gtcaaggactacttccccgaaccggtga |
| | | | Nucleotide | cggtgtcgtggaactcaggcgccctgac |
| | | | Sequence | cagcggcgtgcacaccttcccggctgtc |
| | | | | ctacagtcctcaggactctactccctca |
| | | | | gcagcgtggtgaccgtgccctccagcag |
| | | | | cttgggcacccagacctacatctgcaac |
| | | | | gtgaatcacaagcccagcaacaccaagg |
| | | | | tggacaagaaagttgagcccaaatcttg |
| | | | | tgacaaaactcacacatgcccaccgtgc |
| | | | | ccagcacctgaactcctggggggaccgt |
| | | | | cagtcttcctcttccccccaaaacccaa |
| | | | | ggacaccctcatgatctcccggacccct |
| | | | | gaggtcacatgcgtggtggtggacgtga |
| | | | | gccacgaagaccctgaggtcaagttcaa |
| | | | | ctggtacgtggacggcgtggaggtgcat |
| | | | | aatgccaagacaaagccgcgggaggagc |
| | | | | agtacaacagcacgtaccgtgtggtcag |
| | | | | cgtcctcaccgtcctgcaccaggactgg |
| | | | | ctgaatggcaaggagtacaagtgcaagg |
| | | | | tctccaacaaagccctcccagcccccat |
| | | | | cgagaaaaccatctccaaagccaaaggg |
| | | | | cagccccgagaaccacaggtgtacaccc |
| | | | | tgcccccatcccgggatgagctgaccaa |
| | | | | gaaccaggtcagcctgacctgcctggtc |
| | | | | aaaggcttctatcccagcgacatcgccg |
| | | | | tggagtgggagagcaatgggcagccgga |
| | | | | gaacaactacaagaccacgcctcccgtg |
| | | | | ctggactccgacggctccttcttcctct |
| | | | | acagcaagctcaccgtggacaagagcag |
| | | | | gtggcagcaggggaacatcttctcatgc |
| | | | | tccgtgatgcatgaggctctgcacaacc |
| | | | | actacacgcagaagagcctctccctgtc |
| | | | | tccgggtaaa |
| 424 | | | Human Heavy | ASTKGPSVFPLAPSSKSTSGGTAALGCL |
| | | | Chain Constant | VKDYFPEPVTVSWNSGALTSGVHTFPAV |
| | | | Region | LQSSGLYSLSSVVTVPSSSLGTQTYICN |
| | | | (IGHG1*04) | VNHKPSNTKVDKKVEPKSCDKTHTCPPC |
| | | | Protein Sequence | PAPELLGGPSVFLFPPKPKDTLMISRTP |
| | | | | EVTCVVVDVSHEDPEVKFNWYVDGVEVH |
| | | | | NAKTKPREEQYNSTYRVVSVLTVLHQDW |
| | | | | LNGKEYKCKVSNKALPAPIEKTISKAKG |
| | | | | QPREPQVYTLPPSRDELTKNQVSLTCLV |
| | | | | KGFYPSDIAVEWESNGQPENNYKTTPPV |
| | | | | LDSDGSFFLYSKLTVDKSRWQQGNIFSC |
| | | | | SVMHEALHNHYTQKSLSLSPGK |
| 425 | Disabled | Disabled | Disabled Human | gcctccaccaagggcccatcggtcttcc |
| | Human IgG1 | human | IGHG1*01 Heavy | ccctggcaccctcctccaagagcacctc |
| | heavy chain | IGHG1*01 | Chain Constant | tgggggcacagcggccctgggctgcctg |
| | constant | | Region | gtcaaggactacttccccgaaccggtga |
| | region | | Nucleotide | cggtgtcgtggaactcaggcgccctgac |
| | | | Sequence. | cagcggcgtgcacaccttcccggctgtc |
| | | | | ctacagtcctcaggactctactccctca |
| | | | | gcagcgtggtgaccgtgccctccagcag |
| | | | | cttgggcacccagacctacatctgcaac |
| | | | | gtgaatcacaagcccagcaacaccaagg |
| | | | | tggacaagaaagtggagcccaaatcttg |
| | | | | tgacaaaactcacacatgcccaccgtgc |
| | | | | ccagcacctgaactcgcgggggcaccgt |
| | | | | cagtcttcctcttccccccaaaacccaa |
| | | | | ggacaccctcatgatctcccggacccct |
| | | | | gaggtcacatgcgtggtggtggacgtga |
| | | | | gccacgaagaccctgaggtcaagttcaa |
| | | | | ctggtacgtggacggcgtggaggtgcat |
| | | | | aatgccaagacaaagccgcgggaggagc |
| | | | | agtacaacagcacgtaccgtgtggtcag |
| | | | | cgtcctcaccgtcctgcaccaggactgg |
| | | | | ctgaatggcaaggagtacaagtgcaagg |
| | | | | tctccaacaaagccctcccagcccccat |
| | | | | cgagaaaaccatctccaaagccaaaggg |
| | | | | cagccccgagaaccacaggtgtacaccc |
| | | | | tgcccccatcccgggatgagctgaccaa |
| | | | | gaaccaggtcagcctgacctgcctggtc |
| | | | | aaaggcttctatcccagcgacatcgccg |
| | | | | tggagtgggagagcaatgggcagccgga |
| | | | | gaacaactacaagaccacgcctcccgtg |
| | | | | ctggactccgacggctccttcttcctct |
| | | | | acagcaagctcaccgtggacaagagcag |
| | | | | gtggcagcaggggaacgtcttctcatgc |
| | | | | tccgtgatgcatgaggctctgcacaacc |
| | | | | actacacgcagaagagcctctccctgtc |
| | | | | tccgggtaaa |
| 426 | | | Disabled Human | ASTKGPSVFPLAPSSKSTSGGTAALGCL |
| | | | IGHG1*01 Heavy | VKDYFPEPVTVSWNSGALTSGVHTFPAV |
| | | | Chain Constant | LQSSGLYSLSSVVTVPSSSLGTQTYICN |
| | | | Region Amino | VNHKPSNTKVDKKVEPKSCDKTHTCPPC |
| | | | Acid Sequence. | PAPELAGAPSVFLFPPKPKDTLMISRTP |
| | | | Two residues | EVTCVVVDVSHEDPEVKFNWYVDGVEVH |
| | | | that differ from | NAKTKPREEQYNSTYRVVSVLTVLHQDW |
| | | | the wild-type | LNGKEYKCKVSNKALPAPIEKTISKAKG |
| | | | sequence are | QPREPQVYTLPPSRDELTKNQVSLTCLV |
| | | | identified in | KGFYPSDIAVEWESNGQPENNYKTTPPV |
| | | | bold. | LDSDGSFFLYSKLTVDKSRWQQGNVFSC |
| | | | | SVMHEALHNHYTQKSLSLSPGK |
| 427 | Human IgG2 | IGHG2*01 or | Human Heavy | gcctccaccaagggcccatcggtcttcc |
| | constant | IGHG2*04 or | Chain Constant | ccctggcgccctgctccaggagcacctc |
| | region | IGHG2*05 | Region (IGHG2*01 | cgagagcacagccgccctgggctgcctg |
| | | | or IGHG2*03 or | gtcaaggactacttccccgaaccggtga |
| | | | IGHG2*05) | cggtgtcgtggaactcaggcgctctgac |
| | | | Nucleotide | cagcggcgtgcacaccttcccagctgtc |
| | | | Sequence | ctacagtcctcaggactctactccctca |
| | | | | gcagcgtggtgaccgtgccctccagcaa |
| | | | | cttcggcacccagacctacacctgcaac |
| | | | | gtagatcacaagcccagcaacaccaagg |
| | | | | tggacaagacagttgagcgcaaatgttg |
| | | | | tgtcgagtgcccaccgtgcccagcacca |
| | | | | cctgtggcaggaccgtcagtcttcctct |
| | | | | tccccccaaaacccaaggacaccctcat |
| | | | | gatctcccggacccctgaggtcacgtgc |
| | | | | gtggtggtggacgtgagccacgaagacc |
| | | | | ccgaggtccagttcaactggtacgtgga |
| | | | | cggcgtggaggtgcataatgccaagaca |
| | | | | aagccacgggaggagcagttcaacagca |
| | | | | cgttccgtgtggtcagcgtcctcaccgt |
| | | | | tgtgcaccaggactggctgaacggcaag |
| | | | | gagtacaagtgcaaggtctccaacaaag |
| | | | | gcctcccagcccccatcgagaaaaccat |
| | | | | ctccaaaaccaaagggcagccccgagaa |
| | | | | ccacaggtgtacaccctgcccccatccc |
| | | | | gggaggagatgaccaagaaccaggtcag |
| | | | | cctgacctgcctggtcaaaggcttctac |
| | | | | cccagcgacatcgccgtggagtgggaga |
| | | | | gcaatgggcagccggagaacaactacaa |
| | | | | gaccacacctcccatgctggactccgac |
| | | | | ggctccttcttcctctacagcaagctca |
| | | | | ccgtggacaagagcaggtggcagcaggg |
| | | | | gaacgtcttctcatgctccgtgatgcat |
| | | | | gaggctctgcacaaccactacacgcaga |
| | | | | agagcctctccctgtctccgggtaaa |
| 428 | | | Human Heavy | ASTKGPSVFPLAPCSRSTSESTAALGCL |
| | | | Chain Constant | VKDYFPEPVTVSWNSGALTSGVHTFPAV |
| | | | Region | LQSSGLYSLSSVVTVPSSNFGTQTYTCN |
| | | | (IGHG2*01) | VDHKPSNTKVDKTVERKCCVECPPCPAP |
| | | | Protein Sequence | PVAGPSVFLFPPKPKDTLMISRTPEVTC |
| | | | | VVVDVSHEDPEVQFNWYVDGVEVHNAKT |
| | | | | KPREEQFNSTFRVVSVLTVVHQDWLNGK |
| | | | | EYKCKVSNKGLPAPIEKTISKTKGQPRE |
| | | | | PQVYTLPPSREEMTKNQVSLTCLVKGFY |
| | | | | PSDIAVEWESNGQPENNYKTTPPMLDSD |
| | | | | GSFFLYSKLTVDKSRWQQGNVFSCSVMH |
| | | | | EALHNHYTQKSLSLSPGK |
| 429 | Human IgG2 | IGHG2*02 | Human Heavy | GCCTCCACCAAGGGCCCATCGGTCTTCC |
| | constant | | Chain Constant | CCCTGGCGCCCTGCTCCAGGAGCACCTC |
| | region | | Region | CGAGAGCACAGCGGCCCTGGGCTGCCTG |
| | | | (IGHG2*02) | GTCAAGGACTACTTCCCCGAACCGGTGA |
| | | | Nucleotide | CGGTGTCGTGGAACTCAGGCGCTCTGAC |
| | | | Sequence | CAGCGGCGTGCACACCTTCCCGGCTGTC |
| | | | | CTACAGTCCTCAGGACTCTACTCCCTCA |
| | | | | GCAGCGTGGTGACCGTGACCTCCAGCAA |
| | | | | CTTCGGCACCCAGACCTACACCTGCAAC |
| | | | | GTAGATCACAAGCCCAGCAACACCAAGG |
| | | | | TGGACAAGACAGTTGAGCGCAAATGTTG |
| | | | | TGTCGAGTGCCCACCGTGCCCAGCACCA |
| | | | | CCTGTGGCAGGACCGTCAGTCTTCCTCT |
| | | | | TCCCCCCAAAACCCAAGGACACCCTCAT |
| | | | | GATCTCCCGGACCCCTGAGGTCACGTGC |
| | | | | GTGGTGGTGGACGTGAGCCACGAAGACC |
| | | | | CCGAGGTCCAGTTCAACTGGTACGTGGA |
| | | | | CGGCATGGAGGTGCATAATGCCAAGACA |
| | | | | AAGCCACGGGAGGAGCAGTTCAACAGCA |
| | | | | CGTTCCGTGTGGTCAGCGTCCTCACCGT |
| | | | | CGTGCACCAGGACTGGCTGAACGGCAAG |
| | | | | GAGTACAAGTGCAAGGTCTCCAACAAAG |
| | | | | GCCTCCCAGCCCCCATCGAGAAAACCAT |
| | | | | CTCCAAAACCAAAGGGCAGCCCCGAGAA |
| | | | | CCACAGGTGTACACCCTGCCCCCATCCC |
| | | | | GGGAGGAGATGACCAAGAACCAGGTCAG |
| | | | | CCTGACCTGCCTGGTCAAAGGCTTCTAC |
| | | | | CCCAGCGACATCGCCGTGGAGTGGGAGA |
| | | | | GCAATGGGCAGCCGGAGAACAACTACAA |
| | | | | GACCACACCTCCCATGCTGGACTCCGAC |
| | | | | GGCTCCTTCTTCCTCTACAGCAAGCTCA |
| | | | | CCGTGGACAAGAGCAGGTGGCAGCAGGG |
| | | | | GAACGTCTTCTCATGCTCCGTGATGCAT |
| | | | | GAGGCTCTGCACAACCACTACACACAGA |
| | | | | AGAGCCTCTCCCTGTCTCCGGGTAAA |
| 430 | | | Human Heavy | ASTKGPSVFPLAPCSRSTSESTAALGCL |
| | | | Chain Constant | VKDYFPEPVTVSWNSGALTSGVHTFPAV |
| | | | Region | LQSSGLYSLSSVVTVTSSNFGTQTYTCN |
| | | | (IGHG2*02) | VDHKPSNTKVDKTVERKCCVECPPCPAP |
| | | | Protein Sequence | PVAGPSVFLFPPKPKDTLMISRTPEVTC |
| | | | | VVVDVSHEDPEVQFNWYVDGMEVHNAKT |
| | | | | KPREEQFNSTFRVVSVLTVVHQDWLNGK |
| | | | | EYKCKVSNKGLPAPIEKTISKTKGQPRE |
| | | | | PQVYTLPPSREEMTKNQVSLTCLVKGFY |
| | | | | PSDIAVEWESNGQPENNYKTTPPMLDSD |
| | | | | GSFFLYSKLTVDKSRWQQGNVFSCSVMH |
| | | | | EALHNHYTQKSLSLSPGK |
| 431 | Human IgG2 | IGHG2*04 | Human Heavy | gcctccaccaagggcccatcggtcttcc |
| | constant | | Chain Constant | ccctggcgccctgctccaggagcacctc |
| | region | | Region | cgagagcacagcggccctgggctgcctg |
| | | | (IGHG2*04) | gtcaaggactacttccccgaaccggtga |
| | | | Nucleotide | cggtgtcgtggaactcaggcgctctgac |
| | | | Sequence | cagcggcgtgcacaccttcccagctgtc |
| | | | | ctacagtcctcaggactctactccctca |
| | | | | gcagcgtggtgaccgtgccctccagcag |
| | | | | cttgggcacccagacctacacctgcaac |
| | | | | gtagatcacaagcccagcaacaccaagg |
| | | | | tggacaagacagttgagcgcaaatgttg |
| | | | | tgtcgagtgcccaccgtgcccagcacca |
| | | | | cctgtggcaggaccgtcagtcttcctct |
| | | | | tccccccaaaacccaaggacaccctcat |
| | | | | gatctcccggacccctgaggtcacgtgc |
| | | | | gtggtggtggacgtgagccacgaagacc |
| | | | | ccgaggtccagttcaactggtacgtgga |
| | | | | cggcgtggaggtgcataatgccaagaca |
| | | | | aagccacgggaggagcagttcaacagca |
| | | | | cgttccgtgtggtcagcgtcctcaccgt |
| | | | | tgtgcaccaggactggctgaacggcaag |
| | | | | gagtacaagtgcaaggtctccaacaaag |
| | | | | gcctcccagcccccatcgagaaaaccat |
| | | | | ctccaaaaccaaagggcagccccgagaa |
| | | | | ccacaggtgtacaccctgcccccatccc |
| | | | | gggaggagatgaccaagaaccaggtcag |
| | | | | cctgacctgcctggtcaaaggcttctac |
| | | | | cccagcgacatcgccgtggagtgggaga |
| | | | | gcaatgggcagccggagaacaactacaa |
| | | | | gaccacacctcccatgctggactccgac |
| | | | | ggctccttcttcctctacagcaagctca |
| | | | | ccgtggacaagagcaggtggcagcaggg |
| | | | | gaacgtcttctcatgctccgtgatgcat |
| | | | | gaggctctgcacaaccactacacgcaga |
| | | | | agagcctctccctgtctccgggtaaa |
| 432 | | | Human Heavy | ASTKGPSVFPLAPCSRSTSESTAALGCL |
| | | | Chain Constant | VKDYFPEPVTVSWNSGALTSGVHTFPAV |
| | | | Region | LQSSGLYSLSSVVTVPSSSLGTQTYTCN |
| | | | (IGHG2*04) | VDHKPSNTKVDKTVERKCCVECPPCPAP |
| | | | Protein Sequence | PVAGPSVFLFPPKPKDTLMISRTPEVTC |
| | | | | VVVDVSHEDPEVQFNWYVDGVEVHNAKT |
| | | | | KPREEQFNSTFRVVSVLTVVHQDWLNGK |
| | | | | EYKCKVSNKGLPAPIEKTISKTKGQPRE |
| | | | | PQVYTLPPSREEMTKNQVSLTCLVKGFY |
| | | | | PSDIAVEWESNGQPENNYKTTPPMLDSD |
| | | | | GSFFLYSKLTVDKSRWQQGNVFSCSVMH |
| | | | | EALHNHYTQKSLSLSPGK |
| 433 | Human IgG2 | IGHG2*06 | Human Heavy | GCCTCCACCAAGGGCCCATCGGTCTTCC |
| | constant | | Chain Constant | CCCTGGCGCCCTGCTCCAGGAGCACCTC |
| | region | | Region | CGAGAGCACAGCGGCCCTGGGCTGCCTG |
| | | | (IGHG2*06) | GTCAAGGACTACTTCCCCGAACCGGTGA |
| | | | Nucleotide | CGGTGTCGTGGAACTCAGGCGCTCTGAC |
| | | | Sequence | CAGCGGCGTGCACACCTTCCCGGCTGTC |
| | | | | CTACAGTCCTCAGGACTCTACTCCCTCA |
| | | | | GCAGCGTGGTGACCGTGCCCTCCAGCAA |
| | | | | CTTCGGCACCCAGACCTACACCTGCAAC |
| | | | | GTAGATCACAAGCCCAGCAACACCAAGG |
| | | | | TGGACAAGACAGTTGAGCGCAAATGTTG |
| | | | | TGTCGAGTGCCCACCGTGCCCAGCACCA |
| | | | | CCTGTGGCAGGACCGTCAGTCTTCCTCT |
| | | | | TCCCCCCAAAACCCAAGGACACCCTCAT |
| | | | | GATCTCCCGGACCCCTGAGGTCACGTGC |
| | | | | GTGGTGGTGGACGTGAGCCACGAAGACC |
| | | | | CCGAGGTCCAGTTCAACTGGTACGTGGA |
| | | | | CGGCGTGGAGGTGCATAATGCCAAGACA |
| | | | | AAGCCACGGGAGGAGCAGTTCAACAGCA |
| | | | | CGTTCCGTGTGGTCAGCGTCCTCACCGT |
| | | | | CGTGCACCAGGACTGGCTGAACGGCAAG |
| | | | | GAGTACAAGTGCAAGGTCTCCAACAAAG |
| | | | | GCCTCCCAGCCCCCATCGAGAAAACCAT |
| | | | | CTCCAAAACCAAAGGGCAGCCCCGAGAA |
| | | | | CCACAGGTGTACACCCTGCCCCCATCCC |
| | | | | GGGAGGAGATGACCAAGAACCAGGTCAG |
| | | | | CCTGACCTGCCTGGTCAAAGGCTTCTAC |
| | | | | CCCAGCGACATCTCCGTGGAGTGGGAGA |
| | | | | GCAATGGGCAGCCGGAGAACAACTACAA |
| | | | | GACCACACCTCCCATGCTGGACTCCGAC |
| | | | | GGCTCCTTCTTCCTCTACAGCAAGCTCA |
| | | | | CCGTGGACAAGAGCAGGTGGCAGCAGGG |
| | | | | GAACGTCTTCTCATGCTCCGTGATGCAT |
| | | | | GAGGCTCTGCACAACCACTACACACAGA |
| | | | | AGAGCCTCTCCCTGTCTCCGGGTAAA |
| 434 | | | Human Heavy | ASTKGPSVFPLAPCSRSTSESTAALGCL |
| | | | Chain Constant | VKDYFPEPVTVSWNSGALTSGVHTFPAV |
| | | | Region | LQSSGLYSLSSVVTVPSSNFGTQTYTCN |
| | | | (IGHG2*06) | VDHKPSNTKVDKTVERKCCVECPPCPAP |
| | | | Protein Sequence | PVAGPSVFLFPPKPKDTLMISRTPEVTC |
| | | | | VVVDVSHEDPEVQFNWYVDGVEVHNAKT |
| | | | | KPREEQFNSTFRVVSVLTVVHQDWLNGK |
| | | | | EYKCKVSNKGLPAPIEKTISKTKGQPRE |
| | | | | PQVYTLPPSREEMTKNQVSLTCLVKGFY |
| | | | | PSDISVEWESNGQPENNYKTTPPMLDSD |
| | | | | GSFFLYSKLTVDKSRWQQGNVFSCSVMH |
| | | | | EALHNHYTQKSLSLSPGK |
| 435 | Human IgG4 | IGHG4*01 or | Human Heavy | gcttccaccaagggcccatccgtcttcc |
| | constant | IGHG4*04 | Chain Constant | ccctggcgccctgctccaggagcacctc |
| | region | | Region (IGHG4*01 | cgagagcacagccgccctgggctgcctg |
| | | | or IGHG4*04) | gtcaaggactacttccccgaaccggtga |
| | | | Nucleotide | cggtgtcgtggaactcaggcgccctgac |
| | | | Sequence | cagcggcgtgcacaccttcccggctgtc |
| | | | | ctacagtcctcaggactctactccctca |
| | | | | gcagcgtggtgaccgtgccctccagcag |
| | | | | cttgggcacgaagacctacacctgcaac |
| | | | | gtagatcacaagcccagcaacaccaagg |
| | | | | tggacaagagagttgagtccaaatatgg |
| | | | | tcccccatgcccatcatgcccagcacct |
| | | | | gagttcctggggggaccatcagtcttcc |
| | | | | tgttccccccaaaacccaaggacactct |
| | | | | catgatctcccggacccctgaggtcacg |
| | | | | tgcgtggtggtggacgtgagccaggaag |
| | | | | accccgaggtccagttcaactggtacgt |
| | | | | ggatggcgtggaggtgcataatgccaag |
| | | | | acaaagccgcgggaggagcagttcaaca |
| | | | | gcacgtaccgtgtggtcagcgtcctcac |
| | | | | cgtcctgcaccaggactggctgaacggc |
| | | | | aaggagtacaagtgcaaggtctccaaca |
| | | | | aaggcctcccgtcctccatcgagaaaac |
| | | | | catctccaaagccaaagggcagccccga |
| | | | | gagccacaggtgtacaccctgcccccat |
| | | | | cccaggaggagatgaccaagaaccaggt |
| | | | | cagcctgacctgcctggtcaaaggcttc |
| | | | | taccccagcgacatcgccgtggagtggg |
| | | | | agagcaatgggcagccggagaacaacta |
| | | | | caagaccacgcctcccgtgctggactcc |
| | | | | gacggctccttcttcctctacagcaggc |
| | | | | taaccgtggacaagagcaggtggcagga |
| | | | | ggggaatgtcttctcatgctccgtgatg |
| | | | | catgaggctctgcacaaccactacacac |
| | | | | agaagagcctctccctgtctctgggtaa |
| | | | | a |
| 436 | | | Human Heavy | ASTKGPSVFPLAPCSRSTSESTAALGCL |
| | | | Chain Constant | VKDYFPEPVTVSWNSGALTSGVHTFPAV |
| | | | Region | LQSSGLYSLSSVVTVPSSSLGTKTYTCN |
| | | | (IGHG4*01) | VDHKPSNTKVDKRVESKYGPPCPSCPAP |
| | | | Protein Sequence | EFLGGPSVFLFPPKPKDTLMISRTPEVT |
| | | | (P01861) | CVVVDVSQEDPEVQFNWYVDGVEVHNAK |
| | | | | TKPREEQFNSTYRVVSVLTVLHQDWLNG |
| | | | | KEYKCKVSNKGLPSSIEKTISKAKGQPR |
| | | | | EPQVYTLPPSQEEMTKNQVSLTCLVKGF |
| | | | | YPSDIAVEWESNGQPENNYKTTPPVLDS |
| | | | | DGSFFLYSRLTVDKSRWQEGNVFSCSVM |
| | | | | HEALHNHYTQKSLSLSLGK |
| 437 | Human IgG4 | IGHG4*02 | Human Heavy | gcttccaccaagggcccatccgtcttcc |
| | constant | | Chain Constant | ccctggcgccctgctccaggagcacctc |
| | region | | Region | cgagagcacagccgccctgggctgcctg |
| | | | (IGHG4*02) | gtcaaggactacttccccgaaccggtga |
| | | | Nucleotide | cggtgtcgtggaactcaggcgccctgac |
| | | | Sequence | cagcggcgtgcacaccttcccggctgtc |
| | | | | ctacagtcctcaggactctactccctca |
| | | | | gcagcgtggtgaccgtgccctccagcag |
| | | | | cttgggcacgaagacctacacctgcaac |
| | | | | gtagatcacaagcccagcaacaccaagg |
| | | | | tggacaagagagttgagtccaaatatgg |
| | | | | tcccccgtgcccatcatgcccagcacct |
| | | | | gagttcctggggggaccatcagtcttcc |
| | | | | tgttccccccaaaacccaaggacactct |
| | | | | catgatctcccggacccctgaggtcacg |
| | | | | tgcgtggtggtggacgtgagccaggaag |
| | | | | accccgaggtccagttcaactggtacgt |
| | | | | ggatggcgtggaggtgcataatgccaag |
| | | | | acaaagccgcgggaggagcagttcaaca |
| | | | | gcacgtaccgtgtggtcagcgtcctcac |
| | | | | cgtcgtgcaccaggactggctgaacggc |
| | | | | aaggagtacaagtgcaaggtctccaaca |
| | | | | aaggcctcccgtcctccatcgagaaaac |
| | | | | catctccaaagccaaagggcagccccga |
| | | | | gagccacaggtgtacaccctgcccccat |
| | | | | cccaggaggagatgaccaagaaccaggt |
| | | | | cagcctgacctgcctggtcaaaggcttc |
| | | | | taccccagcgacatcgccgtggagtggg |
| | | | | agagcaatgggcagccggagaacaacta |
| | | | | caagaccacgcctcccgtgctggactcc |
| | | | | gacggctccttcttcctctacagcaggc |
| | | | | taaccgtggacaagagcaggtggcagga |
| | | | | ggggaatgtcttctcatgctccgtgatg |
| | | | | catgaggctctgcacaaccactacacgc |
| | | | | agaagagcctctccctgtctctgggtaa |
| | | | | a |
| 438 | | | Human Heavy | ASTKGPSVFPLAPCSRSTSESTAALGCL |
| | | | Chain Constant | VKDYFPEPVTVSWNSGALTSGVHTFPAV |
| | | | Region | LQSSGLYSLSSVVTVPSSSLGTKTYTCN |
| | | | (IGHG4*02) | VDHKPSNTKVDKRVESKYGPPCPSCPAP |
| | | | Protein Sequence | EFLGGPSVFLFPPKPKDTLMISRTPEVT |
| | | | | CVVVDVSQEDPEVQFNWYVDGVEVHNAK |
| | | | | TKPREEQFNSTYRVVSVLTVVHQDWING |
| | | | | KEYKCKVSNKGLPSSIEKTISKAKGQPR |
| | | | | EPQVYTLPPSQEEMTKNQVSLTCLVKGF |
| | | | | YPSDIAVEWESNGQPENNYKTTPPVLDS |
| | | | | DGSFFLYSRLTVDKSRWQEGNVFSCSVM |
| | | | | HEALHNHYTQKSLSLSLGK |
| 439 | Human IgG4 | IGHG4*03 | Human Heavy | gcttccaccaagggcccatccgtcttcc |
| | constant | | Chain Constant | ccctggcgccctgctccaggagcacctc |
| | region | | Region | cgagagcacagccgccctgggctgcctg |
| | | | (IGHG4*03) | gtcaaggactacttccccgaaccggtga |
| | | | Nucleotide | cggtgtcgtggaactcaggcgccctgac |
| | | | Sequence | cagcggcgtgcacaccttcccggctgtc |
| | | | | ctacagtcctcaggactctactccctca |
| | | | | gcagcgtggtgaccgtgccctccagcag |
| | | | | cttgggcacgaagacctacacctgcaac |
| | | | | gtagatcacaagcccagcaacaccaagg |
| | | | | tggacaagagagttgagtccaaatatgg |
| | | | | tcccccatgcccatcatgcccagcacct |
| | | | | gagttcctggggggaccatcagtcttcc |
| | | | | tgttccccccaaaacccaaggacactct |
| | | | | catgatctcccggacccctgaggtcacg |
| | | | | tgcgtggtggtggacgtgagccaggaag |
| | | | | accccgaggtccagttcaactggtacgt |
| | | | | ggatggcgtggaggtgcataatgccaag |
| | | | | acaaagccgcgggaggagcagttcaaca |
| | | | | gcacgtaccgtgtggtcagcgtcctcac |
| | | | | cgtcctgcaccaggactggctgaacggc |
| | | | | aaggagtacaagtgcaaggtctccaaca |
| | | | | aaggcctcccgtcctccatcgagaaaac |
| | | | | catctccaaagccaaagggcagccccga |
| | | | | gagccacaggtgtacaccctgcccccat |
| | | | | cccaggaggagatgaccaagaaccaggt |
| | | | | cagcctgacctgcctggtcaaaggcttc |
| | | | | taccccagcgacatcgccgtggagtggg |
| | | | | agagcaatgggcagccggagaacaacta |
| | | | | caagaccacgcctcccgtgctggactcc |
| | | | | gacggctccttcttcctctacagcaagc |
| | | | | tcaccgtggacaagagcaggtggcagga |
| | | | | ggggaacgtcttctcatgctccgtgatg |
| | | | | catgaggctctgcacaaccactacacgc |
| | | | | agaagagcctctccctgtctctgggtaa |
| | | | | a |
| 440 | | | Human Heavy | ASTKGPSVFPLAPCSRSTSESTAALGCL |
| | | | Chain Constant | VKDYFPEPVTVSWNSGALTSGVHTFPAV |
| | | | Region | LQSSGLYSLSSVVTVPSSSLGTKTYTCN |
| | | | (IGHG4*03) | VDHKPSNTKVDKRVESKYGPPCPSCPAP |
| | | | Protein Sequence | EFLGGPSVFLFPPKPKDTLMISRTPEVT |
| | | | | CVVVDVSQEDPEVQFNWYVDGVEVHNAK |
| | | | | TKPREEQFNSTYRVVSVLTVLHQDWING |
| | | | | KEYKCKVSNKGLPSSIEKTISKAKGQPR |
| | | | | EPQVYTLPPSQEEMTKNQVSLTCLVKGF |
| | | | | YPSDIAVEWESNGQPENNYKTTPPVLDS |
| | | | | DGSFFLYSKLTVDKSRWQEGNVFSCSVM |
| | | | | HEALHNHYTQKSLSLSLGK |
| 441 | Human IgG4- | IGHG4-PE | Human Heavy | gcctccaccaagggcccatccgtcttcc |
| | PE constant | | Chain Constant | ccctggcgccctgctccaggagcacctc |
| | region | | Region (IGHG4- | cgagagcacggccgccctgggctgcctg |
| | | | PE) Nucleotide | gtcaaggactacttccccgaaccagtga |
| | | | Sequence Version | cggtgtcgtggaactcaggcgccctgac |
| | | | A | cagcggcgtgcacaccttcccggctgtc |
| | | | | ctacagtcctcaggactctactccctca |
| | | | | gcagcgtggtgaccgtgccctccagcag |
| | | | | cttgggcacgaagacctacacctgcaac |
| | | | | gtagatcacaagcccagcaacaccaagg |
| | | | | tggacaagagagttgagtccaaatatgg |
| | | | | tcccccatgcccaccatgcccagcgcct |
| | | | | gaatttgaggggggaccatcagtcttcc |
| | | | | tgttccccccaaaacccaaggacactct |
| | | | | catgatctcccggacccctgaggtcacg |
| | | | | tgcgtggtggtggacgtgagccaggaag |
| | | | | accccgaggtccagttcaactggtacgt |
| | | | | ggatggcgtggaggtgcataatgccaag |
| | | | | acaaagccgcgggaggagcagttcaaca |
| | | | | gcacgtaccgtgtggtcagcgtcctcac |
| | | | | cgtcctgcaccaggactggctgaacggc |
| | | | | aaggagtacaagtgcaaggtctccaaca |
| | | | | aaggcctcccgtcatcgatcgagaaaac |
| | | | | catctccaaagccaaagggcagccccga |
| | | | | gagccacaggtgtacaccctgcccccat |
| | | | | cccaggaggagatgaccaagaaccaggt |
| | | | | cagcctgacctgcctggtcaaaggcttc |
| | | | | taccccagcgacatcgccgtggagtggg |
| | | | | agagcaatgggcagccggagaacaacta |
| | | | | caagaccacgcctcccgtgctggactcc |
| | | | | gacggatccttcttcctctacagcaggc |
| | | | | taaccgtggacaagagcaggtggcagga |
| | | | | ggggaatgtcttctcatgctccgtgatg |
| | | | | catgaggctctgcacaaccactacacac |
| | | | | agaagagcctctccctgtctctgggtaa |
| | | | | a |
| 442 | | | Human Heavy | gcctccaccaagggacctagcgtgttcc |
| | | | Chain Constant | ctctcgccccctgttccaggtccacaag |
| | | | Region (IGHG4- | cgagtccaccgctgccctcggctgtctg |
| | | | PE) Nucleotide | gtgaaagactactttcccgagcccgtga |
| | | | Sequence Version | ccgtctcctggaatagcggagccctgac |
| | | | B | ctccggcgtgcacacatttcccgccgtg |
| | | | | ctgcagagcagcggactgtatagcctga |
| | | | | gcagcgtggtgaccgtgcccagctccag |
| | | | | cctcggcaccaaaacctacacctgcaac |
| | | | | gtggaccacaagccctccaacaccaagg |
| | | | | tggacaagcgggtggagagcaagtacgg |
| | | | | ccccccttgccctccttgtcctgcccct |
| | | | | gagttcgagggaggaccctccgtgttcc |
| | | | | tgtttccccccaaacccaaggacaccct |
| | | | | gatgatctcccggacacccgaggtgacc |
| | | | | tgtgtggtcgtggacgtcagccaggagg |
| | | | | accccgaggtgcagttcaactggtatgt |
| | | | | ggacggcgtggaggtgcacaatgccaaa |
| | | | | accaagcccagggaggagcagttcaatt |
| | | | | ccacctacagggtggtgagcgtgctgac |
| | | | | cgtcctgcatcaggattggctgaacggc |
| | | | | aaggagtacaagtgcaaggtgtccaaca |
| | | | | agggactgcccagctccatcgagaagac |
| | | | | catcagcaaggctaagggccagccgagg |
| | | | | gagccccaggtgtataccctgcctccta |
| | | | | gccaggaagagatgaccaagaaccaagt |
| | | | | gtccctgacctgcctggtgaagggattc |
| | | | | tacccctccgacatcgccgtggagtggg |
| | | | | agagcaatggccagcccgagaacaacta |
| | | | | caaaacaacccctcccgtgctcgatagc |
| | | | | gacggcagcttctttctctacagccggc |
| | | | | tgacagtggacaagagcaggtggcagga |
| | | | | gggcaacgtgttctcctgttccgtgatg |
| | | | | cacgaggccctgcacaatcactacaccc |
| | | | | agaagagcctctccctgtccctgggcaa |
| | | | | g |
| 443 | Human IgG4- | IGHG4-E | Human Heavy | gccagcaccaagggcccttccgtgttcc |
| | PE constant | Inactivated | Chain Constant | ccctggccccttgcagcaggagcacctc |
| | region | IGHG4 | Region (IGHG4- | cgaatccacagctgccctgggctgtctg |
| | Inactivated | | PE) Nucleotide | gtgaaggactactttcccgagcccgtga |
| | Human IgG4 | | Sequence Version | ccgtgagctggaacagcggcgctctgac |
| | constant | | C | atccggcgtccacacctttcctgccgtc |
| | region | | | ctgcagtcctccggcctctactccctgt |
| | | | | cctccgtggtgaccgtgcctagctcctc |
| | | | | cctcggcaccaagacctacacctgtaac |
| | | | | gtggaccacaaaccctccaacaccaagg |
| | | | | tggacaaacgggtcgagagcaagtacgg |
| | | | | ccctccctgccctccttgtcctgccccc |
| | | | | gagttcgaaggcggacccagcgtgttcc |
| | | | | tgttccctcctaagcccaaggacaccct |
| | | | | catgatcagccggacacccgaggtgacc |
| | | | | tgcgtggtggtggatgtgagccaggagg |
| | | | | accctgaggtccagttcaactggtatgt |
| | | | | ggatggcgtggaggtgcacaacgccaag |
| | | | | acaaagccccgggaagagcagttcaact |
| | | | | ccacctacagggtggtcagcgtgctgac |
| | | | | cgtgctgcatcaggactggctgaacggc |
| | | | | aaggagtacaagtgcaaggtcagcaata |
| | | | | agggactgcccagcagcatcgagaagac |
| | | | | catctccaaggctaaaggccagccccgg |
| | | | | gaacctcaggtgtacaccctgcctccca |
| | | | | gccaggaggagatgaccaagaaccaggt |
| | | | | gagcctgacctgcctggtgaagggattc |
| | | | | tacccttccgacatcgccgtggagtggg |
| | | | | agtccaacggccagcccgagaacaatta |
| | | | | taagaccacccctcccgtcctcgacagc |
| | | | | gacggatccttctttctgtactccaggc |
| | | | | tgaccgtggataagtccaggtggcagga |
| | | | | aggcaacgtgttcagctgctccgtgatg |
| | | | | cacgaggccctgcacaatcactacaccc |
| | | | | agaagtccctgagcctgtccctgggaaa |
| | | | | g |
| 444 | | | Human Heavy | ASTKGPSVFPLAPCSRSTSESTAALGCL |
| | | | Chain Constant | VKDYFPEPVTVSWNSGALTSGVHTFPAV |
| | | | Region (IGHG4- | LQSSGLYSLSSVVTVPSSSLGTKTYTCN |
| | | | PE) Protein | VDHKPSNTKVDKRVESKYGPPCPPCPAP |
| | | | Sequence (Amino | EFEGGPSVFLFPPKPKDTLMISRTPEVT |
| | | | acid | CVVVDVSQEDPEVQFNWYVDGVEVHNAK |
| | | | substitution | TKPREEQFNSTYRVVSVLTVLHQDWLNG |
| | | | shown in BOLD) | KEYKCKVSNKGLPSSIEKTISKAKGQPR |
| | | | | EPQVYTLPPSQEEMTKNQVSLTCLVKGF |
| | | | | YPSDIAVEWESNGQPENNYKTTPPVLDS |
| | | | | DGSFFLYSRLTVDKSRWQEGNVFSCSVM |
| | | | | HEALHNHYTQKSLSLSLGK |
| 445 | | | Inactivated | gcctccaccaagggcccatccgtcttcc |
| | | | Human Heavy | ccctggcgccctgctccaggagcacctc |
| | | | Chain Constant | cgagagcacggccgccctgggctgcctg |
| | | | Region (IGHG4) | gtcaaggactacttccccgaaccagtga |
| | | | Nucleotide | cggtgtcgtggaactcaggcgccctgac |
| | | | Sequence | cagcggcgtgcacaccttcccggctgtc |
| | | | | ctacagtcctcaggactctactccctca |
| | | | | gcagcgtggtgaccgtgccctccagcag |
| | | | | cttgggcacgaagacctacacctgcaac |
| | | | | gtagatcacaagcccagcaacaccaagg |
| | | | | tggacaagagagttgagtccaaatatgg |
| | | | | tcccccatgcccaccatgcccagcgcct |
| | | | | ccagttgcggggggaccatcagtcttcc |
| | | | | tgttccccccaaaacccaaggacactct |
| | | | | catgatctcccggacccctgaggtcacg |
| | | | | tgcgtggtggtggacgtgagccaggaag |
| | | | | accccgaggtccagttcaactggtacgt |
| | | | | ggatggcgtggaggtgcataatgccaag |
| | | | | acaaagccgcgggaggagcagttcaaca |
| | | | | gcacgtaccgtgtggtcagcgtcctcac |
| | | | | cgtcctgcaccaggactggctgaacggc |
| | | | | aaggagtacaagtgcaaggtctccaaca |
| | | | | aaggcctcccgtcatcgatcgagaaaac |
| | | | | catctccaaagccaaagggcagccccga |
| | | | | gagccacaggtgtacaccctgcccccat |
| | | | | cccaggaggagatgaccaagaaccaggt |
| | | | | cagcctgacctgcctggtcaaaggcttc |
| | | | | taccccagcgacatcgccgtggagtggg |
| | | | | agagcaatgggcagccggagaacaacta |
| | | | | caagaccacgcctcccgtgctggactcc |
| | | | | gacggatccttcttcctctacagcaggc |
| | | | | taaccgtggacaagagcaggtggcagga |
| | | | | ggggaatgtcttctcatgctccgtgatg |
| | | | | catgaggctctgcacaaccactacacac |
| | | | | agaagagcctctccctgtctctgggtaa |
| | | | | a |
| 446 | | | Inactivated | ASTKGPSVFPLAPCSRSTSESTAALGCL |
| | | | Human Heavy | VKDYFPEPVTVSWNSGALTSGVHTFPAV |
| | | | Chain Constant | LQSSGLYSLSSVVTVPSSSLGTKTYTCN |
| | | | Region (IGHG4) | VDHKPSNTKVDKRVESKYGPPCPPCPAP |
| | | | Protein Sequence | PVAGGPSVFLFPPKPKDTLMISRTPEVT |
| | | | (inactivating | CVVVDVSQEDPEVQFNWYVDGVEVHNAK |
| | | | mutations from | TKPREEQFNSTYRVVSVLTVLHQDWING |
| | | | human IgG4 shown | KEYKCKVSNKGLPSSIEKTISKAKGQPR |
| | | | in bold) | EPQVYTLPPSQEEMTKNQVSLTCLVKGE |
| | | | | YPSDIAVEWESNGQPENNYKTTPPVLDS |
| | | | | DGSFFLYSRLTVDKSRWQEGNVFSCSVM |
| | | | | HEALHNHYTQKSLSLSLGK |
| 447 | Human Cκ | IGKC*01 | Human Cκk Light | cgtacggtggccgctccctccgtgttca |
| | constant | | Chain Constant | tcttcccaccttccgacgagcagctgaa |
| | region | | Region (IGKC*01) | gtccggcaccgcttctgtcgtgtgcctg |
| | | | Nucleotide | ctgaacaacttctacccccgcgaggcca |
| | | | Sequence | aggtgcagtggaaggtggacaacgccct |
| | | | | gcagtccggcaactcccaggaatccgtg |
| | | | | accgagcaggactccaaggacagcacct |
| | | | | actccctgtcctccaccctgaccctgtc |
| | | | | caaggccgactacgagaagcacaaggtg |
| | | | | tacgcctgcgaagtgacccaccagggcc |
| | | | | tgtctagccccgtgaccaagtctttcaa |
| | | | | ccggggcgagtgt |
| 448 | | | Cκ Light Chain | RTVAAPSVFIFPPSDEQLKSGTASVVCL |
| | | | Constant Region | LNNFYPREAKVQWKVDNALQSGNSQESV |
| | | | (IGKC*01) Amino | TEQDSKDSTYSLSSTLTLSKADYEKHKV |
| | | | Acid Sequence | YACEVTHQGLSSPVTKSFNRGEC |
| 449 | Human Cκ | IGKC*02 | Cκ Light Chain | cgaactgtggctgcaccatctgtcttca |
| | constant | | Constant Region | tcttcccgccatctgatgagcagttgaa |
| | region | | (IGKC*02) | atctggaactgcctctgttgtgtgcctg |
| | | | Nucleotide | ctgaataacttctatcccagagaggcca |
| | | | Sequence | aagtacagtggaaggtggataacgccct |
| | | | | ccaatcgggtaactcccaggagagtgtc |
| | | | | acagagcaggagagcaaggacagcacct |
| | | | | acagcctcagcagcaccctgacgctgag |
| | | | | caaagcagactacgagaaacacaaagtc |
| | | | | tacgccggcgaagtcacccatcagggcc |
| | | | | tgagctcgcccgtcacaaagagcttcaa |
| | | | | caggggagagtgt |
| 450 | | | Cκ Light Chain | RTVAAPSVFIFPPSDEQLKSGTASVVCL |
| | | | Constant Region | LNNFYPREAKVQWKVDNALQSGNSQESV |
| | | | (IGKC*02) Amino | TEQESKDSTYSLSSTLTLSKADYEKHKV |
| | | | Acid Sequence | YAGEVTHQGLSSPVTKSFNRGEC |
| 451 | Human Cκ | IGKC*03 | Cκ Light Chain | cgaactgtggctgcaccatctgtcttca |
| | constant | | Constant Region | tcttcccgccatctgatgagcagttgaa |
| | region | | (IGKC*03) | atctggaactgcctctgttgtgtgcctg |
| | | | Nucleotide | ctgaataacttctatcccagagaggcca |
| | | | Sequence | aagtacagcggaaggtggataacgccct |
| | | | | ccaatcgggtaactcccaggagagtgtc |
| | | | | acagagcaggagagcaaggacagcacct |
| | | | | acagcctcagcagcaccctgacgctgag |
| | | | | caaagcagactacgagaaacacaaagtc |
| | | | | tacgcctgcgaagtcacccatcagggcc |
| | | | | tgagctcgcccgtcacaaagagcttcaa |
| | | | | caggggagagtgt |
| 452 | | | Cκ Light Chain | RTVAAPSVFIFPPSDEQLKSGTASVVCL |
| | | | Constant Region | LNNFYPREAKVQRKVDNALQSGNSQESV |
| | | | (IGKC*03) Amino | TEQESKDSTYSLSSTLTLSKADYEKHKV |
| | | | Acid Sequence | YACEVTHQGLSSPVTKSFNRGEC |
| 453 | Human Cκ | IGKC*04 | Cκ Light Chain | cgaactgtggctgcaccatctgtcttca |
| | constant | | Constant Region | tcttcccgccatctgatgagcagttgaa |
| | region | | (IGKC*04) | atctggaactgcctctgttgtgtgcctg |
| | | | Nucleotide | ctgaataacttctatcccagagaggcca |
| | | | Sequence | aagtacagtggaaggtggataacgccct |
| | | | | ccaatcgggtaactcccaggagagtgtc |
| | | | | acagagcaggacagcaaggacagcacct |
| | | | | acagcctcagcagcaccctgacgctgag |
| | | | | caaagcagactacgagaaacacaaactc |
| | | | | tacgcctgcgaagtcacccatcagggcc |
| | | | | tgagctcgcccgtcacaaagagcttcaa |
| | | | | caggggagagtgt |
| 454 | | | Cκ Light Chain | RTVAAPSVFIFPPSDEQLKSGTASVVCL |
| | | | Constant Region | LNNFYPREAKVQWKVDNALQSGNSQESV |
| | | | (IGKC*04) Amino | TEQDSKDSTYSLSSTLTLSKADYEKHKL |
| | | | Acid Sequence | YACEVTHQGLSSPVTKSFNRGEC |
| 455 | Human Cκ | IGKC*05 | Cκ Light Chain | cgaactgtggctgcaccatctgtcttca |
| | constant | | Constant Region | tcttcccgccatctgatgagcagttgaa |
| | region | | (IGKC*05) | atctggaactgcctctgttgtgtgcctg |
| | | | Nucleotide | ctgaataacttctatcccagagaggcca |
| | | | Sequence | aagtacagtggaaggtggataacgccct |
| | | | | ccaatcgggtaactcccaggagagtgtc |
| | | | | acagagcaggacagcaaggacagcacct |
| | | | | acagcctcagcaacaccctgacgctgag |
| | | | | caaagcagactacgagaaacacaaagtc |
| | | | | tacgcctgcgaagtcacccatcagggcc |
| | | | | tgagctcgcccgtcacaaagagcttcaa |
| | | | | caggggagagtgc |
| 456 | | | Cκ Light Chain | RTVAAPSVFIFPPSDEQLKSGTASVVCL |
| | | | Constant Region | LNNFYPREAKVQWKVDNALQSGNSQESV |
| | | | (IGKC*05) Amino | TEQDSKDSTYSLSNTLTLSKADYEKHKV |
| | | | Acid Sequence | YACEVTHQGLSSPVTKSfNRGEC |
| 457 | Human Cλ | IGLC1*01 | Cλ Light Chain | cccaaggccaaccccacggtcactctgt |
| | constant | | Constant Region | tcccgccctcctctgaggagctccaagc |
| | region | | (IGLC1*01) | caacaaggccacactagtgtgtctgatc |
| | | | Nucleotide | agtgacttctacccgggagctgtgacag |
| | | | Sequence | tggcttggaaggcagatggcagccccgt |
| | | | (ENST00000390321.2) | caaggcgggagtggagacgaccaaaccc |
| | | | | tccaaacagagcaacaacaagtacgcgg |
| | | | | ccagcagctacctgagcctgacgcccga |
| | | | | gcagtggaagtcccacagaagctacagc |
| | | | | tgccaggtcacgcatgaagggagcaccg |
| | | | | tggagaagacagtggcccctacagaatg |
| | | | | ttca |
| 458 | | | Cλ Light Chain | PKANPTVTLFPPSSEELQANKATLVCLI |
| | | | Constant Region | SDFYPGAVTVAWKADGSPVKAGVETTKP |
| | | | (IGLC1*01) Amino | SKQSNNKYAASSYLSLTPEQWKSHRSYS |
| | | | Acid Sequence | CQVTHEGSTVEKTVAPTECS |
| | | | (A0A075B6K8) | |
| 459 | Human Cλ | IGLC1*02 | Cλ Light Chain | ggtcagcccaaggccaaccccactgtca |
| | constant | | Constant Region | ctctgttcccgccctcctctgaggagct |
| | region | | (IGLC1*02) | ccaagccaacaaggccacactagtgtgt |
| | | | Nucleotide | ctgatcagtgacttctacccgggagctg |
| | | | Sequence Version | tgacagtggcctggaaggcagatggcag |
| | | | A | ccccgtcaaggcgggagtggagaccacc |
| | | | | aaaccctccaaacagagcaacaacaagt |
| | | | | acgcggccagcagctacctgagcctgac |
| | | | | gcccgagcagtggaagtcccacagaagc |
| | | | | tacagctgccaggtcacgcatgaaggga |
| | | | | gcaccgtggagaagacagtggcccctac |
| | | | | agaatgttca |
| 460 | | | Cλ Light Chain | ggtcagcccaaggccaaccccactgtca |
| | | | Constant Region | ctctgttcccgccctcctctgaggagct |
| | | | (IGLC1*02) | ccaagccaacaaggccacactagtgtgt |
| | | | Nucleotide | ctgatcagtgacttctacccgggagctg |
| | | | Sequence Version | tgacagtggcctggaaggcagatggcag |
| | | | B | ccccgtcaaggcgggagtggagaccacc |
| | | | | aaaccctccaaacagagcaacaacaagt |
| | | | | acgcggccagcagctacctgagcctgac |
| | | | | gcccgagcagtggaagtcccacagaagc |
| | | | | tacagctgccaggtcacgcatgaaggga |
| | | | | gcaccgtggagaagacagtggcccctac |
| | | | | agaatgttca |
| 461 | Human Cλ | IGLC1*02 | Cλ Light Chain | GQPKANPTVTLFPPSSEELQANKATLVC |
| | constant | IGLC2*01 | Constant Region | LISDFYPGAVTVAWKADGSPVKAGVETT |
| | region | | (IGLC1*02) Amino | KPSKQSNNKYAASSYLSLTPEQWKSHRS |
| | Human Cλ | | Acid Sequence | YSCQVTHEGSTVEKTVAPTECS |
| 462 | constant | | Cλ Light Chain | ggccagcctaaggccgctccttctgtga |
| | region | | Constant Region | ccctgttccccccatcctccgaggaact |
| | | | (IGLC2*01) | gcaggctaacaaggccaccctcgtgtgc |
| | | | Nucleotide | ctgatcagcgacttctaccctggcgccg |
| | | | Sequence Version | tgaccgtggcctggaaggctgatagctc |
| | | | A | tcctgtgaaggccggcgtggaaaccacc |
| | | | | accccttccaagcagtccaacaacaaat |
| | | | | acgccgcctcctcctacctgtccctgac |
| | | | | ccctgagcagtggaagtcccaccggtcc |
| | | | | tacagctgccaagtgacccacgagggct |
| | | | | ccaccgtggaaaagaccgtggctcctac |
| | | | | cgagtgctcc |
| 463 | | | Cλ Light Chain | ggccagcctaaagctgcccccagcgtca |
| | | | Constant Region | ccctgtttcctccctccagcgaggagct |
| | | | (IGLC2*01) | ccaggccaacaaggccaccctcgtgtgc |
| | | | Nucleotide | ctgatctccgacttctatcccggcgctg |
| | | | Sequence Version | tgaccgtggcttggaaagccgactccag |
| | | | B | ccctgtcaaagccggcgtggagaccacc |
| | | | | acaccctccaagcagtccaacaacaagt |
| | | | | acgccgcctccagctatctctccctgac |
| | | | | ccctgagcagtggaagtcccaccggtcc |
| | | | | tactcctgtcaggtgacccacgagggct |
| | | | | ccaccgtggaaaagaccgtcgcccccac |
| | | | | cgagtgctcc |
| 464 | Human Cλ | IGLC2*01 | Cλ Light Chain | GQPKAAPSVTLFPPSSEELQANKATLVC |
| | constant | IGLC2*02 or | Constant Region | LISDFYPGAVTVAWKADSSPVKAGVETT |
| | region | IGLC2*03 | (IGLC1*02) Amino | TPSKQSNNKYAASSYLSLTPEQWKSHRS |
| | Human Cλ | | Acid Sequence | YSCQVTHEGSTVEKTVAPTECS |
| 465 | constant | | Cλ Light Chain | ggtcagcccaaggctgccccctcggtca |
| | region | | Constant Region | ctctgttcccgccctcctctgaggagct |
| | | | (IGLC2*02 or | tcaagccaacaaggccacactggtgtgt |
| | | | IGLC2*03) | ctcataagtgacttctacccgggagccg |
| | | | Nucleotide | tgacagtggcctggaaggcagatagcag |
| | | | Sequence | ccccgtcaaggcgggagtggagaccacc |
| | | | | acaccctccaaacaaagcaacaacaagt |
| | | | | acgcggccagcagctatctgagcctgac |
| | | | | gcctgagcagtggaagtcccacagaagc |
| | | | | tacagctgccaggtcacgcatgaaggga |
| | | | | gcaccgtggagaagacagtggcccctac |
| | | | | agaatgttca |
| 467 | | | Cλ Light Chain | GQPKAAPSVTLFPPSSEELQANKATLVC |
| | | | Constant Region | LISDFYPGAVTVAWKADSSPVKAGVETT |
| | | | (IGLC2*02) Amino | TPSKQSNNKYAASSYLSLTPEQWKSHRS |
| | | | Acid Sequence | YSCQVTHEGSTVEKTVAPTECS |
| 468 | Human Cλ | IGLC3*01 | Cλ Light Chain | cccaaggctgccccctcggtcactctgt |
| | constant | | Constant Region | tcccaccctcctctgaggagcttcaagc |
| | region | | (IGLC3*01) | caacaaggccacactggtgtgtctcata |
| | | | Nucleotide | agtgacttctacccgggagccgtgacag |
| | | | Sequence | ttgcctggaaggcagatagcagccccgt |
| | | | | caaggcgggggtggagaccaccacaccc |
| | | | | tccaaacaaagcaacaacaagtacgcgg |
| | | | | ccagcagctacctgagcctgacgcctga |
| | | | | gcagtggaagtcccacaaaagctacagc |
| | | | | tgccaggtcacgcatgaagggagcaccg |
| | | | | tggagaagacagttgcccctacggaatg |
| | | | | ttca |
| 469 | | | Cλ Light Chain | PKAAPSVTLFPPSSEELQANKATLVCLI |
| | | | Constant Region | SDFYPGAVTVAWKADSSPVKAGVETTTP |
| | | | (IGLC3*01) Amino | SKQSNNKYAASSYLSLTPEQWKSHKSYS |
| | | | Acid Sequence | CQVTHEGSTVEKTVAPTECS |
| 470 | Human Cλ | IGLC3*02 | Cλ Light Chain | ggtcagcccaaggctgccccctcggtca |
| | constant | | Constant Region | ctctgttcccaccctcctctgaggagct |
| | region | | (IGLC3*02) | tcaagccaacaaggccacactggtgtgt |
| | | | Nucleotide | ctcataagtgacttctacccggggccag |
| | | | Sequence | tgacagttgcctggaaggcagatagcag |
| | | | | ccccgtcaaggcgggggtggagaccacc |
| | | | | acaccctccaaacaaagcaacaacaagt |
| | | | | acgcggccagcagctacctgagcctgac |
| | | | | gcctgagcagtggaagtcccacaaaagc |
| | | | | tacagctgccaggtcacgcatgaaggga |
| | | | | gcaccgtggagaagacagtggcccctac |
| | | | | ggaatgttca |
| 471 | | | Cλ Light Chain | GQPKAAPSVTLFPPSSEELQANKATLVC |
| | | | Constant Region | LISDFYPGPVTVAWKADSSPVKAGVETT |
| | | | (IGLC1*02) Amino | TPSKQSNNKYAASSYLSLTPEQWKSHKS |
| | | | Acid Sequence | YSCQVTHEGSTVEKTVAPTECS |
| 472 | Human Cλ | IGLC3*03 | Cλ Light Chain | ggtcagcccaaggctgccccctcggtca |
| | constant | | Constant Region | ctctgttcccaccctcctctgaggagct |
| | region | | (IGLC3*03) | tcaagccaacaaggccacactggtgtgt |
| | | | Nucleotide | ctcataagtgacttctacccgggagccg |
| | | | Sequence | tgacagtggcctggaaggcagatagcag |
| | | | | ccccgtcaaggcgggagtggagaccacc |
| | | | | acaccctccaaacaaagcaacaacaagt |
| | | | | acgcggccagcagctacctgagcctgac |
| | | | | gcctgagcagtggaagtcccacaaaagc |
| | | | | tacagctgccaggtcacgcatgaaggga |
| | | | | gcaccgtggagaagacagtggcccctac |
| | | | | agaatgttca |
| 473 | | | Cλ Light Chain | GQPKAAPSVTLFPPSSEELQANKATLVC |
| | | | Constant Region | LISDFYPGAVTVAWKADSSPVKAGVETT |
| | | | (IGLC3*03) Amino | TPSKQSNNKYAASSYLSLTPEQWKSHKS |
| | | | Acid Sequence | YSCQVTHEGSTVEKTVAPTECS |
| 474 | Human Cλ | IGLC3*04 | Cλ Light Chain | ggtcagcccaaggctgccccctcggtca |
| | constant | | Constant Region | ctctgttcccgccctcctctgaggagct |
| | region | | (IGLC3*04) | tcaagccaacaaggccacactggtgtgt |
| | | | Nucleotide | ctcataagtgacttctacccgggagccg |
| | | | Sequence | tgacagtggcctggaaggcagatagcag |
| | | | | ccccgtcaaggcgggagtggagaccacc |
| | | | | acaccctccaaacaaagcaacaacaagt |
| | | | | acgcggccagcagctacctgagcctgac |
| | | | | gcctgagcagtggaagtcccacagaagc |
| | | | | tacagctgccaggtcacgcatgaaggga |
| | | | | gcaccgtggagaagacagtggcccctac |
| | | | | agaatgttca |
| 475 | | | Cλ Light Chain | GQPKAAPSVTLFPPSSEELQANKATLVC |
| | | | Constant Region | LISDFYPGAVTVAWKADSSPVKAGVETT |
| | | | (IGLC3*04) Amino | TPSKQSNNKYAASSYLSLTPEQWKSHRS |
| | | | Acid Sequence | YSCQVTHEGSTVEKTVAPTECS |
| 476 | Human Cλ | IGLC6*01 | Cλ Light Chain | ggtcagcccaaggctgccccatcggtca |
| | constant | | Constant Region | ctctgttcccgccctcctctgaggagct |
| | region | | (IGLC6*01) | tcaagccaacaaggccacactggtgtgc |
| | | | Nucleotide | ctgatcagtgacttctacccgggagctg |
| | | | Sequence | tgaaagtggcctggaaggcagatggcag |
| | | | | ccccgtcaacacgggagtggagaccacc |
| | | | | acaccctccaaacagagcaacaacaagt |
| | | | | acgcggccagcagctacctgagcctgac |
| | | | | gcctgagcagtggaagtcccacagaagc |
| | | | | tacagctgccaggtcacgcatgaaggga |
| | | | | gcaccgtggagaagacagtggcccctgc |
| | | | | agaatgttca |
| 477 | | | Cλ Light Chain | GQPKAAPSVTLFPPSSEELQANKATLVC |
| | | | Constant Region | LISDFYPGAVKVAWKADGSPVNTGVETT |
| | | | (IGLC6*01) Amino | TPSKQSNNKYAASSYLSLTPEQWKSHRS |
| | | | Acid Sequence | YSCQVTHEGSTVEKTVAPAECS |
| 478 | Human Cλ | IGLC7*01 or | Cλ Light Chain | ggtcagcccaaggctgccccatcggtca |
| | constant | IGLC7*02 | Constant Region | ctctgttcccaccctcctctgaggagct |
| | region | | (IGLC7*01 or | tcaagccaacaaggccacactggtgtgt |
| | | | IGLC7*02) | ctcgtaagtgacttctacccgggagccg |
| | | | Nucleotide | tgacagtggcctggaaggcagatggcag |
| | | | Sequence | ccccgtcaaggtgggagtggagaccacc |
| | | | | aaaccctccaaacaaagcaacaacaagt |
| | | | | atgcggccagcagctacctgagcctgac |
| | | | | gcccgagcagtggaagtcccacagaagc |
| | | | | tacagctgccgggtcacgcatgaaggga |
| | | | | gcaccgtggagaagacagtggcccctgc |
| | | | | agaatgctct |
| 479 | | | Cλ Light Chain | GQPKAAPSVTLFPPSSEELQANKATLVC |
| | | | Constant Region | LVSDFYPGAVTVAWKADGSPVKVGVETT |
| | | | (IGLC7*01) Amino | KPSKQSNNKYAASSYLSLTPEQWKSHRS |
| | | | Acid Sequence | YSCRVTHEGSTVEKTVAPAECS |
| 480 | Human Cλ | IGLC7*03 | Cλ Light Chain | GGTCAGCCCAAGGCTGCCCCCTCGGTCA |
| | constant | | Constant Region | CTCTGTTCCCACCCTCCTCTGAGGAGCT |
| | region | | (IGLC7*03) | TCAAGCCAACAAGGCCACACTGGTGTGT |
| | | | Nucleotide | CTCGTAAGTGACTTCAACCCGGGAGCCG |
| | | | Sequence | TGACAGTGGCCTGGAAGGCAGATGGCAG |
| | | | | CCCCGTCAAGGTGGGAGTGGAGACCACC |
| | | | | AAACCCTCCAAACAAAGCAACAACAAGT |
| | | | | ATGCGGCCAGCAGCTACCTGAGCCTGAC |
| | | | | GCCCGAGCAGTGGAAGTCCCACAGAAGC |
| | | | | TACAGCTGCCGGGTCACGCATGAAGGGA |
| | | | | GCACCGTGGAGAAGACAGTGGCCCCTGC |
| | | | | AGAATGCTCT |
| 481 | | | Cλ Light Chain | GQPKAAPSVTLFPPSSEELQANKATLVC |
| | | | Constant Region | LVSDFNPGAVTVAWKADGSPVKVGVETT |
| | | | (IGLC7*03) Amino | KPSKQSNNKYAASSYLSLTPEQWKSHRS |
| | | | Acid Sequence | YSCRVTHEGSTVEKTVAPAECS |
| 482 | Human IgG4- | IGHG4-PE | Human Heavy | GCTTCTACCAAGGGACCCAGCGTGTTCC |
| | PE constant | IGHG4-PE | Chain Constant | CTCTGGCTCCTTGCTCCAGATCCACCTC |
| | region | | Region (IGHG4- | CGAGTCTACAGCTGCTCTGGGCTGCCTG |
| | Human IgG4- | | PE) Nucleotide | GTCAAGGACTACTTTCCTGAGCCTGTGA |
| | PE constant | | Sequence (lysine | CCGTGTCTTGGAACTCTGGCGCTCTGAC |
| | region | | clipped) | ATCTGGCGTGCACACATTCCCTGCTGTG |
| | | | | CTGCAGTCCTCCGGCCTGTACTCTCTGT |
| | | | | CCTCTGTCGTGACCGTGCCTTCCTCTAG |
| | | | | CCTGGGCACCAAGACCTACACCTGTAAT |
| | | | | GTGGACCACAAGCCTTCCAACACCAAGG |
| | | | | TGGACAAGCGCGTGGAATCTAAGTACGG |
| | | | | CCCTCCTTGTCCTCCATGTCCTGCTCCA |
| | | | | GAGTTTGAAGGCGGCCCTTCCGTGTTTC |
| | | | | TGTTCCCTCCAAAGCCTAAGGACACCCT |
| | | | | GATGATCTCTCGGACCCCTGAAGTGACC |
| | | | | TGCGTGGTGGTGGATGTGTCCCAAGAGG |
| | | | | ATCCCGAGGTGCAGTTCAATTGGTACGT |
| | | | | GGACGGCGTGGAAGTGCACAACGCCAAG |
| | | | | ACCAAGCCTAGAGAGGAACAGTTCAACT |
| | | | | CCACCTACAGAGTGGTGTCCGTGCTGAC |
| | | | | CGTGCTGCACCAGGATTGGCTGAACGGC |
| | | | | AAAGAGTACAAGTGCAAGGTGTCCAACA |
| | | | | AGGGCCTGCCTAGCTCCATCGAAAAGAC |
| | | | | CATCTCCAAGGCCAAGGGCCAGCCTCGA |
| | | | | GAACCCCAGGTTTACACCCTGCCTCCAA |
| | | | | GCCAAGAGGAAATGACCAAGAACCAGGT |
| | | | | GTCCCTGACCTGCCTCGTGAAGGGATTC |
| | | | | TACCCCTCCGATATCGCCGTGGAATGGG |
| | | | | AGTCTAATGGCCAGCCAGAGAACAACTA |
| | | | | CAAGACAACCCCTCCTGTGCTGGACTCC |
| | | | | GACGGCTCCTTCTTTCTGTATTCCCGCC |
| | | | | TGACCGTGGACAAGTCCAGATGGCAAGA |
| | | | | GGGCAACGTGTTCTCCTGCAGCGTGATG |
| | | | | CACGAGGCCCTGCACAATCACTACACCC |
| | | | | AGAAGTCCCTGTCTCTGTCCCTGGGC |
| 483 | | | Human Heavy | ASTKGPSVFPLAPCSRSTSESTAALGCL |
| | | | Chain Constant | VKDYFPEPVTVSWNSGALTSGVHTFPAV |
| | | | Region (IGHG4- | LQSSGLYSLSSVVTVPSSSLGTKTYTCN |
| | | | PE) Protein | VDHKPSNTKVDKRVESKYGPPCPPCPAP |
| | | | Sequence (lysine | EFEGGPSVFLFPPKPKDTLMISRTPEVT |
| | | | clipped) | CVVVDVSQEDPEVQFNWYVDGVEVHNAK |
| | | | | TKPREEQFNSTYRVVSVLTVLHQDWLNG |
| | | | | KEYKCKVSNKGLPSSIEKTISKAKGQPR |
| | | | | EPQVYTLPPSQEEMTKNQVSLTCLVKGF |
| | | | | YPSDIAVEWESNGQPENNYKTTPPVLDS |
| | | | | DGSFFLYSRLTVDKSRWQEGNVFSCSVM |
| | | | | HEALHNHYTQKSLSLSLG |
| 484 | Human IGHA1 | | | ASPTSPKVFPLSLCSTQPDGNVVIA |
| | constant | | | CLVQGFFPQEPLSVTWSESGQGVTA |
| | region | | | RNFPPSQDASGDLYTTSSQLTLPAT |
| | | | | QCLAGKSVTCHVKHYTNPSQDVTVP |
| | | | | CPVPSTPPTPSPSTPPTPSPSCCHP |
| | | | | RLSLHRPALEDLLLGSEANLTCTLT |
| | | | | GLRDASGVTFTWTPSSGKSAVQGPP |
| | | | | ERDLCGCYSVSSVLPGCAEPWNHGK |
| | | | | TFTCTAAYPESKTPLTATLSKSGNT |
| | | | | FRPEVHLLPPPSEELALNELVTLTC |
| | | | | LARGFSPKDVLVRWLQGSQELPREK |
| | | | | YLTWASRQEPSQGTTTFAVTSILRV |
| | | | | AAEDWKKGDTFSCMVGHEALPLAFT |
| | | | | QKTIDRLAGKPTHVNVSVVMAEVDG |
| | | | | TCY |
| 485 | Human | | | ASPTSPKVFPLSLDSTPQDGNVVVACLV |
| | IGHA2 | | | QGFFPQEPLSVTWSESGQNVTARNFPPS |
| | constant | | | QDASGDLYTTSSQLTLPATQCPDGKSVT |
| | region | | | CHVKHYTNSSQDVTVPCRVPPPPPCCHP |
| | | | | RLSLHRPALEDLLLGSEANLTCTLTGLR |
| | | | | DASGATFTWTPSSGKSAVQGPPERDLCG |
| | | | | CYSVSSVLPGCAQPWNHGETFTCTAAHP |
| | | | | ELKTPLTANITKSGNTFRPEVHLLPPPS |
| | | | | EELALNELVTLTCLARGFSPKDVLVRWL |
| | | | | QGSQELPREKYLTWASRQEPSQGTTTYA |
| | | | | VTSILRVAAEDWKKGETFSCMVGHEALP |
| | | | | LAFTQKTIDRMAGKPTHINVSVVMAEAD |
| | | | | GTCY |

TABLE 3
Gene segments:

| | heavy | heavy | light | light |
|---|---|---|---|---|
| | chain v | chain j | chain v | chain j |
| | gene | gene | gene | gene |
| Antibody | segment | segment | segment | segment |
| PPB-1345 | IGHV3-23*04 | IGHJ2*01 | IGKV3-15*01 | IGKJ4*01 |
| PPB-1347 | IGHV3-23*04 | IGHJ2*01 | IGKV3D-15*d01 | IGKJ4*01 |
| PPB-1348 | IGHV3-9*01 | IGHJ3*02 | IGKV1-17*01 | IGKJ4*01 |
| PPB-1349 | IGHV2-5*10 | IGHJ4*02 | IGKV1-9*d01 | IGKJ3*01 |
| PPB-1350 | IGHV3-23*04 | IGHJ2*01 | IGKV3D-15*d01 | IGKJ4*01 |
| PPB-1351 | IGHV3-23*04 | IGHJ4*02 | IGKV3-15*01 | IGKJ4*01 |
| PPB-1363 | IGHV3-23*04 | IGHJ2*01 | IGKV3-15*01 | IGKJ4*01 |
| PPB-1364 | IGHV3-23*04 | IGHJ4*02 | IGKV3-15*01 | IGKJ3*01 |
| PPB-1397 | IGHV3-23*04 | IGHJ2*01 | IGKV3D-15*d01 | IGKJ4*01 |
| PPB-1398 | IGHV3-23*04 | IGHJ4*02 | IGKV3-15*01 | IGKJ3*01 |
| PPB-1400 | IGHV3-23*04 | IGHJ4*02 | IGKV3-15*01 | IGKJ3*01 |
| PPB-1401 | IGHV3-23*04 | IGHJ4*02 | IGKV3D-15*d01 | IGKJ4*01 |
| PPB-1402 | IGHV3-23*04 | IGHJ4*02 | IGKV3-15*01 | IGKJ4*01 |
| PPB-1403 | IGHV3-9*01 | IGHJ6*02 | IGKV1D-39*01 | IGKJ4*01 |
| PPB-1404 | IGHV3-23*04 | IGHJ4*02 | IGKV3-15*01 | IGKJ3*01 |
| PPB-1405 | IGHV2-5*10 | IGHJ4*02 | IGKV1-17*01 | IGKJ3*01 |
| PPB-1407 | IGHV2-5*10 | IGHJ4*02 | IGKV1-16*02 | IGKJ3*01 |
| PPB-1408 | IGHV2-5*10 | IGHJ4*02 | IGKV1-6*01 | IGKJ3*01 |
| PPB-1409 | IGHV3-23*04 | IGHJ2*01 | IGKV3-15*01 | IGKJ3*01 |
| PPB-1410 | IGHV3-23*04 | IGHJ4*02 | IGKV3-15*01 | IGKJ4*01 |
| PPB-1412 | IGHV3-23*04 | IGHJ4*02 | IGKV3D-15*d01 | IGKJ3*01 |
| PPB-1413 | IGHV2-5*10 | IGHJ4*02 | IGKV1-17*01 | IGKJ1*01 |
| PPB-1414 | IGHV3-23*04 | IGHJ4*02 | IGKV3-15*01 | IGKJ4*01 |
| PPB-1415 | IGHV3-23*04 | IGHJ2*01 | IGKV3-15*01 | IGKJ4*01 |
| PPB-1416 | IGHV3-23*04 | IGHJ2*01 | IGKV3-15*01 | IGKJ4*01 |
| PPB-1540 | IGHV3-9*01 | IGHJ3*02 | IGKV1-17*01 | IGKJ4*01 |
| PPB-1548 | IGHV3-30*18 | IGHJ5*02 | IGKV1-6*01 | IGKJ2*04 |
| PPB-1550 | IGHV3-30*18 | IGHJ5*02 | IGKV1-12*01 | IGKJ2*04 |
| PPB-1042 | IGHV2-5*10 | IGHJ4*02 | IGKV1-9*d01 | IGKJ3*01 |
| PPB-1043 | IGHV2-5*10 | IGHJ4*02 | IGKV1D-16*01 | IGKJ3*01 |

### 8. CLAUSES WITH ANTIBODY REFERENCES

In clauses herein relating to an antibody which is defined by antibody sequence, the antibody sequence may be defined by reference to an antibody internal reference number or a SEQ ID NO. Any clauses herein reciting an antibody internal reference number should also be taken as disclosure of the same subject-matter where the sequence defined by the antibody internal reference number is replaced by the corresponding SEQ ID NO.
1. An antibody that specifically binds at the surface of Acinetobacter baumannii bacteria.
2. An antibody according to clause 1, wherein the antibody induces complement activation (e.g. as measured by flow cytometry-based assay).
3. An antibody according to clause 1 or clause 2, that specifically binds to Oxa-23 at the surface of Acinetobacter baumannii bacteria.
4. An antibody according to clause 1 or clause 2, that specifically binds Oxa-23 on outer membrane vesicles (OMVs) of Acinetobacter baumannii bacteria.
5. An antibody according to any one of clauses 1 to 4, wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, and wherein the HCDR3 is the HCDR3 of antibody 1348, 1349, 1540, 1548, or 1550.
6. An antibody according to clause 5, wherein the HCDR3 is the HCDR3 of antibody 1348.
7. An antibody according to clause 5, wherein the HCDR3 is the HCDR3 of antibody 1349.
8. An antibody according to clause 5, wherein the HCDR3 is the HCDR3 of antibody 1540.
9. An antibody according to clause 5, wherein the HCDR3 is the HCDR3 of antibody 1548.
10. An antibody according to clause 5, wherein the HCDR3 is the HCDR3 of antibody 1550.
11. An antibody according to any one of the clauses 1 to 4, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, and wherein the 6 CDRs are those of antibody 1348, 1349, 1540, 1548, or 1550.
12. An antibody according to clause 11, wherein the antibody has the 6 CDRs of antibody 1348.
13. An antibody according to clause 11, wherein the antibody has the 6 CDRs of antibody 1349.
14. An antibody according to clause 11, wherein the antibody has the 6 CDRs of antibody 1540.
15. An antibody according to clause 11, wherein the antibody has the 6 CDRs of antibody 1548.
16. An antibody according to clause 11, wherein the antibody has the 6 CDRs of antibody 1550.
17. An antibody according to any one of clauses 1 to 4, wherein the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody 1348, 1349, 1540, 1548, or 1550, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.
18. An antibody according to clause 17 wherein the antibody comprises a variable heavy (VH) domain sequence of antibody 1348, optionally with 1, 2, 3, 4 or 5 amino acid alterations outside the complementarity determining regions (CDRs), and a variable light (VL) domain sequence of antibody 1348, optionally with 1, 2, 3, 4 or 5 amino acid alterations outside the complementarity determining regions (CDRs).
19. An antibody according to clause 17, wherein the antibody comprises a variable heavy (VH) domain sequence of antibody 1349, optionally with 1, 2, 3, 4 or 5 amino acid alterations outside the complementarity determining regions (CDRs), and a variable light (VL) domain sequence of antibody 1349, optionally with 1, 2, 3, 4 or 5 amino acid alterations outside the complementarity determining regions (CDRs).
20. An antibody according to clause 17, wherein the antibody comprises a variable heavy (VH) domain sequence of antibody 1540, optionally with 1, 2, 3, 4 or 5 amino acid alterations outside the complementarity determining regions (CDRs), and a variable light (VL) domain sequence of antibody 1349, optionally with 1, 2, 3, 4 or 5 amino acid alterations outside the complementarity determining regions (CDRs).
21. An antibody according to clause 17, wherein the antibody comprises a variable heavy (VH) domain sequence of antibody 1548, optionally with 1, 2, 3, 4 or 5 amino acid alterations outside the complementarity determining regions (CDRs), and a variable light (VL) domain sequence of antibody 1349, optionally with 1, 2, 3, 4 or 5 amino acid alterations outside the complementarity determining regions (CDRs).
22. An antibody according to clause 17, wherein the antibody comprises a variable heavy (VH) domain sequence of antibody 1550, optionally with 1, 2, 3, 4 or 5 amino acid alterations outside the complementarity determining regions (CDRs), and a variable light (VL) domain sequence of antibody 1349, optionally with 1, 2, 3, 4 or 5 amino acid alterations outside the complementarity determining regions (CDRs).
23. An antibody according to any one of clauses 1 to 4, wherein the antibody comprises a variable heavy (VH) domain sequence and a variable light (VL) domain sequence and wherein the variable heavy (VH) domain and variable light (VL) domain sequences respectively comprise a sequence having at least 90%, at least 95%, at least 98%, or at least 99% identity to the variable heavy (VH) and variable light (VL) domain sequences of antibody 1348, 1349, 1540, 1548, or 1550, provided that the antibody has the CDRs of antibody 1348, 1349, 1540, 1548, or 1550, respectively.
24. An antibody according to clause 23, wherein the variable heavy (VH) domain sequence comprises a sequence having at least 90%, at least 95%, at least 98%, or at least 99% identity to the VH domain sequence of antibody 1348 and the variable light (VL) domain sequence comprises a sequence having at least 90%, at least 95%, at least 98%, or at least 99% identity to the VL domain sequence of antibody 1348, provided that the antibody has the CDRs of antibody 1348.
25. An antibody according to clause 23, wherein the variable heavy (VH) domain sequence comprises a sequence having at least 90%, at least 95%, at least 98%, or at least 99% identity to the VH domain sequence of antibody 1349 and the variable light (VL) domain sequence comprises a sequence having at least 90%, at least 95%, at least 98%, or at least 99% identity to the VL domain sequence of antibody 1349, provided that the antibody has the CDRs of antibody 1349.
26. An antibody according to clause 23, wherein the variable heavy (VH) domain sequence comprises a sequence having at least 90%, at least 95%, at least 98%, or at least 99% identity to the VH domain sequence of antibody 1349 and the variable light (VL) domain sequence comprises a sequence having at least 90%, at least 95%, at least 98%, or at least 99% identity to the VL domain sequence of antibody 1540, provided that the antibody has the CDRs of antibody 1540.
27. An antibody according to clause 23, wherein the variable heavy (VH) domain sequence comprises a sequence having at least 90%, at least 95%, at least 98%, or at least 99% identity to the VH domain sequence of antibody 1349 and the variable light (VL) domain sequence comprises a sequence having at least 90%, at least 95%, at least 98%, or at least 99% identity to the VL domain sequence of antibody 1548, provided that the antibody has the CDRs of antibody 1548.
28. An antibody according to clause 23, wherein the variable heavy (VH) domain sequence comprises a sequence having at least 90%, at least 95%, at least 98%, or at least 99% identity to the VH domain sequence of antibody 1349 and the variable light (VL) domain sequence comprises a sequence having at least 90%, at least 95%, at least 98%, or at least 99% identity to the VL domain sequence of antibody 1550, provided that the antibody has the CDRs of antibody 1550.
29. An antibody according to any one of clauses 1 to 4, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody 1348, 1349, 1540, 1548, or 1550.
30. An antibody according to clause 29, wherein the antibody comprises the VH and VL domain sequences of antibody 1348.
31. An antibody according to clause 29, wherein the antibody comprises the VH and VL domain sequences of antibody 1349.
32. An antibody according to clause 29, wherein the antibody comprises the VH and VL domain sequences of antibody 1540.
33. An antibody according to clause 29, wherein the antibody comprises the VH and VL domain sequences of antibody 1548.
34. An antibody according to clause 29, wherein the antibody comprises the VH and VL domain sequences of antibody 1550.
35. An antibody that binds to the same epitope on Oxa-23 as an antibody according to any one of clauses 1 to 34.
36. An antibody capable of competing for binding to Oxa-23 with an antibody according to any one of clauses 1 to 34.
37. An antibody according to clause 1 or clause 2, that specifically binds OCL1 lipooligosaccharide (LOS) at the surface of Acinetobacter baumannii bacteria.
38. An antibody according to clause 1 or clause 2, that specifically binds OCL1 LOS on outer membrane vesicles (OMVs) of Acinetobacter baumannii bacteria.
39. An antibody according to clause 37 or clause 38, wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, and wherein the HCDR3 is the HCDR3 of antibody 1042, 1043, 1403, 1405, 1407, 1408, or 1413.
40. An antibody according to clause 39, wherein the HCDR3 is the HCDR3 of antibody 1042.
41. An antibody according to clause 39, wherein the HCDR3 is the HCDR3 of antibody 1043.
42. An antibody according to clause 39, wherein the HCDR3 is the HCDR3 of antibody 1403.
43. An antibody according to clause 39, wherein the HCDR3 is the HCDR3 of antibody 1405.
44. An antibody according to clause 39, wherein the HCDR3 is the HCDR3 of antibody 1407.
45. An antibody according to clause 39, wherein the HCDR3 is the HCDR3 of antibody 1408.
46. An antibody according to clause 39, wherein the HCDR3 is the HCDR3 of antibody 1413.
47. An antibody according to clause 37 or clause 38, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, and wherein the 6 CDRs are those of antibody 1042, 1043, 1403, 1405, 1407, 1408, or 1413.
48. An antibody according to clause 47, wherein the antibody has the 6 CDRs of antibody 1042.
49. An antibody according to clause 47, wherein the e antibody has the 6 CDRs of antibody 1043.
50. An antibody according to clause 47, wherein the antibody has the 6 CDRs of antibody 1403.
51. An antibody according to clause 47, wherein the antibody has the 6 CDRs of antibody 1405.
52. An antibody according to clause 47, wherein the antibody has the 6 CDRs of antibody 1407.
53. An antibody according to clause 47, wherein the antibody has the 6 CDRs of antibody 1408.
54. An antibody according to clause 47, wherein the antibody has the 6 CDRs of antibody 1413.
55. An antibody according to clause 37 or clause 38, wherein the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody 1042, 1043, 1403, 1405, 1407, 1408, or 1413, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.
56. An antibody according to clause 55, wherein the antibody comprises a variable heavy (VH) domain sequence of antibody 1042, optionally with 1, 2, 3, 4 or 5 amino acid alterations outside the complementarity determining regions (CDRs), and a variable light (VL) domain sequence of antibody 1042, optionally with 1, 2, 3, 4 or 5 amino acid alterations outside the complementarity determining regions (CDRs).
57. An antibody according to clause 55, wherein the antibody comprises a variable heavy (VH) domain sequence of antibody 1043, optionally with 1, 2, 3, 4 or 5 amino acid alterations outside the complementarity determining regions (CDRs), and a variable light (VL) domain sequence of antibody 1043, optionally with 1, 2, 3, 4 or 5 amino acid alterations outside the complementarity determining regions (CDRs).
58. An antibody according to clause 55, wherein the antibody comprises a variable heavy (VH) domain sequence of antibody 1403, optionally with 1, 2, 3, 4 or 5 amino acid alterations outside the complementarity determining regions (CDRs), and a variable light (VL) domain sequence of antibody 1403, optionally with 1, 2, 3, 4 or 5 amino acid alterations outside the complementarity determining regions (CDRs).
59. An antibody according to clause 55, wherein the antibody comprises a variable heavy (VH) domain sequence of antibody 1405, optionally with 1, 2, 3, 4 or 5 amino acid alterations outside the complementarity determining regions (CDRs), and a variable light (VL) domain sequence of antibody 1405, optionally with 1, 2, 3, 4 or 5 amino acid alterations outside the complementarity determining regions (CDRs).
60. An antibody according to clause 55, wherein the antibody comprises a variable heavy (VH) domain sequence of antibody 1407, optionally with 1, 2, 3, 4 or 5 amino acid alterations outside the complementarity determining regions (CDRs), and a variable light (VL) domain sequence of antibody 1407, optionally with 1, 2, 3, 4 or 5 amino acid alterations outside the complementarity determining regions (CDRs).
61. An antibody according to clause 55, wherein the antibody comprises a variable heavy (VH) domain sequence of antibody 1408, optionally with 1, 2, 3, 4 or 5 amino acid alterations outside the complementarity determining regions (CDRs), and a variable light (VL) domain sequence of antibody 1408, optionally with 1, 2, 3, 4 or 5 amino acid alterations outside the complementarity determining regions (CDRs).
62. An antibody according to clause 55, wherein the antibody comprises a variable heavy (VH) domain sequence of antibody 1413, optionally with 1, 2, 3, 4 or 5 amino acid alterations outside the complementarity determining regions (CDRs), and a variable light (VL) domain sequence of antibody 1413, optionally with 1, 2, 3, 4 or 5 amino acid alterations outside the complementarity determining regions (CDRs).
63. An antibody according to clause 37 or clause 38, wherein the antibody comprises a variable heavy (VH) domain sequence and a variable light (VL) domain sequence and wherein the variable heavy (VH) domain and variable light (VL) domain sequences respectively comprise a sequence having at least 90%, at least 95%, at least 98%, or at least 99% identity to the variable heavy (VH) and variable light (VL) domain sequences of antibody 1042, 1043, 1403, 1405, 1407, 1408, or 1413, provided that the antibody has the 6 CDRs of antibody 1042, 1043, 1403, 1405, 1407, 1408, or 1413, respectively.
64. An antibody according to clause 63, wherein the variable heavy (VH) domain sequence comprises a sequence having at least 90%, at least 95%, at least 98%, or at least 99% identity to the VH domain sequence of antibody 1042 and the variable light (VL) domain sequence comprises a sequence having at least 90%, at least 95%, at least 98%, or at least 99% identity to the VL domain sequence of antibody 1042, provided that the antibody has the 6 CDRs of antibody 1042.
65. An antibody according to clause 63, wherein the variable heavy (VH) domain sequence comprises a sequence having at least 90%, at least 95%, at least 98%, or at least 99% identity to the VH domain sequence of antibody 1043 and the variable light (VL) domain sequence comprises a sequence having at least 90%, at least 95%, at least 98%, or at least 99% identity to the VL domain sequence of antibody 1043, provided that the antibody has the 6 CDRs of antibody 1043.
66. An antibody according to clause 63, wherein the variable heavy (VH) domain sequence comprises a sequence having at least 90%, at least 95%, at least 98%, or at least 99% identity to the VH domain sequence of antibody 1403 and the variable light (VL) domain sequence comprises a sequence having at least 90%, at least 95%, at least 98%, or at least 99% identity to the VL domain sequence of antibody 1403, provided that the antibody has the 6 CDRs of antibody 1403.
67. An antibody according to clause 63, wherein the variable heavy (VH) domain sequence comprises a sequence having at least 90%, at least 95%, at least 98%, or at least 99% identity to the VH domain sequence of antibody 1405 and the variable light (VL) domain sequence comprises a sequence having at least 90%, at least 95%, at least 98%, or at least 99% identity to the VL domain sequence of antibody 1405, provided that the antibody has the 6 CDRs of antibody 1405.
68. An antibody according to clause 63, wherein the variable heavy (VH) domain sequence comprises a sequence having at least 90%, at least 95%, at least 98%, or at least 99% identity to the VH domain sequence of antibody 1407 and the variable light (VL) domain sequence comprises a sequence having at least 90%, at least 95%, at least 98%, or at least 99% identity to the VL domain sequence of antibody 1407, provided that the antibody has the 6 CDRs of antibody 1407.
69. An antibody according to clause 63, wherein the variable heavy (VH) domain sequence comprises a sequence having at least 90%, at least 95%, at least 98%, or at least 99% identity to the VH domain sequence of antibody 1408 and the variable light (VL) domain sequence comprises a sequence having at least 90%, at least 95%, at least 98%, or at least 99% identity to the VL domain sequence of antibody 1408, provided that the antibody has the 6 CDRs of antibody 1408.
70. An antibody according to clause 63, wherein the variable heavy (VH) domain sequence comprises a sequence having at least 90%, at least 95%, at least 98%, or at least 99% identity to the VH domain sequence of antibody 1413 and the variable light (VL) domain sequence comprises a sequence having at least 90%, at least 95%, at least 98%, or at least 99% identity to the VL domain sequence of antibody 1413, provided that the antibody has the 6 CDRs of antibody 1413.
71. An antibody according to clause 37 or clause 38, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody 1042, 1043, 1403, 1405, 1407, 1408, or 1413.
72. An antibody according to clause 71, wherein the antibody comprises the VH and VL domain sequences of antibody 1042.
73. An antibody according to clause 71, wherein the antibody comprises the VH and VL domain sequences of antibody 1043.
74. An antibody according to clause 71, wherein the antibody comprises the VH and VL domain sequences of antibody 1403.
75. An antibody according to clause 71, wherein the antibody comprises the VH and VL domain sequences of antibody 1405.
76. An antibody according to clause 71, wherein the antibody comprises the VH and VL domain sequences of antibody 1407.
77. An antibody according to clause 71, wherein the antibody comprises the VH and VL domain sequences of antibody 1408.
78. An antibody according to clause 71, wherein the antibody comprises the VH and VL domain sequences of antibody 1413.
79. An antibody that binds to the same epitope on OCL1 LOS as an antibody according to any one of clauses 37 to 78.
80. An antibody capable of competing for binding to OCL1 LOS with an antibody according to any one of clauses 37 to 78.
81. An antibody according to clause 1 or clause 2, that specifically binds KL49 at the surface of Acinetobacter baumannii bacteria.
82. An antibody according to clause 1 or clause 2, that specifically binds KL49 on outer membrane vesicles (OMVs) of Acinetobacter baumannii bacteria.
83. An antibody according to clause 81 or clause 82, wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, and wherein the HCDR3 is the HCDR3 of antibody 1345, 1347, 1350, 1351, 1363, 1364, 1397, 1398, 1400, 1401, 1402, 1404, 1409, 1410, 1412, 1414, 1415, or 1416.
84. An antibody according to clause 83, wherein the HCDR3 is the HCDR3 of antibody1345.
85. An antibody according to clause 83, wherein the HCDR3 is the HCDR3 of antibody1347.
86. An antibody according to clause 83, wherein the HCDR3 is the HCDR3 of antibody1350.
87. An antibody according to clause 83, wherein the HCDR3 is the HCDR3 of antibody1351.
88. An antibody according to clause 83, wherein the HCDR3 is the HCDR3 of antibody1363.
89. An antibody according to clause 83, wherein the HCDR3 is the HCDR3 of antibody1364.
90. An antibody according to clause 83, wherein the HCDR3 is the HCDR3 of antibody1397.
91. An antibody according to clause 83, wherein the HCDR3 is the HCDR3 of antibody1398.
92. An antibody according to clause 83, wherein the HCDR3 is the HCDR3 of antibody1400.
93. An antibody according to clause 83, wherein the HCDR3 is the HCDR3 of antibody1401.
94. An antibody according to clause 83, wherein the HCDR3 is the HCDR3 of antibody1402.
95. An antibody according to clause 83, wherein the HCDR3 is the HCDR3 of antibody1404.
96. An antibody according to clause 83, wherein the HCDR3 is the HCDR3 of antibody1409.
97. An antibody according to clause 83, wherein the HCDR3 is the HCDR3 of antibody1410.
98. An antibody according to clause 83, wherein the HCDR3 is the HCDR3 of antibody1412.
99. An antibody according to clause 83, wherein the HCDR3 is the HCDR3 of antibody1414.
100. An antibody according to clause 83, wherein the HCDR3 is the HCDR3 of antibody1415.
101. An antibody according to clause 83, wherein the HCDR3 is the HCDR3 of antibody1416.
102. An antibody according to clause 81 or clause 82, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, and wherein the 6 CDRs are those of antibody 1345, 1347, 1350, 1351, 1363, 1364, 1397, 1398, 1400, 1401, 1402, 1404, 1409, 1410, 1412, 1414, 1415, or 1416.
103. An antibody according to clause 102, wherein the antibody has the 6 CDRs of antibody1345.
104. An antibody according to clause 102, wherein the antibody has the 6 CDRs of antibody1347.
105. An antibody according to clause 102, wherein the antibody has the 6 CDRs of antibody1350.
106. An antibody according to clause 102, wherein the antibody has the 6 CDRs of antibody1351.
107. An antibody according to clause 102, wherein the antibody has the 6 CDRs of antibody1363.
108. An antibody according to clause 102, wherein the antibody has the 6 CDRs of antibody1364.
109. An antibody according to clause 102, wherein the antibody has the 6 CDRs of antibody1397.
110. An antibody according to clause 102, wherein the antibody has the 6 CDRs of antibody1398.
111. An antibody according to clause 102, wherein the antibody has the 6 CDRs of antibody1400.
112. An antibody according to clause 102, wherein the antibody has the 6 CDRs of antibody1401.
113. An antibody according to clause 102, wherein the antibody has the 6 CDRs of antibody1402.
114. An antibody according to clause 102, wherein the antibody has the 6 CDRs of antibody1404.
115. An antibody according to clause 102, wherein the antibody has the 6 CDRs of antibody1409.
116. An antibody according to clause 102, wherein the antibody has the 6 CDRs of antibody1410.
117. An antibody according to clause 102, wherein the antibody has the 6 CDRs of antibody1412.
118. An antibody according to clause 102, wherein the antibody has the 6 CDRs of antibody1414.
119. An antibody according to clause 102, wherein the antibody has the 6 CDRs of antibody1415.
120. An antibody according to clause 102, wherein the antibody has the 6 CDRs of antibody1416.
121. An antibody according to clause 81 or clause 82, wherein the antibody comprises a variable heavy (VH) domain sequence and a variable light (VL) domain sequence and wherein the variable heavy (VH) domain and variable light (VL) domain sequences respectively comprise the variable heavy (VH) domain and variable light (VL) domain sequences of antibody 1345, 1347, 1350, 1351, 1363, 1364, 1397, 1398, 1400, 1401, 1402, 1404, 1409, 1410, 1412, 1414, 1415, or 1416, optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.
122. An antibody according to clause 121, wherein the antibody comprises a variable heavy (VH) domain sequence of antibody 1345, optionally with 1, 2, 3, 4 or 5 amino acid alterations outside the complementarity determining regions (CDRs), and a variable light (VL) domain sequence of antibody 1345, optionally with 1, 2, 3, 4 or 5 amino acid alterations outside the complementarity determining regions (CDRs).
123. An antibody according to clause 121, wherein the antibody comprises a variable heavy (VH) domain sequence of antibody 1347, optionally with 1, 2, 3, 4 or 5 amino acid alterations outside the complementarity determining regions (CDRs), and a variable light (VL) domain sequence of antibody 1347, optionally with 1, 2, 3, 4 or 5 amino acid alterations outside the complementarity determining regions (CDRs).
124. An antibody according to clause 121, wherein the antibody comprises a variable heavy (VH) domain sequence of antibody 1350, optionally with 1, 2, 3, 4 or 5 amino acid alterations outside the complementarity determining regions (CDRs), and a variable light (VL) domain sequence of antibody 1350, optionally with 1, 2, 3, 4 or 5 amino acid alterations outside the complementarity determining regions (CDRs).
125. An antibody according to clause 121, wherein the antibody comprises a variable heavy (VH) domain sequence of antibody 1351, optionally with 1, 2, 3, 4 or 5 amino acid alterations outside the complementarity determining regions (CDRs), and a variable light (VL) domain sequence of antibody 1351, optionally with 1, 2, 3, 4 or 5 amino acid alterations outside the complementarity determining regions (CDRs).
126. An antibody according to clause 121, wherein the antibody comprises a variable heavy (VH) domain sequence of antibody 1363, optionally with 1, 2, 3, 4 or 5 amino acid alterations outside the complementarity determining regions (CDRs), and a variable light (VL) domain sequence of antibody 1363, optionally with 1, 2, 3, 4 or 5 amino acid alterations outside the complementarity determining regions (CDRs).
127. An antibody according to clause 121, wherein the antibody comprises a variable heavy (VH) domain sequence of antibody 1364, optionally with 1, 2, 3, 4 or 5 amino acid alterations outside the complementarity determining regions (CDRs), and a variable light (VL) domain sequence of antibody 1364, optionally with 1, 2, 3, 4 or 5 amino acid alterations outside the complementarity determining regions (CDRs).
128. An antibody according to clause 121, wherein the antibody comprises a variable heavy (VH) domain sequence of antibody 1397, optionally with 1, 2, 3, 4 or 5 amino acid alterations outside the complementarity determining regions (CDRs), and a variable light (VL) domain sequence of antibody 1397, optionally with 1, 2, 3, 4 or 5 amino acid alterations outside the complementarity determining regions (CDRs).
129. An antibody according to clause 121, wherein the antibody comprises a variable heavy (VH) domain sequence of antibody 1398, optionally with 1, 2, 3, 4 or 5 amino acid alterations outside the complementarity determining regions (CDRs), and a variable light (VL) domain sequence of antibody 1398, optionally with 1, 2, 3, 4 or 5 amino acid alterations outside the complementarity determining regions (CDRs).
130. An antibody according to clause 121, wherein the antibody comprises a variable heavy (VH) domain sequence of antibody 1400, optionally with 1, 2, 3, 4 or 5 amino acid alterations outside the complementarity determining regions (CDRs), and a variable light (VL) domain sequence of antibody 1400, optionally with 1, 2, 3, 4 or 5 amino acid alterations outside the complementarity determining regions (CDRs).
131. An antibody according to clause 121, wherein the antibody comprises a variable heavy (VH) domain sequence of antibody 1401, optionally with 1, 2, 3, 4 or 5 amino acid alterations outside the complementarity determining regions (CDRs), and a variable light (VL) domain sequence of antibody 1401, optionally with 1, 2, 3, 4 or 5 amino acid alterations outside the complementarity determining regions (CDRs).
132. An antibody according to clause 121, wherein the antibody comprises a variable heavy (VH) domain sequence of antibody 1402, optionally with 1, 2, 3, 4 or 5 amino acid alterations outside the complementarity determining regions (CDRs), and a variable light (VL) domain sequence of antibody 1402, optionally with 1, 2, 3, 4 or 5 amino acid alterations outside the complementarity determining regions (CDRs).
133. An antibody according to clause 121, wherein the antibody comprises a variable heavy (VH) domain sequence of antibody 1404, optionally with 1, 2, 3, 4 or 5 amino acid alterations outside the complementarity determining regions (CDRs), and a variable light (VL) domain sequence of antibody 1404, optionally with 1, 2, 3, 4 or 5 amino acid alterations outside the complementarity determining regions (CDRs).
134. An antibody according to clause 121, wherein the antibody comprises a variable heavy (VH) domain sequence of antibody 1409, optionally with 1, 2, 3, 4 or 5 amino acid alterations outside the complementarity determining regions (CDRs), and a variable light (VL) domain sequence of antibody 1409, optionally with 1, 2, 3, 4 or 5 amino acid alterations outside the complementarity determining regions (CDRs).
135. An antibody according to clause 121, wherein the antibody comprises a variable heavy (VH) domain sequence of antibody 1410, optionally with 1, 2, 3, 4 or 5 amino acid alterations outside the complementarity determining regions (CDRs), and a variable light (VL) domain sequence of antibody 1410, optionally with 1, 2, 3, 4 or 5 amino acid alterations outside the complementarity determining regions (CDRs).
136. An antibody according to clause 121, wherein the antibody comprises a variable heavy (VH) domain sequence of antibody 1412, optionally with 1, 2, 3, 4 or 5 amino acid alterations outside the complementarity determining regions (CDRs), and a variable light (VL) domain sequence of antibody 1412, optionally with 1, 2, 3, 4 or 5 amino acid alterations outside the complementarity determining regions (CDRs).
137. An antibody according to clause 121, wherein the antibody comprises a variable heavy (VH) domain sequence of antibody 1414, optionally with 1, 2, 3, 4 or 5 amino acid alterations outside the complementarity determining regions (CDRs), and a variable light (VL) domain sequence of antibody 1414, optionally with 1, 2, 3, 4 or 5 amino acid alterations outside the complementarity determining regions (CDRs).
138. An antibody according to clause 121, wherein the antibody comprises a variable heavy (VH) domain sequence of antibody 1415, optionally with 1, 2, 3, 4 or 5 amino acid alterations outside the complementarity determining regions (CDRs), and a variable light (VL) domain sequence of antibody 1415, optionally with 1, 2, 3, 4 or 5 amino acid alterations outside the complementarity determining regions (CDRs).
139. An antibody according to clause 121, wherein the antibody comprises a variable heavy (VH) domain sequence of antibody 1416, optionally with 1, 2, 3, 4 or 5 amino acid alterations outside the complementarity determining regions (CDRs), and a variable light (VL) domain sequence of antibody 1416, optionally with 1, 2, 3, 4 or 5 amino acid alterations outside the complementarity determining regions (CDRs).
140. An antibody according to clause 81 or clause 82, wherein the antibody comprises a variable heavy (VH) domain sequence and a variable light (VL) domain sequence and wherein the variable heavy (VH) domain and variable light (VL) domain sequences respectively comprise a sequence having at least 90%, at least 95%, at least 98%, or at least 99% identity to the variable heavy (VH) and variable light (VL) domain sequences of antibody 1345, 1347, 1350, 1351, 1363, 1364, 1397, 1398, 1400, 1401, 1402, 1404, 1409, 1410, 1412, 1414, 1415, or 1416, provided that the antibody has the 6 CDRs of antibody 1345, 1347, 1350, 1351, 1363, 1364, 1397, 1398, 1400, 1401, 1402, 1404, 1409, 1410, 1412, 1414, 1415, or 1416, respectively.
141. An antibody according to clause 140, wherein the variable heavy (VH) domain sequence comprises a sequence having at least 90%, at least 95%, at least 98%, or at least 99% identity to the VH domain sequence of antibody 1345 and the variable light (VL) domain sequence comprises a sequence having at least 90%, at least 95%, at least 98%, or at least 99% identity to the VL domain sequence of antibody 1345, provided that the antibody has the 6 CDRs of antibody 1345.
142. An antibody according to clause 140, wherein the variable heavy (VH) domain sequence comprises a sequence having at least 90%, at least 95%, at least 98%, or at least 99% identity to the VH domain sequence of antibody 1347 and the variable light (VL) domain sequence comprises a sequence having at least 90%, at least 95%, at least 98%, or at least 99% identity to the VL domain sequence of antibody 1347, provided that the antibody has the 6 CDRs of antibody 1347.
143. An antibody according to clause 140, wherein the variable heavy (VH) domain sequence comprises a sequence having at least 90%, at least 95%, at least 98%, or at least 99% identity to the VH domain sequence of antibody 1350 and the variable light (VL) domain sequence comprises a sequence having at least 90%, at least 95%, at least 98%, or at least 99% identity to the VL domain sequence of antibody 1350, provided that the antibody has the 6 CDRs of antibody 1350.
144. An antibody according to clause 140, wherein the variable heavy (VH) domain sequence comprises a sequence having at least 90%, at least 95%, at least 98%, or at least 99%/identity to the VH domain sequence of antibody 1351 and the variable light (VL) domain sequence comprises a sequence having at least 90%, at least 95%, at least 98%, or at least 99% identity to the VL domain sequence of antibody 1351, provided that the antibody has the 6 CDRs of antibody 1351.
145. An antibody according to clause 140, wherein the variable heavy (VH) domain sequence comprises a sequence having at least 90%, at least 95%, at least 98%, or at least 99% identity to the VH domain sequence of antibody 1363 and the variable light (VL) domain sequence comprises a sequence having at least 90%, at least 95%, at least 98%, or at least 99% identity to the VL domain sequence of antibody 1363, provided that the antibody has the 6 CDRs of antibody 1363.
146. An antibody according to clause 140, wherein the variable heavy (VH) domain sequence comprises a sequence having at least 90%, at least 95%, at least 98%, or at least 99% identity to the VH domain sequence of antibody 1364 and the variable light (VL) domain sequence comprises a sequence having at least 90%, at least 95%, at least 98%, or at least 99% identity to the VL domain sequence of antibody 1364, provided that the antibody has the 6 CDRs of antibody 1364.
147. An antibody according to clause 140, wherein the variable heavy (VH) domain sequence comprises a sequence having at least 90%, at least 95%, at least 98%, or at least 99% identity to the VH domain sequence of antibody 1397 and the variable light (VL) domain sequence comprises a sequence having at least 90%, at least 95%, at least 98%, or at least 99% identity to the VL domain sequence of antibody 1397, provided that the antibody has the 6 CDRs of antibody 1397.
148. An antibody according to clause 140, wherein the variable heavy (VH) domain sequence comprises a sequence having at least 90%, at least 95%, at least 98%, or at least 99% identity to the VH domain sequence of antibody 1398 and the variable light (VL) domain sequence comprises a sequence having at least 90%, at least 95%, at least 98%, or at least 99% identity to the VL domain sequence of antibody 1398, provided that the antibody has the 6 CDRs of antibody 1398.
149. An antibody according to clause 140, wherein the variable heavy (VH) domain sequence comprises a sequence having at least 90%, at least 95%, at least 98%, or at least 99% identity to the VH domain sequence of antibody 1400 and the variable light (VL) domain sequence comprises a sequence having at least 90%, at least 95%, at least 98%, or at least 99% identity to the VL domain sequence of antibody 1400, provided that the antibody has the 6 CDRs of antibody 1400.
150. An antibody according to clause 140, wherein the variable heavy (VH) domain sequence comprises a sequence having at least 90%, at least 95%, at least 98%, or at least 99% identity to the VH domain sequence of antibody 1401 and the variable light (VL) domain sequence comprises a sequence having at least 90%, at least 95%, at least 98%, or at least 99% identity to the VL domain sequence of antibody 1401, provided that the antibody has the 6 CDRs of antibody 1401.
151. An antibody according to clause 140, wherein the variable heavy (VH) domain sequence comprises a sequence having at least 90%, at least 95%, at least 98%, or at least 99% identity to the VH domain sequence of antibody 1402 and the variable light (VL) domain sequence comprises a sequence having at least 90%, at least 95%, at least 98%, or at least 99% identity to the VL domain sequence of antibody 1402, provided that the antibody has the 6 CDRs of antibody 1402.
152. An antibody according to clause 140, wherein the variable heavy (VH) domain sequence comprises a sequence having at least 90%, at least 95%, at least 98%, or at least 99% identity to the VH domain sequence of antibody 1404 and the variable light (VL) domain sequence comprises a sequence having at least 90%, at least 95%, at least 98%, or at least 99% identity to the VL domain sequence of antibody 1404, provided that the antibody has the 6 CDRs of antibody 1404.
153. An antibody according to clause 140, wherein the variable heavy (VH) domain sequence comprises a sequence having at least 90%, at least 95%, at least 98%, or at least 99% identity to the VH domain sequence of antibody 1409 and the variable light (VL) domain sequence comprises a sequence having at least 90%, at least 95%, at least 98%, or at least 99% identity to the VL domain sequence of antibody 1409, provided that the antibody has the 6 CDRs of antibody 1409.
154. An antibody according to clause 140, wherein the variable heavy (VH) domain sequence comprises a sequence having at least 90%, at least 95%, at least 98%, or at least 99% identity to the VH domain sequence of antibody 1410 and the variable light (VL) domain sequence comprises a sequence having at least 90%, at least 95%, at least 98%, or at least 99% identity to the VL domain sequence of antibody 1410, provided that the antibody has the 6 CDRs of antibody 1410.
155. An antibody according to clause 140, wherein the variable heavy (VH) domain sequence comprises a sequence having at least 90%, at least 95%, at least 98%, or at least 99% identity to the VH domain sequence of antibody 1412 and the variable light (VL) domain sequence comprises a sequence having at least 90%, at least 95%, at least 98%, or at least 99% identity to the VL domain sequence of antibody 1412, provided that the antibody has the 6 CDRs of antibody 1412.
156. An antibody according to clause 140, wherein the variable heavy (VH) domain sequence comprises a sequence having at least 90%, at least 95%, at least 98%, or at least 99% identity to the VH domain sequence of antibody 1414 and the variable light (VL) domain sequence comprises a sequence having at least 90%, at least 95%, at least 98%, or at least 99% identity to the VL domain sequence of antibody 1414, provided that the antibody has the 6 CDRs of antibody 1414.
157. An antibody according to clause 140, wherein the variable heavy (VH) domain sequence comprises a sequence having at least 90%, at least 95%, at least 98%, or at least 99% identity to the VH domain sequence of antibody 1415 and the variable light (VL) domain sequence comprises a sequence having at least 90%, at least 95%, at least 98%, or at least 99% identity to the VL domain sequence of antibody 1415, provided that the antibody has the 6 CDRs of antibody 1415.
158. An antibody according to clause 140, wherein the variable heavy (VH) domain sequence comprises a sequence having at least 90%, at least 95%, at least 98%, or at least 99% identity to the VH domain sequence of antibody 1416 and the variable light (VL) domain sequence comprises a sequence having at least 90%, at least 95%, at least 98%, or at least 99% identity to the VL domain sequence of antibody 1416, provided that the antibody has the 6 CDRs of antibody 1416.
159. An antibody according to clause 81 or clause 82, wherein the antibody comprises a variable heavy (VH) domain sequence and a variable light (VL) domain sequence and wherein the variable heavy (VH) domain and variable light (VL) domain sequences respectively comprise the variable heavy (VH) and variable light (VL) domain sequences of antibody 1345, 1347, 1350, 1351, 1363, 1364, 1397, 1398, 1400, 1401, 1402, 1404, 1409, 1410, 1412, 1414, 1415, or 1416.
160. An antibody according to clause 159, wherein the antibody comprises the VH and VL domain sequences of antibody 1345.
161. An antibody according to clause 159, wherein the antibody comprises the VH and VL domain sequences of antibody 1347.
162. An antibody according to clause 159, wherein the antibody comprises the VH and VL domain sequences of antibody 1350.
163. An antibody according to clause 159, wherein the antibody comprises the VH and VL domain sequences of antibody 1351.
164. An antibody according to clause 159, wherein the antibody comprises the VH and VL domain sequences of antibody 1363.
165. An antibody according to clause 159, wherein the antibody comprises the VH and VL domain sequences of antibody 1364.
166. An antibody according to clause 159, wherein the antibody comprises the VH and VL domain sequences of antibody 1397.
167. An antibody according to clause 159, wherein the antibody comprises the VH and VL domain sequences of antibody 1398.
168. An antibody according to clause 159, wherein the antibody comprises the VH and VL domain sequences of antibody 1400.
169. An antibody according to clause 159, wherein the antibody comprises the VH and VL domain sequences of antibody 1401.
170. An antibody according to clause 159, wherein the antibody comprises the VH and VL domain sequences of antibody 1402.
171. An antibody according to clause 159, wherein the antibody comprises the VH and VL domain sequences of antibody 1404.
172. An antibody according to clause 159, wherein the antibody comprises the VH and VL domain sequences of antibody 1409.
173. An antibody according to clause 159, wherein the antibody comprises the VH and VL domain sequences of antibody 1410.
174. An antibody according to clause 159, wherein the antibody comprises the VH and VL domain sequences of antibody 1412.
175. An antibody according to clause 159, wherein the antibody comprises the VH and VL domain sequences of antibody 1414.
176. An antibody according to clause 159, wherein the antibody comprises the VH and VL domain sequences of antibody 1415.
177. An antibody according to clause 159, wherein the antibody comprises the VH and VL domain sequences of antibody 1416.
178. An antibody that binds to the same epitope on KL49 as an antibody according to any one of clauses 81 to 177
179. An antibody capable of competing for binding to KL49 with any an antibody according to any one of clauses. 81 to 177.
180. An antibody according to any one of the preceding clauses, wherein the antibody shows complement-dependant cytotoxic activity (CDC) activity.
181. An antibody according to any one of the preceding clauses, wherein the antibody is a human IgG1 or human IgG4.
182. An antibody according to clause 181, wherein the antibody is a human IgG1, optionally wherein the antibody comprises a constant region sequence of SEQ ID NO: 418.
183. An antibody according to clause 181, wherein the antibody is a human IgG4, optionally wherein the antibody comprises a constant region sequence of SEQ ID NO: 436.
184. An antibody according to any one of clauses 1 to 180, wherein the antibody is a human IgA1 (e.g., comprising a constant region sequence SEQ ID NO: 484) or human IgA2 (e.g., comprising a constant region sequence SEQ ID NO: 485).
185. An antibody according to any one of clauses 1 to 184, wherein the antibody comprises kappa (κ) light chain constant regions, preferably constant domain sequence SEQ ID NO: 448.
186. A nucleic acid sequence comprising a sequence that encodes a VH domain and/or an VL domain of an antibody according to any preceding clause.
187. A nucleic acid sequence comprising a sequence that encodes the heavy chain and/or the light chain of an antibody according to any preceding clause.
188. A vector comprising a nucleic acid according to clause 186 or clause 187, optionally wherein the vector is a CHO vector.
189. A host cell comprising a nucleic acid according to clause 186 or clause 187, or a vector according to clause 188.
190. A pharmaceutical composition comprising (i) an isolated nucleic acid encoding an antibody according to any one of clauses 1 to 185, or (ii) a nucleic acid according clause 186 or clause 187, and a pharmaceutically acceptable excipient.
191. A pharmaceutical composition comprising an antibody according to any one of clauses 1 to 185, and a pharmaceutically acceptable excipient.
192. A pharmaceutical composition according to clause 191, wherein the pharmaceutical composition is formulated for intravenous, intramuscular, or subcutaneous administration.
193. A pharmaceutical composition according to clause 191 or clause 192, wherein the pharmaceutical composition further comprises at least one further therapeutic agent.
194. A pharmaceutical composition according to clause 193, wherein the further therapeutic agent is at least one, preferably one or two, further antibodies.
195. A pharmaceutical composition according to clause 193, wherein the further therapeutic agent is carbapenem.
196. A pharmaceutical composition according to clause 193, wherein the further therapeutic agent is colistin.
197. A kit comprising an antibody according to any one of clause 1 to 185.
198. A kit comprising a pharmaceutical composition according to any one of clauses 190 to 196.
199. A kit according to clause 198, wherein the kit further comprises a label or instructions for use to prevent and/or treat a bacterial infection caused by Acinetobacter baumannii in a human; optionally wherein the label or instructions comprise a marketing authorisation number (e.g., an FDA or EMA authorisation number); optionally wherein the kit comprises an IV or injection device that comprises the antibody.
200. A kit according to clause 198 or clause 199, wherein the antibody is contained in a sealed container.
201. An antibody according to any one of clauses 1 to 185, or a composition according to any one of clauses 190 to 196, or a nucleic acid according to clause 186 or clause 187, for use as a medicament.
202. An antibody according to any one of clauses 1 to 185, or a composition according to any one of clauses 190 to 1%, or a nucleic acid according to clause 186 or clause 187, for use in a method of treating a bacterial infection caused by Acinetobacter baumannii, said method comprising administering the antibody, composition, or nucleic acid to a patient.
203. An antibody according to any one of clauses 1 to 185, or a composition according to any one of clauses 190 to 196, or a nucleic acid according to clause 186 or clause 187, for use in a method of preventing a bacterial infection caused by Acinetobacter baumannii, said method comprising administering the antibody, composition, or nucleic acid to a patient.
204. An antibody according to any one of clauses 1 to 185, or a composition according to any one of clauses 190 to 196, or a nucleic acid according to clause 186 or clause 187, for use according to clause 202 or clause 203, wherein the presence or absence of Oxa-23 has been determined in a sample from the patient prior to administering the antibody, composition, or nucleic acid to the patient.
205. An antibody according to any one of clauses 1 to 185, or a composition according to any one of clauses 190 to 196, or a nucleic acid according to clause 186 or clause 187, for use according to clause 202, clause 203 or clause 204, wherein the presence or absence of OCL1 LOS has been determined in a sample from the patient prior to administering the antibody, composition, or nucleic acid to the patient.
206. An antibody according to any one of clauses 1 to 185, or a composition according to any one of clauses 190 to 196, or a nucleic acid according to clause 186 or clause 187, for use according to any one of clauses 202 to 205, wherein the presence or absence of KL49 has been determined in a sample from the patient prior to administering the antibody, composition, or nucleic acid to the patient.
207. Use of an antibody according to any one of clauses 1 to 185, or a composition according to any one of clauses 190 to 196, or a nucleic acid according to clause 186 or clause 187, in the manufacture of a medicament for use in a method of treating a bacterial infection caused by Acinetobacter baumannii in a patient.
208. Use of an antibody according to any one of clauses 1 to 185, or a composition according to any one of clauses 190 to 196, or a nucleic acid according to clause 186 or clause 187, in the manufacture of a medicament for use in a method of preventing a bacterial infection caused by Acinetobacter baumannii in a patient.
209. A method of treating a bacterial infection caused by Acinetobacter baumannii in a patient comprising administering to said patient a therapeutically effective amount of an antibody according to any one of clauses 1 to 185, or a composition according to any one of clauses 190 to 196, or a nucleic acid according clause 186 or clause 187.
210. A method of preventing a bacterial infection caused by Acinetobacter baumannii in a patient comprising administering to said patient a therapeutically effective amount of an antibody according to any one of clauses 1 to 185, or a composition according to any one of clauses 190 to 196, or a nucleic acid according to clause 186 or clause 187.
211. An antibody, composition, or nucleic acid for use according to any one of clauses 202 to 206, use according to clause 207 or clause 208, or a method according to clause 209 or clause 210, wherein the bacterial infection caused by Acinetobacter baumannii is a nosocomial bacterial infection caused by Acinetobacter baumannii.
212. An antibody, composition, or nucleic acid for use according to any one of clauses 202 to 206, use according to clause 207 or clause 208, or a method according to clause 209 or clause 210, wherein the patient has a lower respiratory tract infection, for example pneumonia.
213. An antibody, composition, or nucleic acid for use according to any one of clauses 202 to 206, use according to clause 207 or clause 208, or a method according to clause 209 or clause 210, wherein the patient has sepsis.
214. An antibody, composition, or nucleic acid for use according to any one of clauses 202 to 206, use according to clause 207 or clause 208, or a method according to clause 209 or clause 210, wherein the patient has bacteremia.
215. An antibody, composition, or nucleic acid for use according to any one of clauses 202 to 206, use according to clause 207 or clause 208, or a method according to clause 209 or clause 210, wherein the method further comprises administering at least one further therapeutic agent.
216. An antibody, composition, or nucleic acid for use according to clause 215, use according to clause 215, or a method according to clause 215, wherein the administration of the further therapeutic agent is simultaneous, separate or sequential.
217. An antibody, composition, or nucleic acid for use according to clause 215 or clause 216, use according to clause 215 or clause 216, or a method according to clause 215 or clause 216, wherein the further therapeutic agent is at least one, preferably one or two, further antibodies.
218. An antibody, composition, or nucleic acid for use according to any one of clauses 215 to 217, use according to any one of clauses 215 to 217, or a method according to any one of clauses 215 to 217, wherein the at least one further therapeutic agent is carbapenem.
219. An antibody, composition, or nucleic acid for use according to any one of clauses 215 to 217, use according to any one of clauses 215 to 217, or a method according to any one of clauses 215 to 217, wherein the at least one further therapeutic agent is colistin.
220. Use of an antibody according to any one of clauses 1 to 185, for determining the presence or absence of Acinetobacter baumannii in a sample.
221. A method of determining the presence or absence of Acinetobacter baumannii in a sample comprising contacting the sample with an antibody according to any one of 1 to 185; and testing for binding between the antibody and Acinetobacter baumannii in the sample; wherein detection of binding indicates the presence of Acinetobacter baumannii in the sample and wherein absence of binding indicates the absence of Acinetobacter baumannii in the sample.
222. Use of an antibody according to any one of clauses 1 to 36 for determining the presence or absence of Oxa-23 in a sample, optionally wherein determining the presence or absence of Oxa-23 in a sample is used for determining a treatment protocol in a patient.
223. Use of an antibody according to any one of clauses 1, 2, or 37 to 80 for determining the presence or absence of OCL1 LOS in a sample, optionally wherein determining the presence or absence of OCL1 LOS in a sample is used for determining a treatment protocol in a patient.
224. Use of an antibody according to any one of clauses 1, 2, or 81 to 179 for determining the presence or absence of KL49 in a sample, optionally wherein determining the presence or absence of KL49 in a sample is used for determining a treatment protocol in a patient.
225. Use according to any one of clauses 222 to 224, wherein the antibody is conjugated to a detectable label.
226. Use according to any one of clauses 222 to 225, wherein the sample has been obtained from a human who has been or is suspected of having been infected with Acinetobacter baumannii.
227. Use according to clause 226, wherein the sample has been obtained from a human who has been or is suspected of having been infected with Acinetobacter baumannii who exhibits one or more symptoms of a bacterial infection.
228. Use according to any one of clauses 222 to 227, wherein the sample is a serum, plasma, or whole blood sample, an oral or nasal swab, urine, faeces, or cerebrospinal fluid (CFS), or wherein the sample is from any suspected Acinetobacter baumannii infected organ or tissue.
229. A diagnostic kit comprising an antibody according to any one of clauses 1 to 185, and optionally one or more buffering solutions.
230. A diagnostic kit according to clause 229, wherein the kit comprises a first reagent comprising the antibody according to any one of clauses 1 to 185, and a second reagent comprising a detector molecule that binds to the first reagent.
231. A diagnostic kit according to clause 230, wherein the detector molecule is an antibody that comprises or is conjugated to a detectable label.

### 9. CLAUSES WITH SEQ ID NOS

1. An antibody that specifically binds at the surface of Acinetobacter baumannii bacteria.
2. An antibody according to clause 1, wherein the antibody induces complement activation (e.g. as measured by flow cytometry-based assay).
3. An antibody according to clause 1 or clause 2, that specifically binds to Oxa-23 at the surface of Acinetobacter baumannii bacteria.
4. An antibody according to clause 1 or clause 2, that specifically binds Oxa-23 on outer membrane vesicles (OMVs) of Acinetobacter baumannii bacteria.
5. An antibody according to any one of clauses 1 to 4, wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, and wherein the HCDR3 is selected from the group consisting of SEQ ID NO: 24, SEQ ID NO: 34, SEQ ID NO: 174, and SEQ ID NO: 182.
6. An antibody according to clause 5, wherein the HCDR3 is SEQ ID NO: 24.
7. An antibody according to clause 5, wherein the HCDR3 is SEQ ID NO: 34.
8. An antibody according to clause 5, wherein the HCDR3 is SEQ ID NO: 24.
9. An antibody according to clause 5, wherein the HCDR3 is SEQ ID NO: 174.
10. An antibody according to clause 5, wherein the HCDR3 is SEQ ID NO: 182.
11. An antibody according to any one of the clauses 1 to 4, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, and wherein:
   (i) HCDR1 consists of SEQ ID NO: 22, HCDR2 consists of SEQ ID NO: 23, HCDR3 consists of SEQ ID NO: 24, LCDR1 consists of SEQ ID NO: 27, LCDR2 consists of SEQ ID NO: 28, and LCDR3 consists of SEQ ID NO: 29,
   (ii) HCDR1 consists of SEQ ID NO: 32, HCDR2 consists of SEQ ID NO: 33, HCDR3 consists of SEQ ID NO: 34, LCDR1 consists of SEQ ID NO: 37, LCDR2 consists of SEQ ID NO: 28, and LCDR3 consists of SEQ ID NO: 38,
   (iii) HCDR1 consists of SEQ ID NO: 3, HCDR2 consists of SEQ ID NO: 173, HCDR3 consists of SEQ ID NO: 174, LCDR1 consists of SEQ ID NO: 149, LCDR2 consists of SEQ ID NO: 177, and LCDR3 consists of SEQ ID NO: 178, or
   (iv) HCDR1 consists of SEQ ID NO: 181, HCDR2 consists of SEQ ID NO: 173, HCDR3 consists of SEQ ID NO: 182, LCDR1 consists of SEQ ID NO: 185, LCDR2 consists of SEQ ID NO: 28, and LCDR3 consists of SEQ ID NO: 186.
12. An antibody according to clause 11, wherein HCDR1 consists of SEQ ID NO: 22, HCDR2 consists of SEQ ID NO: 23, HCDR3 consists of SEQ ID NO: 24, LCDR1 consists of SEQ ID NO: 27, LCDR2 consists of SEQ ID NO: 28, and LCDR3 consists of SEQ ID NO: 29.
13. An antibody according to clause 11, wherein HCDR1 consists of SEQ ID NO: 32, HCDR2 consists of SEQ ID NO: 33, HCDR3 consists of SEQ ID NO: 34, LCDR1 consists of SEQ ID NO: 37, LCDR2 consists of SEQ ID NO: 28, and LCDR3 consists of SEQ ID NO: 38.
14. An antibody according to clause 11, wherein HCDR1 consists of SEQ ID NO: 22, HCDR2 consists of SEQ ID NO: 23, HCDR3 consists of SEQ ID NO: 24, LCDR1 consists of SEQ ID NO: 27, LCDR2 consists of SEQ ID NO: 28, and LCDR3 consists of SEQ ID NO: 29.
15. An antibody according to clause 11, wherein HCDR1 consists of SEQ ID NO: 3, HCDR2 consists of SEQ ID NO: 173, HCDR3 consists of SEQ ID NO: 174, LCDR1 consists of SEQ ID NO: 149, LCDR2 consists of SEQ ID NO: 177, and LCDR3 consists of SEQ ID NO: 178.
16. An antibody according to clause 11, wherein HCDR1 consists of SEQ ID NO: 181, HCDR2 consists of SEQ ID NO: 173, HCDR3 consists of SEQ ID NO: 182, LCDR1 consists of SEQ ID NO: 185, LCDR2 consists of SEQ ID NO: 28, and LCDR3 consists of SEQ ID NO: 186.
17. An antibody according to any one of clauses 1 to 4, wherein the antibody comprises a variable heavy (VH) domain sequence and a variable light (VL) domain sequence and wherein:
   (i) the variable heavy (VH) domain comprises, preferably consists of SEQ ID NO: 21 and the variable light (VL) domain comprises, preferably consists of SEQ ID NO: 26,
   (ii) the variable heavy (VH) domain comprises, preferably consists of SEQ ID NO: 31 and the variable light (VL) domain comprises, preferably consists of SEQ ID NO:36,
   (iii) the variable heavy (VH) domain comprises, preferably consists of SEQ ID NO: 172 and the variable light (VL) domain comprises, preferably consists of SEQ ID NO:176, or
   (iv) the variable heavy (VH) domain comprises, preferably consists of SEQ ID NO: 180 and the variable light (VL) domain comprises, preferably consists of SEQ ID NO:184,
      optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.
18. An antibody according to clause 17 wherein the antibody comprises a variable heavy (VH) domain comprising, preferably consisting of SEQ ID NO: 21, optionally with 1, 2, 3, 4 or 5 amino acid alterations outside the complementarity determining regions (CDRs), and a variable light (VL) domain comprising, preferably consisting of SEQ ID NO: 26, optionally with 1, 2, 3, 4 or 5 amino acid alterations outside the complementarity determining regions (CDRs).
19. An antibody according to clause 17, wherein the antibody comprises a variable heavy (VH) domain comprising, preferably consisting of SEQ ID NO: 31, optionally with 1, 2, 3, 4 or 5 amino acid alterations outside the complementarity determining regions (CDRs), and a variable light (VL) domain comprising, preferably consisting of SEQ ID NO: 36, optionally with 1, 2, 3, 4 or 5 amino acid alterations outside the complementarity determining regions (CDRs).
20. An antibody according to clause 17, wherein the antibody comprises a variable heavy (VH) domain comprising, preferably consisting of SEQ ID NO: 21, optionally with 1, 2, 3, 4 or 5 amino acid alterations outside the complementarity determining regions (CDRs), and a variable light (VL) domain comprising, preferably consisting of SEQ ID NO: 26, optionally with 1, 2, 3, 4 or 5 amino acid alterations outside the complementarity determining regions (CDRs).
21. An antibody according to clause 17, wherein the antibody comprises a variable heavy (VH) domain comprising, preferably consisting of SEQ ID NO: 172, optionally with 1, 2, 3, 4 or 5 amino acid alterations outside the complementarity determining regions (CDRs), and a variable light (VL) domain comprising, preferably consisting of SEQ ID NO: 176, optionally with 1, 2, 3, 4 or 5 amino acid alterations outside the complementarity determining regions (CDRs).
22. An antibody according to clause 17, wherein the antibody comprises a variable heavy (VH) domain comprising, preferably consisting of SEQ ID NO: 180, optionally with 1, 2, 3, 4 or 5 amino acid alterations outside the complementarity determining regions (CDRs), and a variable light (VL) domain comprising, preferably consisting of SEQ ID NO: 184, optionally with 1, 2, 3, 4 or 5 amino acid alterations outside the complementarity determining regions (CDRs).
23. An antibody according to any one of clauses 1 to 4, wherein the antibody comprises a variable heavy (VH) domain sequence and a variable light (VL) domain sequence and wherein:
   (i) the variable heavy (VH) domain comprises, preferably consists of a sequence having at least 90%, at least 95%, at least 98%, or at least 99% identity to SEQ ID NO: 21 and
      the variable light (VL) domain comprises, preferably consists of a sequence having at least 90%, at least 95%, at least 98%, or at least 99% identity to SEQ ID NO: 26,
      provided that HCDR1 consists of SEQ ID NO: 22, HCDR2 consists of SEQ ID NO: 23, HCDR3 consists of SEQ ID NO: 24, LCDR1 consists of SEQ ID NO: 27, LCDR2 consists of SEQ ID NO: 28, and LCDR3 consists of SEQ ID NO: 29,
   (ii) the variable heavy (VH) domain comprises, preferably consists of a sequence having at least 90%, at least 95%, at least 98%, or at least 99% identity to SEQ ID NO: 31 and
      the variable light (VL) domain comprises, preferably consists of a sequence having at least 90%, at least 95%, at least 98%, or at least 99% identity to SEQ ID NO: 36,
      provided that HCDR1 consists of SEQ ID NO: 32, HCDR2 consists of SEQ ID NO: 33, HCDR3 consists of SEQ ID NO: 34, LCDR1 consists of SEQ ID NO: 37, LCDR2 consists of SEQ ID NO: 28, and LCDR3 consists of SEQ ID NO: 38,
   (iii) the variable heavy (VH) domain comprises, preferably consists of a sequence having at least 90%, at least 95%, at least 98%, or at least 99%/identity to SEQ ID NO: 172 and
      the variable light (VL) domain comprises, preferably consists of a sequence having at least 90%, at least 95%, at least 98%, or at least 99% identity to SEQ ID NO: 176,
      provided that HCDR1 consists of SEQ ID NO: 3, HCDR2 consists of SEQ ID NO: 173, HCDR3 consists of SEQ ID NO: 174, LCDR1 consists of SEQ ID NO: 149, LCDR2 consists of SEQ ID NO: 177, and LCDR3 consists of SEQ ID NO: 178, or
   (iv) the variable heavy (VH) domain comprises, preferably consists of a sequence having at least 90%, at least 95%, at least 98%, or at least 99% identity to SEQ ID NO: 180 and
      the variable light (VL) domain comprises, preferably consists of a sequence having at least 90%, at least 95%, at least 98%, or at least 99% identity to SEQ ID NO: 184,
      provided that HCDR1 consists of SEQ ID NO: 181, HCDR2 consists of SEQ ID NO: 173, HCDR3 consists of SEQ ID NO: 182, LCDR1 consists of SEQ ID NO: 185, LCDR2 consists of SEQ ID NO: 28, and LCDR3 consists of SEQ ID NO: 186.
24. An antibody according to clause 23, wherein the variable heavy (VH) domain sequence comprises, preferably consists of a sequence having at least 90%, at least 95%, at least 98%, or at least 99% identity to the VH domain having SEQ ID NO: 21 and the variable light (VL) domain sequence comprises, preferably consists of a sequence having at least 90%, at least 95%, at least 98%, or at least 99% identity to the VL domain having SEQ ID NO: 26, provided that HCDR1 consists of SEQ ID NO: 22, HCDR2 consists of SEQ ID NO: 23, HCDR3 consists of SEQ ID NO: 24, LCDR1 consists of SEQ ID NO: 27, LCDR2 consists of SEQ ID NO: 28, and LCDR3 consists of SEQ ID NO: 29.
25. An antibody according to clause 23, wherein the variable heavy (VH) domain sequence comprises, preferably consists of a sequence having at least 90%, at least 95%, at least 98%, or at least 99% identity to the VH domain having SEQ ID NO: 31 and the variable light (VL) domain sequence comprises, preferably consists of a sequence having at least 90%, at least 95%, at least 98%, or at least 99% identity to the VL domain having SEQ ID NO: 36, provided that HCDR1 consists of SEQ ID NO: 32, HCDR2 consists of SEQ ID NO: 33, HCDR3 consists of SEQ ID NO: 34, LCDR1 consists of SEQ ID NO: 37, LCDR2 consists of SEQ ID NO: 28, and LCDR3 consists of SEQ ID NO: 38.
26. An antibody according to clause 23, wherein the variable heavy (VH) domain sequence comprises, preferably consists of a sequence having at least 90%, at least 95%, at least 98%, or at least 99% identity to the VH domain having SEQ ID NO: 21 and the variable light (VL) domain sequence comprises, preferably consists of a sequence having at least 90%, at least 95%, at least 98%, or at least 99% identity to the VL domain having SEQ ID NO: 26, provided that HCDR1 consists of SEQ ID NO: 22, HCDR2 consists of SEQ ID NO: 23, HCDR3 consists of SEQ ID NO: 24, LCDR1 consists of SEQ ID NO: 27, LCDR2 consists of SEQ ID NO: 28, and LCDR3 consists of SEQ ID NO: 29.
27. An antibody according to clause 23, wherein the variable heavy (VH) domain sequence comprises, preferably consists of a sequence having at least 90%, at least 95%, at least 98%, or at least 99% identity to the VH domain having SEQ ID NO: 172 and the variable light (VL) domain sequence comprises, preferably consists of a sequence having at least 90%, at least 95%, at least 98%, or at least 99% identity to the VL domain having SEQ ID NO: 176, provided that HCDR1 consists of SEQ ID NO: 3, HCDR2 consists of SEQ ID NO: 173, HCDR3 consists of SEQ ID NO: 174, LCDR1 consists of SEQ ID NO: 149, LCDR2 consists of SEQ ID NO: 177, and LCDR3 consists of SEQ ID NO: 178.
28. An antibody according to clause 23, wherein the variable heavy (VH) domain sequence comprises, preferably consists of a sequence having at least 90%, at least 95%, at least 98%, or at least 99% identity to the VH domain having SEQ ID NO: 180 and the variable light (VL) domain sequence comprises, preferably consists of a sequence having at least 90%, at least 95%, at least 98%, or at least 99% identity to the VL domain having SEQ ID NO: 184, provided that HCDR1 consists of SEQ ID NO: 181, HCDR2 consists of SEQ ID NO: 173, HCDR3 consists of SEQ ID NO: 182, LCDR1 consists of SEQ ID NO: 185, LCDR2 consists of SEQ ID NO: 28, and LCDR3 consists of SEQ ID NO: 186.
29. An antibody according to any one of clauses 1 to 4, wherein the antibody comprises a variable heavy (VH) domain sequence and a variable light (VL) domain sequence and wherein:
   (i) the variable heavy (VH) domain comprises, preferably consists of SEQ ID NO: 21 and the variable light (VL) domain comprises, preferably consists of SEQ ID NO: 26,
   (ii) the variable heavy (VH) domain comprises, preferably consists of SEQ ID NO: 31 and the variable light (VL) domain comprises, preferably consists of SEQ ID NO:36,
   (iii) the variable heavy (VH) domain comprises, preferably consists of SEQ ID NO: 172 and the variable light (VL) domain comprises, preferably consists of SEQ ID NO:176, or
   (iv) the variable heavy (VH) domain comprises, preferably consists of SEQ ID NO: 180 and the variable light (VL) domain comprises, preferably consists of SEQ ID NO:184.
30. An antibody according to clause 29, wherein the antibody comprises the VH domain comprising, preferably consisting of SEQ ID NO: 21 and the VL domain comprising, preferably consisting of SEQ ID NO: 26.
31. An antibody according to clause 29, wherein the antibody comprises the VH domain comprising, preferably consisting of SEQ ID NO: 31 and the VL domain comprising, preferably consisting of SEQ ID NO: 36.
32. An antibody according to clause 29, wherein the antibody comprises the VH domain comprising, preferably consisting of SEQ ID NO: 21 and the VL domain comprising, preferably consisting of SEQ ID NO: 26.
33. An antibody according to clause 29, wherein the antibody comprises the VH domain comprising, preferably consisting of SEQ ID NO: 172 and the VL domain comprising, preferably consisting of SEQ ID NO: 176.
34. An antibody according to clause 29, wherein the antibody comprises the VH domain comprising, preferably consisting of SEQ ID NO: 180 and the VL domain comprising, preferably consisting of SEQ ID NO: 184.
35. An antibody that binds to the same epitope on Oxa-23 as an antibody according to any one of clauses 1 to 34.
36. An antibody capable of competing for binding to Oxa-23 with an antibody according to any one of clauses 1 to 34.
37. An antibody according to clause 1 or clause 2, that specifically binds OCL1 lipooligosaccharide (LOS) at the surface of Acinetobacter baumannii bacteria.
38. An antibody according to clause 1 or clause 2, that specifically binds OCL1 LOS on outer membrane vesicles (OMVs) of Acinetobacter baumannii bacteria.
39. An antibody according to clause 37 or clause 38, wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, and wherein the HCDR3 is selected from the group consisting of SEQ ID NO: 189, SEQ ID NO: 196, SEQ ID NO: 90, SEQ ID NO: 105, SEQ ID NO: 112, SEQ ID NO: 119, and SEQ ID NO: 146.
40. An antibody according to clause 39, wherein the HCDR3 is SEQ ID NO: 189.
41. An antibody according to clause 39, wherein the HCDR3 is SEQ ID NO: 196.
42. An antibody according to clause 39, wherein the HCDR3 is SEQ ID NO: 90.
43. An antibody according to clause 39, wherein the HCDR3 is SEQ ID NO: 105.
44. An antibody according to clause 39, wherein the HCDR3 is SEQ ID NO: 112.
45. An antibody according to clause 39, wherein the HCDR3 is SEQ ID NO: 119.
46. An antibody according to clause 39, wherein the HCDR3 is SEQ ID NO: 146.
47. An antibody according to clause 37 or clause 38, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, and wherein:
   (i) HCDR1 consists of SEQ ID NO: 32, HCDR2 consists of SEQ ID NO: 33, HCDR3 consists of SEQ ID NO: 189, LCDR1 consists of SEQ ID NO: 37, LCDR2 consists of SEQ ID NO: 28, and LCDR3 consists of SEQ ID NO: 192,
   (ii) HCDR1 consists of SEQ ID NO: 195, HCDR2 consists of SEQ ID NO: 33, HCDR3 consists of SEQ ID NO: 196, LCDR1 consists of SEQ ID NO: 37, LCDR2 consists of SEQ ID NO: 9, and LCDR3 consists of SEQ ID NO: 199,
   (iii) HCDR1 consists of SEQ ID NO: 22, HCDR2 consists of SEQ ID NO: 89, HCDR3 consists of SEQ ID NO: 90, LCDR1 consists of SEQ ID NO: 93, LCDR2 consists of SEQ ID NO: 28, and LCDR3 consists of SEQ ID NO: 94,
   (iv) HCDR1 consists of SEQ ID NO: 104, HCDR2 consists of SEQ ID NO: 33, HCDR3 consists of SEQ ID NO: 105, LCDR1 consists of SEQ ID NO: 27, LCDR2 consists of SEQ ID NO: 28, and LCDR3 consists of SEQ ID NO: 108,
   (v) HCDR1 consists of SEQ ID NO: 111, HCDR2 consists of SEQ ID NO: 33, HCDR3 consists of SEQ ID NO: 112, LCDR1 consists of SEQ ID NO: 115, LCDR2 consists of SEQ ID NO: 28, and LCDR3 consists of SEQ ID NO: 116,
   (vi) HCDR1 consists of SEQ ID NO: 104, HCDR2 consists of SEQ ID NO: 33, HCDR3 consists of SEQ ID NO: 119, LCDR1 consists of SEQ ID NO: 122, LCDR2 consists of SEQ ID NO: 28, and LCDR3 consists of SEQ ID NO: 123, or
   (vii) HCDR1 consists of SEQ ID NO: 145, HCDR2 consists of SEQ ID NO: 33, HCDR3 consists of SEQ ID NO: 146, LCDR1 consists of SEQ ID NO: 149, LCDR2 consists of SEQ ID NO: 150, and LCDR3 consists of SEQ ID NO: 151.
48. An antibody according to clause 47, wherein HCDR1 consists of SEQ ID NO: 32, HCDR2 consists of SEQ ID NO: 33, HCDR3 consists of SEQ ID NO: 189, LCDR1 consists of SEQ ID NO: 37, LCDR2 consists of SEQ ID NO: 28, and LCDR3 consists of SEQ ID NO: 192.
49. An antibody according to clause 47, wherein HCDR1 consists of SEQ ID NO: 195, HCDR2 consists of SEQ ID NO: 33, HCDR3 consists of SEQ ID NO: 196, LCDR1 consists of SEQ ID NO: 37, LCDR2 consists of SEQ ID NO: 9, and LCDR3 consists of SEQ ID NO: 199.
50. An antibody according to clause 47, wherein HCDR1 consists of SEQ ID NO: 22, HCDR2 consists of SEQ ID NO: 89, HCDR3 consists of SEQ ID NO: 90, LCDR1 consists of SEQ ID NO: 93, LCDR2 consists of SEQ ID NO: 28, and LCDR3 consists of SEQ ID NO: 94.
51. An antibody according to clause 47, wherein HCDR1 consists of SEQ ID NO: 104, HCDR2 consists of SEQ ID NO: 33, HCDR3 consists of SEQ ID NO: 105, LCDR1 consists of SEQ ID NO: 27, LCDR2 consists of SEQ ID NO: 28, and LCDR3 consists of SEQ ID NO: 108.
52. An antibody according to clause 47, wherein HCDR1 consists of SEQ ID NO: 111, HCDR2 consists of SEQ ID NO: 33, HCDR3 consists of SEQ ID NO: 112, LCDR1 consists of SEQ ID NO: 115, LCDR2 consists of SEQ ID NO: 28, and LCDR3 consists of SEQ ID NO: 116.
53. An antibody according to clause 47, wherein HCDR1 consists of SEQ ID NO: 104, HCDR2 consists of SEQ ID NO: 33, HCDR3 consists of SEQ ID NO: 119, LCDR1 consists of SEQ ID NO: 122, LCDR2 consists of SEQ ID NO: 28, and LCDR3 consists of SEQ ID NO: 123.
54. An antibody according to clause 47, wherein HCDR1 consists of SEQ ID NO: 145, HCDR2 consists of SEQ ID NO: 33, HCDR3 consists of SEQ ID NO: 146, LCDR1 consists of SEQ ID NO: 149, LCDR2 consists of SEQ ID NO: 150, and LCDR3 consists of SEQ ID NO: 151.
55. An antibody according to clause 37 or clause 38, wherein the antibody comprises a variable heavy (VH) domain sequence and a variable light (VL) domain sequence and wherein:
   (i) the variable heavy (VH) domain comprises, preferably consists of SEQ ID NO: 188 and the variable light (VL) domain comprises, preferably consists of SEQ ID NO: 191,
   (ii) the variable heavy (VH) domain comprises, preferably consists of SEQ ID NO: 194 and the variable light (VL) domain comprises, preferably consists of SEQ ID NO: 198,
   (iii) the variable heavy (VH) domain comprises, preferably consists of SEQ ID NO: 88 and the variable light (VL) domain comprises, preferably consists of SEQ ID NO: 92,
   (iv) the variable heavy (VH) domain comprises, preferably consists of SEQ ID NO: 103 and the variable light (VL) domain comprises, preferably consists of SEQ ID NO: 107,
   (v) the variable heavy (VH) domain comprises, preferably consists of SEQ ID NO: 110 and the variable light (VL) domain comprises, preferably consists of SEQ ID NO: 114,
   (vi) the variable heavy (VH) domain comprises, preferably consists of SEQ ID NO: 118 and the variable light (VL) domain comprises, preferably consists of SEQ ID NO: 121, or
   (vii) the variable heavy (VH) domain comprises, preferably consists of SEQ ID NO: 144 and the variable light (VL) domain comprises, preferably consists of SEQ ID NO: 148,
   optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.
56. An antibody according to clause 55, wherein the antibody comprises a variable heavy (VH) domain comprising, preferably consisting of SEQ ID NO: 188, optionally with 1, 2, 3, 4 or 5 amino acid alterations outside the complementarity determining regions (CDRs), and a variable light (VL) domain comprising, preferably consisting of SEQ ID NO: 191, optionally with 1, 2, 3, 4 or 5 amino acid alterations outside the complementarity determining regions (CDRs).
57. An antibody according to clause 55, wherein the antibody comprises a variable heavy (VH) domain comprising, preferably consisting of SEQ ID NO: 194, optionally with 1, 2, 3, 4 or 5 amino acid alterations outside the complementarity determining regions (CDRs), and a variable light (VL) domain comprising, preferably consisting of SEQ ID NO: 198, optionally with 1, 2, 3, 4 or 5 amino acid alterations outside the complementarity determining regions (CDRs).
58. An antibody according to clause 55, wherein the antibody comprises a variable heavy (VH) domain comprising, preferably consisting of SEQ ID NO: 88, optionally with 1, 2, 3, 4 or 5 amino acid alterations outside the complementarity determining regions (CDRs), and a variable light (VL) domain comprising, preferably consisting of SEQ ID NO: 92, optionally with 1, 2, 3, 4 or 5 amino acid alterations outside the complementarity determining regions (CDRs).
59. An antibody according to clause 55, wherein the antibody comprises a variable heavy (VH) domain comprising, preferably consisting of SEQ ID NO: 103, optionally with 1, 2, 3, 4 or 5 amino acid alterations outside the complementarity determining regions (CDRs), and a variable light (VL) domain comprising, preferably consisting of SEQ ID NO: 107, optionally with 1, 2, 3, 4 or 5 amino acid alterations outside the complementarity determining regions (CDRs).
60. An antibody according to clause 55, wherein the antibody comprises a variable heavy (VH) domain comprising, preferably consisting of SEQ ID NO: 110, optionally with 1, 2, 3, 4 or 5 amino acid alterations outside the complementarity determining regions (CDRs), and a variable light (VL) domain comprising, preferably consisting of SEQ ID NO: 114, optionally with 1, 2, 3, 4 or 5 amino acid alterations outside the complementarity determining regions (CDRs).
61. An antibody according to clause 55, wherein the antibody comprises a variable heavy (VH) domain comprising, preferably consisting of SEQ ID NO: 118, optionally with 1, 2, 3, 4 or 5 amino acid alterations outside the complementarity determining regions (CDRs), and a variable light (VL) domain comprising, preferably consisting of SEQ ID NO: 121, optionally with 1, 2, 3, 4 or 5 amino acid alterations outside the complementarity determining regions (CDRs).
62. An antibody according to clause 55, wherein the antibody comprises a variable heavy (VH) domain comprising, preferably consisting of SEQ ID NO: 144, optionally with 1, 2, 3, 4 or 5 amino acid alterations outside the complementarity determining regions (CDRs), and a variable light (VL) domain comprising, preferably consisting of SEQ ID NO: 148, optionally with 1, 2, 3, 4 or 5 amino acid alterations outside the complementarity determining regions (CDRs).
63. An antibody according to clause 37 or clause 38, wherein the antibody comprises a variable heavy (VH) domain sequence and a variable light (VL) domain sequence and wherein:
   (i) the variable heavy (VH) domain comprises, preferably consists of a sequence having at least 90%, at least 95%, at least 98%, or at least 99% identity to SEQ ID NO: 188 and
      the variable light (VL) domain comprises, preferably consists of a sequence having at least 90%, at least 95%, at least 98%, or at least 99% identity to SEQ ID NO: 191,
      provided that HCDR1 consists of SEQ ID NO: 32, HCDR2 consists of SEQ ID NO: 33, HCDR3 consists of SEQ ID NO: 189, LCDR1 consists of SEQ ID NO: 37, LCDR2 consists of SEQ ID NO: 28, and LCDR3 consists of SEQ ID NO: 192,
   (ii) the variable heavy (VH) domain comprises, preferably consists of a sequence having at least 90%, at least 95%, at least 98%, or at least 99%/identity to SEQ ID NO: 194 and
      the variable light (VL) domain comprises, preferably consists of a sequence having at least 90%, at least 95%, at least 98%, or at least 99% identity to SEQ ID NO: 198,
      provided that HCDR1 consists of SEQ ID NO: 195, HCDR2 consists of SEQ ID NO: 33, HCDR3 consists of SEQ ID NO: 196, LCDR1 consists of SEQ ID NO: 37, LCDR2 consists of SEQ ID NO: 9, and LCDR3 consists of SEQ ID NO: 199,
   (iii) the variable heavy (VH) domain comprises, preferably consists of a sequence having at least 90%, at least 95%, at least 98%, or at least 99% identity to SEQ ID NO: 88 and
      the variable light (VL) domain comprises, preferably consists of a sequence having at least 90%, at least 95%, at least 98%, or at least 99% identity to SEQ ID NO: 92,
      provided that HCDR1 consists of SEQ ID NO: 22, HCDR2 consists of SEQ ID NO: 89, HCDR3 consists of SEQ ID NO: 90, LCDR1 consists of SEQ ID NO: 93, LCDR2 consists of SEQ ID NO: 28, and LCDR3 consists of SEQ ID NO: 94,
   (iv) the variable heavy (VH) domain comprises, preferably consists of a sequence having at least 90%, at least 95%, at least 98%, or at least 99% identity to SEQ ID NO: 103 and
      the variable light (VL) domain comprises, preferably consists of a sequence having at least 90%, at least 95%, at least 98%, or at least 99% identity to SEQ ID NO: 107,
      provided that HCDR1 consists of SEQ ID NO: 104, HCDR2 consists of SEQ ID NO: 33, HCDR3 consists of SEQ ID NO: 105, LCDR1 consists of SEQ ID NO: 27, LCDR2 consists of SEQ ID NO: 28, and LCDR3 consists of SEQ ID NO: 108,
   (v) the variable heavy (VH) domain comprises, preferably consists of a sequence having at least 90%, at least 95%, at least 98%, or at least 99% identity to SEQ ID NO: 110 and
      the variable light (VL) domain comprises, preferably consists of a sequence having at least 90%, at least 95%, at least 98%, or at least 99% identity to SEQ ID NO: 114,
      provided that HCDR1 consists of SEQ ID NO: 111, HCDR2 consists of SEQ ID NO: 33, HCDR3 consists of SEQ ID NO: 112, LCDR1 consists of SEQ ID NO: 115, LCDR2 consists of SEQ ID NO: 28, and LCDR3 consists of SEQ ID NO: 116,
   (vi) the variable heavy (VH) domain comprises, preferably consists of a sequence having at least 90%, at least 95%, at least 98%, or at least 99% identity to SEQ ID NO: 118 and
      the variable light (VL) domain comprises, preferably consists of a sequence having at least 90%, at least 95%, at least 98%, or at least 99% identity to SEQ ID NO: 121,
      provided that HCDR1 consists of SEQ ID NO: 104, HCDR2 consists of SEQ ID NO: 33, HCDR3 consists of SEQ ID NO: 119, LCDR1 consists of SEQ ID NO: 122, LCDR2 consists of SEQ ID NO: 28, and LCDR3 consists of SEQ ID NO: 123, or
   (vii) the variable heavy (VH) domain comprises, preferably consists of a sequence having at least 90%, at least 95%, at least 98%, or at least 99% identity to SEQ ID NO: 144 and
      the variable light (VL) domain comprises, preferably consists of a sequence having at least 90%, at least 95%, at least 98%, or at least 99% identity to SEQ ID NO: 148,
      provided that HCDR1 consists of SEQ ID NO: 145, HCDR2 consists of SEQ ID NO: 33, HCDR3 consists of SEQ ID NO: 146, LCDR1 consists of SEQ ID NO: 149, LCDR2 consists of SEQ ID NO: 150, and LCDR3 consists of SEQ ID NO: 151.
64. An antibody according to clause 63, wherein the variable heavy (VH) domain sequence comprises, preferably consists of a sequence having at least 90%, at least 95%, at least 98%, or at least 99% identity to the VH domain having SEQ ID NO: 188 and the variable light (VL) domain sequence comprises, preferably consists of a sequence having at least 90%, at least 95%, at least 98%, or at least 99% identity to the VL domain having SEQ ID NO: 191, provided that HCDR1 consists of SEQ ID NO: 32, HCDR2 consists of SEQ ID NO: 33, HCDR3 consists of SEQ ID NO: 189, LCDR1 consists of SEQ ID NO: 37, LCDR2 consists of SEQ ID NO: 28, and LCDR3 consists of SEQ ID NO: 192.
65. An antibody according to clause 63, wherein the variable heavy (VH) domain sequence comprises, preferably consists of a sequence having at least 90%, at least 95%, at least 98%, or at least 99% identity to the VH domain having SEQ ID NO: 194 and the variable light (VL) domain sequence comprises, preferably consists of a sequence having at least 90%, at least 95%, at least 98%, or at least 99% identity to the VL domain having SEQ ID NO: 198, provided that HCDR1 consists of SEQ ID NO: 195, HCDR2 consists of SEQ ID NO: 33, HCDR3 consists of SEQ ID NO: 196, LCDR1 consists of SEQ ID NO: 37, LCDR2 consists of SEQ ID NO: 9, and LCDR3 consists of SEQ ID NO: 199.
66. An antibody according to clause 63, wherein the variable heavy (VH) domain sequence comprises, preferably consists of a sequence having at least 90%, at least 95%, at least 98%, or at least 99% identity to the VH domain having SEQ ID NO: 88 and the variable light (VL) domain sequence comprises, preferably consists of a sequence having at least 90%, at least 95%, at least 98%, or at least 99% identity to the VL domain having SEQ ID NO: 92, provided that HCDR1 consists of SEQ ID NO: 22, HCDR2 consists of SEQ ID NO: 89, HCDR3 consists of SEQ ID NO: 90, LCDR1 consists of SEQ ID NO: 93, LCDR2 consists of SEQ ID NO: 28, and LCDR3 consists of SEQ ID NO: 94.
67. An antibody according to clause 63, wherein the variable heavy (VH) domain sequence comprises, preferably consists of a sequence having at least 90%, at least 95%, at least 98%, or at least 99% identity to the VH domain having SEQ ID NO: 103 and the variable light (VL) domain sequence comprises, preferably consists of a sequence having at least 90%, at least 95%, at least 98%, or at least 99% identity to the VL domain having SEQ ID NO: 107, provided that HCDR1 consists of SEQ ID NO: 104, HCDR2 consists of SEQ ID NO: 33, HCDR3 consists of SEQ ID NO: 105, LCDR1 consists of SEQ ID NO: 27, LCDR2 consists of SEQ ID NO: 28, and LCDR3 consists of SEQ ID NO: 108.
68. An antibody according to clause 63, wherein the variable heavy (VH) domain sequence comprises, preferably consists of a sequence having at least 90%, at least 95%, at least 98%, or at least 99% identity to the VH domain having SEQ ID NO: 110 and the variable light (VL) domain sequence comprises, preferably consists of a sequence having at least 90%, at least 95%, at least 98%, or at least 99% identity to the VL domain having SEQ ID NO: 114, provided that HCDR1 consists of SEQ ID NO: 111, HCDR2 consists of SEQ ID NO: 33, HCDR3 consists of SEQ ID NO: 112, LCDR1 consists of SEQ ID NO: 115, LCDR2 consists of SEQ ID NO: 28, and LCDR3 consists of SEQ ID NO: 116.
69. An antibody according to clause 63, wherein the variable heavy (VH) domain sequence comprises, preferably consists of a sequence having at least 90%, at least 95%, at least 98%, or at least 99% identity to the VH domain having SEQ ID NO: 118 and the variable light (VL) domain sequence comprises, preferably consists of a sequence having at least 90%, at least 95%, at least 98%, or at least 99% identity to the VL domain having SEQ ID NO: 121, provided that HCDR1 consists of SEQ ID NO: 104, HCDR2 consists of SEQ ID NO: 33, HCDR3 consists of SEQ ID NO: 119, LCDR1 consists of SEQ ID NO: 122, LCDR2 consists of SEQ ID NO: 28, and LCDR3 consists of SEQ ID NO: 123.
70. An antibody according to clause 63, wherein the variable heavy (VH) domain sequence comprises, preferably consists of a sequence having at least 90%, at least 95%, at least 98%, or at least 99% identity to the VH domain having SEQ ID NO: 144 and the variable light (VL) domain sequence comprises, preferably consists of a sequence having at least 90%, at least 95%, at least 98%, or at least 99% identity to the VL domain having SEQ ID NO: 148, provided that HCDR1 consists of SEQ ID NO: 145, HCDR2 consists of SEQ ID NO: 33, HCDR3 consists of SEQ ID NO: 146, LCDR1 consists of SEQ ID NO: 149, LCDR2 consists of SEQ ID NO: 150, and LCDR3 consists of SEQ ID NO: 151.
71. An antibody according to clause 37 or clause 38, wherein the antibody comprises a variable heavy (VH) domain sequence and a variable light (VL) domain sequence and wherein:
   (i) the variable heavy (VH) domain comprises, preferably consists of SEQ ID NO: 188 and the variable light (VL) domain comprises, preferably consists of SEQ ID NO: 191,
   (ii) the variable heavy (VH) domain comprises, preferably consists of SEQ ID NO: 194 and the variable light (VL) domain comprises, preferably consists of SEQ ID NO: 198,
   (iii) the variable heavy (VH) domain comprises, preferably consists of SEQ ID NO: 88 and the variable light (VL) domain comprises, preferably consists of SEQ ID NO: 92,
   (iv) the variable heavy (VH) domain comprises, preferably consists of SEQ ID NO: 103 and the variable light (VL) domain comprises, preferably consists of SEQ ID NO: 107,
   (v) the variable heavy (VH) domain comprises, preferably consists of SEQ ID NO: 110 and the variable light (VL) domain comprises, preferably consists of SEQ ID NO: 114,
   (vi) the variable heavy (VH) domain comprises, preferably consists of SEQ ID NO: 118 and the variable light (VL) domain comprises, preferably consists of SEQ ID NO: 121,
   (vii) the variable heavy (VH) domain comprises, preferably consists of SEQ ID NO: 144 and the variable light (VL) domain comprises, preferably consists of SEQ ID NO: 148.
72. An antibody according to clause 71, wherein the antibody comprises the VH domain comprising, preferably consisting of SEQ ID NO: 188 and the VL domain comprising, preferably consisting of SEQ ID NO: 191.
73. An antibody according to clause 71, wherein the antibody comprises the VH domain comprising, preferably consisting of SEQ ID NO: 194 and the VL domain comprising, preferably consisting of SEQ ID NO: 198.
74. An antibody according to clause 71, wherein the antibody comprises the VH domain comprising, preferably consisting of SEQ ID NO: 88 and the VL domain comprising, preferably consisting of SEQ ID NO: 92.
75. An antibody according to clause 71, wherein the antibody comprises the VH domain comprising, preferably consisting of SEQ ID NO: 103 and the VL domain comprising, preferably consisting of SEQ ID NO: 107.
76. An antibody according to clause 71, wherein the antibody comprises the VH domain comprising, preferably consisting of SEQ ID NO: 110 and the VL domain comprising, preferably consisting of SEQ ID NO: 114.
77. An antibody according to clause 71, wherein the antibody comprises the VH domain comprising, preferably consisting of SEQ ID NO: 118 and the VL domain comprising, preferably consisting of SEQ ID NO: 121.
78. An antibody according to clause 71, wherein the antibody comprises the VH domain comprising, preferably consisting of SEQ ID NO: 144 and the VL domain comprising, preferably consisting of SEQ ID NO: 148.
79. An antibody that binds to the same epitope on OCL1 LOS as an antibody according to any one of clauses 37 to 78.
80. An antibody capable of competing for binding to OCL1 LOS with an antibody according to any one of clauses 37 to 78.
81. An antibody according to clause 1 or clause 2, that specifically binds KL49 at the surface of Acinetobacter baumannii bacteria.
82. An antibody according to clause 1 or clause 2, that specifically binds KL49 on outer membrane vesicles (OMVs) of Acinetobacter baumannii bacteria.
83. An antibody according to clause 81 or clause 82, wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, and wherein the HCDR3 is selected from the group consisting of SEQ ID NO: 5, SEQ ID NO: 14, SEQ ID NO: 41, SEQ ID NO: 45, SEQ ID NO: 50, SEQ ID NO: 55, SEQ ID NO: 62, SEQ ID NO: 67, SEQ ID NO: 72, SEQ ID NO: 77, SEQ ID NO: 83, SEQ ID NO: 98, SEQ ID NO: 126, SEQ ID NO: 132, SEQ ID NO: 139, SEQ ID NO: 156, SEQ ID NO: 163, and SEQ ID NO: 168.
84. An antibody according to clause 83, wherein the HCDR3 is SEQ ID NO: 5.
85. An antibody according to clause 83, wherein the HCDR3 is SEQ ID NO: 14.
86. An antibody according to clause 83, wherein the HCDR3 is SEQ ID NO: 41.
87. An antibody according to clause 83, wherein the HCDR3 is SEQ ID NO: 45.
88. An antibody according to clause 83, wherein the HCDR3 is SEQ ID NO: 50.
89. An antibody according to clause 83, wherein the HCDR3 is SEQ ID NO: 55.
90. An antibody according to clause 83, wherein the HCDR3 is SEQ ID NO: 62.
91. An antibody according to clause 83, wherein the HCDR3 is SEQ ID NO: 67.
92. An antibody according to clause 83, wherein the HCDR3 is SEQ ID NO: 72.
93. An antibody according to clause 83, wherein the HCDR3 is SEQ ID NO: 77.
94. An antibody according to clause 83, wherein the HCDR3 is SEQ ID NO: 83.
95. An antibody according to clause 83, wherein the HCDR3 is SEQ ID NO: 98.
96. An antibody according to clause 83, wherein the HCDR3 is SEQ ID NO: 126.
97. An antibody according to clause 83, wherein the HCDR3 is SEQ ID NO: 132.
98. An antibody according to clause 83, wherein the HCDR3 is SEQ ID NO: 139.
99. An antibody according to clause 83, wherein the HCDR3 is SEQ ID NO: 156.
100. An antibody according to clause 83, wherein the HCDR3 is SEQ ID NO: 163.
101. An antibody according to clause 83, wherein the HCDR3 is SEQ ID NO: 168.
102. An antibody according to clause 81 or clause 82, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, and wherein:
   (i) HCDR1 consists of SEQ ID NO: 3, HCDR2 consists of SEQ ID NO: 4, HCDR3 consists of SEQ ID NO: 5, LCDR1 consists of SEQ ID NO: 8, LCDR2 consists of SEQ ID NO: 9, and LCDR3 consists of SEQ ID NO: 10,
   (ii) HCDR1 consists of SEQ ID NO: 3, HCDR2 consists of SEQ ID NO: 13, HCDR3 consists of SEQ ID NO: 14, LCDR1 consists of SEQ ID NO: 17, LCDR2 consists of SEQ ID NO: 18, and LCDR3 consists of SEQ ID NO: 19,
   (iii) HCDR1 consists of SEQ ID NO: 3, HCDR2 consists of SEQ ID NO: 13, HCDR3 consists of SEQ ID NO: 41, LCDR1 consists of SEQ ID NO: 17, LCDR2 consists of SEQ ID NO: 18, and LCDR3 consists of SEQ ID NO: 19,
   (iv) HCDR1 consists of SEQ ID NO: 3, HCDR2 consists of SEQ ID NO: 13, HCDR3 consists of SEQ ID NO: 45, LCDR1 consists of SEQ ID NO: 17, LCDR2 consists of SEQ ID NO: 18, and LCDR3 consists of SEQ ID NO: 10,
   (v) HCDR1 consists of SEQ ID NO: 3, HCDR2 consists of SEQ ID NO: 13, HCDR3 consists of SEQ ID NO: 50, LCDR1 consists of SEQ ID NO: 17, LCDR2 consists of SEQ ID NO: 18, and LCDR3 consists of SEQ ID NO: 19,
   (vi) HCDR1 consists of SEQ ID NO: 3, HCDR2 consists of SEQ ID NO: 13, HCDR3 consists of SEQ ID NO: 55, LCDR1 consists of SEQ ID NO: 58, LCDR2 consists of SEQ ID NO: 18, and LCDR3 consists of SEQ ID NO: 59,
   (vii) HCDR1 consists of SEQ ID NO: 3, HCDR2 consists of SEQ ID NO: 13, HCDR3 consists of SEQ ID NO: 62, LCDR1 consists of SEQ ID NO: 17, LCDR2 consists of SEQ ID NO: 18, and LCDR3 consists of SEQ ID NO: 59,
   (viii) HCDR1 consists of SEQ ID NO: 3, HCDR2 consists of SEQ ID NO: 13, HCDR3 consists of SEQ ID NO: 67, LCDR1 consists of SEQ ID NO: 17, LCDR2 consists of SEQ ID NO: 18, and LCDR3 consists of SEQ ID NO: 59,
   (ix) HCDR1 consists of SEQ ID NO: 3, HCDR2 consists of SEQ ID NO: 13, HCDR3 consists of SEQ ID NO: 72, LCDR1 consists of SEQ ID NO: 17, LCDR2 consists of SEQ ID NO: 18, and LCDR3 consists of SEQ ID NO: 59,
   (x) HCDR1 consists of SEQ ID NO: 3, HCDR2 consists of SEQ ID NO: 13, HCDR3 consists of SEQ ID NO: 77, LCDR1 consists of SEQ ID NO: 17, LCDR2 consists of SEQ ID NO: 18, and LCDR3 consists of SEQ ID NO: 80,
   (xi) HCDR1 consists of SEQ ID NO: 3, HCDR2 consists of SEQ ID NO: 13, HCDR3 consists of SEQ ID NO: 83, LCDR1 consists of SEQ ID NO: 17, LCDR2 consists of SEQ ID NO: 18, and LCDR3 consists of SEQ ID NO: 86,
   (xii) HCDR1 consists of SEQ ID NO: 3, HCDR2 consists of SEQ ID NO: 97, HCDR3 consists of SEQ ID NO: 98, LCDR1 consists of SEQ ID NO: 101, LCDR2 consists of SEQ ID NO: 18, and LCDR3 consists of SEQ ID NO: 59,
   (xiii) HCDR1 consists of SEQ ID NO: 3, HCDR2 consists of SEQ ID NO: 13, HCDR3 consists of SEQ ID NO: 126, LCDR1 consists of SEQ ID NO: 17, LCDR2 consists of SEQ ID NO: 18, and LCDR3 consists of SEQ ID NO: 129,
   (xiv) HCDR1 consists of SEQ ID NO: 3, HCDR2 consists of SEQ ID NO: 13, HCDR3 consists of SEQ ID NO: 132, LCDR1 consists of SEQ ID NO: 17, LCDR2 consists of SEQ ID NO: 18, and LCDR3 consists of SEQ ID NO: 135,
   (xv) HCDR1 consists of SEQ ID NO: 3, HCDR2 consists of SEQ ID NO: 138, HCDR3 consists of SEQ ID NO: 139, LCDR1 consists of SEQ ID NO: 142, LCDR2 consists of SEQ ID NO: 18, and LCDR3 consists of SEQ ID NO: 86,
   (xvi) HCDR1 consists of SEQ ID NO: 154, HCDR2 consists of SEQ ID NO: 155, HCDR3 consists of SEQ ID NO: 156, LCDR1 consists of SEQ ID NO: 17, LCDR2 consists of SEQ ID NO: 18, and LCDR3 consists of SEQ ID NO: 159,
   (xvii) HCDR1 consists of SEQ ID NO: 3, HCDR2 consists of SEQ ID NO: 162, HCDR3 consists of SEQ ID NO: 163, LCDR1 consists of SEQ ID NO: 17, LCDR2 consists of SEQ ID NO: 18, and LCDR3 consists of SEQ ID NO: 135, or
   (xviii) HCDR1 consists of SEQ ID NO: 3, HCDR2 consists of SEQ ID NO: 13, HCDR3 consists of SEQ ID NO: 168, LCDR1 consists of SEQ ID NO: 17, LCDR2 consists of SEQ ID NO: 18, and LCDR3 consists of SEQ ID NO: 10.
103. An antibody according to clause 102, wherein HCDR1 consists of SEQ ID NO: 3, HCDR2 consists of SEQ ID NO: 4, HCDR3 consists of SEQ ID NO: 5, LCDR1 consists of SEQ ID NO: 8, LCDR2 consists of SEQ ID NO: 9, and LCDR3 consists of SEQ ID NO: 10.
104. An antibody according to clause 102, wherein HCDR1 consists of SEQ ID NO: 3, HCDR2 consists of SEQ ID NO: 13, HCDR3 consists of SEQ ID NO: 14, LCDR1 consists of SEQ ID NO: 17, LCDR2 consists of SEQ ID NO: 18, and LCDR3 consists of SEQ ID NO: 19.
105. An antibody according to clause 102, wherein HCDR1 consists of SEQ ID NO: 3, HCDR2 consists of SEQ ID NO: 13, HCDR3 consists of SEQ ID NO: 41, LCDR1 consists of SEQ ID NO: 17, LCDR2 consists of SEQ ID NO: 18, and LCDR3 consists of SEQ ID NO: 19.
106. An antibody according to clause 102, wherein HCDR1 consists of SEQ ID NO: 3, HCDR2 consists of SEQ ID NO: 13, HCDR3 consists of SEQ ID NO: 45, LCDR1 consists of SEQ ID NO: 17, LCDR2 consists of SEQ ID NO: 18, and LCDR3 consists of SEQ ID NO: 10.
107. An antibody according to clause 102, wherein HCDR1 consists of SEQ ID NO: 3, HCDR2 consists of SEQ ID NO: 13, HCDR3 consists of SEQ ID NO: 50, LCDR1 consists of SEQ ID NO: 17, LCDR2 consists of SEQ ID NO: 18, and LCDR3 consists of SEQ ID NO: 19.
108. An antibody according to clause 102, wherein HCDR1 consists of SEQ ID NO: 3, HCDR2 consists of SEQ ID NO: 13, HCDR3 consists of SEQ ID NO: 55, LCDR1 consists of SEQ ID NO: 58, LCDR2 consists of SEQ ID NO: 18, and LCDR3 consists of SEQ ID NO: 59.
109. An antibody according to clause 102, wherein HCDR1 consists of SEQ ID NO: 3, HCDR2 consists of SEQ ID NO: 13, HCDR3 consists of SEQ ID NO: 62, LCDR1 consists of SEQ ID NO: 17, LCDR2 consists of SEQ ID NO: 18, and LCDR3 consists of SEQ ID NO: 59.
110. An antibody according to clause 102, wherein HCDR1 consists of SEQ ID NO: 3, HCDR2 consists of SEQ ID NO: 13, HCDR3 consists of SEQ ID NO: 67, LCDR1 consists of SEQ ID NO: 17, LCDR2 consists of SEQ ID NO: 18, and LCDR3 consists of SEQ ID NO: 59.
111. An antibody according to clause 102, wherein HCDR1 consists of SEQ ID NO: 3, HCDR2 consists of SEQ ID NO: 13, HCDR3 consists of SEQ ID NO: 72, LCDR1 consists of SEQ ID NO: 17, LCDR2 consists of SEQ ID NO: 18, and LCDR3 consists of SEQ ID NO: 59.
112. An antibody according to clause 102, wherein HCDR1 consists of SEQ ID NO: 3, HCDR2 consists of SEQ ID NO: 13, HCDR3 consists of SEQ ID NO: 77, LCDR1 consists of SEQ ID NO: 17, LCDR2 consists of SEQ ID NO: 18, and LCDR3 consists of SEQ ID NO: 80.
113. An antibody according to clause 102, wherein HCDR1 consists of SEQ ID NO: 3, HCDR2 consists of SEQ ID NO: 13, HCDR3 consists of SEQ ID NO: 83, LCDR1 consists of SEQ ID NO: 17, LCDR2 consists of SEQ ID NO: 18, and LCDR3 consists of SEQ ID NO: 86.
114. An antibody according to clause 102, wherein HCDR1 consists of SEQ ID NO: 3, HCDR2 consists of SEQ ID NO: 97, HCDR3 consists of SEQ ID NO: 98, LCDR1 consists of SEQ ID NO: 101, LCDR2 consists of SEQ ID NO: 18, and LCDR3 consists of SEQ ID NO: 59.
115. An antibody according to clause 102, wherein HCDR1 consists of SEQ ID NO: 3, HCDR2 consists of SEQ ID NO: 13, HCDR3 consists of SEQ ID NO: 126, LCDR1 consists of SEQ ID NO: 17, LCDR2 consists of SEQ ID NO: 18, and LCDR3 consists of SEQ ID NO: 129.
116. An antibody according to clause 102, wherein HCDR1 consists of SEQ ID NO: 3, HCDR2 consists of SEQ ID NO: 13, HCDR3 consists of SEQ ID NO: 132, LCDR1 consists of SEQ ID NO: 17, LCDR2 consists of SEQ ID NO: 18, and LCDR3 consists of SEQ ID NO: 135.
117. An antibody according to clause 102, wherein HCDR1 consists of SEQ ID NO: 3, HCDR2 consists of SEQ ID NO: 138, HCDR3 consists of SEQ ID NO: 139, LCDR1 consists of SEQ ID NO: 142, LCDR2 consists of SEQ ID NO: 18, and LCDR3 consists of SEQ ID NO: 86.
118. An antibody according to clause 102, wherein HCDR1 consists of SEQ ID NO: 154, HCDR2 consists of SEQ ID NO: 155, HCDR3 consists of SEQ ID NO: 156, LCDR1 consists of SEQ ID NO: 17, LCDR2 consists of SEQ ID NO: 18, and LCDR3 consists of SEQ ID NO: 159.
119. An antibody according to clause 102, wherein HCDR1 consists of SEQ ID NO: 3, HCDR2 consists of SEQ ID NO: 162, HCDR3 consists of SEQ ID NO: 163, LCDR1 consists of SEQ ID NO: 17, LCDR2 consists of SEQ ID NO: 18, and LCDR3 consists of SEQ ID NO: 135.
120. An antibody according to clause 102, wherein HCDR1 consists of SEQ ID NO: 3, HCDR2 consists of SEQ ID NO: 13, HCDR3 consists of SEQ ID NO: 168, LCDR1 consists of SEQ ID NO: 17, LCDR2 consists of SEQ ID NO: 18, and LCDR3 consists of SEQ ID NO: 10.
121. An antibody according to clause 81 or clause 82, wherein the antibody comprises a variable heavy (VH) domain sequence and a variable light (VL) domain sequence and wherein:
   (i) the variable heavy (VH) domain comprises, preferably consists of SEQ ID NO: 2 and the variable light (VL) domain comprises, preferably consists of SEQ ID NO: 7,
   (ii) the variable heavy (VH) domain comprises, preferably consists of SEQ ID NO: 12 and the variable light (VL) domain comprises, preferably consists of SEQ ID NO: 16,
   (iii) the variable heavy (VH) domain comprises, preferably consists of SEQ ID NO: 40 and the variable light (VL) domain comprises, preferably consists of SEQ ID NO: 16,
   (iv) the variable heavy (VH) domain comprises, preferably consists of SEQ ID NO: 44 and the variable light (VL) domain comprises, preferably consists of SEQ ID NO: 47,
   (v) the variable heavy (VH) domain comprises, preferably consists of SEQ ID NO: 49 and the variable light (VL) domain comprises, preferably consists of SEQ ID NO: 52,
   (vi) the variable heavy (VH) domain comprises, preferably consists of SEQ ID NO: 54 and the variable light (VL) domain comprises, preferably consists of SEQ ID NO: 57,
   (vii) the variable heavy (VH) domain comprises, preferably consists of SEQ ID NO: 61 and the variable light (VL) domain comprises, preferably consists of SEQ ID NO: 64,
   (viii) the variable heavy (VH) domain comprises, preferably consists of SEQ ID NO: 66 and the variable light (VL) domain comprises, preferably consists of SEQ ID NO: 69,
   (ix) the variable heavy (VH) domain comprises, preferably consists of SEQ ID NO: 71 and the variable light (VL) domain comprises, preferably consists of SEQ ID NO: 74,
   (x) the variable heavy (VH) domain comprises, preferably consists of SEQ ID NO: 76 and the variable light (VL) domain comprises, preferably consists of SEQ ID NO: 79,
   (xi) the variable heavy (VH) domain comprises, preferably consists of SEQ ID NO: 82 and the variable light (VL) domain comprises, preferably consists of SEQ ID NO: 85,
   (xii) the variable heavy (VH) domain comprises, preferably consists of SEQ ID NO: 96 and the variable light (VL) domain comprises, preferably consists of SEQ ID NO: 100,
   (xiii) the variable heavy (VH) domain comprises, preferably consists of SEQ ID NO: 125 and the variable light (VL) domain comprises, preferably consists of SEQ ID NO: 128,
   (xiv) the variable heavy (VH) domain comprises, preferably consists of SEQ ID NO: 131 and the variable light (VL) domain comprises, preferably consists of SEQ ID NO: 134,
   (xv) the variable heavy (VH) domain comprises, preferably consists of SEQ ID NO: 137 and the variable light (VL) domain comprises, preferably consists of SEQ ID NO: 141,
   (xvi) the variable heavy (VH) domain comprises, preferably consists of SEQ ID NO: 153 and the variable light (VL) domain comprises, preferably consists of SEQ ID NO: 158,
   (xvii) the variable heavy (VH) domain comprises, preferably consists of SEQ ID NO: 161 and the variable light (VL) domain comprises, preferably consists of SEQ ID NO: 165, or
   (xviii) the variable heavy (VH) domain comprises, preferably consists of SEQ ID NO: 167 and the variable light (VL) domain comprises, preferably consists of SEQ ID NO: 170,
   optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.
122. An antibody according to clause 121, wherein the antibody comprises a variable heavy (VH) domain comprising, preferably consisting of SEQ ID NO: 2, optionally with 1, 2, 3, 4 or 5 amino acid alterations outside the complementarity determining regions (CDRs), and a variable light (VL) domain comprising, preferably consisting of SEQ ID NO: 7, optionally with 1, 2, 3, 4 or 5 amino acid alterations outside the complementarity determining regions (CDRs).
123. An antibody according to clause 121, wherein the antibody comprises a variable heavy (VH) domain comprising, preferably consisting of SEQ ID NO: 12, optionally with 1, 2, 3, 4 or 5 amino acid alterations outside the complementarity determining regions (CDRs), and a variable light (VL) domain comprising, preferably consisting of SEQ ID NO: 16, optionally with 1, 2, 3, 4 or 5 amino acid alterations outside the complementarity determining regions (CDRs).
124. An antibody according to clause 121, wherein the antibody comprises a variable heavy (VH) domain comprising, preferably consisting of SEQ ID NO: 40, optionally with 1, 2, 3, 4 or 5 amino acid alterations outside the complementarity determining regions (CDRs), and a variable light (VL) domain comprising, preferably consisting of SEQ ID NO: 16, optionally with 1, 2, 3, 4 or 5 amino acid alterations outside the complementarity determining regions (CDRs).
125. An antibody according to clause 121, wherein the antibody comprises a variable heavy (VH) domain comprising, preferably consisting of SEQ ID NO: 44, optionally with 1, 2, 3, 4 or 5 amino acid alterations outside the complementarity determining regions (CDRs), and a variable light (VL) domain comprising, preferably consisting of SEQ ID NO: 47, optionally with 1, 2, 3, 4 or 5 amino acid alterations outside the complementarity determining regions (CDRs).
126. An antibody according to clause 121, wherein the antibody comprises a variable heavy (VH) domain comprising, preferably consisting of SEQ ID NO: 49, optionally with 1, 2, 3, 4 or 5 amino acid alterations outside the complementarity determining regions (CDRs), and a variable light (VL) domain comprising, preferably consisting of SEQ ID NO: 52, optionally with 1, 2, 3, 4 or 5 amino acid alterations outside the complementarity determining regions (CDRs).
127. An antibody according to clause 121, wherein the antibody comprises a variable heavy (VH) domain comprising, preferably consisting of SEQ ID NO: 54, optionally with 1, 2, 3, 4 or 5 amino acid alterations outside the complementarity determining regions (CDRs), and a variable light (VL) domain comprising, preferably consisting of SEQ ID NO: 57, optionally with 1, 2, 3, 4 or 5 amino acid alterations outside the complementarity determining regions (CDRs).
128. An antibody according to clause 121, wherein the antibody comprises a variable heavy (VH) domain comprising, preferably consisting of SEQ ID NO: 61, optionally with 1, 2, 3, 4 or 5 amino acid alterations outside the complementarity determining regions (CDRs), and a variable light (VL) domain comprising, preferably consisting of SEQ ID NO: 64, optionally with 1, 2, 3, 4 or 5 amino acid alterations outside the complementarity determining regions (CDRs).
129. An antibody according to clause 121, wherein the antibody comprises a variable heavy (VH) domain comprising, preferably consisting of SEQ ID NO: 66, optionally with 1, 2, 3, 4 or 5 amino acid alterations outside the complementarity determining regions (CDRs), and a variable light (VL) domain comprising, preferably consisting of SEQ ID NO: 69, optionally with 1, 2, 3, 4 or 5 amino acid alterations outside the complementarity determining regions (CDRs).
130. An antibody according to clause 121, wherein the antibody comprises a variable heavy (VH) domain comprising, preferably consisting of SEQ ID NO: 71, optionally with 1, 2, 3, 4 or 5 amino acid alterations outside the complementarity determining regions (CDRs), and a variable light (VL) domain comprising, preferably consisting of SEQ ID NO: 740, optionally with 1, 2, 3, 4 or 5 amino acid alterations outside the complementarity determining regions (CDRs).
131. An antibody according to clause 121, wherein the antibody comprises a variable heavy (VH) domain comprising, preferably consisting of SEQ ID NO: 76, optionally with 1, 2, 3, 4 or 5 amino acid alterations outside the complementarity determining regions (CDRs), and a variable light (VL) domain comprising, preferably consisting of SEQ ID NO: 79, optionally with 1, 2, 3, 4 or 5 amino acid alterations outside the complementarity determining regions (CDRs).
132. An antibody according to clause 121, wherein the antibody comprises a variable heavy (VH) domain comprising, preferably consisting of SEQ ID NO: 82, optionally with 1, 2, 3, 4 or 5 amino acid alterations outside the complementarity determining regions (CDRs), and a variable light (VL) domain comprising, preferably consisting of SEQ ID NO: 85, optionally with 1, 2, 3, 4 or 5 amino acid alterations outside the complementarity determining regions (CDRs).
133. An antibody according to clause 121, wherein the antibody comprises a variable heavy (VH) domain comprising, preferably consisting of SEQ ID NO: 96, optionally with 1, 2, 3, 4 or 5 amino acid alterations outside the complementarity determining regions (CDRs), and a variable light (VL) domain comprising, preferably consisting of SEQ ID NO: 100, optionally with 1, 2, 3, 4 or 5 amino acid alterations outside the complementarity determining regions (CDRs).
134. An antibody according to clause 121, wherein the antibody comprises a variable heavy (VH) domain comprising, preferably consisting of SEQ ID NO: 125, optionally with 1, 2, 3, 4 or 5 amino acid alterations outside the complementarity determining regions (CDRs), and a variable light (VL) domain comprising, preferably consisting of SEQ ID NO: 128, optionally with 1, 2, 3, 4 or 5 amino acid alterations outside the complementarity determining regions (CDRs).
135. An antibody according to clause 121, wherein the antibody comprises a variable heavy (VH) domain comprising, preferably consisting of SEQ ID NO: 131, optionally with 1, 2, 3, 4 or 5 amino acid alterations outside the complementarity determining regions (CDRs), and a variable light (VL) domain comprising, preferably consisting of SEQ ID NO: 134, optionally with 1, 2, 3, 4 or 5 amino acid alterations outside the complementarity determining regions (CDRs).
136. An antibody according to clause 121, wherein the antibody comprises a variable heavy (VH) domain comprising, preferably consisting of SEQ ID NO: 137, optionally with 1, 2, 3, 4 or 5 amino acid alterations outside the complementarity determining regions (CDRs), and a variable light (VL) domain comprising, preferably consisting of SEQ ID NO: 141, optionally with 1, 2, 3, 4 or 5 amino acid alterations outside the complementarity determining regions (CDRs).
137. An antibody according to clause 121, wherein the antibody comprises a variable heavy (VH) domain comprising, preferably consisting of SEQ ID NO: 153, optionally with 1, 2, 3, 4 or 5 amino acid alterations outside the complementarity determining regions (CDRs), and a variable light (VL) domain comprising, preferably consisting of SEQ ID NO: 158, optionally with 1, 2, 3, 4 or 5 amino acid alterations outside the complementarity determining regions (CDRs).
138. An antibody according to clause 121, wherein the antibody comprises a variable heavy (VH) domain comprising, preferably consisting of SEQ ID NO: 161, optionally with 1, 2, 3, 4 or 5 amino acid alterations outside the complementarity determining regions (CDRs), and a variable light (VL) domain comprising, preferably consisting of SEQ ID NO: 165, optionally with 1, 2, 3, 4 or 5 amino acid alterations outside the complementarity determining regions (CDRs).
139. An antibody according to clause 121, wherein the antibody comprises a variable heavy (VH) domain comprising, preferably consisting of SEQ ID NO: 167, optionally with 1, 2, 3, 4 or 5 amino acid alterations outside the complementarity determining regions (CDRs), and a variable light (VL) domain comprising, preferably consisting of SEQ ID NO: 170, optionally with 1, 2, 3, 4 or 5 amino acid alterations outside the complementarity determining regions (CDRs).
140. An antibody according to clause 81 or clause 82, wherein the antibody comprises a variable heavy (VH) domain sequence and a variable light (VL) domain sequence and wherein:
   (i) the variable heavy (VH) comprises, preferably consists of a sequence having at least 90%, at least 95%, at least 98%, or at least 99%/identity to SEQ ID NO: 2 and
      the variable light (VL) domain comprises, preferably consists of a sequence having at least 90%, at least 95%, at least 98%, or at least 99% identity to SEQ ID NO: 7,
      provided that HCDR1 consists of SEQ ID NO: 3, HCDR2 consists of SEQ ID NO: 4, HCDR3 consists of SEQ ID NO: 5, LCDR1 consists of SEQ ID NO: 8, LCDR2 consists of SEQ ID NO: 9, and LCDR3 consists of SEQ ID NO: 10,
   (ii) the variable heavy (VH) comprises, preferably consists of a sequence having at least 90%, at least 95%, at least 98%, or at least 99% identity to SEQ ID NO: 12 and
      the variable light (VL) domain comprises, preferably consists of a sequence having at least 90%, at least 95%, at least 98%, or at least 99% identity to SEQ ID NO: 16,
      provided that HCDR1 consists of SEQ ID NO: 3, HCDR2 consists of SEQ ID NO: 13, HCDR3 consists of SEQ ID NO: 14, LCDR1 consists of SEQ ID NO: 17, LCDR2 consists of SEQ ID NO: 18, and LCDR3 consists of SEQ ID NO: 19,
   (iii) the variable heavy (VH) comprises, preferably consists of a sequence having at least 90%, at least 95%, at least 98%, or at least 99% identity to SEQ ID NO: 40 and
      the variable light (VL) domain comprises, preferably consists of a sequence having at least 90%, at least 95%, at least 98%, or at least 99% identity to SEQ ID NO: 16,
      provided that HCDR1 consists of SEQ ID NO: 3, HCDR2 consists of SEQ ID NO: 13, HCDR3 consists of SEQ ID NO: 41, LCDR1 consists of SEQ ID NO: 17, LCDR2 consists of SEQ ID NO: 18, and LCDR3 consists of SEQ ID NO: 19,
   (iv) the variable heavy (VH) comprises, preferably consists of a sequence having at least 90%, at least 95%, at least 98%, or at least 99% identity to SEQ ID NO: 44 and
      the variable light (VL) domain comprises, preferably consists of a sequence having at least 90%, at least 95%, at least 98%, or at least 99% identity to SEQ ID NO: 47,
      provided that HCDR1 consists of SEQ ID NO: 3, HCDR2 consists of SEQ ID NO: 13, HCDR3 consists of SEQ ID NO: 45, LCDR1 consists of SEQ ID NO: 17, LCDR2 consists of SEQ ID NO: 18, and LCDR3 consists of SEQ ID NO: 10,
   (v) the variable heavy (VH) comprises, preferably consists of a sequence having at least 90%, at least 95%, at least 98%, or at least 99% identity to SEQ ID NO: 49 and
      the variable light (VL) domain comprises, preferably consists of a sequence having at least 90%, at least 95%, at least 98%, or at least 99% identity to SEQ ID NO: 52,
      provided that HCDR1 consists of SEQ ID NO: 3, HCDR2 consists of SEQ ID NO: 13, HCDR3 consists of SEQ ID NO: 50, LCDR1 consists of SEQ ID NO: 17, LCDR2 consists of SEQ ID NO: 18, and LCDR3 consists of SEQ ID NO: 19,
   (vi) the variable heavy (VH) comprises, preferably consists of a sequence having at least 90%, at least 95%, at least 98%, or at least 99%/identity to SEQ ID NO: 54 and
      the variable light (VL) domain comprises, preferably consists of a sequence having at least 90%, at least 95%, at least 98%, or at least 99% identity to SEQ ID NO: 57,
      provided that HCDR1 consists of SEQ ID NO: 3, HCDR2 consists of SEQ ID NO: 13, HCDR3 consists of SEQ ID NO: 55, LCDR1 consists of SEQ ID NO: 58, LCDR2 consists of SEQ ID NO: 18, and LCDR3 consists of SEQ ID NO: 59,
   (vii) the variable heavy (VH) comprises, preferably consists of a sequence having at least 90%, at least 95%, at least 98%, or at least 99% identity to SEQ ID NO: 61 and
      the variable light (VL) domain comprises, preferably consists of a sequence having at least 90%, at least 95%, at least 98%, or at least 99% identity to SEQ ID NO: 64,
      provided that HCDR1 consists of SEQ ID NO: 3, HCDR2 consists of SEQ ID NO: 13, HCDR3 consists of SEQ ID NO: 62, LCDR1 consists of SEQ ID NO: 17, LCDR2 consists of SEQ ID NO: 18, and LCDR3 consists of SEQ ID NO: 59,
   (viii) the variable heavy (VH) comprises, preferably consists of a sequence having at least 90%, at least 95%, at least 98%, or at least 99% identity to SEQ ID NO: 66 and
      the variable light (VL) domain comprises, preferably consists of a sequence having at least 90%, at least 95%, at least 98%, or at least 99% identity to SEQ ID NO: 69,
      provided that HCDR1 consists of SEQ ID NO: 3, HCDR2 consists of SEQ ID NO: 13, HCDR3 consists of SEQ ID NO: 67, LCDR1 consists of SEQ ID NO: 17, LCDR2 consists of SEQ ID NO: 18, and LCDR3 consists of SEQ ID NO: 59,
   (ix) the variable heavy (VH) comprises, preferably consists of a sequence having at least 90%, at least 95%, at least 98%, or at least 99% identity to SEQ ID NO: 71 and
      the variable light (VL) domain comprises, preferably consists of a sequence having at least 90%, at least 95%, at least 98%, or at least 99% identity to SEQ ID NO: 74,
      provided that HCDR1 consists of SEQ ID NO: 3, HCDR2 consists of SEQ ID NO: 13, HCDR3 consists of SEQ ID NO: 72, LCDR1 consists of SEQ ID NO: 17, LCDR2 consists of SEQ ID NO: 18, and LCDR3 consists of SEQ ID NO: 59,
   (x) the variable heavy (VH) comprises, preferably consists of a sequence having at least 90%, at least 95%, at least 98%, or at least 99% identity to SEQ ID NO: 76 and
      the variable light (VL) domain comprises, preferably consists of a sequence having at least 90%, at least 95%, at least 98%, or at least 99% identity to SEQ ID NO: 79,
      provided that HCDR1 consists of SEQ ID NO: 3, HCDR2 consists of SEQ ID NO: 13, HCDR3 consists of SEQ ID NO: 77, LCDR1 consists of SEQ ID NO: 17, LCDR2 consists of SEQ ID NO: 18, and LCDR3 consists of SEQ ID NO: 80,
   (xi) the variable heavy (VH) comprises, preferably consists of a sequence having at least 90%, at least 95%, at least 98%, or at least 99%/identity to SEQ ID NO: 82 and
      the variable light (VL) domain comprises, preferably consists of a sequence having at least 90%, at least 95%, at least 98%, or at least 99% identity to SEQ ID NO: 85,
      provided that HCDR1 consists of SEQ ID NO: 3, HCDR2 consists of SEQ ID NO: 13, HCDR3 consists of SEQ ID NO: 83, LCDR1 consists of SEQ ID NO: 17, LCDR2 consists of SEQ ID NO: 18, and LCDR3 consists of SEQ ID NO: 86,
   (xii) the variable heavy (VH) comprises, preferably consists of a sequence having at least 90%, at least 95%, at least 98%, or at least 99% identity to SEQ ID NO: 96 and
      the variable light (VL) domain comprises, preferably consists of a sequence having at least 90%, at least 95%, at least 98%, or at least 99% identity to SEQ ID NO: 100,
      provided that HCDR1 consists of SEQ ID NO: 3, HCDR2 consists of SEQ ID NO: 97, HCDR3 consists of SEQ ID NO: 98, LCDR1 consists of SEQ ID NO: 101, LCDR2 consists of SEQ ID NO: 18, and LCDR3 consists of SEQ ID NO: 59,
   (xiii) the variable heavy (VH) comprises, preferably consists of a sequence having at least 90%, at least 95%, at least 98%, or at least 99% identity to SEQ ID NO: 125 and
      the variable light (VL) domain comprises, preferably consists of a sequence having at least 90%, at least 95%, at least 98%, or at least 99% identity to SEQ ID NO: 128,
      provided that HCDR1 consists of SEQ ID NO: 3, HCDR2 consists of SEQ ID NO: 13, HCDR3 consists of SEQ ID NO: 126, LCDR1 consists of SEQ ID NO: 17, LCDR2 consists of SEQ ID NO: 18, and LCDR3 consists of SEQ ID NO: 129,
   (xiv) the variable heavy (VH) comprises, preferably consists of a sequence having at least 90%, at least 95%, at least 98%, or at least 99% identity to SEQ ID NO: 131 and
      the variable light (VL) domain comprises, preferably consists of a sequence having at least 90%, at least 95%, at least 98%, or at least 99% identity to SEQ ID NO: 134,
      provided that HCDR1 consists of SEQ ID NO: 3, HCDR2 consists of SEQ ID NO: 13, HCDR3 consists of SEQ ID NO: 132, LCDR1 consists of SEQ ID NO: 17, LCDR2 consists of SEQ ID NO: 18, and LCDR3 consists of SEQ ID NO: 135,
   (xv) the variable heavy (VH) comprises, preferably consists of a sequence having at least 90%, at least 95%, at least 98%, or at least 99% identity to SEQ ID NO: 137 and
      the variable light (VL) domain comprises, preferably consists of a sequence having at least 90%, at least 95%, at least 98%, or at least 99% identity to SEQ ID NO: 141,
      provided that HCDR1 consists of SEQ ID NO: 3, HCDR2 consists of SEQ ID NO: 138, HCDR3 consists of SEQ ID NO: 139, LCDR1 consists of SEQ ID NO: 142, LCDR2 consists of SEQ ID NO: 18, and LCDR3 consists of SEQ ID NO: 86,
   (xvi) the variable heavy (VH) comprises, preferably consists of a sequence having at least 90%, at least 95%, at least 98%, or at least 99%/identity to SEQ ID NO: 153 and
      the variable light (VL) domain comprises, preferably consists of a sequence having at least 90%, at least 95%, at least 98%, or at least 99% identity to SEQ ID NO: 158,
      provided that HCDR1 consists of SEQ ID NO: 154, HCDR2 consists of SEQ ID NO: 155, HCDR3 consists of SEQ ID NO: 156, LCDR1 consists of SEQ ID NO: 17, LCDR2 consists of SEQ ID NO: 18, and LCDR3 consists of SEQ ID NO: 159,
   (xvii) the variable heavy (VH) comprises, preferably consists of a sequence having at least 90%, at least 95%, at least 98%, or at least 99% identity to SEQ ID NO: 161 and
      the variable light (VL) domain comprises, preferably consists of a sequence having at least 90%, at least 95%, at least 98%, or at least 99% identity to SEQ ID NO: 165,
      provided that HCDR1 consists of SEQ ID NO: 3, HCDR2 consists of SEQ ID NO: 162, HCDR3 consists of SEQ ID NO: 163, LCDR1 consists of SEQ ID NO: 17, LCDR2 consists of SEQ ID NO: 18, and LCDR3 consists of SEQ ID NO: 135, or
   (xviii) the variable heavy (VH) comprises, preferably consists of a sequence having at least 90%, at least 95%, at least 98%, or at least 99% identity to SEQ ID NO: 167 and
      the variable light (VL) domain comprises, preferably consists of a sequence having at least 90%, at least 95%, at least 98%, or at least 99% identity to SEQ ID NO: 170,
      provided that HCDR1 consists of SEQ ID NO: 3, HCDR2 consists of SEQ ID NO: 13, HCDR3 consists of SEQ ID NO: 168, LCDR1 consists of SEQ ID NO: 17, LCDR2 consists of SEQ ID NO: 18, and LCDR3 consists of SEQ ID NO: 10.
141. An antibody according to clause 140, wherein the variable heavy (VH) domain sequence comprises, preferably consists of a sequence having at least 90%, at least 95%, at least 98%, or at least 99% identity to the VH domain having SEQ ID NO: 2 and the variable light (VL) domain sequence comprises, preferably consists of a sequence having at least 90%, at least 95%, at least 98%, or at least 99% identity to the VL domain having SEQ ID NO: 7, provided that HCDR1 consists of SEQ ID NO: 3, HCDR2 consists of SEQ ID NO: 4, HCDR3 consists of SEQ ID NO: 5, LCDR1 consists of SEQ ID NO: 8, LCDR2 consists of SEQ ID NO: 9, and LCDR3 consists of SEQ ID NO: 10.
142. An antibody according to clause 140, wherein the variable heavy (VH) domain sequence comprises, preferably consists of a sequence having at least 90%, at least 95%, at least 98%, or at least 99% identity to the VH domain having SEQ ID NO: 12 and the variable light (VL) domain sequence comprises, preferably consists of a sequence having at least 90%, at least 95%, at least 98%, or at least 99% identity to the VL domain having SEQ ID NO: 16, provided that HCDR1 consists of SEQ ID NO: 3, HCDR2 consists of SEQ ID NO: 13, HCDR3 consists of SEQ ID NO: 14, LCDR1 consists of SEQ ID NO: 17, LCDR2 consists of SEQ ID NO: 18, and LCDR3 consists of SEQ ID NO: 19.
143. An antibody according to clause 140, wherein the variable heavy (VH) domain sequence comprises, preferably consists of a sequence having at least 90%, at least 95%, at least 98%, or at least 99% identity to the VH domain having SEQ ID NO: 40 and the variable light (VL) domain sequence comprises, preferably consists of a sequence having at least 90%, at least 95%, at least 98%, or at least 99% identity to the VL domain having SEQ ID NO: 16, provided that HCDR1 consists of SEQ ID NO: 3, HCDR2 consists of SEQ ID NO: 13, HCDR3 consists of SEQ ID NO: 41, LCDR1 consists of SEQ ID NO: 17, LCDR2 consists of SEQ ID NO: 18, and LCDR3 consists of SEQ ID NO: 19.
144. An antibody according to clause 140, wherein the variable heavy (VH) domain sequence comprises, preferably consists of a sequence having at least 90%, at least 95%, at least 98%, or at least 99% identity to the VH domain having SEQ ID NO: 44 and the variable light (VL) domain sequence comprises, preferably consists of a sequence having at least 90%, at least 95%, at least 98%, or at least 99% identity to the VL domain having SEQ ID NO: 47, provided that HCDR1 consists of SEQ ID NO: 3, HCDR2 consists of SEQ ID NO: 13, HCDR3 consists of SEQ ID NO: 45, LCDR1 consists of SEQ ID NO: 17, LCDR2 consists of SEQ ID NO: 18, and LCDR3 consists of SEQ ID NO: 10.
145. An antibody according to clause 140, wherein the variable heavy (VH) domain sequence comprises, preferably consists of a sequence having at least 90%, at least 95%, at least 98%, or at least 99% identity to the VH domain having SEQ ID NO: 49 and the variable light (VL) domain sequence comprises, preferably consists of a sequence having at least 90%, at least 95%, at least 98%, or at least 99% identity to the VL domain having SEQ ID NO: 52, provided that HCDR1 consists of SEQ ID NO: 3, HCDR2 consists of SEQ ID NO: 13, HCDR3 consists of SEQ ID NO: 50, LCDR1 consists of SEQ ID NO: 17, LCDR2 consists of SEQ ID NO: 18, and LCDR3 consists of SEQ ID NO: 19.
146. An antibody according to clause 140, wherein the variable heavy (VH) domain sequence comprises, preferably consists of a sequence having at least 90%, at least 95%, at least 98%, or at least 99% identity to the VH domain having SEQ ID NO: 54 and the variable light (VL) domain sequence comprises, preferably consists of a sequence having at least 90%, at least 95%, at least 98%, or at least 99% identity to the VL domain having SEQ ID NO: 57, provided that HCDR1 consists of SEQ ID NO: 3, HCDR2 consists of SEQ ID NO: 13, HCDR3 consists of SEQ ID NO: 55, LCDR1 consists of SEQ ID NO: 58, LCDR2 consists of SEQ ID NO: 18, and LCDR3 consists of SEQ ID NO: 59
147. An antibody according to clause 140, wherein the variable heavy (VH) domain sequence comprises, preferably consists of a sequence having at least 90%, at least 95%, at least 98%, or at least 99% identity to the VH domain having SEQ ID NO: 61 and the variable light (VL) domain sequence comprises, preferably consists of a sequence having at least 90%, at least 95%, at least 98%, or at least 99% identity to the VL domain having SEQ ID NO: 64, provided that HCDR1 consists of SEQ ID NO: 3, HCDR2 consists of SEQ ID NO: 13, HCDR3 consists of SEQ ID NO: 62, LCDR1 consists of SEQ ID NO: 17, LCDR2 consists of SEQ ID NO: 18, and LCDR3 consists of SEQ ID NO: 59.
148. An antibody according to clause 140, wherein the variable heavy (VH) domain sequence comprises, preferably consists of a sequence having at least 90%, at least 95%, at least 98%, or at least 99% identity to the VH domain having SEQ ID NO: 66 and the variable light (VL) domain sequence comprises, preferably consists of a sequence having at least 90%, at least 95%, at least 98%, or at least 99% identity to the VL domain having SEQ ID NO: 69, provided that HCDR1 consists of SEQ ID NO: 3, HCDR2 consists of SEQ ID NO: 13, HCDR3 consists of SEQ ID NO: 67, LCDR1 consists of SEQ ID NO: 17, LCDR2 consists of SEQ ID NO: 18, and LCDR3 consists of SEQ ID NO: 59.
149. An antibody according to clause 140, wherein the variable heavy (VH) domain sequence comprises, preferably consists of a sequence having at least 90%, at least 95%, at least 98%, or at least 99% identity to the VH domain having SEQ ID NO: 71 and the variable light (VL) domain sequence comprises, preferably consists of a sequence having at least 90%, at least 95%, at least 98%, or at least 99% identity to the VL domain having SEQ ID NO: 74, provided that HCDR1 consists of SEQ ID NO: 3, HCDR2 consists of SEQ ID NO: 13, HCDR3 consists of SEQ ID NO: 72, LCDR1 consists of SEQ ID NO: 17, LCDR2 consists of SEQ ID NO: 18, and LCDR3 consists of SEQ ID NO: 59.
150. An antibody according to clause 140, wherein the variable heavy (VH) domain sequence comprises, preferably consists of a sequence having at least 90%, at least 95%, at least 98%, or at least 99% identity to the VH domain having SEQ ID NO: 76 and the variable light (VL) domain sequence comprises, preferably consists of a sequence having at least 90%, at least 95%, at least 98%, or at least 99/6 identity to the VL domain having SEQ ID NO: 79, provided that HCDR1 consists of SEQ ID NO: 3, HCDR2 consists of SEQ ID NO: 13, HCDR3 consists of SEQ ID NO: 77, LCDR1 consists of SEQ ID NO: 17, LCDR2 consists of SEQ ID NO: 18, and LCDR3 consists of SEQ ID NO: 80.
151. An antibody according to clause 140, wherein the variable heavy (VH) domain sequence comprises, preferably consists of a sequence having at least 90%, at least 95%, at least 98%, or at least 99% identity to the VH domain having SEQ ID NO: 82 and the variable light (VL) domain sequence comprises, preferably consists of a sequence having at least 90%, at least 95%, at least 98%, or at least 99% identity to the VL domain having SEQ ID NO: 85, provided that HCDR1 consists of SEQ ID NO: 3, HCDR2 consists of SEQ ID NO: 13, HCDR3 consists of SEQ ID NO: 83, LCDR1 consists of SEQ ID NO: 17, LCDR2 consists of SEQ ID NO: 18, and LCDR3 consists of SEQ ID NO: 86.
152. An antibody according to clause 140, wherein the variable heavy (VH) domain sequence comprises, preferably consists of a sequence having at least 90%, at least 95%, at least 98%, or at least 99% identity to the VH domain having SEQ ID NO: 96 and the variable light (VL) domain sequence comprises, preferably consists of a sequence having at least 90%, at least 95%, at least 98%, or at least 99% identity to the VL domain having SEQ ID NO: 100, provided that HCDR1 consists of SEQ ID NO: 3, HCDR2 consists of SEQ ID NO: 97, HCDR3 consists of SEQ ID NO: 98, LCDR1 consists of SEQ ID NO: 101, LCDR2 consists of SEQ ID NO: 18, and LCDR3 consists of SEQ ID NO: 59.
153. An antibody according to clause 140, wherein the variable heavy (VH) domain sequence comprises, preferably consists of a sequence having at least 90%, at least 95%, at least 98%, or at least 99% identity to the VH domain having SEQ ID NO: 125 and the variable light (VL) domain sequence comprises, preferably consists of a sequence having at least 90%, at least 95%, at least 98%, or at least 99% identity to the VL domain having SEQ ID NO: 128, provided that HCDR1 consists of SEQ ID NO: 3, HCDR2 consists of SEQ ID NO: 13, HCDR3 consists of SEQ ID NO: 126, LCDR1 consists of SEQ ID NO: 17, LCDR2 consists of SEQ ID NO: 18, and LCDR3 consists of SEQ ID NO: 129.
154. An antibody according to clause 140, wherein the variable heavy (VH) domain sequence comprises, preferably consists of a sequence having at least 90%, at least 95%, at least 98%, or at least 99% identity to the VH domain having SEQ ID NO: 131 and the variable light (VL) domain sequence comprises, preferably consists of a sequence having at least 90%, at least 95%, at least 98%, or at least 99% identity to the VL domain having SEQ ID NO: 134, provided that HCDR1 consists of SEQ ID NO: 3, HCDR2 consists of SEQ ID NO: 13, HCDR3 consists of SEQ ID NO: 132, LCDR1 consists of SEQ ID NO: 17, LCDR2 consists of SEQ ID NO: 18, and LCDR3 consists of SEQ ID NO: 135.
155. An antibody according to clause 140, wherein the variable heavy (VH) domain sequence comprises, preferably consists of a sequence having at least 90%, at least 95%, at least 98%, or at least 99% identity to the VH domain having SEQ ID NO: 137 and the variable light (VL) domain sequence comprises, preferably consists of a sequence having at least 90%, at least 95%, at least 98%, or at least 99% identity to the VL domain having SEQ ID NO: 141, provided that HCDR1 consists of SEQ ID NO: 3, HCDR2 consists of SEQ ID NO: 138, HCDR3 consists of SEQ ID NO: 139, LCDR1 consists of SEQ ID NO: 142, LCDR2 consists of SEQ ID NO: 18, and LCDR3 consists of SEQ ID NO: 86.
156. An antibody according to clause 140, wherein the variable heavy (VH) domain sequence comprises, preferably consists of a sequence having at least 90%, at least 95%, at least 98%, or at least 99% identity to the VH domain having SEQ ID NO: 153 and the variable light (VL) domain sequence comprises, preferably consists of a sequence having at least 90%, at least 95%, at least 98%, or at least 99% identity to the VL domain having SEQ ID NO: 158, provided that HCDR1 consists of SEQ ID NO: 154, HCDR2 consists of SEQ ID NO: 155, HCDR3 consists of SEQ ID NO: 156, LCDR1 consists of SEQ ID NO: 17, LCDR2 consists of SEQ ID NO: 18, and LCDR3 consists of SEQ ID NO: 159.
157. An antibody according to clause 140, wherein the variable heavy (VH) domain sequence comprises, preferably consists of a sequence having at least 90%, at least 95%, at least 98%, or at least 99% identity to the VH domain having SEQ ID NO: 161 and the variable light (VL) domain sequence comprises, preferably consists of a sequence having at least 90%, at least 95%, at least 98%, or at least 99% identity to the VL domain having SEQ ID NO: 165, provided that HCDR1 consists of SEQ ID NO: 3, HCDR2 consists of SEQ ID NO: 162, HCDR3 consists of SEQ ID NO: 163, LCDR1 consists of SEQ ID NO: 17, LCDR2 consists of SEQ ID NO: 18, and LCDR3 consists of SEQ ID NO: 135.
158. An antibody according to clause 140, wherein the variable heavy (VH) domain sequence comprises, preferably consists of a sequence having at least 90%, at least 95%, at least 98%, or at least 99% identity to the VH domain having SEQ ID NO: 167 and the variable light (VL) domain sequence comprises, preferably consists of a sequence having at least 90%, at least 95%, at least 98%, or at least 99% identity to the VL domain having SEQ ID NO: 170, provided that HCDR1 consists of SEQ ID NO: 3, HCDR2 consists of SEQ ID NO: 13, HCDR3 consists of SEQ ID NO: 168, LCDR1 consists of SEQ ID NO: 17, LCDR2 consists of SEQ ID NO: 18, and LCDR3 consists of SEQ ID NO: 10.
159. An antibody according to clause 81 or clause 82, wherein the antibody comprises a variable heavy (VH) domain sequence and a variable light (VL) domain sequence and wherein:
   (i) the variable heavy (VH) domain comprises, preferably consists of SEQ ID NO: 2 and the variable light (VL) domain comprises, preferably consists of SEQ ID NO: 7,
   (ii) the variable heavy (VH) domain comprises, preferably consists of SEQ ID NO: 12 and the variable light (VL) domain comprises, preferably consists of SEQ ID NO: 16,
   (iii) the variable heavy (VH) domain comprises, preferably consists of SEQ ID NO: 40 and the variable light (VL) domain comprises, preferably consists of SEQ ID NO: 16,
   (iv) the variable heavy (VH) domain comprises, preferably consists of SEQ ID NO: 44 and the variable light (VL) domain comprises, preferably consists of SEQ ID NO: 47,
   (v) the variable heavy (VH) domain comprises, preferably consists of SEQ ID NO: 49 and the variable light (VL) domain comprises, preferably consists of SEQ ID NO: 52,
   (vi) the variable heavy (VH) domain comprises, preferably consists of SEQ ID NO: 54 and the variable light (VL) domain comprises, preferably consists of SEQ ID NO: 57,
   (vii) the variable heavy (VH) domain comprises, preferably consists of SEQ ID NO: 61 and the variable light (VL) domain comprises, preferably consists of SEQ ID NO: 64,
   (viii) the variable heavy (VH) domain comprises, preferably consists of SEQ ID NO: 66 and the variable light (VL) domain comprises, preferably consists of SEQ ID NO: 69,
   (ix) the variable heavy (VH) domain comprises, preferably consists of SEQ ID NO: 71 and the variable light (VL) domain comprises, preferably consists of SEQ ID NO: 74,
   (x) the variable heavy (VH) domain comprises, preferably consists of SEQ ID NO: 76 and the variable light (VL) domain comprises, preferably consists of SEQ ID NO: 79,
   (xi) the variable heavy (VH) domain comprises, preferably consists of SEQ ID NO: 82 and the variable light (VL) domain comprises, preferably consists of SEQ ID NO: 85,
   (xii) the variable heavy (VH) domain comprises, preferably consists of SEQ ID NO: 96 and the variable light (VL) domain comprises, preferably consists of SEQ ID NO: 100,
   (xiii) the variable heavy (VH) domain comprises, preferably consists of SEQ ID NO: 125 and the variable light (VL) domain comprises, preferably consists of SEQ ID NO: 128,
   (xiv) the variable heavy (VH) domain comprises, preferably consists of SEQ ID NO: 131 and the variable light (VL) domain comprises, preferably consists of SEQ ID NO: 134,
   (xv) the variable heavy (VH) domain comprises, preferably consists of SEQ ID NO: 137 and the variable light (VL) domain comprises, preferably consists of SEQ ID NO: 141,
   (xvi) the variable heavy (VH) domain comprises, preferably consists of SEQ ID NO: 153 and the variable light (VL) domain comprises, preferably consists of SEQ ID NO: 158,
   (xvii) the variable heavy (VH) domain comprises, preferably consists of SEQ ID NO: 161 and the variable light (VL) domain comprises, preferably consists of SEQ ID NO: 165, or
   (xviii) the variable heavy (VH) domain comprises, preferably consists of SEQ ID NO: 167 and the variable light (VL) domain comprises, preferably consists of SEQ ID NO: 170.
160. An antibody according to clause 159, wherein the antibody comprises the VH domain comprising, preferably consisting of SEQ ID NO: 2 and the VL domain comprising, preferably consisting of SEQ ID NO: 7.
161. An antibody according to clause 159, wherein the antibody comprises the VH domain comprising, preferably consisting of SEQ ID NO: 12 and the VL domain comprising, preferably consisting of SEQ ID NO: 16.
162. An antibody according to clause 159, wherein the antibody comprises the VH domain comprising, preferably consisting of SEQ ID NO: 40 and the VL domain comprising, preferably consisting of SEQ ID NO: 16.
163. An antibody according to clause 159, wherein the antibody comprises the VH domain comprising, preferably consisting of SEQ ID NO: 44 and the VL domain comprising, preferably consisting of SEQ ID NO: 47.
164. An antibody according to clause 159, wherein the antibody comprises the VH domain comprising, preferably consisting of SEQ ID NO: 49 and the VL domain comprising, preferably consisting of SEQ ID NO: 52.
165. An antibody according to clause 159, wherein the antibody comprises the VH domain comprising, preferably consisting of SEQ ID NO: 54 and the VL domain comprising, preferably consisting of SEQ ID NO: 57.
166. An antibody according to clause 159, wherein the antibody comprises the VH domain comprising, preferably consisting of SEQ ID NO: 61 and the VL domain comprising, preferably consisting of SEQ ID NO: 64.
167. An antibody according to clause 159, wherein the antibody comprises the VH domain comprising, preferably consisting of SEQ ID NO: 66 and the VL domain comprising, preferably consisting of SEQ ID NO: 69.
168. An antibody according to clause 159, wherein the antibody comprises the VH domain comprising, preferably consisting of SEQ ID NO: 71 and the VL domain comprising, preferably consisting of SEQ ID NO: 74.
169. An antibody according to clause 159, wherein the antibody comprises the VH domain comprising, preferably consisting of SEQ ID NO: 76 and the VL domain comprising, preferably consisting of SEQ ID NO: 79.
170. An antibody according to clause 159, wherein the antibody comprises the VH domain comprising, preferably consisting of SEQ ID NO: 82 and the VL domain comprising, preferably consisting of SEQ ID NO: 85.
171. An antibody according to clause 159, wherein the antibody comprises the VH domain comprising, preferably consisting of SEQ ID NO: 96 and the VL domain comprising, preferably consisting of SEQ ID NO: 100.
172. An antibody according to clause 159, wherein the antibody comprises the VH domain comprising, preferably consisting of SEQ ID NO: 125 and the VL domain comprising, preferably consisting of SEQ ID NO: 128.
173. An antibody according to clause 159, wherein the antibody comprises the VH domain comprising, preferably consisting of SEQ ID NO: 131 and the VL domain comprising, preferably consisting of SEQ ID NO: 134.
174. An antibody according to clause 159, wherein the antibody comprises the VH domain comprising, preferably consisting of SEQ ID NO: 137 and the VL domain comprising, preferably consisting of SEQ ID NO: 141.
175. An antibody according to clause 159, wherein the antibody comprises the VH domain comprising, preferably consisting of SEQ ID NO: 153 and the VL domain comprising, preferably consisting of SEQ ID NO: 158.
176. An antibody according to clause 159, wherein the antibody comprises the VH domain comprising, preferably consisting of SEQ ID NO: 161 and the VL domain comprising, preferably consisting of SEQ ID NO: 165.
177. An antibody according to clause 159, wherein the antibody comprises the VH domain comprising, preferably consisting of SEQ ID NO: 167 and the VL domain comprising, preferably consisting of SEQ ID NO: 170.
178. An antibody that binds to the same epitope on KL49 as an antibody according to any one of clauses 81 to 177
179. An antibody capable of competing for binding to KL49 with any an antibody according to any one of clauses. 81 to 177.
180. An antibody according to any one of the preceding clauses, wherein the antibody shows complement-dependant cytotoxic activity (CDC) activity.
181. An antibody according to any one of the preceding clauses, wherein the antibody is a human IgG1 or human IgG4.
182. An antibody according to clause 181, wherein the antibody is a human IgG1, optionally wherein the antibody comprises a constant region sequence of SEQ ID NO: 418.
183. An antibody according to clause 181, wherein the antibody is a human IgG4, optionally wherein the antibody comprises a constant region sequence of SEQ ID NO: 436.
184. An antibody according to any one of clauses 1 to 180, wherein the antibody is a human IgA1 (e.g., comprising a constant region sequence SEQ ID NO: 484) or human IgA2 (e.g., comprising a constant region sequence SEQ ID NO: 485).
185. An antibody according to any one of clauses 1 to 184, wherein the antibody comprises kappa (κ) light chain constant regions, preferably constant domain sequence SEQ ID NO: 448.
186. A nucleic acid sequence comprising a sequence that encodes a VH domain and/or an VL domain of an antibody according to any preceding clause.
187. A nucleic acid sequence comprising a sequence that encodes the heavy chain and/or the light chain of an antibody according to any preceding clause.
188. A vector comprising a nucleic acid according to clause 186 or clause 187, optionally wherein the vector is a CHO vector.
189. A host cell comprising a nucleic acid according to clause 186 or clause 187, or a vector according to clause 188.
190. A pharmaceutical composition comprising (i) an isolated nucleic acid encoding an antibody according to any one of clauses 1 to 185, or (ii) a nucleic acid according to clause 186 or clause 187, and a pharmaceutically acceptable excipient.
191. A pharmaceutical composition comprising an antibody according to any one of clauses 1 to 185, and a pharmaceutically acceptable excipient.
192. A pharmaceutical composition according to clause 190 or clause 191, wherein the pharmaceutical composition is formulated for intravenous, intramuscular, or subcutaneous administration.
193. A pharmaceutical composition according to any one of clauses 190 to 192, wherein the pharmaceutical composition further comprises at least one further therapeutic agent.
194. A pharmaceutical composition according to clause 193, wherein the further therapeutic agent is at least one, preferably one or two, further antibodies.
195. A pharmaceutical composition according to clause 193, wherein the further therapeutic agent is carbapenem.
196. A pharmaceutical composition according to clause 193, wherein the further therapeutic agent is colistin.
197. A kit comprising an antibody according to any one of clause 1 to 185.
198. A kit comprising a pharmaceutical composition according to any one of clauses 190 to 196.
199. A kit according to clause 198, wherein the kit further comprises a label or instructions for use to prevent and/or treat a bacterial infection caused by Acinetobacter baumannii in a human; optionally wherein the label or instructions comprise a marketing authorisation number (e.g., an FDA or EMA authorisation number); optionally wherein the kit comprises an IV or injection device that comprises the antibody.
200. A kit according to clause 198 or clause 199, wherein the antibody is contained in a sealed container.
201. An antibody according to any one of clauses 1 to 185, or a pharmaceutical composition according to any one of clauses 190 to 196, or a nucleic acid according to clause 186 or clause 187, for use as a medicament.
202. An antibody according to any one of clauses 1 to 185, or a pharmaceutical composition according to any one of clauses 190 to 196, or a nucleic acid according to clause 186 or clause 187, for use in a method of treating a bacterial infection caused by Acinetobacter baumannii, said method comprising administering the antibody, composition, or nucleic acid to a patient.
203. An antibody according to any one of clauses 1 to 185, or a pharmaceutical composition according to any one of clauses 190 to 196, or a nucleic acid according to clause 186 or clause 187, for use in a method of preventing a bacterial infection caused by Acinetobacter baumannii, said method comprising administering the antibody, composition, or nucleic acid to a patient.
204. An antibody according to any one of clauses 1 to 185, or a pharmaceutical composition according to any one of clauses 190 to 196, or a nucleic acid according to clause 186 or clause 187, for use according to clause 202 or clause 203, wherein the presence or absence of Oxa-23 has been determined in a sample from the patient prior to administering the antibody, composition, or nucleic acid to the patient.
205. An antibody according to any one of clauses 1 to 185, or a pharmaceutical composition according to any one of clauses 190 to 196, or a nucleic acid according to clause 186 or clause 187, for use according to clause 202, clause 203 or clause 204, wherein the presence or absence of OCL1 LOS has been determined in a sample from the patient prior to administering the antibody, composition, or nucleic acid to the patient.
206. An antibody according to any one of clauses 1 to 185, or a pharmaceutical composition according to any one of clauses 190 to 196, or a nucleic acid according to clause 186 or clause 187, for use according to any one of clauses 202 to 205, wherein the presence or absence of KL49 has been determined in a sample from the patient prior to administering the antibody, composition, or nucleic acid to the patient.
207. Use of an antibody according to any one of clauses 1 to 185, or a pharmaceutical composition according to any one of clauses 190 to 196, or a nucleic acid according to clause 186 or clause 187, in the manufacture of a medicament for use in a method of treating a bacterial infection caused by Acinetobacter baumannii in a patient.
208. Use of an antibody according to any one of clauses 1 to 185, or a pharmaceutical composition according to any one of clauses 190 to 196, or a nucleic acid according to clause 186 or clause 187, in the manufacture of a medicament for use in a method of preventing a bacterial infection caused by Acinetobacter baumannii in a patient.
209. A method of treating a bacterial infection caused by Acinetobacter baumannii in a patient comprising administering to said patient a therapeutically effective amount of an antibody according to any one of clauses 1 to 185, or a pharmaceutical composition according to any one of clauses 190 to 196, or a nucleic acid according to clause 186 or clause 187.
210. A method of preventing a bacterial infection caused by Acinetobacter baumannii in a patient comprising administering to said patient a therapeutically effective amount of an antibody according to any one of clauses 1 to 185, or a pharmaceutical composition according to any one of clauses 190 to 196, or a nucleic acid according to clause 186 or clause 187.
211. An antibody, composition, or nucleic acid for use according to any one of clauses 202 to 206, use according to clause 207 or clause 208, or a method according to clause 209 or clause 210, wherein the bacterial infection caused by Acinetobacter baumannii is a nosocomial bacterial infection caused by Acinetobacter baumannii.
212. An antibody, composition, or nucleic acid for use according to any one of clauses 202 to 206, use according to clause 207 or clause 208, or a method according to clause 209 or clause 210, wherein the patient has a lower respiratory tract infection, for example pneumonia.
213. An antibody, composition, or nucleic acid for use according to any one of clauses 202 to 206, use according to clause 207 or clause 208, or a method according to clause 209 or clause 210, wherein the patient has sepsis.
214. An antibody, composition, or nucleic acid for use according to any one of clauses 202 to 206, use according to clause 207 or clause 208, or a method according to clause 209 or clause 210, wherein the patient has bacteremia.
215. An antibody, composition, or nucleic acid for use according to any one of clauses 202 to 206, use according to clause 207 or clause 208, or a method according to clause 209 or clause 210, wherein the method further comprises administering at least one further therapeutic agent.
216. An antibody, composition, or nucleic acid for use according to clause 215, use according to clause 215, or a method according to clause 215, wherein the administration of the further therapeutic agent is simultaneous, separate or sequential.
217. An antibody, composition, or nucleic acid for use according to clause 215 or clause 216, use according to clause 215 or clause 216, or a method according to clause 215 or clause 216, wherein the further therapeutic agent is at least one, preferably one or two, further antibodies.
218. An antibody, composition, or nucleic acid for use according to any one of clauses 215 to 217, use according to any one of clauses 215 to 217, or a method according to any one of clauses 215 to 217, wherein the at least one further therapeutic agent is carbapenem.
219. An antibody, composition, or nucleic acid for use according to any one of clauses 215 to 217, use according to any one of clauses 215 to 217, or a method according to any one of clauses 215 to 217, wherein the at least one further therapeutic agent is colistin.
220. Use of an antibody according to any one of clauses 1 to 185, for determining the presence or absence of Acinetobacter baumannii in a sample.
221. A method of determining the presence or absence of Acinetobacter baumannii in a sample comprising contacting the sample with an antibody according to any one of 1 to 185; and testing for binding between the antibody and Acinetobacter baumannii in the sample; wherein detection of binding indicates the presence of Acinetobacter baumannii in the sample and wherein absence of binding indicates the absence of Acinetobacter baumannii in the sample.
222. Use of an antibody according to any one of clauses 1 to 36 for determining the presence or absence of Oxa-23 in a sample, optionally wherein determining the presence or absence of Oxa-23 in a sample is used for determining a treatment protocol in a patient.
223. Use of an antibody according to any one of clauses 1, 2, or 37 to 80 for determining the presence or absence of OCL1 LOS in a sample, optionally wherein determining the presence or absence of OCL1 LOS in a sample is used for determining a treatment protocol in a patient.
224. Use of an antibody according to any one of clauses 1, 2, or 81 to 179 for determining the presence or absence of KL49 in a sample, optionally wherein determining the presence or absence of KL49 in a sample is used for determining a treatment protocol in a patient.
225. Use according to any one of clauses 222 to 224, wherein the antibody is conjugated to a detectable label.
226. Use according to any one of clauses 222 to 225, wherein the sample has been obtained from a human who has been or is suspected of having been infected with Acinetobacter baumannii.
227. Use according to clause 226, wherein the sample has been obtained from a human who has been or is suspected of having been infected with Acinetobacter baumannii who exhibits one or more symptoms of a bacterial infection.
228. Use according to any one of clauses 222 to 227, wherein the sample is a serum, plasma, or whole blood sample, an oral or nasal swab, urine, faeces, or cerebrospinal fluid (CFS), or wherein the sample is from any suspected Acinetobacter baumannii infected organ or tissue.
229. A diagnostic kit comprising an antibody according to any one of clauses 1 to 185, and optionally one or more buffering solutions.
230. A diagnostic kit according to clause 229, wherein the kit comprises a first reagent comprising the antibody according to any one of clauses 1 to 185, and a second reagent comprising a detector molecule that binds to the first reagent.
231. A diagnostic kit according to clause 230, wherein the detector molecule is an antibody that comprises or is conjugated to a detectable label.

1. An antibody that specifically binds at the surface of Acinetobacter baumannii bacteria.

2. An antibody according to claim 1, wherein the antibody induces complement activation (e.g. as measured by flow cytometry-based assay).

3. An antibody according to claim 1 or claim 2, that specifically binds to Oxa-23 at the surface of Acinetobacter baumannii bacteria.

4. An antibody according to claim 1 or claim 2, that specifically binds Oxa-23 on outer membrane vesicles (OMVs) of Acinetobacter baumannii bacteria.

5. An antibody according to any one of claims 1 to 4, wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, and wherein the HCDR3 is selected from the group consisting of SEQ ID NO: 34, SEQ ID NO: 24, SEQ ID NO: 174, and SEQ ID NO: 182.

6. An antibody according to any one of the claims 1 to 4, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, and wherein:
(i) HCDR1 consists of SEQ ID NO: 32, HCDR2 consists of SEQ ID NO: 33, HCDR3 consists of SEQ ID NO: 34, LCDR1 consists of SEQ ID NO: 37, LCDR2 consists of SEQ ID NO: 28, and LCDR3 consists of SEQ ID NO: 38,
(ii) HCDR1 consists of SEQ ID NO: 22, HCDR2 consists of SEQ ID NO: 23, HCDR3 consists of SEQ ID NO: 24, LCDR1 consists of SEQ ID NO: 27, LCDR2 consists of SEQ ID NO: 28, and LCDR3 consists of SEQ ID NO: 29,
(iii) HCDR1 consists of SEQ ID NO: 3, HCDR2 consists of SEQ ID NO: 173, HCDR3 consists of SEQ ID NO: 174, LCDR1 consists of SEQ ID NO: 149, LCDR2 consists of SEQ ID NO: 177, and LCDR3 consists of SEQ ID NO: 178, or
(iv) HCDR1 consists of SEQ ID NO: 181, HCDR2 consists of SEQ ID NO: 173, HCDR3 consists of SEQ ID NO: 182, LCDR1 consists of SEQ ID NO: 185, LCDR2 consists of SEQ ID NO: 28, and LCDR3 consists of SEQ ID NO: 186.

7. An antibody according to any one of claims 1 to 4, wherein the antibody comprises a variable heavy (VH) domain sequence and a variable light (VL) domain sequence and wherein:
(i) the variable heavy (VH) domain comprises, preferably consists of SEQ ID NO: 31 and the variable light (VL) domain comprises, preferably consists of SEQ ID NO:36,
(ii) the variable heavy (VH) domain comprises, preferably consists of SEQ ID NO: 21 and the variable light (VL) domain comprises, preferably consists of SEQ ID NO: 26,
(iii) the variable heavy (VH) domain comprises, preferably consists of SEQ ID NO: 172 and the variable light (VL) domain comprises, preferably consists of SEQ ID NO:176, or
(iv) the variable heavy (VH) domain comprises, preferably consists of SEQ ID NO: 180 and the variable light (VL) domain comprises, preferably consists of SEQ ID NO:184,
optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

8. An antibody according to any one of claims 1 to 4, wherein the antibody comprises a variable heavy (VH) domain sequence and a variable light (VL) domain sequence and wherein:
(i) the variable heavy (VH) domain comprises, preferably consists of a sequence having at least 90%, at least 95%, at least 98%, or at least 99% identity to SEQ ID NO: 31 and
the variable light (VL) domain comprises, preferably consists of a sequence having at least 90%, at least 95%, at least 98%, or at least 99% identity to SEQ ID NO: 36,
provided that HCDR1 consists of SEQ ID NO: 32, HCDR2 consists of SEQ ID NO: 33, HCDR3 consists of SEQ ID NO: 34, LCDR1 consists of SEQ ID NO: 37, LCDR2 consists of SEQ ID NO: 28, and LCDR3 consists of SEQ ID NO: 38,
(ii) the variable heavy (VH) domain comprises, preferably consists of a sequence having at least 90%, at least 95%, at least 98%, or at least 99% identity to SEQ ID NO: 21 and
the variable light (VL) domain comprises, preferably consists of a sequence having at least 90%, at least 95%, at least 98%, or at least 99% identity to SEQ ID NO: 26,
provided that HCDR1 consists of SEQ ID NO: 22, HCDR2 consists of SEQ ID NO: 23, HCDR3 consists of SEQ ID NO: 24, LCDR1 consists of SEQ ID NO: 27, LCDR2 consists of SEQ ID NO: 28, and LCDR3 consists of SEQ ID NO: 29,
(iii) the variable heavy (VH) domain comprises, preferably consists of a sequence having at least 90%, at least 95%, at least 98%, or at least 99% identity to SEQ ID NO: 172 and
the variable light (VL) domain comprises, preferably consists of a sequence having at least 90%, at least 95%, at least 98%, or at least 99% identity to SEQ ID NO: 176,
provided that HCDR1 consists of SEQ ID NO: 3, HCDR2 consists of SEQ ID NO: 173, HCDR3 consists of SEQ ID NO: 174, LCDR1 consists of SEQ ID NO: 149, LCDR2 consists of SEQ ID NO: 177, and LCDR3 consists of SEQ ID NO: 178, or
(iv) the variable heavy (VH) domain comprises, preferably consists of a sequence having at least 90%, at least 95%, at least 98%, or at least 99% identity to SEQ ID NO: 180 and
the variable light (VL) domain comprises, preferably consists of a sequence having at least 90%, at least 95%, at least 98%, or at least 99% identity to SEQ ID NO: 184,
provided that HCDR1 consists of SEQ ID NO: 181, HCDR2 consists of SEQ ID NO: 173, HCDR3 consists of SEQ ID NO: 182, LCDR1 consists of SEQ ID NO: 185, LCDR2 consists of SEQ ID NO: 28, and LCDR3 consists of SEQ ID NO: 186.

9. An antibody according to any one of claims 1 to 4, wherein the antibody comprises a variable heavy (VH) domain sequence and a variable light (VL) domain sequence and wherein:
(i) the variable heavy (VH) domain comprises, preferably consists of SEQ ID NO: 31 and the variable light (VL) domain comprises, preferably consists of SEQ ID NO:36,
(ii) the variable heavy (VH) domain comprises, preferably consists of SEQ ID NO: 21 and the variable light (VL) domain comprises, preferably consists of SEQ ID NO: 26,
(iii) the variable heavy (VH) domain comprises, preferably consists of SEQ ID NO: 172 and the variable light (VL) domain comprises, preferably consists of SEQ ID NO:176, or
(iv) the variable heavy (VH) domain comprises, preferably consists of SEQ ID NO: 180 and the variable light (VL) domain comprises, preferably consists of SEQ ID NO:184.

10. An antibody that binds to the same epitope on Oxa-23 as an antibody according to any one of claims 1 to 9.

11. An antibody capable of competing for binding to Oxa-23 with an antibody according to any one of claims 1 to 9.

12. An antibody according to claim 1 or claim 2, that specifically binds OCL1 lipooligosaccharide (LOS) at the surface of Acinetobacter baumannii bacteria.

13. An antibody according to claim 1 or claim 2, that specifically binds OCL1 LOS on outer membrane vesicles (OMVs) of Acinetobacter baumannii bacteria.

14. An antibody according to claim 12 or claim 13, wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, and wherein the HCDR3 is selected from the group consisting of SEQ ID NO: 189, SEQ ID NO: 196, SEQ ID NO: 90, SEQ ID NO: 105, SEQ ID NO: 112, SEQ ID NO:
119, and SEQ ID NO: 146.

15. An antibody according to claim 12 or claim 13, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, and wherein:
(i) HCDR1 consists of SEQ ID NO: 32, HCDR2 consists of SEQ ID NO: 33, HCDR3 consists of SEQ ID NO: 189, LCDR1 consists of SEQ ID NO: 37, LCDR2 consists of SEQ ID NO: 28, and LCDR3 consists of SEQ ID NO: 192,
(ii) HCDR1 consists of SEQ ID NO: 195, HCDR2 consists of SEQ ID NO: 33, HCDR3 consists of SEQ ID NO: 196, LCDR1 consists of SEQ ID NO: 37, LCDR2 consists of SEQ ID NO: 9, and LCDR3 consists of SEQ ID NO: 199,
(iii) HCDR1 consists of SEQ ID NO: 22, HCDR2 consists of SEQ ID NO: 89, HCDR3 consists of SEQ ID NO: 90, LCDR1 consists of SEQ ID NO: 93, LCDR2 consists of SEQ ID NO: 28, and LCDR3 consists of SEQ ID NO: 94,
(iv) HCDR1 consists of SEQ ID NO: 104, HCDR2 consists of SEQ ID NO: 33, HCDR3 consists of SEQ ID NO: 105, LCDR1 consists of SEQ ID NO: 27, LCDR2 consists of SEQ ID NO: 28, and LCDR3 consists of SEQ ID NO: 108,
(v) HCDR1 consists of SEQ ID NO: 111, HCDR2 consists of SEQ ID NO: 33, HCDR3 consists of SEQ ID NO: 112, LCDR1 consists of SEQ ID NO: 115, LCDR2 consists of SEQ ID NO: 28, and LCDR3 consists of SEQ ID NO: 116,
(vi) HCDR1 consists of SEQ ID NO: 104, HCDR2 consists of SEQ ID NO: 33, HCDR3 consists of SEQ ID NO: 119, LCDR1 consists of SEQ ID NO: 122, LCDR2 consists of SEQ ID NO: 28, and LCDR3 consists of SEQ ID NO: 123, or
(vii) HCDR1 consists of SEQ ID NO: 145, HCDR2 consists of SEQ ID NO: 33, HCDR3 consists of SEQ ID NO: 146, LCDR1 consists of SEQ ID NO: 149, LCDR2 consists of SEQ ID NO: 150, and LCDR3 consists of SEQ ID NO: 151.

16. An antibody according to claim 12 or claim 13, wherein the antibody comprises a variable heavy (VH) domain sequence and a variable light (VL) domain sequence and wherein:
(i) the variable heavy (VH) domain comprises, preferably consists of SEQ ID NO: 188 and the variable light (VL) domain comprises, preferably consists of SEQ ID NO: 191,
(ii) the variable heavy (VH) domain comprises, preferably consists of SEQ ID NO: 194 and the variable light (VL) domain comprises, preferably consists of SEQ ID NO: 198,
(iii) the variable heavy (VH) domain comprises, preferably consists of SEQ ID NO: 88 and the variable light (VL) domain comprises, preferably consists of SEQ ID NO: 92,
(iv) the variable heavy (VH) domain comprises, preferably consists of SEQ ID NO: 103 and the variable light (VL) domain comprises, preferably consists of SEQ ID NO: 107,
(v) the variable heavy (VH) domain comprises, preferably consists of SEQ ID NO: 110 and the variable light (VL) domain comprises, preferably consists of SEQ ID NO: 114,
(vi) the variable heavy (VH) domain comprises, preferably consists of SEQ ID NO: 118 and the variable light (VL) domain comprises, preferably consists of SEQ ID NO: 121, or
(vii) the variable heavy (VH) domain comprises, preferably consists of SEQ ID NO: 144 and the variable light (VL) domain comprises, preferably consists of SEQ ID NO: 148,
optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

17. An antibody according to claim 12 or claim 13, wherein the antibody comprises a variable heavy (VH) domain sequence and a variable light (VL) domain sequence and wherein:
(i) the variable heavy (VH) domain comprises, preferably consists of a sequence having at least 90%, at least 95%, at least 98%, or at least 99% identity to SEQ ID NO: 188 and
the variable light (VL) domain comprises, preferably consists of a sequence having at least 90%, at least 95%, at least 98%, or at least 99% identity to SEQ ID NO: 191,
provided that HCDR1 consists of SEQ ID NO: 32, HCDR2 consists of SEQ ID NO: 33, HCDR3 consists of SEQ ID NO: 189, LCDR1 consists of SEQ ID NO: 37, LCDR2 consists of SEQ ID NO: 28, and LCDR3 consists of SEQ ID NO: 192,
(ii) the variable heavy (VH) domain comprises, preferably consists of a sequence having at least 90%, at least 95%, at least 98%, or at least 99% identity to SEQ ID NO: 194 and
the variable light (VL) domain comprises, preferably consists of a sequence having at least 90%, at least 95%, at least 98%, or at least 99% identity to SEQ ID NO: 198,
provided that HCDR1 consists of SEQ ID NO: 195, HCDR2 consists of SEQ ID NO: 33, HCDR3 consists of SEQ ID NO: 1%, LCDR1 consists of SEQ ID NO: 37, LCDR2 consists of SEQ ID NO: 9, and LCDR3 consists of SEQ ID NO: 199,
(iii) the variable heavy (VH) domain comprises, preferably consists of a sequence having at least 90%, at least 95%, at least 98%, or at least 99% identity to SEQ ID NO: 88 and
the variable light (VL) domain comprises, preferably consists of a sequence having at least 90%, at least 95%, at least 98%, or at least 99% identity to SEQ ID NO: 92,
provided that HCDR1 consists of SEQ ID NO: 22, HCDR2 consists of SEQ ID NO: 89, HCDR3 consists of SEQ ID NO: 90, LCDR1 consists of SEQ ID NO: 93, LCDR2 consists of SEQ ID NO: 28, and LCDR3 consists of SEQ ID NO: 94,
(iv) the variable heavy (VH) domain comprises, preferably consists of a sequence having at least 90%, at least 95%, at least 98%, or at least 99% identity to SEQ ID NO: 103 and
the variable light (VL) domain comprises, preferably consists of a sequence having at least 90%, at least 95%, at least 98%, or at least 99% identity to SEQ ID NO: 107,
provided that HCDR1 consists of SEQ ID NO: 104, HCDR2 consists of SEQ ID NO: 33, HCDR3 consists of SEQ ID NO: 105, LCDR1 consists of SEQ ID NO: 27, LCDR2 consists of SEQ ID NO: 28, and LCDR3 consists of SEQ ID NO: 108,
(v) the variable heavy (VH) domain comprises, preferably consists of a sequence having at least 90%, at least 95%, at least 98%, or at least 99% identity to SEQ ID NO: 110 and
the variable light (VL) domain comprises, preferably consists of a sequence having at least 90%, at least 95%, at least 98%, or at least 99% identity to SEQ ID NO: 114,
provided that HCDR1 consists of SEQ ID NO: 111, HCDR2 consists of SEQ ID NO: 33, HCDR3 consists of SEQ ID NO: 112, LCDR1 consists of SEQ ID NO: 115, LCDR2 consists of SEQ ID NO: 28, and LCDR3 consists of SEQ ID NO: 116,
(vi) the variable heavy (VH) domain comprises, preferably consists of a sequence having at least 90%, at least 95%, at least 98%, or at least 99% identity to SEQ ID NO: 118 and
the variable light (VL) domain comprises, preferably consists of a sequence having at least 90%, at least 95%, at least 98%, or at least 99% identity to SEQ ID NO: 121,
provided that HCDR1 consists of SEQ ID NO: 104, HCDR2 consists of SEQ ID NO: 33, HCDR3 consists of SEQ ID NO: 119, LCDR1 consists of SEQ ID NO: 122, LCDR2 consists of SEQ ID NO: 28, and LCDR3 consists of SEQ ID NO: 123, or
(vii) the variable heavy (VH) domain comprises, preferably consists of a sequence having at least 90%, at least 95%, at least 98%, or at least 99% identity to SEQ ID NO: 144 and
the variable light (VL) domain comprises, preferably consists of a sequence having at least 90%, at least 95%, at least 98%, or at least 99%/identity to SEQ ID NO: 148,
provided that HCDR1 consists of SEQ ID NO: 145, HCDR2 consists of SEQ ID NO: 33, HCDR3 consists of SEQ ID NO: 146, LCDR1 consists of SEQ ID NO: 149, LCDR2 consists of SEQ ID NO: 150, and LCDR3 consists of SEQ ID NO: 151.

18. An antibody according to claim 12 or claim 13, wherein the antibody comprises a variable heavy (VH) domain sequence and a variable light (VL) domain sequence and wherein:
(i) the variable heavy (VH) domain comprises, preferably consists of SEQ ID NO: 188 and the variable light (VL) domain comprises SEQ ID NO: 191,
(ii) the variable heavy (VH) domain comprises, preferably consists of SEQ ID NO: 194 and the variable light (VL) domain comprises SEQ ID NO: 198,
(iii) the variable heavy (VH) domain comprises, preferably consists of SEQ ID NO: 88 and the variable light (VL) domain comprises SEQ ID NO: 92,
(iv) the variable heavy (VH) domain comprises, preferably consists of SEQ ID NO: 103 and the variable light (VL) domain comprises SEQ ID NO: 107,
(v) the variable heavy (VH) domain comprises, preferably consists of SEQ ID NO: 110 and the variable light (VL) domain comprises SEQ ID NO: 114,
(vi) the variable heavy (VH) domain comprises, preferably consists of SEQ ID NO: 118 and the variable light (VL) domain comprises SEQ ID NO: 121,
(vii) the variable heavy (VH) domain comprises, preferably consists of SEQ ID NO: 144 and the variable light (VL) domain comprises SEQ ID NO: 148.

19. An antibody that binds to the same epitope on OCL1 LOS as an antibody according to any one of claims 12 to 18.

20. An antibody capable of competing for binding to OCL1 LOS with an antibody according to any one of claims 12 to 18.

21. An antibody according to claim 1 or claim 2, that specifically binds KL49 at the surface of Acinetobacter baumannii bacteria.

22. An antibody according to claim 1 or claim 2, that specifically binds KL49 on outer membrane vesicles (OMVs) of Acinetobacter baumannii bacteria.

23. An antibody according to claim 21 or claim 22, wherein the antibody comprises a variable heavy (VH) domain sequence comprising complementarity determining regions (CDRs) HCDR1, HCDR2 and HCDR3, and a variable light (VL) domain sequence comprising complementarity determining regions LCDR1, LCDR2 and LCDR3, and wherein the HCDR3 is selected from the group consisting of SEQ ID NO: 168, SEQ ID NO: 5, SEQ ID NO: 14, SEQ ID NO: 41, SEQ ID NO: 45, SEQ ID NO: 50, SEQ ID NO: 55, SEQ ID NO: 62, SEQ ID NO: 67, SEQ ID NO: 72, SEQ ID NO: 77, SEQ ID NO: 83, SEQ ID NO: 98, SEQ ID NO: 126, SEQ ID NO: 132, SEQ ID NO: 139, SEQ ID NO: 156, and SEQ ID NO: 163.

24. An antibody according to claim 21 or claim 22, wherein the antibody comprises a variable heavy (VH) domain sequence comprising complementarity determining regions (CDRs) HCDR1, HCDR2, and HCDR3, and a variable light (VL) domain sequence comprising complementarity determining regions LCDR1, LCDR2 and LCDR3, and wherein:
(i) HCDR1 consists of SEQ ID NO: 3, HCDR2 consists of SEQ ID NO: 13, HCDR3 consists of SEQ ID NO: 168, LCDR1 consists of SEQ ID NO: 17, LCDR2 consists of SEQ ID NO: 18, and LCDR3 consists of SEQ ID NO: 10,
(ii) HCDR1 consists of SEQ ID NO: 3, HCDR2 consists of SEQ ID NO: 4, HCDR3 consists of SEQ ID NO: 5, LCDR1 consists of SEQ ID NO: 8, LCDR2 consists of SEQ ID NO: 9, and LCDR3 consists of SEQ ID NO: 10,
(iii) HCDR1 consists of SEQ ID NO: 3, HCDR2 consists of SEQ ID NO: 13, HCDR3 consists of SEQ ID NO: 14, LCDR1 consists of SEQ ID NO: 17, LCDR2 consists of SEQ ID NO: 18, and LCDR3 consists of SEQ ID NO: 19,
(iv) HCDR1 consists of SEQ ID NO: 3, HCDR2 consists of SEQ ID NO: 13, HCDR3 consists of SEQ ID NO: 41, LCDR1 consists of SEQ ID NO: 17, LCDR2 consists of SEQ ID NO: 18, and LCDR3 consists of SEQ ID NO: 19,
(v) HCDR1 consists of SEQ ID NO: 3, HCDR2 consists of SEQ ID NO: 13, HCDR3 consists of SEQ ID NO: 45, LCDR1 consists of SEQ ID NO: 17, LCDR2 consists of SEQ ID NO: 18, and LCDR3 consists of SEQ ID NO: 10,
(vi) HCDR1 consists of SEQ ID NO: 3, HCDR2 consists of SEQ ID NO: 13, HCDR3 consists of SEQ ID NO: 50, LCDR1 consists of SEQ ID NO: 17, LCDR2 consists of SEQ ID NO: 18, and LCDR3 consists of SEQ ID NO: 19,
(vii) HCDR1 consists of SEQ ID NO: 3, HCDR2 consists of SEQ ID NO: 13, HCDR3 consists of SEQ ID NO: 55, LCDR1 consists of SEQ ID NO: 58, LCDR2 consists of SEQ ID NO: 18, and LCDR3 consists of SEQ ID NO: 59,
(viii) HCDR1 consists of SEQ ID NO: 3, HCDR2 consists of SEQ ID NO: 13, HCDR3 consists of SEQ ID NO: 62, LCDR1 consists of SEQ ID NO: 17, LCDR2 consists of SEQ ID NO: 18, and LCDR3 consists of SEQ ID NO: 59,
(ix) HCDR1 consists of SEQ ID NO: 3, HCDR2 consists of SEQ ID NO: 13, HCDR3 consists of SEQ ID NO: 67, LCDR1 consists of SEQ ID NO: 17, LCDR2 consists of SEQ ID NO: 18, and LCDR3 consists of SEQ ID NO: 59,
(x) HCDR1 consists of SEQ ID NO: 3, HCDR2 consists of SEQ ID NO: 13, HCDR3 consists of SEQ ID NO: 72, LCDR1 consists of SEQ ID NO: 17, LCDR2 consists of SEQ ID NO: 18, and LCDR3 consists of SEQ ID NO: 59,
(xi) HCDR1 consists of SEQ ID NO: 3, HCDR2 consists of SEQ ID NO: 13, HCDR3 consists of SEQ ID NO: 77, LCDR1 consists of SEQ ID NO: 17, LCDR2 consists of SEQ ID NO: 18, and LCDR3 consists of SEQ ID NO: 80,
(xii) HCDR1 consists of SEQ ID NO: 3, HCDR2 consists of SEQ ID NO: 13, HCDR3 consists of SEQ ID NO: 83, LCDR1 consists of SEQ ID NO: 17, LCDR2 consists of SEQ ID NO: 18, and LCDR3 consists of SEQ ID NO: 86,
(xiii) HCDR1 consists of SEQ ID NO: 3, HCDR2 consists of SEQ ID NO: 97, HCDR3 consists of SEQ ID NO: 98, LCDR1 consists of SEQ ID NO: 101, LCDR2 consists of SEQ ID NO: 18, and LCDR3 consists of SEQ ID NO: 59,
(xiv) HCDR1 consists of SEQ ID NO: 3, HCDR2 consists of SEQ ID NO: 13, HCDR3 consists of SEQ ID NO: 126, LCDR1 consists of SEQ ID NO: 17, LCDR2 consists of SEQ ID NO: 18, and LCDR3 consists of SEQ ID NO: 129,
(xv) HCDR1 consists of SEQ ID NO: 3, HCDR2 consists of SEQ ID NO: 13, HCDR3 consists of SEQ ID NO: 132, LCDR1 consists of SEQ ID NO: 17, LCDR2 consists of SEQ ID NO: 18, and LCDR3 consists of SEQ ID NO: 135,
(xvi) HCDR1 consists of SEQ ID NO: 3, HCDR2 consists of SEQ ID NO: 138, HCDR3 consists of SEQ ID NO: 139, LCDR1 consists of SEQ ID NO: 142, LCDR2 consists of SEQ ID NO: 18, and LCDR3 consists of SEQ ID NO: 86,
(xvii) HCDR1 consists of SEQ ID NO: 154, HCDR2 consists of SEQ ID NO: 155, HCDR3 consists of SEQ ID NO: 156, LCDR1 consists of SEQ ID NO: 17, LCDR2 consists of SEQ ID NO: 18, and LCDR3 consists of SEQ ID NO: 159, or
(xviii) HCDR1 consists of SEQ ID NO: 3, HCDR2 consists of SEQ ID NO: 162, HCDR3 consists of SEQ ID NO: 163, LCDR1 consists of SEQ ID NO: 17, LCDR2 consists of SEQ ID NO: 18, and LCDR3 consists of SEQ ID NO: 135.

25. An antibody according to claim 21 or claim 22, wherein the antibody comprises a variable heavy (VH) domain sequence and a variable light (VL) domain sequence and wherein:
(i) the variable heavy (VH) domain comprises, preferably consists of SEQ ID NO: 167 and the variable light (VL) domain comprises, preferably consists of SEQ ID NO: 170,
(ii) the variable heavy (VH) domain comprises, preferably consists of SEQ ID NO: 2 and the variable light (VL) domain comprises, preferably consists of SEQ ID NO: 7,
(iii) the variable heavy (VH) domain comprises, preferably consists of SEQ ID NO: 12 and the variable light (VL) domain comprises, preferably consists of SEQ ID NO: 16,
(iv) the variable heavy (VH) domain comprises, preferably consists of SEQ ID NO: 40 and the variable light (VL) domain comprises, preferably consists of SEQ ID NO: 16,
(v) the variable heavy (VH) domain comprises, preferably consists of SEQ ID NO: 44 and the variable light (VL) domain comprises, preferably consists of SEQ ID NO: 47,
(vi) the variable heavy (VH) domain comprises, preferably consists of SEQ ID NO: 49 and the variable light (VL) domain comprises, preferably consists of SEQ ID NO: 52,
(vii) the variable heavy (VH) domain comprises, preferably consists of SEQ ID NO: 54 and the variable light (VL) domain comprises, preferably consists of SEQ ID NO: 57,
(viii) the variable heavy (VH) domain comprises, preferably consists of SEQ ID NO: 61 and the variable light (VL) domain comprises, preferably consists of SEQ ID NO: 64,
(ix) the variable heavy (VH) domain comprises, preferably consists of SEQ ID NO: 66 and the variable light (VL) domain comprises, preferably consists of SEQ ID NO: 69,
(x) the variable heavy (VH) domain comprises, preferably consists of SEQ ID NO: 71 and the variable light (VL) domain comprises, preferably consists of SEQ ID NO: 74,
(xi) the variable heavy (VH) domain comprises, preferably consists of SEQ ID NO: 76 and the variable light (VL) domain comprises, preferably consists of SEQ ID NO: 79,
(xii) the variable heavy (VH) domain comprises, preferably consists of SEQ ID NO: 82 and the variable light (VL) domain comprises, preferably consists of SEQ ID NO: 85,
(xiii) the variable heavy (VH) domain comprises, preferably consists of SEQ ID NO: 96 and the variable light (VL) domain comprises, preferably consists of SEQ ID NO: 100,
(xiv) the variable heavy (VH) domain comprises, preferably consists of SEQ ID NO: 125 and the variable light (VL) domain comprises, preferably consists of SEQ ID NO: 128,
(xv) the variable heavy (VH) domain comprises, preferably consists of SEQ ID NO: 131 and the variable light (VL) domain comprises, preferably consists of SEQ ID NO: 134,
(xvi) the variable heavy (VH) domain comprises, preferably consists of SEQ ID NO: 137 and the variable light (VL) domain comprises, preferably consists of SEQ ID NO: 141,
(xvii) the variable heavy (VH) domain comprises, preferably consists of SEQ ID NO: 153 and the variable light (VL) domain comprises, preferably consists of SEQ ID NO: 158, or
(xviii) the variable heavy (VH) domain comprises, preferably consists of SEQ ID NO: 161 and the variable light (VL) domain comprises, preferably consists of SEQ ID NO: 165,
optionally with 1, 2, 3, 4 or 5 amino acid alterations outside the complementarity determining regions (CDRs) in the variable heavy (VH) domain sequence and optionally with 1, 2, 3, 4 or 5 amino acid alterations outside the complementarity determining regions (CDRs) in the variable light (VL) domain sequence.

26. An antibody according to claim 21 or claim 22, wherein the antibody comprises a variable heavy (VH) domain sequence and a variable light (VL) domain sequence and wherein:
(i) the variable heavy (VH) comprises, preferably consists of a sequence having at least 90%, at least 95%, at least 98%, or at least 99% identity to SEQ ID NO: 167 and
the variable light (VL) domain comprises, preferably consists of a sequence having at least 90%, at least 95%, at least 98%, or at least 99% identity to SEQ ID NO: 170,
provided that HCDR1 consists of SEQ ID NO: 3, HCDR2 consists of SEQ ID NO: 13, HCDR3 consists of SEQ ID NO: 168, LCDR1 consists of SEQ ID NO: 17, LCDR2 consists of SEQ ID NO: 18, and LCDR3 consists of SEQ ID NO: 10,
(ii) the variable heavy (VH) comprises, preferably consists of a sequence having at least 90%, at least 95%, at least 98%, or at least 99% identity to SEQ ID NO: 2 and
the variable light (VL) domain comprises, preferably consists of a sequence having at least 90%, at least 95%, at least 98%, or at least 99% identity to SEQ ID NO: 7,
provided that HCDR1 consists of SEQ ID NO: 3, HCDR2 consists of SEQ ID NO: 4, HCDR3 consists of SEQ ID NO: 5, LCDR1 consists of SEQ ID NO: 8, LCDR2 consists of SEQ ID NO: 9, and LCDR3 consists of SEQ ID NO: 10,
(iii) the variable heavy (VH) comprises, preferably consists of a sequence having at least 90%, at least 95%, at least 98%, or at least 99% identity to SEQ ID NO: 12 and
the variable light (VL) domain comprises, preferably consists of a sequence having at least 90%, at least 95%, at least 98%, or at least 99% identity to SEQ ID NO: 16,
provided that HCDR1 consists of SEQ ID NO: 3, HCDR2 consists of SEQ ID NO: 13, HCDR3 consists of SEQ ID NO: 14, LCDR1 consists of SEQ ID NO: 17, LCDR2 consists of SEQ ID NO: 18, and LCDR3 consists of SEQ ID NO: 19,
(iv) the variable heavy (VH) comprises, preferably consists of a sequence having at least 90%, at least 95%, at least 98%, or at least 99% identity to SEQ ID NO: 40 and
the variable light (VL) domain comprises, preferably consists of a sequence having at least 90%, at least 95%, at least 98%, or at least 99% identity to SEQ ID NO: 16,
provided that HCDR1 consists of SEQ ID NO: 3, HCDR2 consists of SEQ ID NO: 13, HCDR3 consists of SEQ ID NO: 41, LCDR1 consists of SEQ ID NO: 17, LCDR2 consists of SEQ ID NO: 18, and LCDR3 consists of SEQ ID NO: 19,
(v) the variable heavy (VH) comprises, preferably consists of a sequence having at least 90%, at least 95%, at least 98%, or at least 99% identity to SEQ ID NO: 44 and
the variable light (VL) domain comprises, preferably consists of a sequence having at least 90%, at least 95%, at least 98%, or at least 99% identity to SEQ ID NO: 47,
provided that HCDR1 consists of SEQ ID NO: 3, HCDR2 consists of SEQ ID NO: 13, HCDR3 consists of SEQ ID NO: 45, LCDR1 consists of SEQ ID NO: 17, LCDR2 consists of SEQ ID NO: 18, and LCDR3 consists of SEQ ID NO: 10,
(vi) the variable heavy (VH) comprises, preferably consists of a sequence having at least 90%, at least 95%, at least 98%, or at least 99% identity to SEQ ID NO: 49 and
the variable light (VL) domain comprises, preferably consists of a sequence having at least 90%, at least 95%, at least 98%, or at least 99% identity to SEQ ID NO: 52,
provided that HCDR1 consists of SEQ ID NO: 3, HCDR2 consists of SEQ ID NO: 13, HCDR3 consists of SEQ ID NO: 50, LCDR1 consists of SEQ ID NO: 17, LCDR2 consists of SEQ ID NO: 18, and LCDR3 consists of SEQ ID NO: 19,
(vii) the variable heavy (VH) comprises, preferably consists of a sequence having at least 90%, at least 95%, at least 98%, or at least 99% identity to SEQ ID NO: 54 and
the variable light (VL) domain comprises, preferably consists of a sequence having at least 90%, at least 95%, at least 98%, or at least 99% identity to SEQ ID NO: 57,
provided that HCDR1 consists of SEQ ID NO: 3, HCDR2 consists of SEQ ID NO: 13, HCDR3 consists of SEQ ID NO: 55, LCDR1 consists of SEQ ID NO: 58, LCDR2 consists of SEQ ID NO: 18, and LCDR3 consists of SEQ ID NO: 59,
(viii) the variable heavy (VH) comprises, preferably consists of a sequence having at least 90%, at least 95%, at least 98%, or at least 99% identity to SEQ ID NO: 61 and
the variable light (VL) domain comprises, preferably consists of a sequence having at least 90%, at least 95%, at least 98%, or at least 99%/identity to SEQ ID NO: 64,
provided that HCDR1 consists of SEQ ID NO: 3, HCDR2 consists of SEQ ID NO: 13, HCDR3 consists of SEQ ID NO: 62, LCDR1 consists of SEQ ID NO: 17, LCDR2 consists of SEQ ID NO: 18, and LCDR3 consists of SEQ ID NO: 59,
(ix) the variable heavy (VH) comprises, preferably consists of a sequence having at least 90%, at least 95%, at least 98%, or at least 99% identity to SEQ ID NO: 66 and
the variable light (VL) domain comprises, preferably consists of a sequence having at least 90%, at least 95%, at least 98%, or at least 99% identity to SEQ ID NO: 69,
provided that HCDR1 consists of SEQ ID NO: 3, HCDR2 consists of SEQ ID NO: 13, HCDR3 consists of SEQ ID NO: 67, LCDR1 consists of SEQ ID NO: 17, LCDR2 consists of SEQ ID NO: 18, and LCDR3 consists of SEQ ID NO: 59,
(x) the variable heavy (VH) comprises, preferably consists of a sequence having at least 90%, at least 95%, at least 98%, or at least 99% identity to SEQ ID NO: 71 and
the variable light (VL) domain comprises, preferably consists of a sequence having at least 90%, at least 95%, at least 98%, or at least 99% identity to SEQ ID NO: 74,
provided that HCDR1 consists of SEQ ID NO: 3, HCDR2 consists of SEQ ID NO: 13, HCDR3 consists of SEQ ID NO: 72, LCDR1 consists of SEQ ID NO: 17, LCDR2 consists of SEQ ID NO: 18, and LCDR3 consists of SEQ ID NO: 59,
(xi) the variable heavy (VH) comprises, preferably consists of a sequence having at least 90%, at least 95%, at least 98%, or at least 99% identity to SEQ ID NO: 76 and
the variable light (VL) domain comprises, preferably consists of a sequence having at least 90%, at least 95%, at least 98%, or at least 99% identity to SEQ ID NO: 79,
provided that HCDR1 consists of SEQ ID NO: 3, HCDR2 consists of SEQ ID NO: 13, HCDR3 consists of SEQ ID NO: 77, LCDR1 consists of SEQ ID NO: 17, LCDR2 consists of SEQ ID NO: 18, and LCDR3 consists of SEQ ID NO: 80,
(xii) the variable heavy (VH) comprises, preferably consists of a sequence having at least 90%, at least 95%, at least 98%, or at least 99% identity to SEQ ID NO: 82 and
the variable light (VL) domain comprises, preferably consists of a sequence having at least 90%, at least 95%, at least 98%, or at least 99% identity to SEQ ID NO: 85,
provided that HCDR1 consists of SEQ ID NO: 3, HCDR2 consists of SEQ ID NO: 13, HCDR3 consists of SEQ ID NO: 83, LCDR1 consists of SEQ ID NO: 17, LCDR2 consists of SEQ ID NO: 18, and LCDR3 consists of SEQ ID NO: 86,
(xiii) the variable heavy (VH) comprises, preferably consists of a sequence having at least 90%, at least 95%, at least 98%, or at least 99% identity to SEQ ID NO: 96 and
the variable light (VL) domain comprises, preferably consists of a sequence having at least 90%, at least 95%, at least 98%, or at least 99%/identity to SEQ ID NO: 100,
provided that HCDR1 consists of SEQ ID NO: 3, HCDR2 consists of SEQ ID NO: 97, HCDR3 consists of SEQ ID NO: 98, LCDR1 consists of SEQ ID NO: 101, LCDR2 consists of SEQ ID NO: 18, and LCDR3 consists of SEQ ID NO: 59,
(xiv) the variable heavy (VH) comprises, preferably consists of a sequence having at least 90%, at least 95%, at least 98%, or at least 99% identity to SEQ ID NO: 125 and
the variable light (VL) domain comprises, preferably consists of a sequence having at least 90%, at least 95%, at least 98%, or at least 99% identity to SEQ ID NO: 128,
provided that HCDR1 consists of SEQ ID NO: 3, HCDR2 consists of SEQ ID NO: 13, HCDR3 consists of SEQ ID NO: 126, LCDR1 consists of SEQ ID NO: 17, LCDR2 consists of SEQ ID NO: 18, and LCDR3 consists of SEQ ID NO: 129,
(xv) the variable heavy (VH) comprises, preferably consists of a sequence having at least 90%, at least 95%, at least 98%, or at least 99% identity to SEQ ID NO: 131 and
the variable light (VL) domain comprises, preferably consists of a sequence having at least 90%, at least 95%, at least 98%, or at least 99% identity to SEQ ID NO: 134,
provided that HCDR1 consists of SEQ ID NO: 3, HCDR2 consists of SEQ ID NO: 13, HCDR3 consists of SEQ ID NO: 132, LCDR1 consists of SEQ ID NO: 17, LCDR2 consists of SEQ ID NO: 18, and LCDR3 consists of SEQ ID NO: 135,
(xvi) the variable heavy (VH) comprises, preferably consists of a sequence having at least 90%, at least 95%, at least 98%, or at least 99% identity to SEQ ID NO: 137 and
the variable light (VL) domain comprises, preferably consists of a sequence having at least 90%, at least 95%, at least 98%, or at least 99% identity to SEQ ID NO: 141,
provided that HCDR1 consists of SEQ ID NO: 3, HCDR2 consists of SEQ ID NO: 138, HCDR3 consists of SEQ ID NO: 139, LCDR1 consists of SEQ ID NO: 142, LCDR2 consists of SEQ ID NO: 18, and LCDR3 consists of SEQ ID NO: 86,
(xvii) the variable heavy (VH) comprises, preferably consists of a sequence having at least 90%, at least 95%, at least 98%, or at least 99% identity to SEQ ID NO: 153 and
the variable light (VL) domain comprises, preferably consists of a sequence having at least 90%, at least 95%, at least 98%, or at least 99% identity to SEQ ID NO: 158,
provided that HCDR1 has SEQ ID NO: 154, HCDR2 has SEQ ID NO: 155, HCDR3 has SEQ ID NO: 156, LCDR1 has SEQ ID NO: 17, LCDR2 has SEQ ID NO: 18, and LCDR3 has SEQ ID NO: 159, or
(xviii) the variable heavy (VH) comprises, preferably consists of a sequence having at least 90%, at least 95%, at least 98%, or at least 99% identity to SEQ ID NO: 161 and
the variable light (VL) domain comprises, preferably consists of a sequence having at least 90%, at least 95%, at least 98%, or at least 99%/identity to SEQ ID NO: 165,
provided that HCDR1 consists of SEQ ID NO: 3, HCDR2 consists of SEQ ID NO: 162, HCDR3 consists of SEQ ID NO: 163, LCDR1 consists of SEQ ID NO: 17, LCDR2 consists of SEQ ID NO: 18, and LCDR3 consists of SEQ ID NO: 135.

27. An antibody according to claim 21 or claim 22, wherein the antibody comprises a variable heavy (VH) domain sequence and a variable light (VL) domain sequence and wherein:
(i) the variable heavy (VH) domain comprises, preferably consists of SEQ ID NO: 167 and the variable light (VL) domain comprises, preferably consists of SEQ ID NO: 170,
(ii) the variable heavy (VH) domain comprises, preferably consists of SEQ ID NO: 2 and the variable light (VL) domain comprises, preferably consists of SEQ ID NO: 7,
(iii) the variable heavy (VH) domain comprises, preferably consists of SEQ ID NO: 12 and the variable light (VL) domain comprises, preferably consists of SEQ ID NO: 16,
(iv) the variable heavy (VH) domain comprises, preferably consists of SEQ ID NO: 40 and the variable light (VL) domain comprises, preferably consists of SEQ ID NO: 16,
(v) the variable heavy (VH) domain comprises, preferably consists of SEQ ID NO: 44 and the variable light (VL) domain comprises, preferably consists of SEQ ID NO: 47,
(vi) the variable heavy (VH) domain comprises, preferably consists of SEQ ID NO: 49 and the variable light (VL) domain comprises, preferably consists of SEQ ID NO: 52,
(vii) the variable heavy (VH) domain comprises, preferably consists of SEQ ID NO: 54 and the variable light (VL) domain comprises, preferably consists of SEQ ID NO: 57,
(viii) the variable heavy (VH) domain comprises, preferably consists of SEQ ID NO: 61 and the variable light (VL) domain comprises, preferably consists of SEQ ID NO: 64,
(ix) the variable heavy (VH) domain comprises, preferably consists of SEQ ID NO: 66 and the variable light (VL) domain comprises, preferably consists of SEQ ID NO: 69,
(x) the variable heavy (VH) domain comprises, preferably consists of SEQ ID NO: 71 and the variable light (VL) domain comprises, preferably consists of SEQ ID NO: 74,
(xi) the variable heavy (VH) domain comprises, preferably consists of SEQ ID NO: 76 and the variable light (VL) domain comprises, preferably consists of SEQ ID NO: 79,
(xii) the variable heavy (VH) domain comprises, preferably consists of SEQ ID NO: 82 and the variable light (VL) domain comprises, preferably consists of SEQ ID NO: 85,
(xiii) the variable heavy (VH) domain comprises, preferably consists of SEQ ID NO: 96 and the variable light (VL) domain comprises, preferably consists of SEQ ID NO: 100,
(xiv) the variable heavy (VH) domain comprises, preferably consists of SEQ ID NO: 125 and the variable light (VL) domain comprises, preferably consists of SEQ ID NO: 128,
(xv) the variable heavy (VH) domain comprises, preferably consists of SEQ ID NO: 131 and the variable light (VL) domain comprises, preferably consists of SEQ ID NO: 134,
(xvi) the variable heavy (VH) domain comprises, preferably consists of SEQ ID NO: 137 and the variable light (VL) domain comprises, preferably consists of SEQ ID NO: 141,
(xvii) the variable heavy (VH) domain comprises, preferably consists of SEQ ID NO: 153 and the variable light (VL) domain comprises, preferably consists of SEQ ID NO: 158, or
(xviii) the variable heavy (VH) domain comprises, preferably consists of SEQ ID NO: 161 and the variable light (VL) domain comprises, preferably consists of SEQ ID NO: 165.

28. An antibody that binds to the same epitope on KL49 as an antibody according to any one of claims 21 to 27.

29. An antibody capable of competing for binding to KL49 with any an antibody according to any one of claims 21 to 27.

30. An antibody according to any one of the preceding claims, wherein the antibody shows complement-dependant cytotoxic activity (CDC) activity.

31. An antibody according to any one of the preceding claims, wherein the antibody is a human IgG1 or human IgG4.

32. An antibody according to claim 31, wherein the antibody is a human IgG1, optionally wherein the antibody comprises a constant region sequence of SEQ ID NO: 418.

33. An antibody according to claim 31, wherein the antibody is a human IgG4, optionally wherein the antibody comprises a constant region sequence of SEQ ID NO: 436.

34. An antibody according to any one of claims 1 to 30, wherein the antibody is a human IgA1 (e.g., comprising a constant region sequence SEQ ID NO: 484) or human IgA2 (e.g., comprising a constant region sequence SEQ ID NO: 485).

35. An antibody according to any one of claims 1 to 34, wherein the antibody comprises kappa (κ) light chain constant regions, preferably constant domain sequence SEQ ID NO: 448.

36. A nucleic acid sequence comprising a sequence that encodes a VH domain and/or an VL domain of an antibody according to any preceding claim.

37. A nucleic acid sequence comprising a sequence that encodes the heavy chain and/or the light chain of an antibody according to any preceding claim.

38. A vector comprising a nucleic acid according to claim 36 or claim 37, optionally wherein the vector is a CHO vector.

39. A host cell comprising a nucleic acid according to claim 36 or claim 37, or a vector according to claim 38.

40. A pharmaceutical composition comprising (i) an isolated nucleic acid encoding an antibody according to any one of claims 1 to 35, or (ii) a nucleic acid according to claim 36 or claim 37, and a pharmaceutically acceptable excipient.

41. A pharmaceutical composition comprising an antibody according to any one of claims 1 to 35, and a pharmaceutically acceptable excipient.

42. A pharmaceutical composition according to claim 40 or 41, wherein the pharmaceutical composition is formulated for intravenous, intramuscular, or subcutaneous administration.

43. A pharmaceutical composition according to any one of claims 40 to 42, wherein the pharmaceutical composition further comprises at least one further therapeutic agent.

44. A pharmaceutical composition according to claim 43, wherein the further therapeutic agent is at least one, preferably one or two, further antibodies.

45. A pharmaceutical composition according to claim 43, wherein the further therapeutic agent is carbapenem.

46. A pharmaceutical composition according to claim 43, wherein the further therapeutic agent is colistin.

47. A kit comprising an antibody according to any one of claim 1 to 35.

48. A kit comprising a pharmaceutical composition according to any one of claims 40 to 46.

49. A kit according to claim 48, wherein the kit further comprises a label or instructions for use to prevent and/or treat a bacterial infection caused by Acinetobacter baumannii in a human; optionally wherein the label or instructions comprise a marketing authorisation number (e.g., an FDA or EMA authorisation number); optionally wherein the kit comprises an IV or injection device that comprises the antibody.

50. A kit according to claim 47 or claim 49, wherein the antibody is contained in a sealed container.

51. An antibody according to any one of claims 1 to 35, or a pharmaceutical composition according to any one of claims 40 to 46, or a nucleic acid according to claim 36 or claim 37, for use as a medicament.

52. An antibody according to any one of claims 1 to 35, or a pharmaceutical composition according to any one of claims 40 to 46, or a nucleic acid according to claim 36 or claim 37, for use in a method of treating a bacterial infection caused by Acinetobacter baumannii, said method comprising administering the antibody, composition, or nucleic acid to a patient.

53. An antibody according to any one of claims 1 to 35, or a pharmaceutical composition according to any one of claims 40 to 46, or a nucleic acid according to claim 36 or claim 37, for use in a method of preventing a bacterial infection caused by Acinetobacter baumannii, said method comprising administering the antibody, composition, or nucleic acid to a patient.

54. An antibody according to any one of claims 1 to 35, or a pharmaceutical composition according to any one of claims 40 to 46, or a nucleic acid according to claim 36 or claim 37, for use according to claim 52 or claim 53, wherein the presence or absence of Oxa-23 has been determined in a sample from the patient prior to administering the antibody, composition, or nucleic acid to the patient.

55. An antibody according to any one of claims 1 to 35, or a pharmaceutical composition according to any one of claims 40 to 46, or a nucleic acid according to claim 36 or claim 37, for use according to claim 52, claim 53 or claim 54, wherein the presence or absence of OCL1 LOS has been determined in a sample from the patient prior to administering the antibody, composition, or nucleic acid to the patient.

56. An antibody according to any one of claims 1 to 35, or a pharmaceutical composition according to any one of claims 40 to 46, or a nucleic acid according to claim 36 or claim 37, for use according to any one of claims 52 to 55, wherein the presence or absence of KL49 has been determined in a sample from the patient prior to administering the antibody, composition, or nucleic acid to the patient.

57. Use of an antibody according to any one of claims 1 to 35, or a pharmaceutical composition according to any one of claims 40 to 46, or a nucleic acid according to claim 36 or claim 37, in the manufacture of a medicament for use in a method of treating a bacterial infection caused by Acinetobacter baumannii in a patient.

58. Use of an antibody according to any one of claims 1 to 35, or a pharmaceutical composition according to any one of claims 40 to 46, or a nucleic acid according to claim 36 or claim 37, in the manufacture of a medicament for use in a method of preventing a bacterial infection caused by Acinetobacter baumannii in a patient.

59. A method of treating a bacterial infection caused by Acinetobacter baumannii in a patient comprising administering to said patient a therapeutically effective amount of an antibody according to any one of claims 1 to 35, or a pharmaceutical composition according to any one of claims 40 to 46, or a nucleic acid according to claim 36 or claim 37.

60. A method of preventing a bacterial infection caused by Acinetobacter baumannii in a patient comprising administering to said patient a therapeutically effective amount of an antibody according to any one of claims 1 to 35, or a pharmaceutical composition according to any one of claims 40 to 46, or a nucleic acid according to claim 36 or claim 37.

61. An antibody, composition, or nucleic acid for use according to any one of claims 52 to 56, use according to claim 57 or claim 58, or a method according to claim 59 or claim 60, wherein the bacterial infection caused by Acinetobacter baumannii is a nosocomial bacterial infection caused by Acinetobacter baumannii.

62. An antibody, composition, or nucleic acid for use according to any one of claims 52 to 56, use according to claim 57 or claim 58, or a method according to claim 59 or claim 60, wherein the patient has a lower respiratory tract infection, for example pneumonia.

63. An antibody, composition, or nucleic acid for use according to any one of claims 52 to 56, use according to claim 57 or claim 58, or a method according to claim 59 or claim 60, wherein the patient has sepsis.

64. An antibody, composition, or nucleic acid for use according to any one of claims 52 to 56, use according to claim 57 or claim 58, or a method according to claim 59 or claim 60, wherein the patient has bacteremia.

65. An antibody, composition, or nucleic acid for use according to any one of claims 52 to 56, use according to claim 57 or claim 58, or a method according to claim 59 or claim 60, wherein the method further comprises administering at least one further therapeutic agent.

66. An antibody, composition, or nucleic acid for use according to claim 65, use according to claim 65, or a method according to claim 65, wherein the administration of the further therapeutic agent is simultaneous, separate or sequential.

67. An antibody, composition, or nucleic acid for use according to claim 65 or claim 66, use according to claim 65 or claim 66, or a method according to claim 65 or claim 66, wherein the further therapeutic agent is at least one, preferably one or two, further antibodies.

68. An antibody, composition, or nucleic acid for use according to any one of claims 65 to 67, use according to any one of claims 65 to 67, or a method according to any one of claims 65 to 67, wherein the at least one further therapeutic agent is carbapenem.

69. An antibody, composition, or nucleic acid for use according to any one of claims 65 to 67, use according to any one of claims 65 to 67, or a method according to any one of claims 65 to 67, wherein the at least one further therapeutic agent is colistin.

70. Use of an antibody according to any one of claims 1 to 35, for determining the presence or absence of Acinetobacter baumannii in a sample.

71. A method of determining the presence or absence of Acinetobacter baumannii in a sample comprising contacting the sample with an antibody according to any one of 1 to 35; and testing for binding between the antibody and Acinetobacter baumannii in the sample; wherein detection of binding indicates the presence of Acinetobacter baumannii in the sample and wherein absence of binding indicates the absence of Acinetobacter baumannii in the sample.

72. Use of an antibody according to any one of claims 1 to 11 for determining the presence or absence of Oxa-23 in a sample, optionally wherein determining the presence or absence of Oxa-23 in a sample is used for determining a treatment protocol in a patient.

73. Use of an antibody according to any one of claims 1, 2, or 12 to 20 for determining the presence or absence of OCL1 LOS in a sample, optionally wherein determining the presence or absence of OCL1 LOS in a sample is used for determining a treatment protocol in a patient.

74. Use of an antibody according to any one of claims 1, 2, or 21 to 29 for determining the presence or absence of KL49 in a sample, optionally wherein determining the presence or absence of KL49 in a sample is used for determining a treatment protocol in a patient.

75. Use according to any one of claims 72 to 74, wherein the antibody is conjugated to a detectable label.

76. Use according to any one of claims 72 to 75, wherein the sample has been obtained from a human who has been or is suspected of having been infected with Acinetobacter baumannii.

77. Use according to claim 76, wherein the sample has been obtained from a human who has been or is suspected of having been infected with Acinetobacter baumannii who exhibits one or more symptoms of a bacterial infection.

78. Use according to any one of claims 72 to 77, wherein the sample is a serum, plasma, or whole blood sample, an oral or nasal swab, urine, faeces, or cerebrospinal fluid (CFS), or wherein the sample is from any suspected Acinetobacter baumannii infected organ or tissue.

79. A diagnostic kit comprising an antibody according to any one of claims 1 to 35, and optionally one or more buffering solutions.

80. A diagnostic kit according to claim 79, wherein the kit comprises a first reagent comprising the antibody according to any one of claims 1 to 35, and a second reagent comprising a detector molecule that binds to the first reagent.

81. A diagnostic kit according to claim 80, wherein the detector molecule is an antibody that comprises or is conjugated to a detectable label.
